(19) United States
(12) Patent Application Publication
(10) Pub. No.: US 2025/0051423 A1
(43) Pub. Date: Feb. 13, 2025

(54) POLYOMAVIRUS NEUTRALIZING ANTIBODIES

(71) Applicant: Novartis AG, Basel (CH)

(72) Inventors: Johanna ABEND, Emeryville, CA (US); Zorica DRAGIC, Basel (CH); Adam Lloyd FEIRE, Hull, MA (US); Mark KNAPP, Oakland, CA (US); Steven KOVACS, Randolph, NJ (US); Elisabetta TRAGGIAI, Basel (CH); Lichun WANG, Shanghai (CN); Yongqiang WANG, Shanghai (CN); Danqing WU, Shanghai (CN); Qilong WU, Shanghai (CN); Fangmin XU, Belmont, MA (US)

(21) Appl. No.: 18/780,282

(22) Filed: Jul. 22, 2024

Related U.S. Application Data
(63) Continuation of application No. 18/186,064, filed on Mar. 17, 2023, now Pat. No. 12,077,571.
Application No. 18/186,064 is a continuation of application No. 17/502,910, filed on Oct. 15, 2021, now Pat. No. 11,639,378.
Application No. 17/502,910 is a continuation of application No. 16/657,855, filed on Oct. 18, 2019, now Pat. No. 11,161,894.
Application No. 16/657,855 is a continuation of application No. 15/758,491, filed on Mar. 8, 2018, now Pat. No. 10,450,366.

(30) Foreign Application Priority Data
Sep. 16, 2015 (WO) PCT/CN2015/089764

(51) Int. Cl. C07K 16/08; A61K 9/19; A61K 39/42; A61K 45/06

(52) U.S. Cl. CPC C07K 16/084; A61K 9/19; A61K 39/42; A61K 45/06; C07K 2317/20; C07K 2317/21; C07K 2317/24; C07K 2317/33; C07K 2317/34; C07K 2317/41; C07K 2317/56; C07K 2317/565; C07K 2317/622; C07K 2317/76; C07K 2317/92

(57) ABSTRACT

The present invention relates to anti-VP1 antibodies, antibody fragments, and their uses for the prevention and treatment of polyoma virus infection and associated diseases.

### FIELD OF THE INVENTION

The present disclosure is directed to anti-VP1 antibodies, antibody fragments, and their uses for the reducing the likelihood or treatment of polyoma virus infection.

### BACKGROUND OF THE INVENTION

Of the human polyomaviruses, BK virus (BKV) and JC virus (JCV) were the first two identified. These two polyomavirus were isolated from immunosuppressed patients and published in the same issue of Lancet in 1971 (Gardner et al., Lancet 1971 1:1253-1527, and Padgett et al., Lancet 1971 1:1257-1260). Polyomaviruses are icosahedral, non-enveloped, double-stranded DNA viruses. They measure 40-45 nm in diameter and are comprised of 88% protein and 12% DNA.

The BKV genome is a circular double-stranded DNA of approximately 5 Kb in length and contains three major divisions: the early coding region, the late coding region, and a non-coding control region. The early coding region encodes for the three regulatory proteins (large tumor antigen [TAg], small tumor antigen [tAg], and truncated tumor antigen [truncTAg]), which are the first viral proteins expressed in a newly infected cell and are responsible for facilitating viral DNA replication and establishing a favorable cellular environment. The late coding region encodes the three structural proteins (VP1, VP2, and VP3) that make up the viral capsid, as well as the agnoprotein, the role of which during viral replication is less well-defined. The non-coding control region contains the origin of replication as well as the early and late promoters that drive expression of the viral gene products.

BKV has been detected in many different cell types including epithelial cells of the kidney, bladder, and ureter (typical sites of persistence), tonsillar tissue, and lymphocytes (proposed sites of primary infection and dissemination) (Chatterjee et al., J. Med. Virol. 2000; 60:353-362, Goudsmit et al., J. Med. Virol. 1982; 10:91-99, Heritage et al., J. Med. Virol. 1981; 8:143-150, Shinohara et al., J. Med. Virol. 1993; 41(4):301-305). The primary cell surface receptors for BKV are the gangliosides GT1b, GD1b, and GD3, all of which have a terminal α2,8-linked sialic acid and are fairly ubiquitous, allowing infection of various cell types (Neu et al., PLos Patholog. 2013; 9(10):e1003714 and e1003688, see also, O'Hara et al., Virus Res. 2014; 189:208-285). The non-enveloped icosahedral virion of BKV is composed of three different viral proteins: 360 copies of the major viral capsid protein VP1 arranged in 72 pentamers and 72 copies combined of the minor viral capsid proteins VP2 and VP3, with one VP2 or VP3 molecule associated with each VP1 pentamer. Only VP1 is exposed on the virion surface at entry and each pentamer has five low affinity binding sites for the ganglioside receptor. Binding of VP1 pentamers to ganglioside receptors on the cell surface initiates internalization through a caveolae-mediated endocytic pathway, followed by trafficking of the virus to the endoplasmic reticulum and finally to the nucleus (Tsai and Qian, J. Virol 2010; 84(19):9840-9852).

Infection with the human polyomavirus BK (BKV) is essentially ubiquitous, with estimates ranging between 80 and 90% of the population globally infected (Knowles W. A., Adv. Exp. Med. Biol. 2006; 577:19-45). Primary infection most often occurs during childhood (i.e., before age 10) and results in either a mild, non-specific, self-limited illness or no symptoms at all. Persistent infection is established in the epithelial cells of the renal tubules, ureters, and bladder, and is effectively controlled by the immune system. Transient asymptomatic viral shedding in the urine of immunocompetent adults occurs sporadically but results in no disease or sequelae. However, compromised immune function, particularly with immunosuppression following renal or hematopoietic stem cell transplantation, can lead to uncontrolled BKV replication and ultimately to BKV-associated nephropathy (BKVAN) or hemorrhagic cystitis (HC), a painful disease of the bladder. There are no effective antiviral therapies against BKV and the current standard of care is reduction of immunosuppression, which increases the risk of acute rejection. Even with the current, more aggressive approaches to monitoring and prevention, up to 10% of renal transplant recipients will develop BKVAN and 15-30% of those patients will suffer graft loss due to BKVAN. Among those undergoing reduction in immunosuppressive regimen upon detection of BKV viremia, up to 30% will experience an acute rejection episode as a result.

Although BKV was first described in 1971 (supra), it was not until the 1990s that BK associated nephropathy (BKVAN) was reported in the literature as a cause of kidney transplant injury (Purighalla et al., Am. J. Kidney Dis. 1995; 26:671-673 and Randhawa et al., Transplantation 1999; 67:103-109). In early management of BKVAN, testing positive for BK had severe consequences, with more than 50% of the patients having graft dysfunction and graft loss (Hirsch et al., New Engl. J. Med. 2002; 347:488-496). BK viral reactivation may begin after transplantation, and is seen in about 30%-50% of the patients by 3 months post-transplantation (Bressollette-Bodin et al., Am J. Transplant. 2005; 5(8):1926-1933 and Brennan et al., Am. J. Transplant. 2004; 4(12):2132-2134). BK viral reactivation can be first seen by virus and viral DNA in the urine, then in the plasma and finally in the kidney. (Brennan et al., Am. J. Transplant. 2005; 5(3):582-594 and Hirsch et al., N Eng. J. Med. 2002; 347(7):488-496). About 80% of kidney transplant patients have BK virus in the urine (BK viruria) and 5-10% of these patients progress to BKVAN (Binet et al., Transplantation 1999; 67(6):918-922 and Bressollette-Bodin et al., Am J. Transplant. 2005; 5(8):1926-1933). BKV effects the renal tubular epithelial cells causing necrosis and lytic destruction with denudation of the basement membrane, which allows tubular fluid to accumulate in the interstitum, which results in interstitial fibrosis and tubular atrophy (Nickeleit et al., J. Am. Soc. Neprol. 1999; 10(5):1080-1089) all of which can affect the condition of the transplant. Patients may present with deterioration of renal function, tubule-interstitial nephritis and ureteric stenosis (Garner et al., Lancet 1971; 1(7712):1253-1257 and Hirsch Am. J. Transplant 2002; 2(1)25-30).

BKV can also cause pneumonitis, retinitis and meningoencephalitis in immunocompromised hosts (Reploeg et al., Clin. Infect. Dis. 2001; 33(2):191-202). BKV disease in hematopoietic stem cell transplant (HSCT) recipients typically manifests as hemorrhagic cystitis (HC), which can vary in severity. Viruria (but not viremia) and painful hematuria are associated with the clinical presentation of HC. The current standard of care is supportive in nature, involving primarily forced hydration/diuresis and pain management measures. The most severe cases require blood transfusions, clot evacuation, and can lead to death in some instances. HC of any cause (e.g. drug, radiation, viral) is relatively common among HSCT recipients but BKV-associated HC occurs in approximately 10-12% of patients usually within 6 months after transplantation. There are other viral etiologies of HC, with adenovirus being a more common cause of HC among pediatric HSCT recipients compared with adult HSCT recipients. BK virus has also been observed in other immunocompromised conditions such as systemic lupus erythromatosis, other solid organ transplants and in HIV/AIDS patients (Jiang et al., Virol. 2009; 384:266-273).

At this point, the treatment of BK nephropathy associated with organ transplantation is the reduction of immunosuppression in an attempt to prevent graft dysfunction and graft loss (Wiseman et al., Am. J. Kidney Dis. 2009; 54(1): 131-142 and Hirsch et al., Transplantation 2005; 79(1): 1277-1286). There are no fixed clinical regimes for the reduction, as reduction of the immunosuppression may help to prevent progression from viremia to the extensive damage associated with clinical nephropathy, but this also increases the risk of acute organ rejection (Brennan et al., Am. J. Transplant 2005; 5(3):582-594). Clinicians have reported the use of therapeutics such as cidofovir, leflunomide or quinolones in combination with the reduction of immunosuppressants, however the reports find this approach ineffective, with the added burden of managing additional side effects (Randhawa and Brennan Am. J. Transplant 2006; 6(9):2000-2005). As such, there is an unmet and useful need in the field for therapies that neutralize polyoma viruses such as BK and that can be used in an immunocompromised host.

JC virus is also a polyoma virus which is also highly prevalent in the population (80%), although JC virus is generally acquired later than BK virus (Padgett et al., J. Infect. Dis. 1973; 127(4):467-470 and Sabath et al., J. Infect. Dis. 2002; 186 Suppl. 2:5180-5186). After initial infection, JC virus establishes latency in the lymphoid organs and kidneys and when reactivated, invades the central nervous system via infected B-lymphocytes. Once in the CNS, the JC virus causes progressive multifocal leukoencephalopathy (PML), which is a progressive demylenating central nervous system disorder. PML most often presents as an opportunistic infection in HIV/AIDS patients and has also been reported in immunosuppressed patients (Angstrom et al., Brain 1958; 81(1):93-111 and Garcia-Suarez et al., Am. J. Hematol. 2005; 80(4):271-281). PML patients present with confusion, mental status changes, gait ataxia, focal neurological defects such as hemi paresis, limb paresis and visual changes (Richardson E. P., N. Eng. J. Med. 1961; 265:815-823). The prognosis of patients with PML is poor and is especially poor in patients with HIV/AIDS (Antinori et al., J. Neurovirol. 2003; 9 suppl. 1:47-53). This further highlights the unmet and useful need in the field for therapies that neutralize polyoma viruses such as JC.

### SUMMARY OF THE INVENTION

The present disclosure is directed to neutralizing antibodies to human polyomaviruses and/or fragments thereof, antibodies that recognize BK virus and/or JC virus and their respective VP1 pentamers and fragments thereof.

An antibody, wherein said antibody or antigen binding fragment thereof specifically binds VP1.

The antibody wherein said antibody or antigen binding fragment thereof specifically binds BK virus serotype I-serotype IV VP1. In one embodiment, the antibody or antigen binding fragment thereof binds to BKV serotype I VP1 with a binding affinity of 5.0 pM or less, binds to BKV serotype II VP1 with a binding affinity of 29.0 pM or less, binds to BKV serotype III VP1 with a binding affinity of 6.0 pM or less and/or binds to BKV serotype IV VP1 with a binding affinity of 185.0 pM or less. In another embodiment, the antibody or antigen binding fragment thereof further binds to JCV VP1 and specific JCV VP1 mutants with a binding affinity in the high nanomolar range.

The antibody wherein said antibody or antigen binding fragment specifically binds to a VP1 of Table 1. In one embodiment, the antibody or antigen binding fragment thereof binds to two or more of the VP1s of Table 1. In one embodiment, the antibody or antigen binding fragment thereof binds to BKV VP1 serotype I and BKV VP1 serotype II. In another embodiment, the antibody or antigen binding fragment thereof binds to BKV VP1 serotype I and BKV VP1 serotype III. In another embodiment, the antibody or antigen binding fragment thereof binds to BKV VP1 serotype I and BKV VP1 serotype IV. In another embodiment, the antibody or antigen binding fragment thereof binds to BKV VP1 serotype II and BKV VP1 serotype III. In another embodiment, the antibody or antigen binding fragment thereof binds to BKV VP1 serotype II and BKV VP1 serotype IV. In another embodiment, the antibody or antigen binding fragment thereof binds to BKV VP1 serotype I and JCV VP1. In a preferred embodiment, the antibody or antigen binding fragment thereof binds to BKV VP1 serotypes I, II, III and IV. Furthermore, the antibody or antigen binding fragment thereof binds to BKV VP1 serotypes I, II, III and IV and JCV VP1.

The antibody wherein said antibody or antigen binding fragment specifically binds to one or more amino acids residues of a VP1 epitope (SEQ ID NO:500 or SEQ ID NO:501). In one embodiment, the antibody or antigen binding fragment specifically binds to one or more of amino acids Y169, R170 and K172, e.g., binds to Y169 and R170, e.g., as determined by scanning alanine mutagenesis, as described herein.

The antibody wherein said antibody or antigen binding fragment comprises the sequence GFTFXNYWMT (SEQ ID NO. 507), wherein X can be any amino acid (Xaa). In another embodiment, X can be N (Asn), S (Ser), K (Lys) or Q (Gln).

An antibody, wherein said antibody or antigen binding fragment thereof comprises:(i) a heavy chain variable region that comprises (a) a HCDR1 (CDR-Complementarity Determining Region) of SEQ ID NO: 6, (b) a HCDR2 of SEQ ID NO:7, (c) a HCDR3 of SEQ ID NO:8 and a light chain variable region that comprises: (d) a LCDR1 of SEQ ID NO:16, (e) a LCDR2 of SEQ ID NO:17, and (f) a LCDR3 of SEQ ID NO:18;
(ii) a heavy chain variable region that comprises (a) a HCDR1 of SEQ ID NO:26, (b) a HCDR2 of SEQ ID NO:27, (c) a HCDR3 of SEQ ID NO:28; and a light chain variable region that comprises: (d) a LCDR1 of SEQ ID NO:36, (e) a LCDR2 of SEQ ID NO:37, and (f) a LCDR3 of SEQ ID NO:38;
(iii) a heavy chain variable region that comprises (a) a HCDR1 of SEQ ID NO:46, (b) a HCDR2 of SEQ ID NO:47, (c) a HCDR3 of SEQ ID NO:48; and a light chain variable region that comprises: (d) a LCDR1 of SEQ ID NO:56, (e) a LCDR2 of SEQ ID NO:57, and (f) a LCDR3 of SEQ ID NO:58;
(iv) a heavy chain variable region that comprises: (a) a HCDR1 of SEQ ID NO:66, (b) a HCDR2 of SEQ ID NO:67, (c) a HCDR3 of SEQ ID NO:68; and a light chain variable region that comprises: (d) a LCDR1 of SEQ ID NO:76, (e) a LCDR2 of SEQ ID NO:77, and (f) a LCDR3 of SEQ ID NO:78;
(v) a heavy chain variable region that comprises: (a) a HCDR1 of SEQ ID NO:86, (b) a HCDR2 of SEQ ID NO:87, (c) a HCDR3 of SEQ ID NO:88; and a light chain variable region that comprises: (d) a LCDR1 of SEQ ID NO:96, (e) a LCDR2 of SEQ ID NO:97, and (f) a LCDR3 of SEQ ID NO:98;
(vi) a heavy chain variable region that comprises: (a) a HCDR1 of SEQ ID NO:106, (b) a HCDR2 of SEQ ID NO: 107, (c) a HCDR3 of SEQ ID NO:108; and a light chain variable region that comprises: (d) a LCDR1 of SEQ ID NO:116, (e) a LCDR2 of SEQ ID NO:117, and (f) a LCDR3 of SEQ ID NO:118;
(vii) a heavy chain variable region that comprises: (a) a HCDR1 of SEQ ID NO:126, (b) a HCDR2 of SEQ ID NO: 127, (c) a HCDR3 of SEQ ID NO:128; and a light chain variable region that comprises: (d) a LCDR1 of SEQ ID NO:136, (e) a LCDR2 of SEQ ID NO:137, and (f) a LCDR3 of SEQ ID NO:138;
(viii) a heavy chain variable region that comprises: (a) a HCDR1 of SEQ ID NO:146, (b) a HCDR2 of SEQ ID NO:147, (c) a HCDR3 of SEQ ID NO:148; and a light chain variable region that comprises: (d) a LCDR1 of SEQ ID NO:156, (e) a LCDR2 of SEQ ID NO:157, and (f) a LCDR3 of SEQ ID NO:158;
(ix) a heavy chain variable region that comprises: (a) a HCDR1 of SEQ ID NO:166, (b) a HCDR2 of SEQ ID NO: 167, (c) a HCDR3 of SEQ ID NO:168; and a light chain variable region that comprises: (d) a LCDR1 of SEQ ID NO:176, (e) a LCDR2 of SEQ ID NO:177, and (f) a LCDR3 of SEQ ID NO: 178;
(x) a heavy chain variable region that comprises: (a) a HCDR1 of SEQ ID NO:186, (b) a HCDR2 of SEQ ID NO:187, (c) a HCDR3 of SEQ ID NO:188; and a light chain variable region that comprises: (d) a LCDR1 of SEQ ID NO:196, (e) a LCDR2 of SEQ ID NO:197, and (f) a LCDR3 of SEQ ID NO:198;
(xi) a heavy chain variable region that comprises: (a) a HCDR1 of SEQ ID NO:206, (b) a HCDR2 of SEQ ID NO:207, (c) a HCDR3 of SEQ ID NO:208; and a light chain variable region that comprises: (d) a LCDR1 of SEQ ID NO:216, (e) a LCDR2 of SEQ ID NO:217, and (f) a LCDR3 of SEQ ID NO:218;
(xii) a heavy chain variable region that comprises: (a) a HCDR1 of SEQ ID NO:226, (b) a HCDR2 of SEQ ID NO:227, (c) a HCDR3 of SEQ ID NO:228; and a light chain variable region that comprises: (d) a LCDR1 of SEQ ID NO:236, (e) a LCDR2 of SEQ ID NO:237, and (f) a LCDR3 of SEQ ID NO:238;
(xiii) a heavy chain variable region that comprises: (a) a HCDR1 of SEQ ID NO:246, (b) a HCDR2 of SEQ ID NO:247, (c) a HCDR3 of SEQ ID NO:248; and a light chain variable region that comprises: (d) a LCDR1 of SEQ ID NO:256, (e) a LCDR2 of SEQ ID NO:257, and (f) a LCDR3 of SEQ ID NO:258;
(xiv) a heavy chain variable region that comprises: (a) a HCDR1 of SEQ ID NO:266, (b) a HCDR2 of SEQ ID NO:267, (c) a HCDR3 of SEQ ID NO:268; and a light chain variable region that comprises: (d) a LCDR1 of SEQ ID NO: 276, (e) a LCDR2 of SEQ ID NO:277, and (f) a LCDR3 of SEQ ID NO:278;
(xv) a heavy chain variable region that comprises: (a) a HCDR1 of SEQ ID NO:286, (b) a HCDR2 of SEQ ID NO:287, (c) a HCDR3 of SEQ ID NO:288; and a light chain variable region that comprises: (d) a LCDR1 of SEQ ID NO:296, (e) a LCDR2 of SEQ ID NO:297, and (f) a LCDR3 of SEQ ID NO:298;
(xvi) a heavy chain variable region that comprises: (a) a HCDR1 of SEQ ID NO:306, (b) a HCDR2 of SEQ ID NO:307, (c) a HCDR3 of SEQ ID NO:308; and a light chain variable region that comprises: (d) a LCDR1 of SEQ ID NO:314, (e) a LCDR2 of SEQ ID NO:315, and (f) a LCDR3 of SEQ ID NO:316;
(xvii) a heavy chain variable region that comprises: (a) a HCDR1 of SEQ ID NO:322, (b) a HCDR2 of SEQ ID NO:323, (c) a HCDR3 of SEQ ID NO:324; and a light chain variable region that comprises: (d) a LCDR1 of SEQ ID NO:332, (e) a LCDR2 of SEQ ID NO:333, and (f) a LCDR3 of SEQ ID NO:334;
(xviii) a heavy chain variable region that comprises: (a) a HCDR1 of SEQ ID NO:342, (b) a HCDR2 of SEQ ID NO:343, (c) a HCDR3 of SEQ ID NO:344; and a light chain variable region that comprises: (d) a LCDR1 of SEQ ID NO:349, (e) a LCDR2 of SEQ ID NO:350, and (f) a LCDR3 of SEQ ID NO:351;
(xix) a heavy chain variable region that comprises: (a) a HCDR1 of SEQ ID NO:356, (b) a HCDR2 of SEQ ID NO:357, (c) a HCDR3 of SEQ ID NO:358; and a light chain variable region that comprises: (d) a LCDR1 of SEQ ID NO:363, (e) a LCDR2 of SEQ ID NO:364, and (f) a LCDR3 of SEQ ID NO:365;
(xx) a heavy chain variable region that comprises: (a) a HCDR1 of SEQ ID NO:370, (b) a HCDR2 of SEQ ID NO:371, (c) a HCDR3 of SEQ ID NO:372; and a light chain variable region that comprises: (d) a LCDR1 of SEQ ID NO:377, (e) a LCDR2 of SEQ ID NO:378, and (f) a LCDR3 of SEQ ID NO:379;
(xxi) a heavy chain variable region that comprises: (a) a HCDR1 of SEQ ID NO:384, (b) a HCDR2 of SEQ ID NO:385, (c) a HCDR3 of SEQ ID NO:386; and a light chain variable region that comprises: (d) a LCDR1 of SEQ ID NO:391, (e) a LCDR2 of SEQ ID NO:392, and (f) a LCDR3 of SEQ ID NO:393;
(xxii) a heavy chain variable region that comprises: (a) a HCDR1 of SEQ ID NO:398, (b) a HCDR2 of SEQ ID NO:399, (c) a HCDR3 of SEQ ID NO:400; and a light chain variable region that comprises: (d) a LCDR1 of SEQ ID NO:405, (e) a LCDR2 of SEQ ID NO:406, and (f) a LCDR3 of SEQ ID NO:407;
(xxiii) a heavy chain variable region that comprises: (a) a HCDR1 of SEQ ID NO:412, (b) a HCDR2 of SEQ ID NO:413, (c) a HCDR3 of SEQ ID NO:414; and a light chain variable region that comprises: (d) a LCDR1 of SEQ ID NO:419, (e) a LCDR2 of SEQ ID NO:420, and (f) a LCDR3 of SEQ ID NO:421;
(xxiv) a heavy chain variable region that comprises: (a) a HCDR1 of SEQ ID NO:426, (b) a HCDR2 of SEQ ID NO:427, (c) a HCDR3 of SEQ ID NO:428; and a light chain variable region that comprises: (d) a LCDR1 of SEQ ID NO:433, (e) a LCDR2 of SEQ ID NO:434, and (f) a LCDR3 of SEQ ID NO:435;
(xxv) a heavy chain variable region that comprises: (a) a HCDR1 of SEQ ID NO:440, (b) a HCDR2 of SEQ ID NO:441, (c) a HCDR3 of SEQ ID NO:442; and a light chain variable region that comprises: (d) a LCDR1 of SEQ ID NO:447, (e) a LCDR2 of SEQ ID NO:448, and (f) a LCDR3 of SEQ ID NO:449;
(xxvi) a heavy chain variable region that comprises: (a) a HCDR1 of SEQ ID NO:454, (b) a HCDR2 of SEQ ID NO:455, (c) a HCDR3 of SEQ ID NO:456; and a light chain variable region that comprises: (d) a LCDR1 of SEQ ID NO:461, (e) a LCDR2 of SEQ ID NO:462, and (f) a LCDR3 of SEQ ID NO:463;
(xxvii) a heavy chain variable region that comprises: (a) a HCDR1 of SEQ ID NO:468, (b) a HCDR2 of SEQ ID NO:469, (c) a HCDR3 of SEQ ID NO:470; and a light chain variable region that comprises: (d) a LCDR1 of SEQ ID NO:475, (e) a LCDR2 of SEQ ID NO:476, and (f) a LCDR3 of SEQ ID NO:477;
(xxviii) a heavy chain variable region that comprises: (a) a HCDR1 of SEQ ID NO:482, (b) a HCDR2 of SEQ ID NO:483, (c) a HCDR3 of SEQ ID NO:484; and a light chain variable region that comprises: (d) a LCDR1 of SEQ ID NO:489, (e) a LCDR2 of SEQ ID NO:490, and (f) a LCDR3 of SEQ ID NO:491.

An antibody, wherein said antibody or antigen binding fragment thereof comprises:(i) a heavy chain variable region that comprises (a) a HCDR1 (CDR-Complementarity Determining Region) of SEQ ID NO: 508, (b) a HCDR2 of SEQ ID NO:509, (c) a HCDR3 of SEQ ID NO:510 and a light chain variable region that comprises: (d) a LCDR1 of SEQ ID NO:511, (e) a LCDR2 of SEQ ID NO:512, and (f) a LCDR3 of SEQ ID NO:513;
(ii) a heavy chain variable region that comprises (a) a HCDR1 of SEQ ID NO:514, (b) a HCDR2 of SEQ ID NO:515, (c) a HCDR3 of SEQ ID NO:516; and a light chain variable region that comprises: (d) a LCDR1 of SEQ ID NO:517, (e) a LCDR2 of SEQ ID NO:518, and (f) a LCDR3 of SEQ ID NO:519;
(iii) a heavy chain variable region that comprises (a) a HCDR1 of SEQ ID NO:520, (b) a HCDR2 of SEQ ID NO:521, (c) a HCDR3 of SEQ ID NO:522; and a light chain variable region that comprises: (d) a LCDR1 of SEQ ID NO:523, (e) a LCDR2 of SEQ ID NO:524, and (f) a LCDR3 of SEQ ID NO:525;
(iv) a heavy chain variable region that comprises: (a) a HCDR1 of SEQ ID NO:526, (b) a HCDR2 of SEQ ID NO:527, (c) a HCDR3 of SEQ ID NO:528; and a light chain variable region that comprises: (d) a LCDR1 of SEQ ID NO:529, (e) a LCDR2 of SEQ ID NO:530, and (f) a LCDR3 of SEQ ID NO:531;
(v) a heavy chain variable region that comprises: (a) a HCDR1 of SEQ ID NO:532, (b) a HCDR2 of SEQ ID NO:533, (c) a HCDR3 of SEQ ID NO:534; and a light chain variable region that comprises: (d) a LCDR1 of SEQ ID NO:535, (e) a LCDR2 of SEQ ID NO:536, and (f) a LCDR3 of SEQ ID NO:537;
(vi) a heavy chain variable region that comprises: (a) a HCDR1 of SEQ ID NO:538, (b) a HCDR2 of SEQ ID NO:539, (c) a HCDR3 of SEQ ID NO:540; and a light chain variable region that comprises: (d) a LCDR1 of SEQ ID NO:541, (e) a LCDR2 of SEQ ID NO:542, and (f) a LCDR3 of SEQ ID NO:543.

The antibody wherein at least one amino acid within a CDR is substituted by a corresponding residue of a corresponding CDR of another anti-VP1 antibody of Table 2.

The antibody wherein one or two amino acids within a CDR have been modified, deleted or substituted.

The antibody that retains at least 90, 91, 92, 93, 94, 95, 96, 97, 98 or 99% identity over either the variable heavy chain region or the variable light chain region.

The antibody that comprises the modifications in Table 3.

The antibody wherein the antibody is a monoclonal antibody, a chimeric antibody, a humanized antibody, a human engineered antibody, a human antibody, a single chain antibody(scFv) or an antibody fragment.

The antibody wherein said antibody or antigen binding fragment thereof comprises:
(i) a heavy chain variable region (vH) that comprises SEQ ID NO:12, and a light chain variable region (vL) that comprises SEQ ID NO: 22;
(ii) a heavy chain variable region (vH) that comprises SEQ ID NO: 32, and a light chain variable region (vL) that comprises SEQ ID NO: 42;
(iii) a heavy chain variable region (vH) that comprises SEQ ID NO: 52, and a light chain variable region (vL) that comprises SEQ ID NO: 62;
(iv) a heavy chain variable region (vH) that comprises SEQ ID NO: 72, and a light chain variable region (vL) that comprises SEQ ID NO: 82;
(v) a heavy chain variable region (vH) that comprises SEQ ID NO:92, and a light chain variable region (vL) that comprises SEQ ID NO:102;
(vi) a heavy chain variable region (vH) that comprises SEQ ID NO:112, and a light chain variable region (vL) that comprises SEQ ID NO:122;
(vii) a heavy chain variable region (vH) that comprises SEQ ID NO: 132, and a light chain variable region (vL) that comprises SEQ ID NO:142;
(viii) a heavy chain variable region (vH) that comprises SEQ ID NO: 152, and a light chain variable region (vL) that comprises SEQ ID NO:162;
(ix) a heavy chain variable region (vH) that comprises SEQ ID NO:172, and a light chain variable region (vL) that comprises SEQ ID NO:182;
(x) a heavy chain variable region (vH) that comprises SEQ ID NO: 192, and a light chain variable region (vL) that comprises SEQ ID NO:202;
(xi) a heavy chain variable region (vH) that comprises SEQ ID NO:212, and a light chain variable region (vL) that comprises SEQ ID NO:222;
(xii) a heavy chain variable region (vH) that comprises SEQ ID NO:232, and a light chain variable region (vL) that comprises SEQ ID NO:242;
(xiii) a heavy chain variable region (vH) that comprises SEQ ID NO:252, and a light chain variable region (vL) that comprises SEQ ID NO:262;
(xiv) a heavy chain variable region (vH) that comprises SEQ ID NO:272, and a light chain variable region (vL) that comprises SEQ ID NO:282;
(xv) a heavy chain variable region (vH) that comprises SEQ ID NO:292, and a light chain variable region (vL) that comprises SEQ ID NO:302;
(xvi) a heavy chain variable region (vH) that comprises SEQ ID NO:312, and a light chain variable region (vL) that comprises SEQ ID NO:320;
(xvii) a heavy chain variable region (vH) that comprises SEQ ID NO:328, and a light chain variable region (vL) that comprises SEQ ID NO:338;
(xviii) a heavy chain variable region (vH) that comprises SEQ ID NO:348, and a light chain variable region (vL) that comprises SEQ ID NO:355;
(xix) a heavy chain variable region (vH) that comprises SEQ ID NO:362, and a light chain variable region (vL) that comprises SEQ ID NO:369;
(xx) a heavy chain variable region (vH) that comprises SEQ ID NO:376, and a light chain variable region (vL) that comprises SEQ ID NO:383;
(xxi) a heavy chain variable region (vH) that comprises SEQ ID NO:390, and a light chain variable region (vL) that comprises SEQ ID NO:397;
(xxii) a heavy chain variable region (vH) that comprises SEQ ID NO:404, and a light chain variable region (vL) that comprises SEQ ID NO:411;
(xxiii) a heavy chain variable region (vH) that comprises SEQ ID NO:418, and a light chain variable region (vL) that comprises SEQ ID NO:425;
(xxiv) a heavy chain variable region (vH) that comprises SEQ ID NO:432, and a light chain variable region (vL) that comprises SEQ ID NO:439;
(xxv) a heavy chain variable region (vH) that comprises SEQ ID NO: 446, and a light chain variable region (vL) that comprises SEQ ID NO:453;
(xxvi) a heavy chain variable region (vH) that comprises SEQ ID NO:460, and a light chain variable region (vL) that comprises SEQ ID NO:467;
(xxvii) a heavy chain variable region (vH) that comprises SEQ ID NO:474, and a light chain variable region (vL) that comprises SEQ ID NO:481; or
(xxviii) a heavy chain variable region (vH) that comprises SEQ ID NO:488, and a light chain variable region (vL) that comprises SEQ ID NO:495.

The antibody that retains at least 90, 91, 92, 93, 94, 95, 96, 97, 98 or 99% identity over either the variable light or variable heavy region.

The antibody wherein one, two, three, four or five, but less than 10 amino acids within the variable light or variable heavy region have been modified, deleted or substituted.

The antibody wherein the antibody is a monoclonal antibody, a chimeric antibody, a humanized antibody, a human engineered antibody, a human antibody, a single chain antibody(scFv) or an antibody fragment.

The antibody of any of the preceding embodiments wherein the antibody or fragment thereof has reduced glycosylation or no glycosylation or is hypofucosylated.

A composition comprising a plurality of an antibody or antigen binding fragment of any of the preceding embodiments, wherein at least 0.05%, 0.1%, 0.5%, 1%, 2%, 3%, 5% or more of the antibodies in the composition have an α2,3-linked sialic acid residue, and wherein said antibody or antigen binding fragment thereof comprises:(i) a heavy chain variable region that comprises (a) a HCDR1 (CDR-Complementarity Determining Region) of SEQ ID NO: 6, (b) a HCDR2 of SEQ ID NO:7, (c) a HCDR3 of SEQ ID NO:8 and a light chain variable region that comprises: (d) a LCDR1 of SEQ ID NO:16, (e) a LCDR2 of SEQ ID NO:17, and (f) a LCDR3 of SEQ ID NO:18;
(ii) a heavy chain variable region that comprises (a) a HCDR1 of SEQ ID NO:26, (b) a HCDR2 of SEQ ID NO:27, (c) a HCDR3 of SEQ ID NO:28; and a light chain variable region that comprises: (d) a LCDR1 of SEQ ID NO:36, (e) a LCDR2 of SEQ ID NO:37, and (f) a LCDR3 of SEQ ID NO:38;
(iii) a heavy chain variable region that comprises (a) a HCDR1 of SEQ ID NO:46, (b) a HCDR2 of SEQ ID NO:47, (c) a HCDR3 of SEQ ID NO:48; and a light chain variable region that comprises: (d) a LCDR1 of SEQ ID NO:56, (e) a LCDR2 of SEQ ID NO:57, and (f) a LCDR3 of SEQ ID NO:58;
(iv) a heavy chain variable region that comprises: (a) a HCDR1 of SEQ ID NO:66, (b) a HCDR2 of SEQ ID NO:67, (c) a HCDR3 of SEQ ID NO:68; and a light chain variable region that comprises: (d) a LCDR1 of SEQ ID NO:76, (e) a LCDR2 of SEQ ID NO:77, and (f) a LCDR3 of SEQ ID NO:78;
(v) a heavy chain variable region that comprises: (a) a HCDR1 of SEQ ID NO:86, (b) a HCDR2 of SEQ ID NO:87, (c) a HCDR3 of SEQ ID NO:88; and a light chain variable region that comprises: (d) a LCDR1 of SEQ ID NO:96, (e) a LCDR2 of SEQ ID NO:97, and (f) a LCDR3 of SEQ ID NO:98;
(vi) a heavy chain variable region that comprises: (a) a HCDR1 of SEQ ID NO:106, (b) a HCDR2 of SEQ ID NO: 107, (c) a HCDR3 of SEQ ID NO:108; and a light chain variable region that comprises: (d) a LCDR1 of SEQ ID NO:116, (e) a LCDR2 of SEQ ID NO:117, and (f) a LCDR3 of SEQ ID NO:118;
(vii) a heavy chain variable region that comprises: (a) a HCDR1 of SEQ ID NO:126, (b) a HCDR2 of SEQ ID NO: 127, (c) a HCDR3 of SEQ ID NO:128; and a light chain variable region that comprises: (d) a LCDR1 of SEQ ID NO:136, (e) a LCDR2 of SEQ ID NO:137, and (f) a LCDR3 of SEQ ID NO:138;
(viii) a heavy chain variable region that comprises: (a) a HCDR1 of SEQ ID NO:146, (b) a HCDR2 of SEQ ID NO:147, (c) a HCDR3 of SEQ ID NO:148; and a light chain variable region that comprises: (d) a LCDR1 of SEQ ID NO:156, (e) a LCDR2 of SEQ ID NO:157, and (f) a LCDR3 of SEQ ID NO:158;
(ix) a heavy chain variable region that comprises: (a) a HCDR1 of SEQ ID NO:166, (b) a HCDR2 of SEQ ID NO: 167, (c) a HCDR3 of SEQ ID NO:168; and a light chain variable region that comprises: (d) a LCDR1 of SEQ ID NO:176, (e) a LCDR2 of SEQ ID NO:177, and (f) a LCDR3 of SEQ ID NO: 178;
(x) a heavy chain variable region that comprises: (a) a HCDR1 of SEQ ID NO:186, (b) a HCDR2 of SEQ ID NO:187, (c) a HCDR3 of SEQ ID NO:188; and a light chain variable region that comprises: (d) a LCDR1 of SEQ ID NO:196, (e) a LCDR2 of SEQ ID NO:197, and (f) a LCDR3 of SEQ ID NO:198;
(xi) a heavy chain variable region that comprises: (a) a HCDR1 of SEQ ID NO:206, (b) a HCDR2 of SEQ ID NO:207, (c) a HCDR3 of SEQ ID NO:208; and a light chain variable region that comprises: (d) a LCDR1 of SEQ ID NO:216, (e) a LCDR2 of SEQ ID NO:217, and (f) a LCDR3 of SEQ ID NO:218;
(xii) a heavy chain variable region that comprises: (a) a HCDR1 of SEQ ID NO:226, (b) a HCDR2 of SEQ ID NO:227, (c) a HCDR3 of SEQ ID NO:228; and a light chain variable region that comprises: (d) a LCDR1 of SEQ ID NO:236, (e) a LCDR2 of SEQ ID NO:237, and (f) a LCDR3 of SEQ ID NO:238;
(xiii) a heavy chain variable region that comprises: (a) a HCDR1 of SEQ ID NO:246, (b) a HCDR2 of SEQ ID NO:247, (c) a HCDR3 of SEQ ID NO:248; and a light chain variable region that comprises: (d) a LCDR1 of SEQ ID NO:256, (e) a LCDR2 of SEQ ID NO:257, and (f) a LCDR3 of SEQ ID NO:258;
(xiv) a heavy chain variable region that comprises: (a) a HCDR1 of SEQ ID NO:266, (b) a HCDR2 of SEQ ID NO:267, (c) a HCDR3 of SEQ ID NO:268; and a light chain variable region that comprises: (d) a LCDR1 of SEQ ID NO: 276, (e) a LCDR2 of SEQ ID NO:277, and (f) a LCDR3 of SEQ ID NO:278;
(xv) a heavy chain variable region that comprises: (a) a HCDR1 of SEQ ID NO:286, (b) a HCDR2 of SEQ ID NO:287, (c) a HCDR3 of SEQ ID NO:288; and a light chain variable region that comprises: (d) a LCDR1 of SEQ ID NO:296, (e) a LCDR2 of SEQ ID NO:297, and (f) a LCDR3 of SEQ ID NO:298;
(xvi) a heavy chain variable region that comprises: (a) a HCDR1 of SEQ ID NO:306, (b) a HCDR2 of SEQ ID NO:307, (c) a HCDR3 of SEQ ID NO:308; and a light chain variable region that comprises: (d) a LCDR1 of SEQ ID NO:314, (e) a LCDR2 of SEQ ID NO:315, and (f) a LCDR3 of SEQ ID NO:316;
(xvii) a heavy chain variable region that comprises: (a) a HCDR1 of SEQ ID NO:322, (b) a HCDR2 of SEQ ID NO:323, (c) a HCDR3 of SEQ ID NO:324; and a light chain variable region that comprises: (d) a LCDR1 of SEQ ID NO:332, (e) a LCDR2 of SEQ ID NO:333, and (f) a LCDR3 of SEQ ID NO:334;
(xviii) a heavy chain variable region that comprises: (a) a HCDR1 of SEQ ID NO:342, (b) a HCDR2 of SEQ ID NO:343, (c) a HCDR3 of SEQ ID NO:344; and a light chain variable region that comprises: (d) a LCDR1 of SEQ ID NO:349, (e) a LCDR2 of SEQ ID NO:350, and (f) a LCDR3 of SEQ ID NO:351;
(xix) a heavy chain variable region that comprises: (a) a HCDR1 of SEQ ID NO:356, (b) a HCDR2 of SEQ ID NO:357, (c) a HCDR3 of SEQ ID NO:358; and a light chain variable region that comprises: (d) a LCDR1 of SEQ ID NO:363, (e) a LCDR2 of SEQ ID NO:364, and (f) a LCDR3 of SEQ ID NO:365;
(xx) a heavy chain variable region that comprises: (a) a HCDR1 of SEQ ID NO:370, (b) a HCDR2 of SEQ ID NO:371, (c) a HCDR3 of SEQ ID NO:372; and a light chain variable region that comprises: (d) a LCDR1 of SEQ ID NO:377, (e) a LCDR2 of SEQ ID NO:378, and (f) a LCDR3 of SEQ ID NO:379;
(xxi) a heavy chain variable region that comprises: (a) a HCDR1 of SEQ ID NO:384, (b) a HCDR2 of SEQ ID NO:385, (c) a HCDR3 of SEQ ID NO:386; and a light chain variable region that comprises: (d) a LCDR1 of SEQ ID NO:391, (e) a LCDR2 of SEQ ID NO:392, and (f) a LCDR3 of SEQ ID NO:393;
(xxii) a heavy chain variable region that comprises: (a) a HCDR1 of SEQ ID NO:398, (b) a HCDR2 of SEQ ID NO:399, (c) a HCDR3 of SEQ ID NO:400; and a light chain variable region that comprises: (d) a LCDR1 of SEQ ID NO:405, (e) a LCDR2 of SEQ ID NO:406, and (f) a LCDR3 of SEQ ID NO:407;
(xxiii) a heavy chain variable region that comprises: (a) a HCDR1 of SEQ ID NO:412, (b) a HCDR2 of SEQ ID NO:413, (c) a HCDR3 of SEQ ID NO:414; and a light chain variable region that comprises: (d) a LCDR1 of SEQ ID NO:419, (e) a LCDR2 of SEQ ID NO:420, and (f) a LCDR3 of SEQ ID NO:421;
(xxiv) a heavy chain variable region that comprises: (a) a HCDR1 of SEQ ID NO:426, (b) a HCDR2 of SEQ ID NO:427, (c) a HCDR3 of SEQ ID NO:428; and a light chain variable region that comprises: (d) a LCDR1 of SEQ ID NO:433, (e) a LCDR2 of SEQ ID NO:434, and (f) a LCDR3 of SEQ ID NO:435;
(xxv) a heavy chain variable region that comprises: (a) a HCDR1 of SEQ ID NO:440, (b) a HCDR2 of SEQ ID NO:441, (c) a HCDR3 of SEQ ID NO:442; and a light chain variable region that comprises: (d) a LCDR1 of SEQ ID NO:447, (e) a LCDR2 of SEQ ID NO:448, and (f) a LCDR3 of SEQ ID NO:449;
(xxvi) a heavy chain variable region that comprises: (a) a HCDR1 of SEQ ID NO:454, (b) a HCDR2 of SEQ ID NO:455, (c) a HCDR3 of SEQ ID NO:456; and a light chain variable region that comprises: (d) a LCDR1 of SEQ ID NO:461, (e) a LCDR2 of SEQ ID NO:462, and (f) a LCDR3 of SEQ ID NO:463;
(xxvii) a heavy chain variable region that comprises: (a) a HCDR1 of SEQ ID NO:468, (b) a HCDR2 of SEQ ID NO:469, (c) a HCDR3 of SEQ ID NO:470; and a light chain variable region that comprises: (d) a LCDR1 of SEQ ID NO:475, (e) a LCDR2 of SEQ ID NO:476, and (f) a LCDR3 of SEQ ID NO:477;
(xxviii) a heavy chain variable region that comprises: (a) a HCDR1 of SEQ ID NO:482, (b) a HCDR2 of SEQ ID NO:483, (c) a HCDR3 of SEQ ID NO:484; and a light chain variable region that comprises: (d) a LCDR1 of SEQ ID NO:489, (e) a LCDR2 of SEQ ID NO:490, and (f) a LCDR3 of SEQ ID NO:491.

A composition comprising a plurality of an antibody or antigen binding fragment of any of the preceding embodiments, wherein none of the antibodies comprise a bisecting GlcNAc.

A pharmaceutical composition comprising the antibody or fragment thereof, of any of the preceding embodiments wherein the composition is prepared as a lyophilisate.

A pharmaceutical composition comprising the antibody or fragment thereof of any of the preceeding embodiments and a pharmaceutically acceptable carrier. In one embodiment, the carrier is a histidine buffer. In one embodiment, the pharmaceutical composition comprises a sugar (e.g., sucrose).

A method of neutralizing a BK virus or JC virus infection comprising administering via injection or infusion to a patient in need an effective amount of the antibody or the pharmaceutical composition. The method wherein the antibody or antigen binding fragment thereof neutralizes BKV serotype I and BKV serotype II. In another embodiment, the antibody or antigen binding fragment thereof neutralizes BKV serotype I and BKV serotype III. In another embodiment, the antibody or antigen binding fragment thereof neutralizes BKV serotype I and BKV serotype IV. In another embodiment, the antibody or antigen binding fragment thereof neutralizes BKV serotype II and BKV serotype III. In another embodiment, the antibody or antigen binding fragment thereof neutralizes BKV serotype II and BKV serotype IV. In another embodiment, the antibody or antigen binding fragment thereof neutralizes BKV serotype I and JCV. In a specific embodiment, the antibody or antigen binding fragment thereof neutralizes BKV serotypes I, II, III and IV. Furthermore, the antibody or antigen binding fragment thereof neutralizes BKV serotypes I, II, III and IV and JCV. In a preferred embodiment, anti-VP1 antibodies neutralized infection by all four serotypes of BKV (I-IV), these anti-VP1 antibodies specifically include P8D11, the modifications of P8D11, and EBB-C1975-B5.

A method of treating or reducing the likelihood of a BK virus or JC virus associated disorder, comprising administering via injection or infusion to a patient in need an effective amount of the antibody or the pharmaceutical composition, and wherein the disorder is: nephropathy, BKVAN, hemorrhagic cystitis (HC), Progressive Multifocal Leukoencephalopathy (PML), granule cell neuronopathy (GCN), interstitial kidney disease, ureteral stenosis, vasculitis, colitis, retinitis, meningitis, and immune reconstitution inflammatory syndrome (IRIS).

The method wherein the antibody or composition is reconstituted prior to injection or infusion.

The method wherein the antibody or the pharmaceutical composition is administered in combination with another therapeutic agent.

The method wherein the therapeutic agent is an immunosuppressive agent.

The method wherein the immunosuppressive agent is a monophosphate dehydrogenase inhibitor, a purine synthesis inhibitor, a calcineurin inhibitor or an mTOR inhibitor.

The method wherein the immunosuppressive agent is mycophenolate mofetil (MMF), mycophenolate sodium, azathioprine, tacrolimus, sirolimus or cyclosporine.

The method wherein the therapeutic agent is an additional anti-VP1 antibody.

The antibody or fragment thereof of any of the preceding embodiments for use as a medicament.

The antibody or fragment thereof or the pharmaceutical composition, for use in the neutralization of a BK virus or JC virus infection.

The antibody or fragment thereof, or the pharmaceutical composition, for use in the treatment or reducing the likelihood of: nephropathy, BKVAN, hemorrhagic cystitis (HC), Progressive Multifocal Leukoencephalopathy (PML), granule cell neuronopathy (GCN), interstitial kidney disease, ureteral stenosis, vasculitis, colitis, retinitis, meningitis, and immune reconstitution inflammatory syndrome (IRIS).

The use of the antibody or fragment thereof, administered in combination with another therapeutic agent.

The use of the antibody or fragment thereof wherein the therapeutic agent is an immunosuppressive agent.

The use of the antibody or fragment thereof wherein the immunosuppressive agent is a monophosphate dehydrogenase inhibitor, a purine synthesis inhibitor, a calcineurin inhibitor or an mTOR inhibitor.

The use of the antibody or fragment thereof, wherein the immunosuppressive agent is: mycophenolate mofetil (MMF), mycophenolate sodium, azathioprine, tacrolimus, sirolimus or cyclosporine.

A nucleic acid that encodes the antibody or antigen binding fragment of any of the preceding embodiments.

A vector comprising the nucleic acid.

A host cell comprising the vector.

A process for producing an antibody or antigen binding fragment comprising cultivating the host cell and recovering the antibody from the culture.

A diagnostic reagent comprising the antibody or antigen binding fragment thereof which is labeled.

The diagnostic reagent wherein the label is selected from the group consisting of a radiolabel, a fluorophore, a chromophore, an imaging agent, and a metal ion.

### Definitions

Unless stated otherwise, the following terms and phrases as used herein are intended to have the following meanings:

The term “antibody” as used herein refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

“Complementarity-determining domains” or “complementary-determining regions (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W. H. Freeman & Co., New York, 2000).

The positions of the CDRs and framework regions can be determined using various well known definitions in the art, e.g., Kabat, Chothia, and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J.Mol.Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HC CDR1), 50-65 (HC CDR2), and 95-102 (HC CDR3) in a VH, e.g., a mammalian VH, e.g., a human VH; and amino acid residues 24-34 (LC CDR1), 50-56 (LC CDR2), and 89-97 (LC CDR3) in a VL, e.g., a mammalian VL, e.g., a human VL.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

The term “antigen binding fragment,” as used herein, refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen binding fragment.” These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can be grafted into scaffolds based on polypeptides such as fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH-CH1-VH-CH1) which, together with complementary light chain polypeptides, form a pair of antigen binding regions (Zapata et al., Protein Eng. 8:1057-1062, 1995; and U.S. Pat. No. 5,641,870).

The term “monoclonal antibody” or “monoclonal antibody composition” as used herein refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

The term “human antibody,” as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000).

The human antibodies of the present disclosure can include amino acid residues not encoded by human sequences (e.g., mutations introduced by random or site-specific mutagenesis in vitro or by somatic mutation in vivo, or a conservative substitution to promote stability or manufacturing).

The term “recognize” as used herein refers to an antibody or antigen binding fragment thereof that finds and interacts (e.g., binds) with its epitope, whether that epitope is linear or conformational. The term “epitope” refers to a site on an antigen to which an antibody or antigen binding fragment of the disclosure specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)). A “paratope” is the part of the antibody which recognizes the epitope of the antigen.

The phrase “specifically binds” or “selectively binds,” when used in the context of describing the interaction between an antigen (e.g., a protein) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two times the background and do not substantially bind in a significant amount to other antigens present in the sample. In one aspect, under designated immunoassay conditions, the antibody or binding agent with a particular binding specificity binds to a particular antigen at least ten (10) times the background and does not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes. Alternatively, in some aspects, antibodies or antibody fragments are selected that cross-react with certain desired molecules.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91% 92%, 93%, 94%, 95%, 96%, 97%, 98%, 99%, or 100% sequence identity with the reference variable region nucleic acid or amino acid sequence.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

The term “equilibrium dissociation constant (KD, M)” refers to the dissociation rate constant (kd, time−1) divided by the association rate constant (ka, time−1, M−1). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

As used herein, the phrase “consisting essentially of” refers to the genera or species of active pharmaceutical agents included in a method or composition, as well as any excipients inactive for the intended purpose of the methods or compositions. In some aspects, the phrase “consisting essentially of” expressly excludes the inclusion of one or more additional active agents other than an anti-VP1 antibody of the present disclosure. In some aspects, the phrase “consisting essentially of” expressly excludes the inclusion of one or more additional active agents other than an anti-VP1 antibody of the present disclosure and a second co-administered agent.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

The term “optimized” as used herein refers to a nucleotide sequence that has been altered to encode an amino acid sequence using codons that are preferred in the production cell or organism, generally a eukaryotic cell, for example, a yeast cell, a Pichia cell, a fungal cell, a Trichoderma cell, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

The terms “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, (Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, (J. Mol. Biol. 48:444-453, 1970), algorithm which has been incorporated into the GAP program in the GCG software package (available from University of South Florida), using either a BLOSUM 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “BKV” or “BK virus” refer to a member of the family Polyomaviridae, genus Orthopolyomavirus. Polyomaviruses are icosahedral, non-enveloped, double-stranded DNA viruses with a genome of approximately 5,000 base pairs. They measure approximately 40-45 nM in diameter (Bennett et al., Microbes and Infection. 2012:14(9):672-683).

“JCV” or “JC virus” refers to a member of the family Polyomaviridae, genus Orthopolyomavirus. JCV is related to BKV, and is also an icosahedral, non-enveloped, double-stranded DNA virus with a genome of approximately 5,000 base pairs. They measure approximately 40-45 nM in diameter (Johne et al., Arch. Virol. 2011; 156(9):1627-1634).

The terms “BKV nephropathy” or “BKV-associated nephropathy” or “BKVAN” refer to the inflammatory interstitial nephropathy resulting from the lytic infection with BKV, characterized by viral cytopathogenic changes and viral gene expression, primarily in the renal tubular epithelium.

The term “VP1” refers to the major polyoma virus capsid subunit protein. “VP1 pentamers” are composed of five monomers of VP1.

TABLE 1-VP1
sequences

| Name | Sequence | SEQ ID NO |
|---|---|---|
| VP1 BKV | MAPTKRKGECPGAAPKKPKEPVQVPKLLIKGGVEVLEV | (SEQ ID |
| serotype I | KTGVDAITEVECFLNPEMGDPDENLRGFSLKLSAENDFS | NO: 1) |
| | SDSPERKMLPCYSTARIPLPNLNEDLTCGNLLMWEAVTV | |
| | QTEVIGITSMLNLHAGSQKVHEHGGGKPIQGSNFHFFAV | |
| | GGDPLEMQGVLMNYRTKYPEGTITPKNPTAQSQVMN | |
| | TDHKAYLDKNNAYPVECWIPDPSRNENTRYFGTFTGGE | |
| | NVPPVLHVTNTATTVLLDEQGVGPLCKADSLYVSAADIC | |
| | GLFTNSSGTQQWRGLARYFKIRLRKRSVKNPYPISFLLSD | |
| | LINRRTQRVDGQPMYGMESQVEEVRVFDGTERLPGDPD | |
| | MIRYIDKQGQLQTKML | |
| VP1 BKV | MAPTKRKGECPGAAPKKPKEPVQVPKLLIKGGVEVLEV | (SEQ ID |
| serotype II | KTGVDAITEVECFLNPEMGDPDDNLRGYSLKLTAENAFD | NO: 2) |
| | SDSPDKKMLPCYSTARIPLPNLNEDLTCGNLLMWEAVTV | |
| | KTEVIGITSMLNLHAGSQKVHENGGGKPVQGSNFHFFAV | |
| | GGDPLEMQGVLMNYRTKYPQGTITPKNPTAQSQVMN | |
| | TDHKAYLDKNNAYPVECWIPDPSRNENTRYFGTYTGGE | |
| | NVPPVLHVTNTATTVLLDEQGVGPLCKADSLYVSAADIC | |
| | GLFTNSSGTQQWRGLARYFKIRLRKRSVKNPYPISFLLSD | |
| | LINRRTQKVDGQPMYGMESQVEEVRVFDGTEQLPGDPD | |
| | MIRYIDRQGQLQTKMV | |
| VP1 BKV | MAPTKRKGECPGAAPKKPKEPVQVPKLLIKGGVEVLEV | (SEQ ID |
| serotype III | KTGVDAITEVECFLNPEMGDPDDHLRGYSQHLSAENAF | NO: 3) |
| | DSDSPDKKMLPCYSTARIPLPNLNEDLTCGNLLMWEAVT | |
| | VKTEVIGITSMLNLHAGSQKVHENGGGKPVQGSNFHFFA | |
| | VGGDPLEMQGVLMNYRTKYPQGTITPKNPTAQSQVM | |
| | NTDHKAYLDKNNAYPVECWIPDPSKNENTRYFGTYTGG | |
| | ENVPPVLHVTNTATTVLLDEQGVGPLCKADSLYVSAADI | |
| | CGLFTNSSGTQQWRGLARYFKIRLRKRSVKNPYPISFLLS | |
| | DLINRRTQKVDGQPMYGMESQVEEVRVFDGTEQLPGDP | |
| | DMIRYIDRQGQLQTKMV | |
| VP1 BKV | MAPTKRKGECPGAAPKKPKEPVQVPKLLIKGGVEVLEV | (SEQ ID |
| serotype IV | KTGVDAITEVECFLNPEMGDPDNDLRGYSLRLTAETAFD | NO: 4) |
| | SDSPDRKMLPCYSTARIPLPNLNEDLTCGNLLMWEAVTV | |
| | KTEVIGITSMLNLHAGSQKVHENGGGKPIQGSNFHFFAV | |
| | GGDPLEMQGVLMNYRTKYPEGTVTPKNPTAQSQVMN | |
| | TDHKAYLDKNNAYPVECWIPDPSRNENTRYFGTYTGGE | |
| | NVPPVLHVTNTATTVLLDEQGVGPLCKADSLYVSAADIC | |
| | GLFTNSSGTQQWRGLPRYFKIRLRKRSVKNPYPISFLLSD | |
| | LINRRTQRVDGQPMYGMESQVEEVRVFDGTEQLPGDPD | |
| | MIRYIDRQGQLQTKMV | |
| JCV VP1 | MAPTKRKGERKDPVQVPKLLIRGGVEVLEVKTGVDSITE | (SEQ ID |
| | VECFLTPEMGDPDEHLRGFSKSISISDTFESDSPNKDMLP | NO: 5) |
| | CYSVARIPLPNLNEDLTCGNILMWEAVTLKTEVIGVTTL | |
| | MNVHSNGQATHDNGAGKPVQGTSFHFFSVGGEALELQG | |
| | VVFNYRTKYPDGTIFPKNATVQSQVMNTEHKAYLDKN | |
| | KAYPVECWVPDPTRNENTRYFGTLTGGENVPPVLHITNT | |
| | ATTVLLDEFGVGPLCKGDNLYLSAVDVCGMFTNRSGSQ | |
| | QWRGLSRYFKVQLRKRRVKNPYPISFLLTDLINRRTPRV | |
| | DGQPMYGMDAQVEEVRVFEGTEELPGDPDMMRYVDRY | |
| | GQLQTKML | |

“Virus-like particles” or “VLP” are an assembly of VP1 pentamers into viral capsids. VLPs are composed of 72 VP1 pentamers. VLPs are structurally very similar to actual virus but lack the minor capsid proteins (VP2 and VP3) as well as the viral DNA genome, and therefore are non-infectious. VLPs are useful as viral epitopes are presented in a similar conformation to the actual virus.

“IC50” (half-maximal inhibitory concentration) refers to the concentration of a particular antibody which induces a signal halfway (50%) between the baseline control and the maximum possible signal. For example, the IC50 is the concentration of antibody at which 50% of the available binding sites on the VP1 antigen are occupied.

“EC50” (half-maximal effective concentration) refers to the concentration of a particular antibody which induces a response halfway (50%) between the baseline control and the maximum possible effect after a specific exposure or treatment time. For example, the EC50 is the concentration of antibody at which virus infection is neutralized by 50%.

“EC90” refers to the concentration of a particular antibody which induces a response corresponding to 90% of the maximum possible effect after a specific exposure or treatment time. For example, the EC90 is the concentration of antibody at which virus infection is neutralized by 90%.

“Neutralization” refers to the inhibition of viral infection of a host cell, as demonstrated by the absence of viral gene expression. Without being held to any one theory, mechanisms of neutralization by a particular antibody could include blocking the interaction of viral capsid proteins with cell surface receptors or disruption of any stage of the entry and trafficking process prior to delivery of the viral genome to the nucleus of the host cell.

As used herein, the terms “treat,” “treating,” or “treatment” of any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both.

The phrase “reducing the likelihood” refers to delaying the onset or development or progression of the disease, infection or disorder.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of, or result in a decrease in severity of, respectively, disease symptoms, including symptoms associated polyoma viral infection.

The term “co-administer” refers to the simultaneous presence of two active agents in the blood of an individual. Active agents that are co-administered can be concurrently or sequentially delivered.

### BRIEF DESCRIPTION OF THE DRAWINGS

FIG. 1A-1D graphically represents affinity measurements for anti-VP1 antibodies on VP1 pentamers for BKV serotypes I-IV by SET assay.

FIG. 2 is a table of SET affinity values (K_{D}) for anti-VP1 antibodies on VP1 pentamers for BKV serotypes I-IV.

FIG. 3A-3E graphically represents affinity measurements for anti-VP1 antibodies on VP1 pentamers or VLPs for BKV serotypes I-IV by Biacore.

FIG. 4 is a graph of anti-VP1 antibodies binding to BKV serotype I VLPs as measured by ELISA.

FIG. 5 is a graph of anti-VP1 antibodies binding to BKV serotype IV VLPs as measured by ELISA.

FIG. 6 is a graph of anti-VP1 antibodies binding to BKV serotype IV VP1 pentamers as measured by ELISA.

FIG. 7 is a table of IC50 values generated by ELISA for anti-VP1 antibodies binding to VLPs or VP1 pentamers for BKV serotypes I and IV.

FIG. 8 is a graph of anti-VP1 antibodies binding to BKV serotype I VLPs as measured by ELISA.

FIG. 9 is a table of IC50 values generated by ELISA for anti-VP1 antibodies binding to BKV serotype I VLPs.

FIG. 10 is a graph of anti-VP1 antibodies binding to JC virus VLPs as measured by ELISA.

FIG. 11 is a table of IC50 values generated by ELISA for anti-VP1 antibodies binding to JCV VLPs.

FIG. 12A-B shows two blots. The upper panel (FIG. 12A) is a Western blot demonstrating no binding of anti-VP1 antibodies to denatured BKV VP1. The lower panel (FIG. 12B) is a dot-blot of non-denatured BKV VP1 pentamers, demonstrating binding of anti-VP1 antibodies to non-denatured VP1 pentamers.

FIG. 13A-13F graphically represents binding of anti-VP1 antibodies to wild type BKV serotype I VP1 pentamers by Biacore, but that point mutations in the VP1 can disrupt binding.

FIG. 14 is a table summarizing the key residues for binding identified in the epitopes of anti-VP1 antibodies.

FIG. 15 is a graph of anti-VP1 antibodies neutralizing BKV serotype I infection.

FIG. 16 is a graph of anti-VP1 antibodies neutralizing BKV serotype II infection.

FIG. 17 is a graph of anti-VP1 antibodies neutralizing BKV serotype III infection.

FIG. 18 is a graph of anti-VP1 antibodies neutralizing BKV serotype IV infection.

FIG. 19 is a table summarizing the neutralizing activity (EC50 and EC90) of anti-VP1 antibodies on BKV serotypes I-IV and JC virus.

FIG. 20 is a graph of anti-VP1 antibodies neutralizing infection with BKV serotypes I, II, and IV.

FIG. 21 is a table summarizing the neutralizing activity (EC50 and EC90) of anti-VP1 antibodies on BKV serotypes I-IV.

FIG. 22 is a graph of anti-VP1 antibodies neutralizing infection with BKV serotype I.

FIG. 23 is a table summarizing the neutralizing activity (EC50 and EC90) of anti-VP1 antibodies on BKV serotype I.

FIG. 24 is a graph of anti-VP1 antibodies neutralizing infection with JCV.

FIG. 25 is a graph of anti-VP1 antibodies neutralizing infection with JCV.

FIG. 26 is table summarizing the neutralizing activity (EC50 and EC90) of anti-VP1 antibodies on JCV infection.

FIG. 27 is table of antibody P8D11 affinity on JC virus VLPs and VLPs containing point mutations.

FIG. 28 is deuterium exchange epitope mapping of a P8D11 Fab bound to BKV VP1 pentamers.

FIG. 29A is a table that shows anti-BKV antibody contact residues in the EF loop when certain mutations are introduced by alanine scanning. FIG. 29B-29C shows the SPR graphs of anti-BKV antibody binding to wild type and mutated residues in VP1.

FIG. 30 is an X-ray crystal structure of P8D11 in complex with BKV VP1 pentamer.

FIG. 31A-B is a graphical representation of how P8D11 contacts the residues of the VP1 pentamer.

### DETAILED DESCRIPTION

The present disclosure provides for antibodies, antibody fragments (e.g., antigen binding fragments), that bind and neutralize BKV. In particular, the present disclosure is directed to antibodies and antibody fragments (e.g., antigen binding fragments) that bind to VP1 proteins, and neutralize viral infection upon such binding. Furthermore, the present disclosure provides antibodies that have desirable pharmacokinetic characteristics and other desirable attributes, and thus can be used for reducing the likelihood of or treating BK virus-associated nephropathy (e.g. BKVAN). The present disclosure further provides pharmaceutical compositions comprising the antibodies and methods of making and using such pharmaceutical compositions for the prevention and treatment of polyoma virus infection and associated disorders.

### Anti-VP1 Antibodies

The present disclosure provides for antibodies or antibody fragments (e.g., antigen binding fragments) that specifically bind to VP1. Antibodies or antibody fragments (e.g., antigen binding fragments) of the present disclosure include, but are not limited to, the human monoclonal antibodies or fragments thereof, isolated as described, in the Examples below.

The present disclosure in certain aspects provides antibodies or antibody fragments (e.g., antigen binding fragments) that specifically bind to VP1, said antibodies or antibody fragments (e.g., antigen binding fragments) comprise a VH domain having an amino acid sequence of SEQ ID NO:12, 32, 52, 72, 92, 112, 132, 152, 172, 192, 212, 232, 252, 272, 292, 312, 328, 348, 362, 376, 390, 404, 418, 432, 446, 460, 474, and 488 (Table 2). The present disclosure also provides antibodies or antibody fragments (e.g., antigen binding fragments) that specifically bind to VP1, said antibodies or antibody fragments (e.g., antigen binding fragments) comprise a VH CDR having an amino acid sequence of any one of the VH CDRs listed in Table 2. In particular aspects, the present disclosure provides antibodies or antibody fragments (e.g., antigen binding fragments) that specifically bind to VP1, said antibodies comprising (or alternatively, consist of) one, two, three, or more VH CDRs having an amino acid sequence of any of the VH CDRs listed in Table 2.

The present disclosure provides antibodies or antibody fragments (e.g., antigen binding fragments) that specifically bind to VP1, said antibodies or antibody fragments (e.g., antigen binding fragments) comprise a VL domain having an amino acid sequence of SEQ ID NO: 22, 42, 62, 82, 102, 122, 142, 162, 182, 202, 222, 242, 262, 282, 302, 320, 338, 355, 369, 383, 397, 411, 425, 439, 453, 467, 481, and 495 (Table 2). The present disclosure also provides antibodies or antibody fragments (e.g., antigen binding fragments) that specifically bind to VP1, said antibodies or antibody fragments (e.g., antigen binding fragments) comprise a VL CDR having an amino acid sequence of any one of the VL CDRs listed in Table 2. In particular, the disclosure provides antibodies or antibody fragments (e.g., antigen binding fragments) that specifically bind to VP1, said antibodies or antibody fragments (e.g., antigen binding fragments) comprise (or alternatively, consist of) one, two, three or more VL CDRs having an amino acid sequence of any of the VL CDRs listed in Table 2.

Other antibodies or antibody fragments (e.g., antigen binding fragments) of the present disclosure include amino acids that have been mutated, yet have at least 60, 70, 80, 90 or 95 percent identity in the CDR regions with the CDR regions depicted in the sequences described in Table 2. In some aspects, it includes mutant amino acid sequences wherein no more than 1, 2, 3, 4 or 5 amino acids have been mutated in the CDR regions when compared with the CDR regions depicted in the sequence described in Table 2.

The present disclosure also provides nucleic acid sequences that encode VH, VL, the full length heavy chain, and the full length light chain of the antibodies that specifically bind to VP1. Such nucleic acid sequences can be optimized for expression in mammalian cells.

TABLE 2
anti-VP1 Antibodies

| | | |
|---|---|---|
| P8D11 | | |
| SEQ ID NO: 6 | HCDR1 | NYWMT |
| (Kabat) | | |
| SEQ ID NO: 7 | HCDR2 | NIKKDGSEKYYVDSVRG |
| (Kabat) | | |
| SEQ ID NO: 8 | HCDR3 | VRSGRYFALDD |
| (Kabat) | | |
| SEQ ID NO: 9 | HCDR1 | GFTFNNY |
| (Chothia) | | |
| SEQ ID NO: 10 | HCDR2 | KKDGSE |
| (Chothia) | | |
| SEQ ID NO: 11 | HCDR3 | VRSGRYFALDD |
| (Chothia) | | |
| SEQ ID NO: 12 | VH | QVQLVESGGTLVQPGGSLRLSCAASGFTFNNYWM |
| | | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSS |
| SEQ ID NO: 13 | DNA | CAGGTGCAGCTGGTGGAATCAGGCGGCACACTG |
| | VH | GTGCAGCCTGGCGGTAGCCTGAGACTGAGCTGCG |
| | | CTGCTAGTGGCTTCACCTTTAACAACTACTGGAT |
| | | GACCTGGGTTAGGCAGGCCCCTGGTAAAGGCCTC |
| | | GAGTGGGTGGCAAATATCAAGAAGGACGGTAGC |
| | | GAGAAGTACTACGTGGACTCAGTCAGAGGCCGG |
| | | TTCACTATCTCTAGGGATAACGCTAAGAATAGCC |
| | | TGTTCCTGCAGATGAACTCACTGAGGCCCGAGGA |
| | | TACCGCCGTCTACTTCTGTGCTACCGTCAGATCA |
| | | GGCCGCTACTTCGCCCTGGACGACTGGGGTCAAG |
| | | GCACACTGGTCACCGTGTCTAGC |
| SEQ ID NO: 14 | Heavy | QVQLVESGGTLVQPGGSLRLSCAASGFTFNNYWM |
| | Chain | TWVRQAPGKGLEWVANIKKDGSEKYYVDSVRGRF |
| | | TISRDNAKNSLFLQMNSLRPEDTAVYFCATVRSGR |
| | | YFALDDWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPS |
| | | NTKVDKRVEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| SEQ ID NO: 15 | DNA | CAGGTGCAGCTGGTGGAATCAGGCGGCACACTG |
| | Heavy | GTGCAGCCTGGCGGTAGCCTGAGACTGAGCTGC |
| | Chain | GCTGCTAGTGGCTTCACCTTTAACAACTACTGGA |
| | | TGACCTGGGTTAGGCAGGCCCCTGGTAAAGGCCT |
| | | CGAGTGGGTGGCAAATATCAAGAAGGACGGTAG |
| | | CGAGAAGTACTACGTGGACTCAGTCAGAGGCCG |
| | | GTTCACTATCTCTAGGGATAACGCTAAGAATAGC |
| | | CTGTTCCTGCAGATGAACTCACTGAGGCCCGAGG |
| | | ATACCGCCGTCTACTTCTGTGCTACCGTCAGATC |
| | | AGGCCGCTACTTCGCCCTGGACGACTGGGGTCAA |
| | | GGCACACTGGTCACCGTGTCTAGCGCTAGCACTA |
| | | AGGGCCCAAGTGTGTTTCCCCTGGCCCCCAGCAG |
| | | CAAGTCTACTTCCGGCGGAACTGCTGCCCTGGGT |
| | | TGCCTGGTGAAGGACTACTTCCCCGAGCCCGTGA |
| | | CAGTGTCCTGGAACTCTGGGGCTCTGACTTCCGG |
| | | CGTGCACACCTTCCCCGCCGTGCTGCAGAGCAGC |
| | | GGCCTGTACAGCCTGAGCAGCGTGGTGACAGTG |
| | | CCCTCCAGCTCTCTGGGAACCCAGACCTATATCT |
| | | GCAACGTGAACCACAAGCCCAGCAACACCAAGG |
| | | TGGACAAGAGAGTGGAGCCCAAGAGCTGCGACA |
| | | AGACCCACACCTGCCCCCCCTGCCCAGCTCCAGA |
| | | ACTGCTGGGAGGGCCTTCCGTGTTCCTGTTCCCC |
| | | CCCAAGCCCAAGGACACCCTGATGATCAGCAGG |
| | | ACCCCCGAGGTGACCTGCGTGGTGGTGGACGTGT |
| | | CCCACGAGGACCCAGAGGTGAAGTTCAACTGGT |
| | | ACGTGGACGGCGTGGAGGTGCACAACGCCAAGA |
| | | CCAAGCCCAGAGAGGAGCAGTACAACAGCACCT |
| | | ACAGGGTGGTGTCCGTGCTGACCGTGCTGCACCA |
| | | GGACTGGCTGAACGGCAAAGAATACAAGTGCAA |
| | | AGTCTCCAACAAGGCCCTGCCAGCCCCAATCGA |
| | | AAAGACAATCAGCAAGGCCAAGGGCCAGCCACG |
| | | GGAGCCCCAGGTGTACACCCTGCCCCCCAGCCG |
| | | GGAGGAGATGACCAAGAACCAGGTGTCCCTGAC |
| | | CTGTCTGGTGAAGGGCTTCTACCCCAGCGATATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAAGCTGA |
| | | CCGTGGACAAGTCCAGGTGGCAGCAGGGCAACG |
| | | TGTTCAGCTGCAGCGTGATGCACGAGGCCCTGCA |
| | | CAACCACTACACCCAGAAGTCCCTGAGCCTGAG |
| | | CCCCGGCAAG |
| SEQ ID NO: 16 | LCDR1 | GGDNIGSRPVH |
| (Kabat) | | |
| SEQ ID NO: 17 | LCDR2 | DDSNRPS |
| (Kabat) | | |
| SEQ ID NO: 18 | LCDR3 | QVWSSSTDHP |
| (Kabat) | | |
| SEQ ID NO: 19 | LCDR1 | DNIGSRP |
| (Chothia) | | |
| SEQ ID NO: 20 | LCDR2 | DDS |
| (Chothia) | | |
| SEQ ID NO: 21 | LCDR3 | WSSSTDH |
| (Chothia) | | |
| SEQ ID NO: 22 | VL | QSVLTQPPSVSVAPGKTARITCGGDNIGSRPVHWY |
| | | QQKPGQAPILVVYDDSNRPSGIPERFSGSNSGNTAT |
| | | LTISRVEAGDEADYYCQVWSSSTDHPFGGGTKVTV |
| | | L |
| SEQ ID NO: 23 | DNA VL | CAGTCAGTCCTGACTCAGCCCCCTAGCGTCAGCG |
| | | TGGCCCCTGGTAAAACCGCTAGAATCACCTGTGG |
| | | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTG |
| SEQ ID NO: 24 | Light | QSVLTQPPSVSVAPGKTARITCGGDNIGSRPVHWY |
| | Chain | QQKPGQAPILVVYDDSNRPSGIPERFSGSNSGNTAT |
| | | LTISRVEAGDEADYYCQVWSSSTDHPFGGGTKVTV |
| | | LGQPKAAPSVTLFPPSSEELQANKATLVCLISDFYP |
| | | GAVTVAWKADSSPVKAGVETTTPSKQSNNKYAAS |
| | | SYLSLTPEQWKSHRSYSCQVTHEGSTVEKTVAPTE |
| | | CS |
| SEQ ID NO: 25 | DNA | CAGTCAGTCCTGACTCAGCCCCCTAGCGTCAGCG |
| | Light | TGGCCCCTGGTAAAACCGCTAGAATCACCTGTGG |
| | Chain | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTGGGTCAACCTAAGGCTGCCC |
| | | CCAGCGTGACCCTGTTCCCCCCCAGCAGCGAGGA |
| | | GCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |
| P8D11A | | |
| SEQ ID NO: 26 | HCDR1 | NYWMT |
| (Kabat) | | |
| SEQ ID NO: 27 | HCDR2 | NIKKDGSEKYYVDSVRG |
| (Kabat) | | |
| SEQ ID NO: 28 | HCDR3 | VRSGRYFALDD |
| (Kabat) | | |
| SEQ ID NO: 29 | HCDR1 | GFTFSNY |
| (Chothia) | | |
| SEQ ID NO: 30 | HCDR2 | KKDGSE |
| (Chothia) | | |
| SEQ ID NO: 31 | HCDR3 | VRSGRYFALDD |
| (Chothia) | | |
| SEQ ID NO: 32 | VH | QVQLVESGGTLVQPGGSLRLSCAASGFTFSNYWM |
| | | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSS |
| SEQ ID NO: 33 | DNA VH | CAGGTGCAGCTGGTGGAATCAGGCGGCACACTG |
| | | GTGCAGCCTGGCGGTAGCCTGAGACTGAGCTGC |
| | | GCTGCTAGTGGCTTCACCTTCTCTAACTACTGGA |
| | | TGACCTGGGTCAGGCAGGCCCCTGGTAAAGGCC |
| | | TCGAGTGGGTGGCAAATATCAAGAAGGACGGTA |
| | | GCGAGAAGTACTACGTGGACTCAGTCAGAGGCC |
| | | GGTTCACTATCTCTAGGGATAACGCTAAGAATAG |
| | | CCTGTTCCTGCAGATGAACTCACTGAGGCCCGAG |
| | | GATACCGCCGTCTACTTCTGTGCTACCGTCAGAT |
| | | CAGGCCGCTACTTCGCCCTGGACGACTGGGGTCA |
| | | AGGCACACTGGTCACCGTGTCTAGC |
| SEQ ID NO: 34 | Heavy | QVQLVESGGTLVQPGGSLRLSCAASGFTFSNYWM |
| | Chain | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 35 | DNA | CAGGTGCAGCTGGTGGAATCAGGCGGCACACTG |
| | Heavy | GTGCAGCCTGGCGGTAGCCTGAGACTGAGCTGC |
| | Chain | GCTGCTAGTGGCTTCACCTTCTCTAACTACTGGA |
| | | TGACCTGGGTCAGGCAGGCCCCTGGTAAAGGCC |
| | | TCGAGTGGGTGGCAAATATCAAGAAGGACGGTA |
| | | GCGAGAAGTACTACGTGGACTCAGTCAGAGGCC |
| | | GGTTCACTATCTCTAGGGATAACGCTAAGAATAG |
| | | CCTGTTCCTGCAGATGAACTCACTGAGGCCCGAG |
| | | GATACCGCCGTCTACTTCTGTGCTACCGTCAGAT |
| | | CAGGCCGCTACTTCGCCCTGGACGACTGGGGTCA |
| | | AGGCACACTGGTCACCGTGTCTAGCGCTAGCACT |
| | | AAGGGCCCAAGTGTGTTTCCCCTGGCCCCCAGCA |
| | | GCAAGTCTACTTCCGGCGGAACTGCTGCCCTGGG |
| | | TTGCCTGGTGAAGGACTACTTCCCCGAGCCCGTG |
| | | ACAGTGTCCTGGAACTCTGGGGCTCTGACTTCCG |
| | | GCGTGCACACCTTCCCCGCCGTGCTGCAGAGCAG |
| | | CGGCCTGTACAGCCTGAGCAGCGTGGTGACAGT |
| | | GCCCTCCAGCTCTCTGGGAACCCAGACCTATATC |
| | | TGCAACGTGAACCACAAGCCCAGCAACACCAAG |
| | | GTGGACAAGAGAGTGGAGCCCAAGAGCTGCGAC |
| | | AAGACCCACACCTGCCCCCCCTGCCCAGCTCCAG |
| | | AACTGCTGGGAGGGCCTTCCGTGTTCCTGTTCCC |
| | | CCCCAAGCCCAAGGACACCCTGATGATCAGCAG |
| | | GACCCCCGAGGTGACCTGCGTGGTGGTGGACGT |
| | | GTCCCACGAGGACCCAGAGGTGAAGTTCAACTG |
| | | GTACGTGGACGGCGTGGAGGTGCACAACGCCAA |
| | | GACCAAGCCCAGAGAGGAGCAGTACAACAGCAC |
| | | CTACAGGGTGGTGTCCGTGCTGACCGTGCTGCAC |
| | | CAGGACTGGCTGAACGGCAAAGAATACAAGTGC |
| | | AAAGTCTCCAACAAGGCCCTGCCAGCCCCAATC |
| | | GAAAAGACAATCAGCAAGGCCAAGGGCCAGCCA |
| | | CGGGAGCCCCAGGTGTACACCCTGCCCCCCAGCC |
| | | GGGAGGAGATGACCAAGAACCAGGTGTCCCTGA |
| | | CCTGTCTGGTGAAGGGCTTCTACCCCAGCGATAT |
| | | CGCCGTGGAGTGGGAGAGCAACGGCCAGCCCGA |
| | | GAACAACTACAAGACCACCCCCCCAGTGCTGGA |
| | | CAGCGACGGCAGCTTCTTCCTGTACAGCAAGCTG |
| | | ACCGTGGACAAGTCCAGGTGGCAGCAGGGCAAC |
| | | GTGTTCAGCTGCAGCGTGATGCACGAGGCCCTGC |
| | | ACAACCACTACACCCAGAAGTCCCTGAGCCTGA |
| | | GCCCCGGCAAG |
| SEQ ID NO: 36 | LCDR1 | GGDNIGSRPVH |
| (Kabat) | | |
| SEQ ID NO: 37 | LCDR2 | DDSNRPS |
| (Kabat) | | |
| SEQ ID NO: 38 | LCDR3 | QVWSSSTDHP |
| (Kabat) | | |
| SEQ ID NO: 39 | LCDR1 | DNIGSRP |
| (Chothia) | | |
| SEQ ID NO: 40 | LCDR2 | DDS |
| (Chothia) | | |
| SEQ ID NO: 41 | LCDR3 | WSSSTDH |
| (Chothia) | | |
| SEQ ID NO: 42 | VL | QSVLTQPPSVSVAPGKTARITCGGDNIGSRPVHWY |
| | | QQKPGQAPILVVYDDSNRPSGIPERFSGSNSGNTAT |
| | | LTISRVEAGDEADYYCQVWSSSTDHPFGGGTKVTV |
| | | L |
| SEQ ID NO: 43 | DNA VL | CAGTCAGTCCTGACTCAGCCCCCTAGCGTCAGCG |
| | | TGGCCCCTGGTAAAACCGCTAGAATCACCTGTGG |
| | | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTG |
| SEQ ID NO: 44 | Light | QSVLTQPPSVSVAPGKTARITCGGDNIGSRPVHWY |
| | Chain | QQKPGQAPILVVYDDSNRPSGIPERFSGSNSGNTAT |
| | | LTISRVEAGDEADYYCQVWSSSTDHPFGGGTKVTV |
| | | LGQPKAAPSVTLFPPSSEELQANKATLVCLISDFYP |
| | | GAVTVAWKADSSPVKAGVETTTPSKQSNNKYAAS |
| | | SYLSLTPEQWKSHRSYSCQVTHEGSTVEKTVAPTE |
| | | CS |
| SEQ ID NO: 45 | DNA | CAGTCAGTCCTGACTCAGCCCCCTAGCGTCAGCG |
| | Light | TGGCCCCTGGTAAAACCGCTAGAATCACCTGTGG |
| | Chain | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTGGGTCAACCTAAGGCTGCCC |
| | | CCAGCGTGACCCTGTTCCCCCCCAGCAGCGAGGA |
| | | GCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |
| P8D11B | | |
| SEQ ID NO: 46 | HCDR1 | NYWMT |
| (Kabat) | | |
| SEQ ID NO: 47 | HCDR2 | NIKKDGSEKYYVDSVRG |
| (Kabat) | | |
| SEQ ID NO: 48 | HCDR3 | VRSGRYFALDD |
| (Kabat) | | |
| SEQ ID NO: 49 | HCDR1 | GFTFKNY |
| (Chothia) | | |
| SEQ ID NO: 50 | HCDR2 | KKDGSE |
| (Chothia) | | |
| SEQ ID NO: 51 | HCDR3 | VRSGRYFALDD |
| (Chothia) | | |
| SEQ ID NO: 52 | VH | QVQLVESGGTLVQPGGSLRLSCAASGFTFKNYWM |
| | | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSS |
| SEQ ID NO: 53 | DNA VH | CAGGTGCAGCTGGTGGAATCAGGCGGCACACTG |
| | | GTGCAGCCTGGCGGTAGCCTGAGACTGAGCTGC |
| | | GCTGCTAGTGGCTTCACCTTTAAGAACTACTGGA |
| | | TGACCTGGGTCAGGCAGGCCCCTGGTAAAGGCC |
| | | TCGAGTGGGTGGCAAATATCAAGAAGGACGGTA |
| | | GCGAGAAGTACTACGTGGACTCAGTCAGAGGCC |
| | | GGTTCACTATCTCTAGGGATAACGCTAAGAATAG |
| | | CCTGTTCCTGCAGATGAACTCACTGAGGCCCGAG |
| | | GATACCGCCGTCTACTTCTGTGCTACCGTCAGAT |
| | | CAGGCCGCTACTTCGCCCTGGACGACTGGGGTCA |
| | | AGGCACACTGGTCACCGTGTCTAGC |
| SEQ ID NO: 54 | Heavy | QVQLVESGGTLVQPGGSLRLSCAASGFTFKNYWM |
| | Chain | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 55 | DNA | CAGGTGCAGCTGGTGGAATCAGGCGGCACACTG |
| | Heavy | GTGCAGCCTGGCGGTAGCCTGAGACTGAGCTGC |
| | Chain | GCTGCTAGTGGCTTCACCTTTAAGAACTACTGGA |
| | | TGACCTGGGTCAGGCAGGCCCCTGGTAAAGGCC |
| | | TCGAGTGGGTGGCAAATATCAAGAAGGACGGTA |
| | | GCGAGAAGTACTACGTGGACTCAGTCAGAGGCC |
| | | GGTTCACTATCTCTAGGGATAACGCTAAGAATAG |
| | | CCTGTTCCTGCAGATGAACTCACTGAGGCCCGAG |
| | | GATACCGCCGTCTACTTCTGTGCTACCGTCAGAT |
| | | CAGGCCGCTACTTCGCCCTGGACGACTGGGGTCA |
| | | AGGCACACTGGTCACCGTGTCTAGCGCTAGCACT |
| | | AAGGGCCCAAGTGTGTTTCCCCTGGCCCCCAGCA |
| | | GCAAGTCTACTTCCGGCGGAACTGCTGCCCTGGG |
| | | TTGCCTGGTGAAGGACTACTTCCCCGAGCCCGTG |
| | | ACAGTGTCCTGGAACTCTGGGGCTCTGACTTCCG |
| | | GCGTGCACACCTTCCCCGCCGTGCTGCAGAGCAG |
| | | CGGCCTGTACAGCCTGAGCAGCGTGGTGACAGT |
| | | GCCCTCCAGCTCTCTGGGAACCCAGACCTATATC |
| | | TGCAACGTGAACCACAAGCCCAGCAACACCAAG |
| | | GTGGACAAGAGAGTGGAGCCCAAGAGCTGCGAC |
| | | AAGACCCACACCTGCCCCCCCTGCCCAGCTCCAG |
| | | AACTGCTGGGAGGGCCTTCCGTGTTCCTGTTCCC |
| | | CCCCAAGCCCAAGGACACCCTGATGATCAGCAG |
| | | GACCCCCGAGGTGACCTGCGTGGTGGTGGACGT |
| | | GTCCCACGAGGACCCAGAGGTGAAGTTCAACTG |
| | | GTACGTGGACGGCGTGGAGGTGCACAACGCCAA |
| | | GACCAAGCCCAGAGAGGAGCAGTACAACAGCAC |
| | | CTACAGGGTGGTGTCCGTGCTGACCGTGCTGCAC |
| | | CAGGACTGGCTGAACGGCAAAGAATACAAGTGC |
| | | AAAGTCTCCAACAAGGCCCTGCCAGCCCCAATC |
| | | GAAAAGACAATCAGCAAGGCCAAGGGCCAGCCA |
| | | CGGGAGCCCCAGGTGTACACCCTGCCCCCCAGCC |
| | | GGGAGGAGATGACCAAGAACCAGGTGTCCCTGA |
| | | CCTGTCTGGTGAAGGGCTTCTACCCCAGCGATAT |
| | | CGCCGTGGAGTGGGAGAGCAACGGCCAGCCCGA |
| | | GAACAACTACAAGACCACCCCCCCAGTGCTGGA |
| | | CAGCGACGGCAGCTTCTTCCTGTACAGCAAGCTG |
| | | ACCGTGGACAAGTCCAGGTGGCAGCAGGGCAAC |
| | | GTGTTCAGCTGCAGCGTGATGCACGAGGCCCTGC |
| | | ACAACCACTACACCCAGAAGTCCCTGAGCCTGA |
| | | GCCCCGGCAAG |
| SEQ ID NO: 56 | LCDR1 | GGDNIGSRPVH |
| (Kabat) | | |
| SEQ ID NO: 57 | LCDR2 | DDSNRPS |
| (Kabat) | | |
| SEQ ID NO: 58 | LCDR3 | QVWSSSTDHP |
| (Kabat) | | |
| SEQ ID NO: 59 | LCDR1 | DNIGSRP |
| (Chothia) | | |
| SEQ ID NO: 60 | LCDR2 | DDS |
| (Chothia) | | |
| SEQ ID NO: 61 | LCDR3 | WSSSTDH |
| (Chothia) | | |
| SEQ ID NO: 62 | VL | QSVLTQPPSVSVAPGKTARITCGGDNIGSRPVHWY |
| | | QQKPGQAPILVVYDDSNRPSGIPERFSGSNSGNTAT |
| | | LTISRVEAGDEADYYCQVWSSSTDHPFGGGTKVTV |
| | | L |
| SEQ ID NO: 63 | DNA VL | CAGTCAGTCCTGACTCAGCCCCCTAGCGTCAGCG |
| | | TGGCCCCTGGTAAAACCGCTAGAATCACCTGTGG |
| | | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTG |
| SEQ ID NO: 64 | Light | QSVLTQPPSVSVAPGKTARITCGGDNIGSRPVHWY |
| | Chain | QQKPGQAPILVVYDDSNRPSGIPERFSGSNSGNTAT |
| | | LTISRVEAGDEADYYCQVWSSSTDHPFGGGTKVTV |
| | | LGQPKAAPSVTLFPPSSEELQANKATLVCLISDFYP |
| | | GAVTVAWKADSSPVKAGVETTTPSKQSNNKYAAS |
| | | SYLSLTPEQWKSHRSYSCQVTHEGSTVEKTVAPTE |
| | | CS |
| SEQ ID NO: 65 | DNA | CAGTCAGTCCTGACTCAGCCCCCTAGCGTCAGCG |
| | Light | TGGCCCCTGGTAAAACCGCTAGAATCACCTGTGG |
| | Chain | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTGGGTCAACCTAAGGCTGCCC |
| | | CCAGCGTGACCCTGTTCCCCCCCAGCAGCGAGGA |
| | | GCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |
| P8D11C | | |
| SEQ ID NO: 66 | HCDR1 | NYWMT |
| (Kabat) | | |
| SEQ ID NO: 67 | HCDR2 | NIKKDGSEKYYVDSVRG |
| (Kabat) | | |
| SEQ ID NO: 68 | HCDR3 | VRSGRYFALDD |
| (Kabat) | | |
| SEQ ID NO: 69 | HCDR1 | GFTFQNY |
| (Chothia) | | |
| SEQ ID NO: 70 | HCDR2 | KKDGSE |
| (Chothia) | | |
| SEQ ID NO: 71 | HCDR3 | VRSGRYFALDD |
| (Chothia) | | |
| SEQ ID NO: 72 | VH | QVQLVESGGTLVQPGGSLRLSCAASGFTFQNYWM |
| | | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSS |
| SEQ ID NO: 73 | DNA VH | CAGGTGCAGCTGGTGGAATCAGGCGGCACACTG |
| | | GTGCAGCCTGGCGGTAGCCTGAGACTGAGCTGC |
| | | GCCGCTAGTGGATTCACCTTTCAGAACTACTGGA |
| | | TGACCTGGGTCAGACAGGCCCCTGGTAAAGGCC |
| | | TCGAGTGGGTGGCAAATATCAAGAAGGACGGTA |
| | | GCGAGAAGTACTACGTGGACTCAGTCAGAGGCC |
| | | GGTTCACTATCTCTAGGGATAACGCTAAGAATAG |
| | | CCTGTTCCTGCAGATGAACTCACTGAGGCCCGAG |
| | | GATACCGCCGTCTACTTCTGTGCTACCGTCAGAT |
| | | CAGGCCGCTACTTCGCCCTGGACGACTGGGGTCA |
| | | AGGCACACTGGTCACCGTGTCTAGC |
| SEQ ID NO: 74 | Heavy | QVQLVESGGTLVQPGGSLRLSCAASGFTFQNYWM |
| | Chain | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 75 | DNA | CAGGTGCAGCTGGTGGAATCAGGCGGCACACTG |
| | Heavy | GTGCAGCCTGGCGGTAGCCTGAGACTGAGCTGC |
| | Chain | GCCGCTAGTGGATTCACCTTTCAGAACTACTGGA |
| | | TGACCTGGGTCAGACAGGCCCCTGGTAAAGGCC |
| | | TCGAGTGGGTGGCAAATATCAAGAAGGACGGTA |
| | | GCGAGAAGTACTACGTGGACTCAGTCAGAGGCC |
| | | GGTTCACTATCTCTAGGGATAACGCTAAGAATAG |
| | | CCTGTTCCTGCAGATGAACTCACTGAGGCCCGAG |
| | | GATACCGCCGTCTACTTCTGTGCTACCGTCAGAT |
| | | CAGGCCGCTACTTCGCCCTGGACGACTGGGGTCA |
| | | AGGCACACTGGTCACCGTGTCTAGCGCTAGCACT |
| | | AAGGGCCCAAGTGTGTTTCCCCTGGCCCCCAGCA |
| | | GCAAGTCTACTTCCGGCGGAACTGCTGCCCTGGG |
| | | TTGCCTGGTGAAGGACTACTTCCCCGAGCCCGTG |
| | | ACAGTGTCCTGGAACTCTGGGGCTCTGACTTCCG |
| | | GCGTGCACACCTTCCCCGCCGTGCTGCAGAGCAG |
| | | CGGCCTGTACAGCCTGAGCAGCGTGGTGACAGT |
| | | GCCCTCCAGCTCTCTGGGAACCCAGACCTATATC |
| | | TGCAACGTGAACCACAAGCCCAGCAACACCAAG |
| | | GTGGACAAGAGAGTGGAGCCCAAGAGCTGCGAC |
| | | AAGACCCACACCTGCCCCCCCTGCCCAGCTCCAG |
| | | AACTGCTGGGAGGGCCTTCCGTGTTCCTGTTCCC |
| | | CCCCAAGCCCAAGGACACCCTGATGATCAGCAG |
| | | GACCCCCGAGGTGACCTGCGTGGTGGTGGACGT |
| | | GTCCCACGAGGACCCAGAGGTGAAGTTCAACTG |
| | | GTACGTGGACGGCGTGGAGGTGCACAACGCCAA |
| | | GACCAAGCCCAGAGAGGAGCAGTACAACAGCAC |
| | | CTACAGGGTGGTGTCCGTGCTGACCGTGCTGCAC |
| | | CAGGACTGGCTGAACGGCAAAGAATACAAGTGC |
| | | AAAGTCTCCAACAAGGCCCTGCCAGCCCCAATC |
| | | GAAAAGACAATCAGCAAGGCCAAGGGCCAGCCA |
| | | CGGGAGCCCCAGGTGTACACCCTGCCCCCCAGCC |
| | | GGGAGGAGATGACCAAGAACCAGGTGTCCCTGA |
| | | CCTGTCTGGTGAAGGGCTTCTACCCCAGCGATAT |
| | | CGCCGTGGAGTGGGAGAGCAACGGCCAGCCCGA |
| | | GAACAACTACAAGACCACCCCCCCAGTGCTGGA |
| | | CAGCGACGGCAGCTTCTTCCTGTACAGCAAGCTG |
| | | ACCGTGGACAAGTCCAGGTGGCAGCAGGGCAAC |
| | | GTGTTCAGCTGCAGCGTGATGCACGAGGCCCTGC |
| | | ACAACCACTACACCCAGAAGTCCCTGAGCCTGA |
| | | GCCCCGGCAAG |
| SEQ ID NO: 76 | LCDR1 | GGDNIGSRPVH |
| (Kabat) | | |
| SEQ ID NO: 77 | LCDR2 | DDSNRPS |
| (Kabat) | | |
| SEQ ID NO: 78 | LCDR3 | QVWSSSTDHP |
| (Kabat) | | |
| SEQ ID NO: 79 | LCDR1 | DNIGSRP |
| (Chothia) | | |
| SEQ ID NO: 80 | LCDR2 | DDS |
| (Chothia) | | |
| SEQ ID NO: 81 | LCDR3 | WSSSTDH |
| (Chothia) | | |
| SEQ ID NO: 82 | VL | QSVLTQPPSVSVAPGKTARITCGGDNIGSRPVHWY |
| | | QQKPGQAPILVVYDDSNRPSGIPERFSGSNSGNTAT |
| | | LTISRVEAGDEADYYCQVWSSSTDHPFGGGTKVTV |
| | | L |
| SEQ ID NO: 83 | DNA VL | CAGTCAGTCCTGACTCAGCCCCCTAGCGTCAGCG |
| | | TGGCCCCTGGTAAAACCGCTAGAATCACCTGTGG |
| | | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTG |
| SEQ ID NO: 84 | Light | QSVLTQPPSVSVAPGKTARITCGGDNIGSRPVHWY |
| | Chain | QQKPGQAPILVVYDDSNRPSGIPERFSGSNSGNTAT |
| | | LTISRVEAGDEADYYCQVWSSSTDHPFGGGTKVTV |
| | | LGQPKAAPSVTLFPPSSEELQANKATLVCLISDFYP |
| | | GAVTVAWKADSSPVKAGVETTTPSKQSNNKYAAS |
| | | SYLSLTPEQWKSHRSYSCQVTHEGSTVEKTVAPTE |
| | | CS |
| SEQ ID NO: 85 | DNA | CAGTCAGTCCTGACTCAGCCCCCTAGCGTCAGCG |
| | Light | TGGCCCCTGGTAAAACCGCTAGAATCACCTGTGG |
| | Chain | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTGGGTCAACCTAAGGCTGCCC |
| | | CCAGCGTGACCCTGTTCCCCCCCAGCAGCGAGGA |
| | | GCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |
| P8D11D | | |
| SEQ ID NO: 86 | HCDR1 | NYWMT |
| (Kabat) | | |
| SEQ ID NO: 87 | HCDR2 | NIKKDGSEKYYVDSVRG |
| (Kabat) | | |
| SEQ ID NO: 88 | HCDR3 | VRSGRYFALDD |
| (Kabat) | | |
| SEQ ID NO: 89 | HCDR1 | GFTFNNY |
| (Chothia) | | |
| SEQ ID NO: 90 | HCDR2 | KKDGSE |
| (Chothia) | | |
| SEQ ID NO: 91 | HCDR3 | VRSGRYFALDD |
| (Chothia) | | |
| SEQ ID NO: 92 | VH | QVQLQESGPGLVQPGGSLRLSCAASGFTFNNYWM |
| | | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSS |
| SEQ ID NO: 93 | DNA VH | CAGGTGCAGCTGCAGGAATCAGGCCCAGGACTG |
| | | GTGCAGCCTGGCGGTAGCCTGAGACTGAGCTGC |
| | | GCTGCTAGTGGCTTCACCTTTAACAACTACTGGA |
| | | TGACCTGGGTCCGCCAGGCCCCTGGCAAAGGCCT |
| | | GGAGTGGGTGGCAAATATCAAGAAGGACGGTAG |
| | | CGAGAAGTACTACGTGGACTCAGTCAGAGGCCG |
| | | GTTCACTATCTCTAGGGATAACGCTAAGAATAGC |
| | | CTGTTCCTGCAGATGAACTCACTGAGGCCCGAGG |
| | | ATACCGCCGTCTACTTCTGTGCTACCGTCAGATC |
| | | AGGCCGCTACTTCGCCCTGGACGACTGGGGCCA |
| | | GGGCACCCTGGTCACCGTGTCTTCC |
| SEQ ID NO: 94 | Heavy | QVQLQESGPGLVQPGGSLRLSCAASGFTFNNYWM |
| | Chain | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 95 | DNA | CAGGTGCAGCTGCAGGAATCAGGCCCAGGACTG |
| | Heavy | GTGCAGCCTGGCGGTAGCCTGAGACTGAGCTGC |
| | Chain | GCTGCTAGTGGCTTCACCTTTAACAACTACTGGA |
| | | TGACCTGGGTCCGCCAGGCCCCTGGCAAAGGCCT |
| | | GGAGTGGGTGGCAAATATCAAGAAGGACGGTAG |
| | | CGAGAAGTACTACGTGGACTCAGTCAGAGGCCG |
| | | GTTCACTATCTCTAGGGATAACGCTAAGAATAGC |
| | | CTGTTCCTGCAGATGAACTCACTGAGGCCCGAGG |
| | | ATACCGCCGTCTACTTCTGTGCTACCGTCAGATC |
| | | AGGCCGCTACTTCGCCCTGGACGACTGGGGCCA |
| | | GGGCACCCTGGTCACCGTGTCTTCCGCTAGCACT |
| | | AAGGGCCCAAGTGTGTTTCCCCTGGCCCCCAGCA |
| | | GCAAGTCTACTTCCGGCGGAACTGCTGCCCTGGG |
| | | TTGCCTGGTGAAGGACTACTTCCCCGAGCCCGTG |
| | | ACAGTGTCCTGGAACTCTGGGGCTCTGACTTCCG |
| | | GCGTGCACACCTTCCCCGCCGTGCTGCAGAGCAG |
| | | CGGCCTGTACAGCCTGAGCAGCGTGGTGACAGT |
| | | GCCCTCCAGCTCTCTGGGAACCCAGACCTATATC |
| | | TGCAACGTGAACCACAAGCCCAGCAACACCAAG |
| | | GTGGACAAGAGAGTGGAGCCCAAGAGCTGCGAC |
| | | AAGACCCACACCTGCCCCCCCTGCCCAGCTCCAG |
| | | AACTGCTGGGAGGGCCTTCCGTGTTCCTGTTCCC |
| | | CCCCAAGCCCAAGGACACCCTGATGATCAGCAG |
| | | GACCCCCGAGGTGACCTGCGTGGTGGTGGACGT |
| | | GTCCCACGAGGACCCAGAGGTGAAGTTCAACTG |
| | | GTACGTGGACGGCGTGGAGGTGCACAACGCCAA |
| | | GACCAAGCCCAGAGAGGAGCAGTACAACAGCAC |
| | | CTACAGGGTGGTGTCCGTGCTGACCGTGCTGCAC |
| | | CAGGACTGGCTGAACGGCAAAGAATACAAGTGC |
| | | AAAGTCTCCAACAAGGCCCTGCCAGCCCCAATC |
| | | GAAAAGACAATCAGCAAGGCCAAGGGCCAGCCA |
| | | CGGGAGCCCCAGGTGTACACCCTGCCCCCCAGCC |
| | | GGGAGGAGATGACCAAGAACCAGGTGTCCCTGA |
| | | CCTGTCTGGTGAAGGGCTTCTACCCCAGCGATAT |
| | | CGCCGTGGAGTGGGAGAGCAACGGCCAGCCCGA |
| | | GAACAACTACAAGACCACCCCCCCAGTGCTGGA |
| | | CAGCGACGGCAGCTTCTTCCTGTACAGCAAGCTG |
| | | ACCGTGGACAAGTCCAGGTGGCAGCAGGGCAAC |
| | | GTGTTCAGCTGCAGCGTGATGCACGAGGCCCTGC |
| | | ACAACCACTACACCCAGAAGTCCCTGAGCCTGA |
| | | GCCCCGGCAAG |
| SEQ ID NO: 96 | LCDR1 | GGDNIGSRPVH |
| (Kabat) | | |
| SEQ ID NO: 97 | LCDR2 | DDSNRPS |
| (Kabat) | | |
| SEQ ID NO: 98 | LCDR3 | QVWSSSTDHP |
| (Kabat) | | |
| SEQ ID NO: 99 | LCDR1 | DNIGSRP |
| (Chothia) | | |
| SEQ ID NO: 100 | LCDR2 | DDS |
| (Chothia) | | |
| SEQ ID NO: 101 | LCDR3 | WSSSTDH |
| (Chothia) | | |
| SEQ ID NO: 102 | VL | QSVLTQPPSVSVAPGKTARITCGGDNIGSRPVHWY |
| | | QQKPGQAPILVVYDDSNRPSGIPERFSGSNSGNTAT |
| | | LTISRVEAGDEADYYCQVWSSSTDHPFGGGTKVTV |
| | | L |
| SEQ ID NO: 103 | DNA VL | CAGTCAGTCCTGACTCAGCCCCCTAGCGTCAGCG |
| | | TGGCCCCTGGTAAAACCGCTAGAATCACCTGTGG |
| | | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTG |
| SEQ ID NO: 104 | Light | QSVLTQPPSVSVAPGKTARITCGGDNIGSRPVHWY |
| | Chain | QQKPGQAPILVVYDDSNRPSGIPERFSGSNSGNTAT |
| | | LTISRVEAGDEADYYCQVWSSSTDHPFGGGTKVTV |
| | | LGQPKAAPSVTLFPPSSEELQANKATLVCLISDFYP |
| | | GAVTVAWKADSSPVKAGVETTTPSKQSNNKYAAS |
| | | SYLSLTPEQWKSHRSYSCQVTHEGSTVEKTVAPTE |
| | | CS |
| SEQ ID NO: 105 | DNA | CAGTCAGTCCTGACTCAGCCCCCTAGCGTCAGCG |
| | Light | TGGCCCCTGGTAAAACCGCTAGAATCACCTGTGG |
| | Chain | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTGGGTCAACCTAAGGCTGCCC |
| | | CCAGCGTGACCCTGTTCCCCCCCAGCAGCGAGGA |
| | | GCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |
| P8D11E | | |
| SEQ ID NO: 106 | HCDR1 | NYWMT |
| (Kabat) | | |
| SEQ ID NO: 107 | HCDR2 | NIKKDGSEKYYVDSVRG |
| (Kabat) | | |
| SEQ ID NO: 108 | HCDR3 | VRSGRYFALDD |
| (Kabat) | | |
| SEQ ID NO: 109 | HCDR1 | GFTFNNY |
| (Chothia) | | |
| SEQ ID NO: 110 | HCDR2 | KKDGSE |
| (Chothia) | | |
| SEQ ID NO: 111 | HCDR3 | VRSGRYFALDD |
| (Chothia) | | |
| SEQ ID NO: 112 | VH | QVQLVESGGGLVQPGGSLRLSCAASGFTFNNYWM |
| | | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSS |
| SEQ ID NO: 113 | DNA VH | CAGGTGCAGCTGGTGGAATCAGGCGGCGGACTG |
| | | GTGCAGCCTGGCGGTAGCCTGAGACTGAGCTGC |
| | | GCTGCTAGTGGCTTCACCTTTAACAACTACTGGA |
| | | TGACCTGGGTTAGGCAGGCCCCTGGTAAAGGCCT |
| | | CGAGTGGGTGGCAAATATCAAGAAGGACGGTAG |
| | | CGAGAAGTACTACGTGGACTCAGTCAGAGGCCG |
| | | GTTCACTATCTCTAGGGATAACGCTAAGAATAGC |
| | | CTGTTCCTGCAGATGAACTCACTGAGGCCCGAGG |
| | | ATACCGCCGTCTACTTCTGTGCTACCGTCAGATC |
| | | AGGCCGCTACTTCGCCCTGGACGACTGGGGTCAA |
| | | GGCACACTGGTCACCGTGTCTAGC |
| SEQ ID NO: 114 | Heavy | QVQLVESGGGLVQPGGSLRLSCAASGFTENNYWM |
| | Chain | TWVRQAPGKGLEWVANIKKDGSEKYYVDSVRGR |
| | | FTISRDNAKNSLFLQMNSLRPEDTAVYFCATVRSG |
| | | RYFALDDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKEN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 115 | DNA | CAGGTGCAGCTGGTGGAATCAGGCGGCGGACTG |
| | Heavy | GTGCAGCCTGGCGGTAGCCTGAGACTGAGCTGC |
| | Chain | GCTGCTAGTGGCTTCACCTTTAACAACTACTGGA |
| | | TGACCTGGGTTAGGCAGGCCCCTGGTAAAGGCCT |
| | | CGAGTGGGTGGCAAATATCAAGAAGGACGGTAG |
| | | CGAGAAGTACTACGTGGACTCAGTCAGAGGCCG |
| | | GTTCACTATCTCTAGGGATAACGCTAAGAATAGC |
| | | CTGTTCCTGCAGATGAACTCACTGAGGCCCGAGG |
| | | ATACCGCCGTCTACTTCTGTGCTACCGTCAGATC |
| | | AGGCCGCTACTTCGCCCTGGACGACTGGGGTCAA |
| | | GGCACACTGGTCACCGTGTCTAGCGCTAGCACTA |
| | | AGGGCCCAAGTGTGTTTCCCCTGGCCCCCAGCAG |
| | | CAAGTCTACTTCCGGCGGAACTGCTGCCCTGGGT |
| | | TGCCTGGTGAAGGACTACTTCCCCGAGCCCGTGA |
| | | CAGTGTCCTGGAACTCTGGGGCTCTGACTTCCGG |
| | | CGTGCACACCTTCCCCGCCGTGCTGCAGAGCAGC |
| | | GGCCTGTACAGCCTGAGCAGCGTGGTGACAGTG |
| | | CCCTCCAGCTCTCTGGGAACCCAGACCTATATCT |
| | | GCAACGTGAACCACAAGCCCAGCAACACCAAGG |
| | | TGGACAAGAGAGTGGAGCCCAAGAGCTGCGACA |
| | | AGACCCACACCTGCCCCCCCTGCCCAGCTCCAGA |
| | | ACTGCTGGGAGGGCCTTCCGTGTTCCTGTTCCCC |
| | | CCCAAGCCCAAGGACACCCTGATGATCAGCAGG |
| | | ACCCCCGAGGTGACCTGCGTGGTGGTGGACGTGT |
| | | CCCACGAGGACCCAGAGGTGAAGTTCAACTGGT |
| | | ACGTGGACGGCGTGGAGGTGCACAACGCCAAGA |
| | | CCAAGCCCAGAGAGGAGCAGTACAACAGCACCT |
| | | ACAGGGTGGTGTCCGTGCTGACCGTGCTGCACCA |
| | | GGACTGGCTGAACGGCAAAGAATACAAGTGCAA |
| | | AGTCTCCAACAAGGCCCTGCCAGCCCCAATCGA |
| | | AAAGACAATCAGCAAGGCCAAGGGCCAGCCACG |
| | | GGAGCCCCAGGTGTACACCCTGCCCCCCAGCCG |
| | | GGAGGAGATGACCAAGAACCAGGTGTCCCTGAC |
| | | CTGTCTGGTGAAGGGCTTCTACCCCAGCGATATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAAGCTGA |
| | | CCGTGGACAAGTCCAGGTGGCAGCAGGGCAACG |
| | | TGTTCAGCTGCAGCGTGATGCACGAGGCCCTGCA |
| | | CAACCACTACACCCAGAAGTCCCTGAGCCTGAG |
| | | CCCCGGCAAG |
| SEQ ID NO: 116 | LCDR1 | GGDNIGSRPVH |
| (Kabat) | | |
| SEQ ID NO: 117 | LCDR2 | DDSNRPS |
| (Kabat) | | |
| SEQ ID NO: 118 | LCDR3 | QVWSSSTDHP |
| (Kabat) | | |
| SEQ ID NO: 119 | LCDR1 | DNIGSRP |
| (Chothia) | | |
| SEQ ID NO: 120 | LCDR2 | DDS |
| (Chothia) | | |
| SEQ ID NO: 121 | LCDR3 | WSSSTDH |
| (Chothia) | | |
| SEQ ID NO: 122 | VL | QSVLTQPPSVSVAPGKTARITCGGDNIGSRPVHWY |
| | | QQKPGQAPILVVYDDSNRPSGIPERFSGSNSGNTAT |
| | | LTISRVEAGDEADYYCQVWSSSTDHPFGGGTKVTV |
| | | L |
| SEQ ID NO: 123 | DNA VL | CAGTCAGTCCTGACTCAGCCCCCTAGCGTCAGCG |
| | | TGGCCCCTGGTAAAACCGCTAGAATCACCTGTGG |
| | | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTG |
| SEQ ID NO: 124 | Light | QSVLTQPPSVSVAPGKTARITCGGDNIGSRPVHWY |
| | Chain | QQKPGQAPILVVYDDSNRPSGIPERFSGSNSGNTAT |
| | | LTISRVEAGDEADYYCQVWSSSTDHPFGGGTKVTV |
| | | LGQPKAAPSVTLFPPSSEELQANKATLVCLISDFYP |
| | | GAVTVAWKADSSPVKAGVETTTPSKQSNNKYAAS |
| | | SYLSLTPEQWKSHRSYSCQVTHEGSTVEKTVAPTE |
| | | CS |
| SEQ ID NO: 125 | DNA | CAGTCAGTCCTGACTCAGCCCCCTAGCGTCAGCG |
| | Light | TGGCCCCTGGTAAAACCGCTAGAATCACCTGTGG |
| | Chain | CGGCGATAATATCGGCTCTAGGCCCGTGCACTGG |
| | | TATCAGCAGAAGCCCGGTCAAGCCCCTATCCTGG |
| | | TGGTCTACGACGACTCTAATAGACCTAGCGGAAT |
| | | CCCCGAGCGGTTTAGCGGCTCTAATTCTGGTAAT |
| | | ACCGCTACCCTGACTATCTCTAGGGTGGAAGCCG |
| | | GCGACGAGGCCGACTACTACTGTCAAGTCTGGTC |
| | | TAGCTCTACCGATCACCCCTTCGGCGGAGGCACT |
| | | AAGGTTACAGTGCTGGGTCAACCTAAGGCTGCCC |
| | | CCAGCGTGACCCTGTTCCCCCCCAGCAGCGAGGA |
| | | GCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |
| P165E2 | | |
| SEQ ID NO: 126 | HCDR1 | RDYWT |
| (Kabat) | | |
| SEQ ID NO: 127 | HCDR2 | NIYYSGSTNYNPSLKS |
| (Kabat) | | |
| SEQ ID NO: 128 | HCDR3 | VPGCSSTSCIDGWFDP |
| (Kabat) | | |
| SEQ ID NO: 129 | HCDR1 | GGSISRD |
| (Chothia) | | |
| SEQ ID NO: 130 | HCDR2 | YYSGS |
| (Chothia) | | |
| SEQ ID NO: 131 | HCDR3 | VPGCSSTSCIDGWFDP |
| (Chothia) | | |
| SEQ ID NO: 132 | VH | QVQLQESGPGLVKPSETLSLTCTVSGGSISRDYWT |
| | | WVRQPPGEGLEWIGNIYYSGSTNYNPSLKSRVTISV |
| | | AASKKQFSLKLTSVTAADTAVYYCARVPGCSSTSC |
| | | IDGWFDPWGQGILVTVSS |
| SEQ ID NO: 133 | DNA VH | CAGGTGCAGCTGCAAGAATCAGGCCCTGGCCTG |
| | | GTCAAGCCTAGCGAGACACTGAGCCTGACCTGC |
| | | ACCGTCAGCGGCGGCTCTATCTCTAGGGACTACT |
| | | GGACCTGGGTCCGACAGCCTCCTGGCGAGGGCC |
| | | TCGAGTGGATCGGTAATATCTACTATAGCGGCTC |
| | | TACTAACTATAACCCTAGCCTGAAGTCTAGGGTC |
| | | ACAATTAGCGTGGCCGCCTCTAAGAAGCAGTTTA |
| | | GCCTGAAGCTGACTAGCGTGACCGCCGCTGACA |
| | | CCGCCGTCTACTACTGCGCTAGAGTGCCCGGCTG |
| | | CTCTAGCACTAGCTGTATCGACGGCTGGTTTGAC |
| | | CCTTGGGGTCAAGGGATCCTGGTCACCGTGTCTA |
| | | GC |
| SEQ ID NO: 134 | Heavy | QVQLQESGPGLVKPSETLSLTCTVSGGSISRDYWT |
| | Chain | WVRQPPGEGLEWIGNIYYSGSTNYNPSLKSRVTISV |
| | | AASKKQFSLKLTSVTAADTAVYYCARVPGCSSTSC |
| | | IDGWFDPWGQGILVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKRVEPKSCDKTHTCPPCPAPELLGGPSVFL |
| | | FPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPGK |
| SEQ ID NO: 135 | DNA | CAGGTGCAGCTGCAAGAATCAGGCCCTGGCCTG |
| | Heavy | GTCAAGCCTAGCGAGACACTGAGCCTGACCTGC |
| | Chain | ACCGTCAGCGGCGGCTCTATCTCTAGGGACTACT |
| | | GGACCTGGGTCCGACAGCCTCCTGGCGAGGGCC |
| | | TCGAGTGGATCGGTAATATCTACTATAGCGGCTC |
| | | TACTAACTATAACCCTAGCCTGAAGTCTAGGGTC |
| | | ACAATTAGCGTGGCCGCCTCTAAGAAGCAGTTTA |
| | | GCCTGAAGCTGACTAGCGTGACCGCCGCTGACA |
| | | CCGCCGTCTACTACTGCGCTAGAGTGCCCGGCTG |
| | | CTCTAGCACTAGCTGTATCGACGGCTGGTTTGAC |
| | | CCTTGGGGTCAAGGGATCCTGGTCACCGTGTCTA |
| | | GCGCTAGCACTAAGGGCCCAAGTGTGTTTCCCCT |
| | | GGCCCCCAGCAGCAAGTCTACTTCCGGCGGAACT |
| | | GCTGCCCTGGGTTGCCTGGTGAAGGACTACTTCC |
| | | CCGAGCCCGTGACAGTGTCCTGGAACTCTGGGGC |
| | | TCTGACTTCCGGCGTGCACACCTTCCCCGCCGTG |
| | | CTGCAGAGCAGCGGCCTGTACAGCCTGAGCAGC |
| | | GTGGTGACAGTGCCCTCCAGCTCTCTGGGAACCC |
| | | AGACCTATATCTGCAACGTGAACCACAAGCCCA |
| | | GCAACACCAAGGTGGACAAGAGAGTGGAGCCCA |
| | | AGAGCTGCGACAAGACCCACACCTGCCCCCCCT |
| | | GCCCAGCTCCAGAACTGCTGGGAGGGCCTTCCGT |
| | | GTTCCTGTTCCCCCCCAAGCCCAAGGACACCCTG |
| | | ATGATCAGCAGGACCCCCGAGGTGACCTGCGTG |
| | | GTGGTGGACGTGTCCCACGAGGACCCAGAGGTG |
| | | AAGTTCAACTGGTACGTGGACGGCGTGGAGGTG |
| | | CACAACGCCAAGACCAAGCCCAGAGAGGAGCAG |
| | | TACAACAGCACCTACAGGGTGGTGTCCGTGCTGA |
| | | CCGTGCTGCACCAGGACTGGCTGAACGGCAAAG |
| | | AATACAAGTGCAAAGTCTCCAACAAGGCCCTGC |
| | | CAGCCCCAATCGAAAAGACAATCAGCAAGGCCA |
| | | AGGGCCAGCCACGGGAGCCCCAGGTGTACACCC |
| | | TGCCCCCCAGCCGGGAGGAGATGACCAAGAACC |
| | | AGGTGTCCCTGACCTGTCTGGTGAAGGGCTTCTA |
| | | CCCCAGCGATATCGCCGTGGAGTGGGAGAGCAA |
| | | CGGCCAGCCCGAGAACAACTACAAGACCACCCC |
| | | CCCAGTGCTGGACAGCGACGGCAGCTTCTTCCTG |
| | | TACAGCAAGCTGACCGTGGACAAGTCCAGGTGG |
| | | CAGCAGGGCAACGTGTTCAGCTGCAGCGTGATG |
| | | CACGAGGCCCTGCACAACCACTACACCCAGAAG |
| | | TCCCTGAGCCTGAGCCCCGGCAAG |
| SEQ ID NO: 136 | LCDR1 | SGSSSNIGNTYVS |
| (Kabat) | | |
| SEQ ID NO: 137 | LCDR2 | DNNKRPS |
| (Kabat) | | |
| SEQ ID NO: 138 | LCDR3 | GTWDSSLSAWV |
| (Kabat) | | |
| SEQ ID NO: 139 | LCDR1 | SSSNIGNTY |
| (Chothia) | | |
| SEQ ID NO: 140 | LCDR2 | DNN |
| (Chothia) | | |
| SEQ ID NO: 141 | LCDR3 | WDSSLSAW |
| (Chothia) | | |
| SEQ ID NO: 142 | VL | QSVLTQPPSLSAAPGQRVTISCSGSSSNIGNTYVSW |
| | | YQQLPGTAPKLLIYDNNKRPSGIPGRFSGSKSGTSA |
| | | TLGITGLQTGDEAAYYCGTWDSSLSAWVFGGGTR |
| | | LTVL |
| SEQ ID NO: 143 | DNA VL | CAGTCAGTCCTGACTCAGCCCCCTAGCCTGAGCG |
| | | CCGCTCCCGGTCAAAGAGTGACTATTAGCTGTAG |
| | | CGGCTCTAGCTCTAATATCGGTAATACCTACGTC |
| | | AGCTGGTATCAGCAGCTGCCCGGCACCGCCCCTA |
| | | AGCTGCTGATCTACGATAACAACAAGCGGCCTA |
| | | GCGGAATCCCTGGTCGCTTTAGCGGATCTAAATC |
| | | AGGCACTAGCGCTACCCTGGGAATCACCGGCCT |
| | | GCAGACCGGCGACGAAGCCGCCTACTACTGCGG |
| | | CACCTGGGACTCTAGTCTGAGCGCCTGGGTGTTC |
| | | GGCGGAGGCACTAGACTGACCGTGCTG |
| SEQ ID NO: 144 | Light | QSVLTQPPSLSAAPGQRVTISCSGSSSNIGNTYVSW |
| | Chain | YQQLPGTAPKLLIYDNNKRPSGIPGRFSGSKSGTSA |
| | | TLGITGLQTGDEAAYYCGTWDSSLSAWVFGGGTR |
| | | LTVLGQPKAAPSVTLFPPSSEELQANKATLVCLISD |
| | | FYPGAVTVAWKADSSPVKAGVETTTPSKQSNNKY |
| | | AASSYLSLTPEQWKSHRSYSCQVTHEGSTVEKTVA |
| | | PTECS |
| SEQ ID NO: 145 | DNA | CAGTCAGTCCTGACTCAGCCCCCTAGCCTGAGCG |
| | Light | |
| | Chain | CCGCTCCCGGTCAAAGAGTGACTATTAGCTGTAG |
| | | CGGCTCTAGCTCTAATATCGGTAATACCTACGTC |
| | | AGCTGGTATCAGCAGCTGCCCGGCACCGCCCCTA |
| | | AGCTGCTGATCTACGATAACAACAAGCGGCCTA |
| | | GCGGAATCCCTGGTCGCTTTAGCGGATCTAAATC |
| | | AGGCACTAGCGCTACCCTGGGAATCACCGGCCT |
| | | GCAGACCGGCGACGAAGCCGCCTACTACTGCGG |
| | | CACCTGGGACTCTAGTCTGAGCGCCTGGGTGTTC |
| | | GGCGGAGGCACTAGACTGACCGTGCTGGGTCAA |
| | | CCTAAGGCTGCCCCCAGCGTGACCCTGTTCCCCC |
| | | CCAGCAGCGAGGAGCTGCAGGCCAACAAGGCCA |
| | | CCCTGGTGTGCCTGATCAGCGACTTCTACCCAGG |
| | | CGCCGTGACCGTGGCCTGGAAGGCCGACAGCAG |
| | | CCCCGTGAAGGCCGGCGTGGAGACCACCACCCC |
| | | CAGCAAGCAGAGCAACAACAAGTACGCCGCCAG |
| | | CAGCTACCTGAGCCTGACCCCCGAGCAGTGGAA |
| | | GAGCCACAGGTCCTACAGCTGCCAGGTGACCCA |
| | | CGAGGGCAGCACCGTGGAAAAGACCGTGGCCCC |
| | | AACCGAGTGCAGC |
| NEG447 | | |
| SEQ ID NO: 146 | HCDR1 | RDYWS |
| (Kabat) | | |
| SEQ ID NO: 147 | HCDR2 | NIYYSGSTNYNPSLKS |
| (Kabat) | | |
| SEQ ID NO: 148 | HCDR3 | VPGCSSTSCIDGWFDP |
| (Kabat) | | |
| SEQ ID NO: 149 | HCDR1 | GGSISRD |
| (Chothia) | | |
| SEQ ID NO: 150 | HCDR2 | YYSGS |
| (Chothia) | | |
| SEQ ID NO: 151 | HCDR3 | VPGCSSTSCIDGWFDP |
| (Chothia) | | |
| SEQ ID NO: 152 | VH | QVQLQESGPGLVKPSETLSLTCTVSGGSISRDYWS |
| | | WVRQPPGAGLEWIGNIYYSGSTNYNPSLKSRVTISV |
| | | ATNKKQFSLKLTSVTAADTAVYYCARVPGCSSTSC |
| | | IDGWFDPWGQGILVTVSS |
| SEQ ID NO: 153 | DNA VH | CAGGTGCAGCTGCAAGAATCAGGCCCTGGCCTG |
| | | GTCAAGCCTAGCGAGACACTGAGCCTGACCTGC |
| | | ACCGTCAGCGGCGGCTCTATCTCTAGGGACTACT |
| | | GGTCCTGGGTCCGACAACCTCCTGGCGCTGGCCT |
| | | CGAGTGGATCGGTAATATCTACTATAGCGGCTCT |
| | | ACTAACTATAACCCTAGCCTGAAGTCTAGGGTCA |
| | | CAATTAGTGTGGCTACTAACAAGAAGCAGTTTAG |
| | | CCTGAAGCTGACTAGCGTGACCGCCGCTGACACC |
| | | GCCGTCTACTACTGCGCTAGAGTGCCCGGCTGCT |
| | | CTAGCACTAGCTGTATCGACGGTTGGTTTGACCC |
| | | TTGGGGTCAAGGGATCCTGGTCACCGTGTCTAGC |
| SEQ ID NO: 154 | Heavy | QVQLQESGPGLVKPSETLSLTCTVSGGSISRDYWS |
| | Chain | WVRQPPGAGLEWIGNIYYSGSTNYNPSLKSRVTISV |
| | | ATNKKQFSLKLTSVTAADTAVYYCARVPGCSSTSC |
| | | IDGWFDPWGQGILVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKRVEPKSCDKTHTCPPCPAPELLGGPSVFL |
| | | FPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPGK |
| SEQ ID NO: 155 | DNA | CAGGTGCAGCTGCAAGAATCAGGCCCTGGCCTG |
| | Heavy | GTCAAGCCTAGCGAGACACTGAGCCTGACCTGC |
| | Chain | ACCGTCAGCGGCGGCTCTATCTCTAGGGACTACT |
| | | GGTCCTGGGTCCGACAACCTCCTGGCGCTGGCCT |
| | | CGAGTGGATCGGTAATATCTACTATAGCGGCTCT |
| | | ACTAACTATAACCCTAGCCTGAAGTCTAGGGTCA |
| | | CAATTAGTGTGGCTACTAACAAGAAGCAGTTTAG |
| | | CCTGAAGCTGACTAGCGTGACCGCCGCTGACACC |
| | | GCCGTCTACTACTGCGCTAGAGTGCCCGGCTGCT |
| | | CTAGCACTAGCTGTATCGACGGTTGGTTTGACCC |
| | | TTGGGGTCAAGGGATCCTGGTCACCGTGTCTAGC |
| | | GCTAGCACTAAGGGCCCAAGTGTGTTTCCCCTGG |
| | | CCCCCAGCAGCAAGTCTACTTCCGGCGGAACTGC |
| | | TGCCCTGGGTTGCCTGGTGAAGGACTACTTCCCC |
| | | GAGCCCGTGACAGTGTCCTGGAACTCTGGGGCTC |
| | | TGACTTCCGGCGTGCACACCTTCCCCGCCGTGCT |
| | | GCAGAGCAGCGGCCTGTACAGCCTGAGCAGCGT |
| | | GGTGACAGTGCCCTCCAGCTCTCTGGGAACCCAG |
| | | ACCTATATCTGCAACGTGAACCACAAGCCCAGC |
| | | AACACCAAGGTGGACAAGAGAGTGGAGCCCAAG |
| | | AGCTGCGACAAGACCCACACCTGCCCCCCCTGCC |
| | | CAGCTCCAGAACTGCTGGGAGGGCCTTCCGTGTT |
| | | CCTGTTCCCCCCCAAGCCCAAGGACACCCTGATG |
| | | ATCAGCAGGACCCCCGAGGTGACCTGCGTGGTG |
| | | GTGGACGTGTCCCACGAGGACCCAGAGGTGAAG |
| | | TTCAACTGGTACGTGGACGGCGTGGAGGTGCAC |
| | | AACGCCAAGACCAAGCCCAGAGAGGAGCAGTAC |
| | | AACAGCACCTACAGGGTGGTGTCCGTGCTGACC |
| | | GTGCTGCACCAGGACTGGCTGAACGGCAAAGAA |
| | | TACAAGTGCAAAGTCTCCAACAAGGCCCTGCCA |
| | | GCCCCAATCGAAAAGACAATCAGCAAGGCCAAG |
| | | GGCCAGCCACGGGAGCCCCAGGTGTACACCCTG |
| | | CCCCCCAGCCGGGAGGAGATGACCAAGAACCAG |
| | | GTGTCCCTGACCTGTCTGGTGAAGGGCTTCTACC |
| | | CCAGCGATATCGCCGTGGAGTGGGAGAGCAACG |
| | | GCCAGCCCGAGAACAACTACAAGACCACCCCCC |
| | | CAGTGCTGGACAGCGACGGCAGCTTCTTCCTGTA |
| | | CAGCAAGCTGACCGTGGACAAGTCCAGGTGGCA |
| | | GCAGGGCAACGTGTTCAGCTGCAGCGTGATGCA |
| | | CGAGGCCCTGCACAACCACTACACCCAGAAGTC |
| | | CCTGAGCCTGAGCCCCGGCAAG |
| SEQ ID NO: 156 | LCDR1 | SGSSSNIGNTYVS |
| (Kabat) | | |
| SEQ ID NO: 157 | LCDR2 | DNNKRPS |
| (Kabat) | | |
| SEQ ID NO: 158 | LCDR3 | GTWDSSLSAWV |
| (Kabat) | | |
| SEQ ID NO: 159 | LCDR1 | SSSNIGNTY |
| (Chothia) | | |
| SEQ ID NO: 160 | LCDR2 | DNN |
| (Chothia) | | |
| SEQ ID NO: 161 | LCDR3 | WDSSLSAW |
| (Chothia) | | |
| SEQ ID NO: 162 | VL | QSVLTQPPSLSAAPGQKVTISCSGSSSNIGNTYVSW |
| | | YQQLPGTAPKLLIYDNNKRPSGIPDRFSGSKSGTSA |
| | | TLGITGLQTGDEAVYYCGTWDSSLSAWVFGGGTR |
| | | LTVL |
| SEQ ID NO: 163 | DNA VL | CAGTCAGTCCTGACTCAGCCCCCTAGCCTGAGCG |
| | | CCGCTCCCGGTCAAAAAGTGACTATTAGCTGTAG |
| | | CGGCTCTAGCTCTAATATCGGTAATACCTACGTC |
| | | AGCTGGTATCAGCAGCTGCCCGGCACCGCCCCTA |
| | | AGCTGCTGATCTACGATAACAACAAGCGGCCTA |
| | | GCGGAATCCCCGATAGGTTTAGCGGATCTAAGTC |
| | | AGGCACTAGCGCTACCCTGGGAATCACCGGCCT |
| | | GCAGACCGGCGACGAGGCCGTCTACTACTGCGG |
| | | CACCTGGGACTCTAGTCTGAGCGCCTGGGTGTTC |
| | | GGCGGAGGCACTAGACTGACCGTGCTG |
| SEQ ID NO: 164 | Light | QSVLTQPPSLSAAPGQKVTISCSGSSSNIGNTYVSW |
| | Chain | YQQLPGTAPKLLIYDNNKRPSGIPDRFSGSKSGTSA |
| | | TLGITGLQTGDEAVYYCGTWDSSLSAWVFGGGTR |
| | | LTVLGQPKAAPSVTLFPPSSEELQANKATLVCLISD |
| | | FYPGAVTVAWKADSSPVKAGVETTTPSKQSNNKY |
| | | AASSYLSLTPEQWKSHRSYSCQVTHEGSTVEKTVA |
| | | PTECS |
| SEQ ID NO: 165 | DNA | CAGTCAGTCCTGACTCAGCCCCCTAGCCTGAGCG |
| | Light | CCGCTCCCGGTCAAAAAGTGACTATTAGCTGTAG |
| | Chain | CGGCTCTAGCTCTAATATCGGTAATACCTACGTC |
| | | AGCTGGTATCAGCAGCTGCCCGGCACCGCCCCTA |
| | | AGCTGCTGATCTACGATAACAACAAGCGGCCTA |
| | | GCGGAATCCCCGATAGGTTTAGCGGATCTAAGTC |
| | | AGGCACTAGCGCTACCCTGGGAATCACCGGCCT |
| | | GCAGACCGGCGACGAGGCCGTCTACTACTGCGG |
| | | CACCTGGGACTCTAGTCTGAGCGCCTGGGTGTTC |
| | | GGCGGAGGCACTAGACTGACCGTGCTGGGTCAA |
| | | CCTAAGGCTGCCCCCAGCGTGACCCTGTTCCCCC |
| | | CCAGCAGCGAGGAGCTGCAGGCCAACAAGGCCA |
| | | CCCTGGTGTGCCTGATCAGCGACTTCTACCCAGG |
| | | CGCCGTGACCGTGGCCTGGAAGGCCGACAGCAG |
| | | CCCCGTGAAGGCCGGCGTGGAGACCACCACCCC |
| | | CAGCAAGCAGAGCAACAACAAGTACGCCGCCAG |
| | | CAGCTACCTGAGCCTGACCCCCGAGCAGTGGAA |
| | | GAGCCACAGGTCCTACAGCTGCCAGGTGACCCA |
| | | CGAGGGCAGCACCGTGGAAAAGACCGTGGCCCC |
| | | AACCGAGTGCAGC |
| NEG447A | | |
| SEQ ID NO: 166 | HCDR1 | RDYWS |
| (Kabat) | | |
| SEQ ID NO: 167 | HCDR2 | NIYYSGSTNYNPSLKS |
| (Kabat) | | |
| SEQ ID NO: 168 | HCDR3 | VPGCSSTSCIDGWFDP |
| (Kabat) | | |
| SEQ ID NO: 169 | HCDR1 | GGSISRD |
| (Chothia) | | |
| SEQ ID NO: 170 | HCDR2 | YYSGS |
| (Chothia) | | |
| SEQ ID NO: 171 | HCDR3 | VPGCSSTSCIDGWFDP |
| (Chothia) | | |
| SEQ ID NO: 172 | VH | QVQLQESGPGLVKPSETLSLTCTVSGGSISRDYWS |
| | | WVRQPPGAGLEWIGNIYYSGSTNYNPSLKSRVTISV |
| | | ATNKKQFSLKLTSVTAADTAVYYCARVPGCSSTSC |
| | | IDGWFDPWGQGILVTVSS |
| SEQ ID NO: 173 | DNA VH | CAGGTGCAATTGCAGGAAAGCGGCCCTGGCCTC |
| | | GTGAAGCCCAGCGAGACACTGAGCCTGACCTGT |
| | | ACCGTGTCCGGCGGCAGCATCAGCAGAGACTAC |
| | | TGGAGCTGGGTTCGCCAGCCTCCAGGCGCAGGA |
| | | CTGGAATGGATCGGCAACATCTACTACAGCGGC |
| | | AGCACCAACTACAACCCCAGCCTGAAGTCCAGA |
| | | GTGACCATCAGCGTGGCCACAAACAAGAAACAG |
| | | TTCTCCCTGAAGCTGACCAGCGTGACAGCCGCCG |
| | | ATACCGCCGTGTACTACTGCGCCAGAGTGCCTGG |
| | | CTGTAGCAGCACCAGCTGCATCGACGGATGGTTC |
| | | GACCCTTGGGGCCAGGGCATTCTCGTGACCGTCA |
| | | GCTCA |
| SEQ ID NO: 174 | Heavy | QVQLQESGPGLVKPSETLSLTCTVSGGSISRDYWS |
| | Chain | WVRQPPGAGLEWIGNIYYSGSTNYNPSLKSRVTISV |
| | | ATNKKQFSLKLTSVTAADTAVYYCARVPGCSSTSC |
| | | IDGWFDPWGQGILVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKRVEPKSCDKTHTCPPCPAPELLGGPSVFL |
| | | FPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPGK |
| SEQ ID NO: 175 | DNA | CAGGTGCAATTGCAGGAAAGCGGCCCTGGCCTC |
| | Heavy | GTGAAGCCCAGCGAGACACTGAGCCTGACCTGT |
| | Chain | ACCGTGTCCGGCGGCAGCATCAGCAGAGACTAC |
| | | TGGAGCTGGGTTCGCCAGCCTCCAGGCGCAGGA |
| | | CTGGAATGGATCGGCAACATCTACTACAGCGGC |
| | | AGCACCAACTACAACCCCAGCCTGAAGTCCAGA |
| | | GTGACCATCAGCGTGGCCACAAACAAGAAACAG |
| | | TTCTCCCTGAAGCTGACCAGCGTGACAGCCGCCG |
| | | ATACCGCCGTGTACTACTGCGCCAGAGTGCCTGG |
| | | CTGTAGCAGCACCAGCTGCATCGACGGATGGTTC |
| | | GACCCTTGGGGCCAGGGCATTCTCGTGACCGTCA |
| | | GCTCAGCTAGCACCAAGGGCCCCAGCGTGTTCCC |
| | | CCTGGCCCCCAGCAGCAAGAGCACCAGCGGCGG |
| | | CACAGCCGCCCTGGGCTGCCTGGTGAAGGACTA |
| | | CTTCCCCGAGCCCGTGACCGTGTCCTGGAACAGC |
| | | GGAGCCCTGACCTCCGGCGTGCACACCTTCCCCG |
| | | CCGTGCTGCAGAGCAGCGGCCTGTACAGCCTGTC |
| | | CAGCGTGGTGACAGTGCCCAGCAGCAGCCTGGG |
| | | CACCCAGACCTACATCTGCAACGTGAACCACAA |
| | | GCCCAGCAACACCAAGGTGGACAAGAGAGTGGA |
| | | GCCCAAGAGCTGCGACAAGACCCACACCTGCCC |
| | | CCCCTGCCCAGCCCCAGAGCTGCTGGGCGGACCC |
| | | TCCGTGTTCCTGTTCCCCCCCAAGCCCAAGGACA |
| | | CCCTGATGATCAGCAGGACCCCCGAGGTGACCT |
| | | GCGTGGTGGTGGACGTGAGCCACGAGGACCCAG |
| | | AGGTGAAGTTCAACTGGTACGTGGACGGCGTGG |
| | | AGGTGCACAACGCCAAGACCAAGCCCAGAGAGG |
| | | AGCAGTACAACAGCACCTACAGGGTGGTGTCCG |
| | | TGCTGACCGTGCTGCACCAGGACTGGCTGAACG |
| | | GCAAGGAATACAAGTGCAAGGTCTCCAACAAGG |
| | | CCCTGCCAGCCCCCATCGAAAAGACCATCAGCA |
| | | AGGCCAAGGGCCAGCCACGGGAGCCCCAGGTGT |
| | | ACACCCTGCCCCCCTCCCGGGAGGAGATGACCA |
| | | AGAACCAGGTGTCCCTGACCTGTCTGGTGAAGG |
| | | GCTTCTACCCCAGCGACATCGCCGTGGAGTGGGA |
| | | GAGCAACGGCCAGCCCGAGAACAACTACAAGAC |
| | | CACCCCCCCAGTGCTGGACAGCGACGGCAGCTTC |
| | | TTCCTGTACAGCAAGCTGACCGTGGACAAGTCCA |
| | | GGTGGCAGCAGGGCAACGTGTTCAGCTGCAGCG |
| | | TGATGCACGAGGCCCTGCACAACCACTACACCC |
| | | AGAAGAGCCTGAGCCTGTCCCCCGGCAAG |
| SEQ ID NO: 176 | LCDR1 | SGSSSNIGNTYVS |
| (Kabat) | | |
| SEQ ID NO: 177 | LCDR2 | DNNKRPS |
| (Kabat) | | |
| SEQ ID NO: 178 | LCDR3 | GTWDSSLSAWV |
| (Kabat) | | |
| SEQ ID NO: 179 | LCDR1 | SSSNIGNTY |
| (Chothia) | | |
| SEQ ID NO: 180 | LCDR2 | DNN |
| (Chothia) | | |
| SEQ ID NO: 181 | LCDR3 | WDSSLSAW |
| (Chothia) | | |
| SEQ ID NO: 182 | VL | QSVLTQPPSVSAAPGQKVTISCSGSSSNIGNTYVSW |
| | | YQQLPGTAPKLLIYDNNKRPSGIPDRFSGSKSGTSA |
| | | TLGITGLQTGDEADYYCGTWDSSLSAWVFGGGTR |
| | | LTVL |
| SEQ ID NO: 183 | DNA VL | CAAAGCGTGCTGACCCAGCCTCCTAGCGTGTCTG |
| | | CTGCCCCTGGCCAGAAGGTGACCATCAGCTGTAG |
| | | CGGCAGCAGCTCCAACATCGGCAACACCTACGT |
| | | GTCCTGGTATCAGCAGCTGCCCGGCACCGCCCCC |
| | | AAACTGCTGATCTACGACAACAACAAGCGGCCC |
| | | AGCGGCATCCCCGATAGATTTTCTGGCAGCAAGA |
| | | GCGGCACCAGCGCCACCCTGGGAATCACAGGAC |
| | | TGCAGACAGGGGACGAGGCCGATTACTACTGTG |
| | | GCACCTGGGATTCTAGCCTGAGCGCCTGGGTGTT |
| | | CGGCGGAGGCACAAGACTGACAGTGCTG |
| SEQ ID NO: 184 | Light | QSVLTQPPSVSAAPGQKVTISCSGSSSNIGNTYVSW |
| | Chain | YQQLPGTAPKLLIYDNNKRPSGIPDRFSGSKSGTSA |
| | | TLGITGLQTGDEADYYCGTWDSSLSAWVFGGGTR |
| | | LTVLGQPKAAPSVTLFPPSSEELQANKATLVCLISD |
| | | FYPGAVTVAWKADSSPVKAGVETTTPSKQSNNKY |
| | | AASSYLSLTPEQWKSHRSYSCQVTHEGSTVEKTVA |
| | | PTECS |
| SEQ ID NO: 185 | DNA | CAAAGCGTGCTGACCCAGCCTCCTAGCGTGTCTG |
| | Light | CTGCCCCTGGCCAGAAGGTGACCATCAGCTGTAG |
| | Chain | CGGCAGCAGCTCCAACATCGGCAACACCTACGT |
| | | GTCCTGGTATCAGCAGCTGCCCGGCACCGCCCCC |
| | | AAACTGCTGATCTACGACAACAACAAGCGGCCC |
| | | AGCGGCATCCCCGATAGATTTTCTGGCAGCAAGA |
| | | GCGGCACCAGCGCCACCCTGGGAATCACAGGAC |
| | | TGCAGACAGGGGACGAGGCCGATTACTACTGTG |
| | | GCACCTGGGATTCTAGCCTGAGCGCCTGGGTGTT |
| | | CGGCGGAGGCACAAGACTGACAGTGCTGGGTCA |
| | | GCCTAAGGCCGCTCCCTCCGTGACCCTGTTCCCC |
| | | CCCAGCTCCGAGGAACTGCAGGCCAACAAGGCC |
| | | ACCCTGGTGTGCCTGATCAGCGACTTCTACCCTG |
| | | GCGCCGTGACCGTGGCCTGGAAGGCCGACAGCA |
| | | GCCCCGTGAAGGCCGGCGTGGAGACAACCACCC |
| | | CCAGCAAGCAGAGCAACAACAAGTACGCCGCCA |
| | | GCAGCTACCTGAGCCTGACCCCCGAGCAGTGGA |
| | | AGAGCCACAGAAGCTACAGCTGCCAGGTCACCC |
| | | ACGAGGGCAGCACCGTGGAGAAAACCGTGGCCC |
| | | CCACCGAGTGCAGC |
| P7G11 | | |
| SEQ ID NO: 186 | HCDR1 | SGGYSWS |
| (Kabat) | | |
| SEQ ID NO: 187 | HCDR2 | YIYYRGTTYYNPSLKS |
| (Kabat) | | |
| SEQ ID NO: 188 | HCDR3 | ALTHLVGVGWFDP |
| (Kabat) | | |
| SEQ ID NO: 189 | HCDR1 | GGSISSGGY |
| (Chothia) | | |
| SEQ ID NO: 190 | HCDR2 | YYRGT |
| (Chothia) | | |
| SEQ ID NO: 191 | HCDR3 | ALTHLVGVGWFDP |
| (Chothia) | | |
| SEQ ID NO: 192 | VH | QVQLQESGPGLAKPSQTLSLTCSVSGGSISSGGYSW |
| | | SWIRQPPGKGLEYIGYIYYRGTTYYNPSLKSRITMS |
| | | VDTSNNQISLKLTSVTAADTAVYYCARALTHLVGV |
| | | GWFDPWGQGTMVTVSS |
| SEQ ID NO: 193 | DNA VH | CAGGTGCAGCTGCAAGAATCAGGCCCTGGCCTG |
| | | GCTAAGCCTAGTCAGACCCTGAGCCTGACCTGTA |
| | | GCGTCAGCGGAGGCTCTATCTCTAGCGGCGGCTA |
| | | TAGCTGGTCCTGGATTAGACAGCCCCCAGGTAAA |
| | | GGCCTCGAGTATATCGGCTATATCTACTATAGGG |
| | | GCACTACCTACTATAACCCTAGCCTGAAGTCTAG |
| | | GATCACTATGAGCGTGGACACCTCTAACAATCAG |
| | | ATTAGCCTGAAGCTGACTAGCGTGACCGCCGCTG |
| | | ACACCGCCGTCTACTACTGCGCTAGAGCCCTGAC |
| | | TCACCTCGTTGGAGTGGGCTGGTTTGACCCTTGG |
| | | GGTCAAGGCACTATGGTCACCGTGTCTAGC |
| SEQ ID NO: 194 | Heavy | QVQLQESGPGLAKPSQTLSLTCSVSGGSISSGGYSW |
| | Chain | SWIRQPPGKGLEYIGYIYYRGTTYYNPSLKSRITMS |
| | | VDTSNNQISLKLTSVTAADTAVYYCARALTHLVGV |
| | | GWFDPWGQGTMVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPS |
| | | NTKVDKRVEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| SEQ ID NO: 195 | DNA | CAGGTGCAGCTGCAAGAATCAGGCCCTGGCCTG |
| | Heavy | GCTAAGCCTAGTCAGACCCTGAGCCTGACCTGTA |
| | Chain | GCGTCAGCGGAGGCTCTATCTCTAGCGGCGGCTA |
| | | TAGCTGGTCCTGGATTAGACAGCCCCCAGGTAAA |
| | | GGCCTCGAGTATATCGGCTATATCTACTATAGGG |
| | | GCACTACCTACTATAACCCTAGCCTGAAGTCTAG |
| | | GATCACTATGAGCGTGGACACCTCTAACAATCAG |
| | | ATTAGCCTGAAGCTGACTAGCGTGACCGCCGCTG |
| | | ACACCGCCGTCTACTACTGCGCTAGAGCCCTGAC |
| | | TCACCTCGTTGGAGTGGGCTGGTTTGACCCTTGG |
| | | GGTCAAGGCACTATGGTCACCGTGTCTAGCGCTA |
| | | GCACTAAGGGCCCAAGTGTGTTTCCCCTGGCCCC |
| | | CAGCAGCAAGTCTACTTCCGGCGGAACTGCTGCC |
| | | CTGGGTTGCCTGGTGAAGGACTACTTCCCCGAGC |
| | | CCGTGACAGTGTCCTGGAACTCTGGGGCTCTGAC |
| | | TTCCGGCGTGCACACCTTCCCCGCCGTGCTGCAG |
| | | AGCAGCGGCCTGTACAGCCTGAGCAGCGTGGTG |
| | | ACAGTGCCCTCCAGCTCTCTGGGAACCCAGACCT |
| | | ATATCTGCAACGTGAACCACAAGCCCAGCAACA |
| | | CCAAGGTGGACAAGAGAGTGGAGCCCAAGAGCT |
| | | GCGACAAGACCCACACCTGCCCCCCCTGCCCAGC |
| | | TCCAGAACTGCTGGGAGGGCCTTCCGTGTTCCTG |
| | | TTCCCCCCCAAGCCCAAGGACACCCTGATGATCA |
| | | GCAGGACCCCCGAGGTGACCTGCGTGGTGGTGG |
| | | ACGTGTCCCACGAGGACCCAGAGGTGAAGTTCA |
| | | ACTGGTACGTGGACGGCGTGGAGGTGCACAACG |
| | | CCAAGACCAAGCCCAGAGAGGAGCAGTACAACA |
| | | GCACCTACAGGGTGGTGTCCGTGCTGACCGTGCT |
| | | GCACCAGGACTGGCTGAACGGCAAAGAATACAA |
| | | GTGCAAAGTCTCCAACAAGGCCCTGCCAGCCCC |
| | | AATCGAAAAGACAATCAGCAAGGCCAAGGGCCA |
| | | GCCACGGGAGCCCCAGGTGTACACCCTGCCCCCC |
| | | AGCCGGGAGGAGATGACCAAGAACCAGGTGTCC |
| | | CTGACCTGTCTGGTGAAGGGCTTCTACCCCAGCG |
| | | ATATCGCCGTGGAGTGGGAGAGCAACGGCCAGC |
| | | CCGAGAACAACTACAAGACCACCCCCCCAGTGC |
| | | TGGACAGCGACGGCAGCTTCTTCCTGTACAGCAA |
| | | GCTGACCGTGGACAAGTCCAGGTGGCAGCAGGG |
| | | CAACGTGTTCAGCTGCAGCGTGATGCACGAGGC |
| | | CCTGCACAACCACTACACCCAGAAGTCCCTGAGC |
| | | CTGAGCCCCGGCAAG |
| SEQ ID NO: 196 | LCDR1 | SGGSSNLGSNYVS |
| (Kabat) | | |
| SEQ ID NO: 197 | LCDR2 | DNNKRPS |
| (Kabat) | | |
| SEQ ID NO: 198 | LCDR3 | GTWDGSLSAWV |
| (Kabat) | | |
| SEQ ID NO: 199 | LCDR1 | GSSNLGSNY |
| (Chothia) | | |
| SEQ ID NO: 200 | LCDR2 | DNN |
| (Chothia) | | |
| SEQ ID NO: 201 | LCDR3 | WDGSLSAW |
| (Chothia) | | |
| SEQ ID NO: 202 | VL | QSVLTQPPSVSAAPGQKVTISCSGGSSNLGSNYVS |
| | | WYQQLPGTAPKLLIYDNNKRPSGIPDRFSGSKSGTS |
| | | ATLGITGLQTGDEADYYCGTWDGSLSAWVFGGGT |
| | | KVTVL |
| SEQ ID NO: 203 | DNA VL | CAGTCAGTCCTGACTCAGCCCCCTAGCGTCAGCG |
| | | CCGCTCCCGGTCAAAAAGTGACTATTAGCTGTAG |
| | | CGGCGGCTCCTCTAACCTGGGCTCTAACTACGTC |
| | | AGCTGGTATCAGCAGCTGCCCGGCACCGCCCCTA |
| | | AGCTGCTGATCTACGATAACAACAAGCGGCCTA |
| | | GCGGAATCCCCGATAGGTTTAGCGGCTCTAAGTC |
| | | AGGCACTAGCGCTACCCTGGGAATCACCGGCCT |
| | | GCAGACCGGCGACGAGGCCGACTACTACTGTGG |
| | | CACCTGGGACGGTAGCCTGAGCGCCTGGGTGTTC |
| | | GGCGGAGGCACTAAAGTCACAGTGCTG |
| SEQ ID NO: 204 | Light | QSVLTQPPSVSAAPGQKVTISCSGGSSNLGSNYVS |
| | Chain | WYQQLPGTAPKLLIYDNNKRPSGIPDRFSGSKSGTS |
| | | ATLGITGLQTGDEADYYCGTWDGSLSAWVFGGGT |
| | | KVTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKTV |
| | | APTECS |
| SEQ ID NO: 205 | DNA | CAGTCAGTCCTGACTCAGCCCCCTAGCGTCAGCG |
| | Light | CCGCTCCCGGTCAAAAAGTGACTATTAGCTGTAG |
| | Chain | CGGCGGCTCCTCTAACCTGGGCTCTAACTACGTC |
| | | AGCTGGTATCAGCAGCTGCCCGGCACCGCCCCTA |
| | | AGCTGCTGATCTACGATAACAACAAGCGGCCTA |
| | | GCGGAATCCCCGATAGGTTTAGCGGCTCTAAGTC |
| | | AGGCACTAGCGCTACCCTGGGAATCACCGGCCT |
| | | GCAGACCGGCGACGAGGCCGACTACTACTGTGG |
| | | CACCTGGGACGGTAGCCTGAGCGCCTGGGTGTTC |
| | | GGCGGAGGCACTAAAGTCACAGTGCTGGGTCAA |
| | | CCTAAGGCTGCCCCCAGCGTGACCCTGTTCCCCC |
| | | CCAGCAGCGAGGAGCTGCAGGCCAACAAGGCCA |
| | | CCCTGGTGTGCCTGATCAGCGACTTCTACCCAGG |
| | | CGCCGTGACCGTGGCCTGGAAGGCCGACAGCAG |
| | | CCCCGTGAAGGCCGGCGTGGAGACCACCACCCC |
| | | CAGCAAGCAGAGCAACAACAAGTACGCCGCCAG |
| | | CAGCTACCTGAGCCTGACCCCCGAGCAGTGGAA |
| | | GAGCCACAGGTCCTACAGCTGCCAGGTGACCCA |
| | | CGAGGGCAGCACCGTGGAAAAGACCGTGGCCCC |
| | | AACCGAGTGCAGC |
| P7G11A | | |
| SEQ ID NO: 206 | HCDR1 | SGGYSWS |
| (Kabat) | | |
| SEQ ID NO: 207 | HCDR2 | YIYYRGTTYYNPSLKS |
| (Kabat) | | |
| SEQ ID NO: 208 | HCDR3 | ALTHLVGVGWFDP |
| (Kabat) | | |
| SEQ ID NO: 209 | HCDR1 | GGSISSGGY |
| (Chothia) | | |
| SEQ ID NO: 210 | HCDR2 | YYRGT |
| (Chothia) | | |
| SEQ ID NO: 211 | HCDR3 | ALTHLVGVGWFDP |
| (Chothia) | | |
| SEQ ID NO: 212 | VH | QVQLQESGPGLVKPSQTLSLTCTVSGGSISSGGYSW |
| | | SWIRQPPGKGLEYIGYIYYRGTTYYNPSLKSRVTIS |
| | | VDTSNNQISLKLSSVTAADTAVYYCARALTHLVGV |
| | | GWFDPWGQGTMVTVSS |
| SEQ ID NO: 213 | DNA VH | CAGGTGCAGCTGCAAGAATCAGGCCCTGGCCTG |
| | | GTCAAGCCTAGTCAGACCCTGAGCCTGACCTGCA |
| | | CCGTCAGCGGAGGCTCTATCTCTAGCGGCGGCTA |
| | | TAGCTGGTCCTGGATTAGACAGCCCCCAGGTAAA |
| | | GGCCTCGAGTATATCGGCTATATCTACTATAGGG |
| | | GCACTACCTACTATAACCCTAGCCTGAAGTCTAG |
| | | GGTCACAATTAGCGTGGACACCTCTAACAATCAG |
| | | ATTAGCCTGAAGCTGTCTAGCGTGACCGCCGCTG |
| | | ACACCGCCGTCTACTACTGCGCTAGAGCCCTGAC |
| | | TCACCTCGTCGGAGTGGGCTGGTTTGACCCTTGG |
| | | GGTCAAGGCACTATGGTCACCGTGTCTAGC |
| SEQ ID NO: 214 | Heavy | QVQLQESGPGLVKPSQTLSLTCTVSGGSISSGGYSW |
| | Chain | SWIRQPPGKGLEYIGYIYYRGTTYYNPSLKSRVTIS |
| | | VDTSNNQISLKLSSVTAADTAVYYCARALTHLVGV |
| | | GWFDPWGQGTMVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPS |
| | | NTKVDKRVEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| SEQ ID NO: 215 | DNA | CAGGTGCAGCTGCAAGAATCAGGCCCTGGCCTG |
| | Heavy | GTCAAGCCTAGTCAGACCCTGAGCCTGACCTGCA |
| | Chain | CCGTCAGCGGAGGCTCTATCTCTAGCGGCGGCTA |
| | | TAGCTGGTCCTGGATTAGACAGCCCCCAGGTAAA |
| | | GGCCTCGAGTATATCGGCTATATCTACTATAGGG |
| | | GCACTACCTACTATAACCCTAGCCTGAAGTCTAG |
| | | GGTCACAATTAGCGTGGACACCTCTAACAATCAG |
| | | ATTAGCCTGAAGCTGTCTAGCGTGACCGCCGCTG |
| | | ACACCGCCGTCTACTACTGCGCTAGAGCCCTGAC |
| | | TCACCTCGTCGGAGTGGGCTGGTTTGACCCTTGG |
| | | GGTCAAGGCACTATGGTCACCGTGTCTAGCGCTA |
| | | GCACTAAGGGCCCAAGTGTGTTTCCCCTGGCCCC |
| | | CAGCAGCAAGTCTACTTCCGGCGGAACTGCTGCC |
| | | CTGGGTTGCCTGGTGAAGGACTACTTCCCCGAGC |
| | | CCGTGACAGTGTCCTGGAACTCTGGGGCTCTGAC |
| | | TTCCGGCGTGCACACCTTCCCCGCCGTGCTGCAG |
| | | AGCAGCGGCCTGTACAGCCTGAGCAGCGTGGTG |
| | | ACAGTGCCCTCCAGCTCTCTGGGAACCCAGACCT |
| | | ATATCTGCAACGTGAACCACAAGCCCAGCAACA |
| | | CCAAGGTGGACAAGAGAGTGGAGCCCAAGAGCT |
| | | GCGACAAGACCCACACCTGCCCCCCCTGCCCAGC |
| | | TCCAGAACTGCTGGGAGGGCCTTCCGTGTTCCTG |
| | | TTCCCCCCCAAGCCCAAGGACACCCTGATGATCA |
| | | GCAGGACCCCCGAGGTGACCTGCGTGGTGGTGG |
| | | ACGTGTCCCACGAGGACCCAGAGGTGAAGTTCA |
| | | ACTGGTACGTGGACGGCGTGGAGGTGCACAACG |
| | | CCAAGACCAAGCCCAGAGAGGAGCAGTACAACA |
| | | GCACCTACAGGGTGGTGTCCGTGCTGACCGTGCT |
| | | GCACCAGGACTGGCTGAACGGCAAAGAATACAA |
| | | GTGCAAAGTCTCCAACAAGGCCCTGCCAGCCCC |
| | | AATCGAAAAGACAATCAGCAAGGCCAAGGGCCA |
| | | GCCACGGGAGCCCCAGGTGTACACCCTGCCCCCC |
| | | AGCCGGGAGGAGATGACCAAGAACCAGGTGTCC |
| | | CTGACCTGTCTGGTGAAGGGCTTCTACCCCAGCG |
| | | ATATCGCCGTGGAGTGGGAGAGCAACGGCCAGC |
| | | CCGAGAACAACTACAAGACCACCCCCCCAGTGC |
| | | TGGACAGCGACGGCAGCTTCTTCCTGTACAGCAA |
| | | GCTGACCGTGGACAAGTCCAGGTGGCAGCAGGG |
| | | CAACGTGTTCAGCTGCAGCGTGATGCACGAGGC |
| | | CCTGCACAACCACTACACCCAGAAGTCCCTGAGC |
| | | CTGAGCCCCGGCAAG |
| SEQ ID NO: 216 | LCDR1 | SGGSSNLGSNYVS |
| (Kabat) | | |
| SEQ ID NO: 217 | LCDR2 | DNNKRPS |
| (Kabat) | | |
| SEQ ID NO: 218 | LCDR3 | GTWDGSLSAWV |
| (Kabat) | | |
| SEQ ID NO: 219 | LCDR1 | GSSNLGSNY |
| (Chothia) | | |
| SEQ ID NO: 220 | LCDR2 | DNN |
| (Chothia) | | |
| SEQ ID NO: 221 | LCDR3 | WDGSLSAW |
| (Chothia) | | |
| SEQ ID NO: 222 | VL | QSVLTQPPSVSAAPGQKVTISCSGGSSNLGSNYVS |
| | | WYQQLPGTAPKLLIYDNNKRPSGIPDRFSGSKSGTS |
| | | ATLGITGLQTGDEADYYCGTWDGSLSAWVFGGGT |
| | | KVTVL |
| SEQ ID NO: 223 | DNA VL | CAGTCAGTCCTGACTCAGCCCCCTAGCGTCAGCG |
| | | CCGCTCCCGGTCAAAAAGTGACTATTAGCTGTAG |
| | | CGGCGGCTCCTCTAACCTGGGCTCTAACTACGTC |
| | | AGCTGGTATCAGCAGCTGCCCGGCACCGCCCCTA |
| | | AGCTGCTGATCTACGATAACAACAAGCGGCCTA |
| | | GCGGAATCCCCGATAGGTTTAGCGGCTCTAAGTC |
| | | AGGCACTAGCGCTACCCTGGGAATCACCGGCCT |
| | | GCAGACCGGCGACGAGGCCGACTACTACTGTGG |
| | | CACCTGGGACGGTAGCCTGAGCGCCTGGGTGTTC |
| | | GGCGGAGGCACTAAAGTCACAGTGCTG |
| SEQ ID NO: 224 | Light | QSVLTQPPSVSAAPGQKVTISCSGGSSNLGSNYVS |
| | Chain | WYQQLPGTAPKLLIYDNNKRPSGIPDRFSGSKSGTS |
| | | ATLGITGLQTGDEADYYCGTWDGSLSAWVFGGGT |
| | | KVTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKTV |
| | | APTECS |
| SEQ ID NO: 225 | DNA | CAGTCAGTCCTGACTCAGCCCCCTAGCGTCAGCG |
| | Light | CCGCTCCCGGTCAAAAAGTGACTATTAGCTGTAG |
| | Chain | CGGCGGCTCCTCTAACCTGGGCTCTAACTACGTC |
| | | AGCTGGTATCAGCAGCTGCCCGGCACCGCCCCTA |
| | | AGCTGCTGATCTACGATAACAACAAGCGGCCTA |
| | | GCGGAATCCCCGATAGGTTTAGCGGCTCTAAGTC |
| | | AGGCACTAGCGCTACCCTGGGAATCACCGGCCT |
| | | GCAGACCGGCGACGAGGCCGACTACTACTGTGG |
| | | CACCTGGGACGGTAGCCTGAGCGCCTGGGTGTTC |
| | | GGCGGAGGCACTAAAGTCACAGTGCTGGGTCAA |
| | | CCTAAGGCTGCCCCCAGCGTGACCCTGTTCCCCC |
| | | CCAGCAGCGAGGAGCTGCAGGCCAACAAGGCCA |
| | | CCCTGGTGTGCCTGATCAGCGACTTCTACCCAGG |
| | | CGCCGTGACCGTGGCCTGGAAGGCCGACAGCAG |
| | | CCCCGTGAAGGCCGGCGTGGAGACCACCACCCC |
| | | CAGCAAGCAGAGCAACAACAAGTACGCCGCCAG |
| | | CAGCTACCTGAGCCTGACCCCCGAGCAGTGGAA |
| | | GAGCCACAGGTCCTACAGCTGCCAGGTGACCCA |
| | | CGAGGGCAGCACCGTGGAAAAGACCGTGGCCCC |
| | | AACCGAGTGCAGC |
| EBB-C1975-B5- | | |
| SEQ ID NO: 226 | HCDR1 | AYYWT |
| (Kabat) | | |
| SEQ ID NO: 227 | HCDR2 | YISHSGSTNYNPSLKS |
| (Kabat) | | |
| SEQ ID NO: 228 | HCDR3 | LGDTASLSRFYYYIDV |
| (Kabat) | | |
| SEQ ID NO: 229 | HCDR1 | GGSTSAY |
| (Chothia) | | |
| SEQ ID NO: 230 | HCDR2 | SHSGS |
| (Chothia) | | |
| SEQ ID NO: 231 | HCDR3 | LGDTASLSRFYYYIDV |
| (Chothia) | | |
| SEQ ID NO: 232 | VH | QVQLVQSGPGLVKPSETLSLTCTVSGGSTSAYYWT |
| | | WIRQPPGKGLEWIGYISHSGSTNYNPSLKSRVTISA |
| | | DTSKNQLSLKVNSVTAADTAVYYCARLGDTASLS |
| | | RFYYYIDVWGKGTTVTVSS |
| SEQ ID NO: 233 | DNA VH | CAGGTGCAGCTGGTGCAGTCTGGCCCAGGACTG |
| | | GTGAAGCCTTCGGAGACCCTGTCCCTCACCTGCA |
| | | CTGTCTCTGGTGGCTCCACCAGTGCTTACTACTG |
| | | GACCTGGATTCGGCAGCCCCCAGGGAAGGGACT |
| | | GGAGTGGATTGGGTATATCTCTCACAGTGGGAGC |
| | | ACCAACTACAACCCCTCCCTCAAGAGTCGAGTCA |
| | | CCATATCAGCAGACACGTCCAAGAACCAGCTCTC |
| | | CCTGAAGGTGAACTCTGTGACCGCCGCAGACAC |
| | | GGCCGTGTATTACTGTGCGAGACTTGGGGATACA |
| | | GCTTCACTTAGCCGCTTCTACTACTACATTGACG |
| | | TCTGGGGCAAAGGGACCACGGTCACCGTCTCCTC |
| | | A |
| SEQ ID NO: 234 | Heavy | QVQLVQSGPGLVKPSETLSLTCTVSGGSTSAYYWT |
| | Chain | WIRQPPGKGLEWIGYISHSGSTNYNPSLKSRVTISA |
| | | DTSKNQLSLKVNSVTAADTAVYYCARLGDTASLS |
| | | RFYYYIDVWGKGTTVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 235 | DNA | CAGGTGCAGCTGGTGCAGTCTGGCCCAGGACTG |
| | Heavy | GTGAAGCCTTCGGAGACCCTGTCCCTCACCTGCA |
| | Chain | CTGTCTCTGGTGGCTCCACCAGTGCTTACTACTG |
| | | GACCTGGATTCGGCAGCCCCCAGGGAAGGGACT |
| | | GGAGTGGATTGGGTATATCTCTCACAGTGGGAGC |
| | | ACCAACTACAACCCCTCCCTCAAGAGTCGAGTCA |
| | | CCATATCAGCAGACACGTCCAAGAACCAGCTCTC |
| | | CCTGAAGGTGAACTCTGTGACCGCCGCAGACAC |
| | | GGCCGTGTATTACTGTGCGAGACTTGGGGATACA |
| | | GCTTCACTTAGCCGCTTCTACTACTACATTGACG |
| | | TCTGGGGCAAAGGGACCACGGTCACCGTCTCCTC |
| | | AGCTAGCACCAAGGGCCCATCGGTCTTCCCCCTG |
| | | GCACCCTCCTCCAAGAGCACCTCTGGGGGCACA |
| | | GCGGCCCTGGGCTGCCTGGTCAAGGACTACTTCC |
| | | CCGAACCGGTGACGGTGTCGTGGAACTCAGGCG |
| | | CCCTGACCAGCGGCGTGCACACCTTCCCGGCTGT |
| | | CCTACAGTCCTCAGGACTCTACTCCCTCAGCAGC |
| | | GTGGTGACCGTGCCCTCCAGCAGCTTGGGCACCC |
| | | AGACCTACATCTGCAACGTGAATCACAAGCCCA |
| | | GCAACACCAAGGTGGACAAGAGAGTTGAGCCCA |
| | | AATCTTGTGACAAAACTCACACATGCCCACCGTG |
| | | CCCAGCACCTGAACTCCTGGGGGGACCGTCAGTC |
| | | TTCCTCTTCCCCCCAAAACCCAAGGACACCCTCA |
| | | TGATCTCCCGGACCCCTGAGGTCACATGCGTGGT |
| | | GGTGGACGTGAGCCACGAAGACCCTGAGGTCAA |
| | | GTTCAACTGGTACGTGGACGGCGTGGAGGTGCA |
| | | TAATGCCAAGACAAAGCCGCGGGAGGAGCAGTA |
| | | CAACAGCACGTACCGTGTGGTCAGCGTCCTCACC |
| | | GTCCTGCACCAGGACTGGCTGAATGGCAAGGAG |
| | | TACAAGTGCAAGGTCTCCAACAAAGCCCTCCCA |
| | | GCCCCCATCGAGAAAACCATCTCCAAAGCCAAA |
| | | GGGCAGCCCCGAGAACCACAGGTGTACACCCTG |
| | | CCCCCATCCCGGGAGGAGATGACCAAGAACCAG |
| | | GTCAGCCTGACCTGCCTGGTCAAAGGCTTCTATC |
| | | CCAGCGACATCGCCGTGGAGTGGGAGAGCAATG |
| | | GGCAGCCGGAGAACAACTACAAGACCACGCCTC |
| | | CCGTGCTGGACTCCGACGGCTCCTTCTTCCTCTA |
| | | CAGCAAGCTCACCGTGGACAAGAGCAGGTGGCA |
| | | GCAGGGGAACGTCTTCTCATGCTCCGTGATGCAT |
| | | GAGGCTCTGCACAACCACTACACGCAGAAGAGC |
| | | CTCTCCCTGTCTCCGGGTAAA |
| SEQ ID NO: 236 | LCDR1 | RASQSVSSNYLA |
| (Kabat) | | |
| SEQ ID NO: 237 | LCDR2 | GASSRAT |
| (Kabat) | | |
| SEQ ID NO: 238 | LCDR3 | QQYGSSPPYT |
| (Kabat) | | |
| SEQ ID NO: 239 | LCDR1 | SQSVSSNY |
| (Chothia) | | |
| SEQ ID NO: 240 | LCDR2 | GAS |
| (Chothia) | | |
| SEQ ID NO: 241 | LCDR3 | YGSSPPY |
| (Chothia) | | |
| SEQ ID NO: 242 | VL | EIVMTQSPDTLSLSPGERATLSCRASQSVSSNYLAW |
| | | YQQKPGEAPRLLIYGASSRATGIPDRFSGSGSGTDF |
| | | TLTISRLEPEDFAVYYCQQYGSSPPYTFGQGTRLEI |
| | | K |
| SEQ ID NO: 243 | DNA VL | GAAATTGTAATGACGCAGTCTCCAGACACCCTGT |
| | | CTTTGTCTCCAGGGGAAAGAGCCACCCTCTCCTG |
| | | CAGGGCCAGTCAGAGTGTTAGCAGCAACTACTT |
| | | AGCCTGGTACCAGCAGAAACCTGGCGAGGCTCC |
| | | CAGGCTCCTCATCTATGGTGCATCCAGCAGGGCC |
| | | ACTGGCATCCCAGACAGGTTCAGTGGCAGTGGG |
| | | TCTGGGACAGACTTCACTCTCACCATCAGCAGAC |
| | | TGGAGCCTGAAGATTTTGCAGTGTATTACTGTCA |
| | | GCAGTATGGTAGCTCACCTCCGTACACTTTTGGC |
| | | CAGGGGACACGACTGGAGATTAAAC |
| SEQ ID NO: 244 | Light | EIVMTQSPDTLSLSPGERATLSCRASQSVSSNYLAW |
| | Chain | YQQKPGEAPRLLIYGASSRATGIPDRFSGSGSGTDF |
| | | TLTISRLEPEDFAVYYCQQYGSSPPYTFGQGTRLEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| SEQ ID NO: 245 | DNA | GAAATTGTAATGACGCAGTCTCCAGACACCCTGT |
| | Light | CTTTGTCTCCAGGGGAAAGAGCCACCCTCTCCTG |
| | Chain | CAGGGCCAGTCAGAGTGTTAGCAGCAACTACTT |
| | | AGCCTGGTACCAGCAGAAACCTGGCGAGGCTCC |
| | | CAGGCTCCTCATCTATGGTGCATCCAGCAGGGCC |
| | | ACTGGCATCCCAGACAGGTTCAGTGGCAGTGGG |
| | | TCTGGGACAGACTTCACTCTCACCATCAGCAGAC |
| | | TGGAGCCTGAAGATTTTGCAGTGTATTACTGTCA |
| | | GCAGTATGGTAGCTCACCTCCGTACACTTTTGGC |
| | | CAGGGGACACGACTGGAGATTAAACGTACGGTG |
| | | GCTGCACCATCTGTCTTCATCTTCCCGCCATCTGA |
| | | TGAGCAGTTGAAATCTGGAACTGCCTCTGTTGTG |
| | | TGCCTGCTGAATAACTTCTATCCCCGCGAGGCCA |
| | | AAGTACAGTGGAAGGTGGATAACGCCCTCCAAT |
| | | CGGGTAACTCCCAGGAGAGTGTCACAGAGCAGG |
| | | ACAGCAAGGACAGCACCTACAGCCTCAGCAGCA |
| | | CCCTGACGCTGAGCAAAGCAGACTACGAGAAAC |
| | | ACAAAGTCTACGCCTGCGAAGTCACCCATCAGG |
| | | GCCTGAGCTCGCCCGTCACAAAGAGCTTCAACCG |
| | | CGGAGAGTGT |
| EBB-C1975-A3 | | |
| SEQ ID NO: 246 | HCDR1 | RNYMS |
| (Kabat) | | |
| SEQ ID NO: 247 | HCDR2 | GIYSGGSTYYADSVKG |
| (Kabat) | | |
| SEQ ID NO: 248 | HCDR3 | EDEFWSGYSAGVD |
| (Kabat) | | |
| SEQ ID NO: 249 | HCDR1 | GFTVRRN |
| (Chothia) | | |
| SEQ ID NO: 250 | HCDR2 | YSGGS |
| (Chothia) | | |
| SEQ ID NO: 251 | HCDR3 | EDEFWSGYSAGVD |
| (Chothia) | | |
| SEQ ID NO: 252 | VH | EVQLVETGGGLVQPGGSLRLSCAASGFTVRRNYM |
| | | SWVRQAPGKGLEWVSGIYSGGSTYYADSVKGRFTI |
| | | SRDYSKNTLSLQMNTLRVEDTAVYFCAREDEFWS |
| | | GYSAGVDWGQGTLVTVSS |
| SEQ ID NO: 253 | DNA VH | GAGGTGCAGCTGGTGGAGACTGGAGGAGGCTTG |
| | | GTCCAGCCGGGGGGGTCCCTGAGACTCTCATGTG |
| | | CAGCCTCTGGATTCACCGTCAGACGCAATTACAT |
| | | GAGTTGGGTCCGCCAGGCTCCGGGGAAGGGACT |
| | | GGAGTGGGTCTCAGGGATCTACAGTGGTGGTAG |
| | | CACATACTACGCAGACTCCGTGAAGGGCCGATTC |
| | | ACCATCTCCAGAGACTATTCCAAGAACACACTGT |
| | | CTCTTCAAATGAACACCCTGAGAGTCGAGGACA |
| | | CGGCCGTGTATTTCTGTGCGAGAGAAGACGAATT |
| | | TTGGAGCGGGTATTCCGCTGGGGTCGACTGGGGC |
| | | CAGGGAACCCTGGTCACCGTCTCCTCAG |
| SEQ ID NO: 254 | Heavy | EVQLVETGGGLVQPGGSLRLSCAASGFTVRRNYM |
| | Chain | SWVRQAPGKGLEWVSGIYSGGSTYYADSVKGRFTI |
| | | SRDYSKNTLSLQMNTLRVEDTAVYFCAREDEFWS |
| | | GYSAGVDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 255 | DNA | GAGGTGCAGCTGGTGGAGACTGGAGGAGGCTTG |
| | Heavy | GTCCAGCCGGGGGGGTCCCTGAGACTCTCATGTG |
| | Chain | CAGCCTCTGGATTCACCGTCAGACGCAATTACAT |
| | | GAGTTGGGTCCGCCAGGCTCCGGGGAAGGGACT |
| | | GGAGTGGGTCTCAGGGATCTACAGTGGTGGTAG |
| | | CACATACTACGCAGACTCCGTGAAGGGCCGATTC |
| | | ACCATCTCCAGAGACTATTCCAAGAACACACTGT |
| | | CTCTTCAAATGAACACCCTGAGAGTCGAGGACA |
| | | CGGCCGTGTATTTCTGTGCGAGAGAAGACGAATT |
| | | TTGGAGCGGGTATTCCGCTGGGGTCGACTGGGGC |
| | | CAGGGAACCCTGGTCACCGTCTCCTCAGCTAGCA |
| | | CCAAGGGCCCATCGGTCTTCCCCCTGGCACCCTC |
| | | CTCCAAGAGCACCTCTGGGGGCACAGCGGCCCT |
| | | GGGCTGCCTGGTCAAGGACTACTTCCCCGAACCG |
| | | GTGACGGTGTCGTGGAACTCAGGCGCCCTGACC |
| | | AGCGGCGTGCACACCTTCCCGGCTGTCCTACAGT |
| | | CCTCAGGACTCTACTCCCTCAGCAGCGTGGTGAC |
| | | CGTGCCCTCCAGCAGCTTGGGCACCCAGACCTAC |
| | | ATCTGCAACGTGAATCACAAGCCCAGCAACACC |
| | | AAGGTGGACAAGAGAGTTGAGCCCAAATCTTGT |
| | | GACAAAACTCACACATGCCCACCGTGCCCAGCA |
| | | CCTGAACTCCTGGGGGGACCGTCAGTCTTCCTCT |
| | | TCCCCCCAAAACCCAAGGACACCCTCATGATCTC |
| | | CCGGACCCCTGAGGTCACATGCGTGGTGGTGGA |
| | | CGTGAGCCACGAAGACCCTGAGGTCAAGTTCAA |
| | | CTGGTACGTGGACGGCGTGGAGGTGCATAATGC |
| | | CAAGACAAAGCCGCGGGAGGAGCAGTACAACAG |
| | | CACGTACCGTGTGGTCAGCGTCCTCACCGTCCTG |
| | | CACCAGGACTGGCTGAATGGCAAGGAGTACAAG |
| | | TGCAAGGTCTCCAACAAAGCCCTCCCAGCCCCCA |
| | | TCGAGAAAACCATCTCCAAAGCCAAAGGGCAGC |
| | | CCCGAGAACCACAGGTGTACACCCTGCCCCCATC |
| | | CCGGGAGGAGATGACCAAGAACCAGGTCAGCCT |
| | | GACCTGCCTGGTCAAAGGCTTCTATCCCAGCGAC |
| | | ATCGCCGTGGAGTGGGAGAGCAATGGGCAGCCG |
| | | GAGAACAACTACAAGACCACGCCTCCCGTGCTG |
| | | GACTCCGACGGCTCCTTCTTCCTCTACAGCAAGC |
| | | TCACCGTGGACAAGAGCAGGTGGCAGCAGGGGA |
| | | ACGTCTTCTCATGCTCCGTGATGCATGAGGCTCT |
| | | GCACAACCACTACACGCAGAAGAGCCTCTCCCT |
| | | GTCTCCGGGTAAA |
| SEQ ID NO: 256 | LCDR1 | RASQSISSYLN |
| (Kabat) | | |
| SEQ ID NO: 257 | LCDR2 | AASSLQS |
| (Kabat) | | |
| SEQ ID NO: 258 | LCDR3 | QQSYNTPRT |
| (Kabat) | | |
| SEQ ID NO: 259 | LCDR1 | SQSISSY |
| (Chothia) | | |
| SEQ ID NO: 260 | LCDR2 | AAS |
| (Chothia) | | |
| SEQ ID NO: 261 | LCDR3 | SYNTPR |
| (Chothia) | | |
| SEQ ID NO: 262 | VL | DIRLTQSPSSLSASVGDRVTITCRASQSISSYLNWYQ |
| | | QKPGKAPKLLIYAASSLQSGVPSRFSGSGSGTDFTL |
| | | TISSLQPEDFATYYCQQSYNTPRTFGQGTKVEIK |
| SEQ ID NO: 263 | DNA VL | GACATCCGGTTGACCCAGTCTCCATCCTCCCTGT |
| | | CTGCATCTGTAGGAGACAGAGTCACCATCACTTG |
| | | CCGGGCAAGTCAGAGCATTAGCAGCTATTTGAAT |
| | | TGGTATCAGCAGAAACCAGGGAAAGCCCCTAAG |
| | | CTCCTGATCTATGCTGCATCCAGTTTGCAAAGTG |
| | | GGGTCCCATCAAGGTTCAGTGGCAGTGGATCTGG |
| | | GACAGATTTCACTCTCACCATCAGCAGTCTGCAA |
| | | CCTGAAGATTTTGCAACTTACTACTGTCAACAGA |
| | | GTTACAATACCCCTCGAACGTTCGGCCAAGGGAC |
| | | CAAGGTGGAGATCAAACG |
| SEQ ID NO: 264 | Light | DIRLTQSPSSLSASVGDRVTITCRASQSISSYLNWYQ |
| | Chain | QKPGKAPKLLIYAASSLQSGVPSRFSGSGSGTDFTL |
| | | TISSLQPEDFATYYCQQSYNTPRTFGQGTKVEIKRT |
| | | VAAPSVFIFPPSDEQLKSGTASVVCLLNNFYPREAK |
| | | VQWKVDNALQSGNSQESVTEQDSKDSTYSLSSTLT |
| | | LSKADYEKHKVYACEVTHQGLSSPVTKSFNRGEC |
| SEQ ID NO: 265 | DNA | GACATCCGGTTGACCCAGTCTCCATCCTCCCTGT |
| | Light | CTGCATCTGTAGGAGACAGAGTCACCATCACTTG |
| | Chain | CCGGGCAAGTCAGAGCATTAGCAGCTATTTGAAT |
| | | TGGTATCAGCAGAAACCAGGGAAAGCCCCTAAG |
| | | CTCCTGATCTATGCTGCATCCAGTTTGCAAAGTG |
| | | GGGTCCCATCAAGGTTCAGTGGCAGTGGATCTGG |
| | | GACAGATTTCACTCTCACCATCAGCAGTCTGCAA |
| | | CCTGAAGATTTTGCAACTTACTACTGTCAACAGA |
| | | GTTACAATACCCCTCGAACGTTCGGCCAAGGGAC |
| | | CAAGGTGGAGATCAAACGTACGGTGGCTGCACC |
| | | ATCTGTCTTCATCTTCCCGCCATCTGATGAGCAG |
| | | TTGAAATCTGGAACTGCCTCTGTTGTGTGCCTGC |
| | | TGAATAACTTCTATCCCCGCGAGGCCAAAGTACA |
| | | GTGGAAGGTGGATAACGCCCTCCAATCGGGTAA |
| | | CTCCCAGGAGAGTGTCACAGAGCAGGACAGCAA |
| | | GGACAGCACCTACAGCCTCAGCAGCACCCTGAC |
| | | GCTGAGCAAAGCAGACTACGAGAAACACAAAGT |
| | | CTACGCCTGCGAAGTCACCCATCAGGGCCTGAGC |
| | | TCGCCCGTCACAAAGAGCTTCAACCGCGGAGAG |
| | | TGT |
| EBB-C1975-A7 | | |
| SEQ ID NO: 266 | HCDR1 | RNYMS |
| (Kabat) | | |
| SEQ ID NO: 267 | HCDR2 | GIYSGGSTYYADSVKG |
| (Kabat) | | |
| SEQ ID NO: 268 | HCDR3 | EDEFWSGYSAGVD |
| (Kabat) | | |
| SEQ ID NO: 269 | HCDR1 | GFTVSRN |
| (Chothia) | | |
| SEQ ID NO: 270 | HCDR2 | YSGGS |
| (Chothia) | | |
| SEQ ID NO: 271 | HCDR3 | EDEFWSGYSAGVD |
| (Chothia) | | |
| SEQ ID NO: 272 | VH | QVQLVESGGGLVQPGGSLRLSCAASGFTVSRNYMS |
| | | WVRQAPGKGLEWVSGIYSGGSTYYADSVKGRFTIS |
| | | RDYSKNTLSLQMNTLRVEDTAVYFCAREDEFWSG |
| | | YSAGVDWGQGTLVTVSS |
| SEQ ID NO: 273 | DNA VH | CAGGTGCAGCTGGTGGAATCTGGAGGAGGCTTG |
| | | GTCCAGCCTGGGGGGTCCCTGAGACTCTCATGTG |
| | | CAGCCTCTGGATTCACCGTCAGTCGCAATTACAT |
| | | GAGTTGGGTCCGCCAGGCTCCGGGGAAGGGACT |
| | | GGAGTGGGTCTCAGGGATTTACAGTGGTGGTAG |
| | | CACATACTACGCAGACTCCGTGAAGGGCCGATTC |
| | | ACCATCTCCAGAGACTATTCCAAGAACACACTGT |
| | | CTCTTCAAATGAACACCCTGAGAGTCGAGGACA |
| | | CGGCCGTGTATTTCTGTGCGAGAGAAGACGAATT |
| | | TTGGAGTGGGTATTCCGCTGGGGTCGACTGGGGC |
| | | CAGGGAACCCTGGTCACCGTCTCCTCAGC |
| SEQ ID NO: 274 | Heavy | QVQLVESGGGLVQPGGSLRLSCAASGFTVSRNYMS |
| | Chain | WVRQAPGKGLEWVSGIYSGGSTYYADSVKGRFTIS |
| | | RDYSKNTLSLQMNTLRVEDTAVYFCAREDEFWSG |
| | | YSAGVDWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKRVEPKSCDKTHTCPPCPAPELLGGPSVFL |
| | | FPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPGK |
| SEQ ID NO: 275 | DNA | CAGGTGCAGCTGGTGGAATCTGGAGGAGGCTTG |
| | Heavy | GTCCAGCCTGGGGGGTCCCTGAGACTCTCATGTG |
| | Chain | CAGCCTCTGGATTCACCGTCAGTCGCAATTACAT |
| | | GAGTTGGGTCCGCCAGGCTCCGGGGAAGGGACT |
| | | GGAGTGGGTCTCAGGGATTTACAGTGGTGGTAG |
| | | CACATACTACGCAGACTCCGTGAAGGGCCGATTC |
| | | ACCATCTCCAGAGACTATTCCAAGAACACACTGT |
| | | CTCTTCAAATGAACACCCTGAGAGTCGAGGACA |
| | | CGGCCGTGTATTTCTGTGCGAGAGAAGACGAATT |
| | | TTGGAGTGGGTATTCCGCTGGGGTCGACTGGGGC |
| | | CAGGGAACCCTGGTCACCGTCTCCTCAGCTAGCA |
| | | CCAAGGGCCCATCGGTCTTCCCCCTGGCACCCTC |
| | | CTCCAAGAGCACCTCTGGGGGCACAGCGGCCCT |
| | | GGGCTGCCTGGTCAAGGACTACTTCCCCGAACCG |
| | | GTGACGGTGTCGTGGAACTCAGGCGCCCTGACC |
| | | AGCGGCGTGCACACCTTCCCGGCTGTCCTACAGT |
| | | CCTCAGGACTCTACTCCCTCAGCAGCGTGGTGAC |
| | | CGTGCCCTCCAGCAGCTTGGGCACCCAGACCTAC |
| | | ATCTGCAACGTGAATCACAAGCCCAGCAACACC |
| | | AAGGTGGACAAGAGAGTTGAGCCCAAATCTTGT |
| | | GACAAAACTCACACATGCCCACCGTGCCCAGCA |
| | | CCTGAACTCCTGGGGGGACCGTCAGTCTTCCTCT |
| | | TCCCCCCAAAACCCAAGGACACCCTCATGATCTC |
| | | CCGGACCCCTGAGGTCACATGCGTGGTGGTGGA |
| | | CGTGAGCCACGAAGACCCTGAGGTCAAGTTCAA |
| | | CTGGTACGTGGACGGCGTGGAGGTGCATAATGC |
| | | CAAGACAAAGCCGCGGGAGGAGCAGTACAACAG |
| | | CACGTACCGTGTGGTCAGCGTCCTCACCGTCCTG |
| | | CACCAGGACTGGCTGAATGGCAAGGAGTACAAG |
| | | TGCAAGGTCTCCAACAAAGCCCTCCCAGCCCCCA |
| | | TCGAGAAAACCATCTCCAAAGCCAAAGGGCAGC |
| | | CCCGAGAACCACAGGTGTACACCCTGCCCCCATC |
| | | CCGGGAGGAGATGACCAAGAACCAGGTCAGCCT |
| | | GACCTGCCTGGTCAAAGGCTTCTATCCCAGCGAC |
| | | ATCGCCGTGGAGTGGGAGAGCAATGGGCAGCCG |
| | | GAGAACAACTACAAGACCACGCCTCCCGTGCTG |
| | | GACTCCGACGGCTCCTTCTTCCTCTACAGCAAGC |
| | | TCACCGTGGACAAGAGCAGGTGGCAGCAGGGGA |
| | | ACGTCTTCTCATGCTCCGTGATGCATGAGGCTCT |
| | | GCACAACCACTACACGCAGAAGAGCCTCTCCCT |
| | | GTCTCCGGGTAAA |
| SEQ ID NO: 276 | LCDR1 | RASQSISSYLN |
| (Kabat) | | |
| SEQ ID NO: 277 | LCDR2 | AASSLQS |
| (Kabat) | | |
| SEQ ID NO: 278 | LCDR3 | QQSYSTPRT |
| (Kabat) | | |
| SEQ ID NO: 279 | LCDR1 | SQSISSY |
| (Chothia) | | |
| SEQ ID NO: 280 | LCDR2 | AAS |
| (Chothia) | | |
| SEQ ID NO: 281 | LCDR3 | SYSTPR |
| (Chothia) | | |
| SEQ ID NO: 282 | VL | DIRMTQSPSSLSASVGDRVTITCRASQSISSYLNWY |
| | | QQKPGKAPTLLIYAASSLQSGVPSRFSGSGSGTDFT |
| | | LTISSLQPEDFATYYCQQSYSTPRTFGQGTKVEIK |
| SEQ ID NO: 283 | DNA VL | GACATCCGGATGACCCAGTCTCCATCCTCCCTGT |
| | | CTGCATCTGTAGGAGACAGAGTCACCATCACTTG |
| | | CCGGGCAAGTCAGAGCATTAGCAGCTATTTAAAT |
| | | TGGTATCAGCAGAAACCAGGGAAAGCCCCTACG |
| | | CTCCTGATCTATGCTGCATCCAGTTTGCAAAGTG |
| | | GGGTCCCATCAAGGTTCAGTGGCAGTGGATCTGG |
| | | GACAGATTTCACTCTCACCATCAGCAGTCTGCAA |
| | | CCTGAAGATTTTGCAACTTACTACTGTCAACAGA |
| | | GTTACAGTACCCCTCGGACGTTCGGCCAAGGGAC |
| | | CAAGGTGGAGATCAAAC |
| SEQ ID NO: 284 | Light | DIRMTQSPSSLSASVGDRVTITCRASQSISSYLNWY |
| | Chain | QQKPGKAPTLLIYAASSLQSGVPSRFSGSGSGTDFT |
| | | LTISSLQPEDFATYYCQQSYSTPRTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| SEQ ID NO: 285 | DNA | GACATCCGGATGACCCAGTCTCCATCCTCCCTGT |
| | Light | CTGCATCTGTAGGAGACAGAGTCACCATCACTTG |
| | Chain | CCGGGCAAGTCAGAGCATTAGCAGCTATTTAAAT |
| | | TGGTATCAGCAGAAACCAGGGAAAGCCCCTACG |
| | | CTCCTGATCTATGCTGCATCCAGTTTGCAAAGTG |
| | | GGGTCCCATCAAGGTTCAGTGGCAGTGGATCTGG |
| | | GACAGATTTCACTCTCACCATCAGCAGTCTGCAA |
| | | CCTGAAGATTTTGCAACTTACTACTGTCAACAGA |
| | | GTTACAGTACCCCTCGGACGTTCGGCCAAGGGAC |
| | | CAAGGTGGAGATCAAACGTACGGTGGCTGCACC |
| | | ATCTGTCTTCATCTTCCCGCCATCTGATGAGCAG |
| | | TTGAAATCTGGAACTGCCTCTGTTGTGTGCCTGC |
| | | TGAATAACTTCTATCCCCGCGAGGCCAAAGTACA |
| | | GTGGAAGGTGGATAACGCCCTCCAATCGGGTAA |
| | | CTCCCAGGAGAGTGTCACAGAGCAGGACAGCAA |
| | | GGACAGCACCTACAGCCTCAGCAGCACCCTGAC |
| | | GCTGAGCAAAGCAGACTACGAGAAACACAAAGT |
| | | CTACGCCTGCGAAGTCACCCATCAGGGCCTGAGC |
| | | TCGCCCGTCACAAAGAGCTTCAACCGCGGAGAG |
| | | TGT |
| EBB-C1975-E7 | | |
| SEQ ID NO: 286 | HCDR1 | RNYMS |
| (Kabat) | | |
| SEQ ID NO: 287 | HCDR2 | GIYGGGRTYYAESVKG |
| (Kabat) | | |
| SEQ ID NO: 288 | HCDR3 | EDEFWSGYSAGVD |
| (Kabat) | | |
| SEQ ID NO: 289 | HCDR1 | GFTVSRN |
| (Chothia) | | |
| SEQ ID NO: 290 | HCDR2 | YGGGR |
| (Chothia) | | |
| SEQ ID NO: 291 | HCDR3 | EDEFWSGYSAGVD |
| (Chothia) | | |
| SEQ ID NO: 292 | VH | EVQLLESGGGLVRPGGSLRVSCAASGFTVSRNYMS |
| | | WVRQAPGKGLEWVSGIYGGGRTYYAESVKGRFTI |
| | | SRDYSKNTLFLQMNTLRVEDTALYFCAREDEFWS |
| | | GYSAGVDWGQGTLVTVSS |
| SEQ ID NO: 293 | DNA VH | GAGGTGCAGCTGTTGGAGTCCGGGGGAGGCTTG |
| | | GTCCGGCCTGGGGGGTCCCTGAGAGTCTCATGTG |
| | | CAGCCTCTGGATTCACCGTCAGTCGCAATTACAT |
| | | GAGTTGGGTCCGCCAGGCTCCGGGGAAGGGACT |
| | | GGAGTGGGTCTCAGGGATTTACGGTGGTGGTAG |
| | | GACTTACTACGCAGAGTCCGTGAAGGGCCGATTC |
| | | ACCATCTCCAGAGACTATTCCAAGAACACACTGT |
| | | TTCTTCAAATGAACACCCTGAGAGTCGAGGACAC |
| | | GGCCCTGTATTTCTGTGCGAGAGAAGACGAATTT |
| | | TGGAGTGGGTATTCTGCTGGGGTCGACTGGGGCC |
| | | AGGGAACCCTGGTCACTGTCTCCTCA |
| SEQ ID NO: 294 | Heavy | EVQLLESGGGLVRPGGSLRVSCAASGFTVSRNYMS |
| | Chain | WVRQAPGKGLEWVSGIYGGGRTYYAESVKGRFTI |
| | | SRDYSKNTLFLQMNTLRVEDTALYFCAREDEFWS |
| | | GYSAGVDWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| SEQ ID NO: 295 | DNA | GAGGTGCAGCTGTTGGAGTCCGGGGGAGGCTTG |
| | Heavy | GTCCGGCCTGGGGGGTCCCTGAGAGTCTCATGTG |
| | Chain | CAGCCTCTGGATTCACCGTCAGTCGCAATTACAT |
| | | GAGTTGGGTCCGCCAGGCTCCGGGGAAGGGACT |
| | | GGAGTGGGTCTCAGGGATTTACGGTGGTGGTAG |
| | | GACTTACTACGCAGAGTCCGTGAAGGGCCGATTC |
| | | ACCATCTCCAGAGACTATTCCAAGAACACACTGT |
| | | TTCTTCAAATGAACACCCTGAGAGTCGAGGACAC |
| | | GGCCCTGTATTTCTGTGCGAGAGAAGACGAATTT |
| | | TGGAGTGGGTATTCTGCTGGGGTCGACTGGGGCC |
| | | AGGGAACCCTGGTCACTGTCTCCTCAGCTAGCAC |
| | | CAAGGGCCCATCGGTCTTCCCCCTGGCACCCTCC |
| | | TCCAAGAGCACCTCTGGGGGCACAGCGGCCCTG |
| | | GGCTGCCTGGTCAAGGACTACTTCCCCGAACCGG |
| | | TGACGGTGTCGTGGAACTCAGGCGCCCTGACCA |
| | | GCGGCGTGCACACCTTCCCGGCTGTCCTACAGTC |
| | | CTCAGGACTCTACTCCCTCAGCAGCGTGGTGACC |
| | | GTGCCCTCCAGCAGCTTGGGCACCCAGACCTACA |
| | | TCTGCAACGTGAATCACAAGCCCAGCAACACCA |
| | | AGGTGGACAAGAGAGTTGAGCCCAAATCTTGTG |
| | | ACAAAACTCACACATGCCCACCGTGCCCAGCAC |
| | | CTGAACTCCTGGGGGGACCGTCAGTCTTCCTCTT |
| | | CCCCCCAAAACCCAAGGACACCCTCATGATCTCC |
| | | CGGACCCCTGAGGTCACATGCGTGGTGGTGGAC |
| | | GTGAGCCACGAAGACCCTGAGGTCAAGTTCAAC |
| | | TGGTACGTGGACGGCGTGGAGGTGCATAATGCC |
| | | AAGACAAAGCCGCGGGAGGAGCAGTACAACAGC |
| | | ACGTACCGTGTGGTCAGCGTCCTCACCGTCCTGC |
| | | ACCAGGACTGGCTGAATGGCAAGGAGTACAAGT |
| | | GCAAGGTCTCCAACAAAGCCCTCCCAGCCCCCAT |
| | | CGAGAAAACCATCTCCAAAGCCAAAGGGCAGCC |
| | | CCGAGAACCACAGGTGTACACCCTGCCCCCATCC |
| | | CGGGAGGAGATGACCAAGAACCAGGTCAGCCTG |
| | | ACCTGCCTGGTCAAAGGCTTCTATCCCAGCGACA |
| | | TCGCCGTGGAGTGGGAGAGCAATGGGCAGCCGG |
| | | AGAACAACTACAAGACCACGCCTCCCGTGCTGG |
| | | ACTCCGACGGCTCCTTCTTCCTCTACAGCAAGCT |
| | | CACCGTGGACAAGAGCAGGTGGCAGCAGGGGAA |
| | | CGTCTTCTCATGCTCCGTGATGCATGAGGCTCTG |
| | | CACAACCACTACACGCAGAAGAGCCTCTCCCTGT |
| | | CTCCGGGTAAA |
| SEQ ID NO: 296 | LCDR1 | RASQSISSYLN |
| (Kabat) | | |
| SEQ ID NO: 297 | LCDR2 | AASTLQT |
| (Kabat) | | |
| SEQ ID NO: 298 | LCDR3 | QQSYNTPRT |
| (Kabat) | | |
| SEQ ID NO: 299 | LCDR1 | SQSISSY |
| (Chothia) | | |
| SEQ ID NO: 300 | LCDR2 | AAS |
| (Chothia) | | |
| SEQ ID NO: 301 | LCDR3 | SYNTPR |
| (Chothia) | | |
| SEQ ID NO: 302 | VL | DIQVTQSPSSLSASVGDRVTITCRASQSISSYLNWY |
| | | QQEPGKAPKLLIYAASTLQTGVPSRFSGSGSGTDFT |
| | | LTISSLQPEDFATYYCQQSYNTPRTFGQGTKVEIK |
| SEQ ID NO: 303 | DNA VL | GACATCCAGGTGACCCAGTCTCCATCCTCCCTGT |
| | | CTGCATCTGTAGGAGACAGAGTCACCATCACTTG |
| | | CCGGGCAAGTCAGAGCATTAGCAGCTATTTAAAT |
| | | TGGTATCAGCAGGAACCAGGGAAAGCCCCTAAA |
| | | CTCCTGATCTACGCTGCATCCACTTTGCAAACTG |
| | | GGGTCCCATCACGGTTCAGTGGTAGTGGATCTGG |
| | | GACAGATTTCACTCTCACCATCAGCAGTCTGCAA |
| | | CCTGAAGATTTTGCAACTTATTACTGTCAACAGA |
| | | GTTACAATACCCCTCGAACCTTCGGCCAAGGGAC |
| | | CAAGGTGGAAATCAAACG |
| SEQ ID NO: 304 | Light | DIQVTQSPSSLSASVGDRVTITCRASQSISSYLNWY |
| | Chain | QQEPGKAPKLLIYAASTLQTGVPSRFSGSGSGTDFT |
| | | LTISSLQPEDFATYYCQQSYNTPRTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| SEQ ID NO: 305 | DNA | GACATCCAGGTGACCCAGTCTCCATCCTCCCTGT |
| | Light | CTGCATCTGTAGGAGACAGAGTCACCATCACTTG |
| | Chain | CCGGGCAAGTCAGAGCATTAGCAGCTATTTAAAT |
| | | TGGTATCAGCAGGAACCAGGGAAAGCCCCTAAA |
| | | CTCCTGATCTACGCTGCATCCACTTTGCAAACTG |
| | | GGGTCCCATCACGGTTCAGTGGTAGTGGATCTGG |
| | | GACAGATTTCACTCTCACCATCAGCAGTCTGCAA |
| | | CCTGAAGATTTTGCAACTTATTACTGTCAACAGA |
| | | GTTACAATACCCCTCGAACCTTCGGCCAAGGGAC |
| | | CAAGGTGGAAATCAAACGTACGGTGGCTGCACC |
| | | ATCTGTCTTCATCTTCCCGCCATCTGATGAGCAG |
| | | TTGAAATCTGGAACTGCCTCTGTTGTGTGCCTGC |
| | | TGAATAACTTCTATCCCCGCGAGGCCAAAGTACA |
| | | GTGGAAGGTGGATAACGCCCTCCAATCGGGTAA |
| | | CTCCCAGGAGAGTGTCACAGAGCAGGACAGCAA |
| | | GGACAGCACCTACAGCCTCAGCAGCACCCTGAC |
| | | GCTGAGCAAAGCAGACTACGAGAAACACAAAGT |
| | | CTACGCCTGCGAAGTCACCCATCAGGGCCTGAGC |
| | | TCGCCCGTCACAAAGAGCTTCAACCGCGGAGAG |
| | | TGT |
| P46F4 | | |
| SEQ ID NO: 306 | HCDR1 | NGGYYWS |
| (Kabat) | | |
| SEQ ID NO: 307 | HCDR2 | CIHYSGGTYYNPSLKS |
| (Kabat) | | |
| SEQ ID NO: 308 | HCDR3 | ALIAAPGISDWFDP |
| (Kabat) | | |
| SEQ ID NO: 309 | HCDR1 | GGSISNGGY |
| (Chothia) | | |
| SEQ ID NO: 310 | HCDR2 | HYSGG |
| (Chothia) | | |
| SEQ ID NO: 311 | HCDR3 | ALIAAPGISDWFDP |
| (Chothia) | | |
| SEQ ID NO: 312 | VH | QVQLQESGPGLVKPSQTLSLTCTVSGGSISNGGYY |
| | | WSWIRLHPGKGLEWIGCIHYSGGTYYNPSLKSRVT |
| | | VSLDTSKNQFSLNLISVTAADTAIYFCARALIAAPGI |
| | | SDWFDPWGQGTLVTVSS |
| SEQ ID NO: 313 | Heavy | QVQLQESGPGLVKPSQTLSLTCTVSGGSISNGGYY |
| | Chain | WSWIRLHPGKGLEWIGCIHYSGGTYYNPSLKSRVT |
| | | VSLDTSKNQFSLNLISVTAADTAIYFCARALIAAPGI |
| | | SDWFDPWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPS |
| | | NTKVDKKVEPKSCDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| SEQ ID NO: 314 | LCDR1 | SGSNSNVGHNYVS |
| (Kabat) | | |
| SEQ ID NO: 315 | LCDR2 | DNNKRPS |
| (Kabat) | | |
| SEQ ID NO: 316 | LCDR3 | GTWDSSLSAGV |
| (Kabat) | | |
| SEQ ID NO: 317 | LCDR1 | SNSNVGHNY |
| (Chothia) | | |
| SEQ ID NO: 318 | LCDR2 | DNN |
| (Chothia) | | |
| SEQ ID NO: 319 | LCDR3 | WDSSLSAG |
| (Chothia) | | |
| SEQ ID NO: 320 | VL | QSVLTQPPSVSAAPGQKVTISCSGSNSNVGHNYVS |
| | | WYQQLPGTAPKLLIYDNNKRPSGIPDRFSGSKSGTS |
| | | ATLGITGLQTGDEADYYCGTWDSSLSAGVFGGGT |
| | | KVTVL |
| SEQ ID NO: 321 | Light | QSVLTQPPSVSAAPGQKVTISCSGSNSNVGHNYVS |
| | Chain | WYQQLPGTAPKLLIYDNNKRPSGIPDRFSGSKSGTS |
| | | ATLGITGLQTGDEADYYCGTWDSSLSAGVFGGGT |
| | | KVTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKTV |
| | | APTECS |
| 2081-20-8 hz53 | | |
| SEQ ID NO: 322 | HCDR1 | SSWMN |
| (Kabat) | | |
| SEQ ID NO: 323 | HCDR2 | RIYPGDADTYYSGKFKG |
| (Kabat) | | |
| SEQ ID NO: 324 | HCDR3 | HSSGFTY |
| (Kabat) | | |
| SEQ ID NO: 325 | HCDR1 | GYTFSSS |
| (Chothia) | | |
| SEQ ID NO: 326 | HCDR2 | YPGDAD |
| (Chothia) | | |
| SEQ ID NO: 327 | HCDR3 | HSSGFTY |
| (Chothia) | | |
| SEQ ID NO: 328 | VH | QVQLVQSGAEVKKPGASVKVSCKASGYTFSSSWM |
| | | NWVRQAPGQRLEWMGRIYPGDADTYYSGKFKGR |
| | | VTITADSSARTAYMELSSLRSEDTAVYYCAIHSSGF |
| | | TYWGQGTLVTVSS |
| SEQ ID NO: 329 | DNA VH | CAGGTCCAGCTTGTGCAGTCTGGGGCTGAGGTGA |
| | | AGAAGCCTGGGGCCTCAGTGAAGGTTTCCTGCA |
| | | AGGCTTCTGGCTATACATTCAGCAGCTCTTGGAT |
| | | GAACTGGGTGCGCCAGGCCCCCGGACAAAGGCT |
| | | TGAGTGGATGGGACGGATCTATCCAGGAGACGC |
| | | CGATACTTACTACAGTGGGAAATTCAAGGGCAG |
| | | AGTCACCATTACCGCCGACAGCTCCGCGAGAAC |
| | | AGCCTACATGGAGCTGAGCAGCCTGAGATCTGA |
| | | AGACACGGCTGTGTATTACTGTGCGATCCACAGC |
| | | TCGGGCTTTACTTACTGGGGCCAGGGCACCCTGG |
| | | TCACCGTCTCCTCAGC |
| SEQ ID NO: 330 | Heavy | QVQLVQSGAEVKKPGASVKVSCKASGYTFSSSWM |
| | Chain | NWVRQAPGQRLEWMGRIYPGDADTYYSGKFKGR |
| | | VTITADSSARTAYMELSSLRSEDTAVYYCAIHSSGF |
| | | TYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKRVEPKSCDKTHTCPPCPAPELLGGPSVFLFPPK |
| | | PKDTLMISRTPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVY |
| | | TLPPSREEMTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| SEQ ID NO: 331 | DNA | CAGGTCCAGCTTGTGCAGTCTGGGGCTGAGGTGA |
| | Heavy | AGAAGCCTGGGGCCTCAGTGAAGGTTTCCTGCA |
| | Chain | AGGCTTCTGGCTATACATTCAGCAGCTCTTGGAT |
| | | GAACTGGGTGCGCCAGGCCCCCGGACAAAGGCT |
| | | TGAGTGGATGGGACGGATCTATCCAGGAGACGC |
| | | CGATACTTACTACAGTGGGAAATTCAAGGGCAG |
| | | AGTCACCATTACCGCCGACAGCTCCGCGAGAAC |
| | | AGCCTACATGGAGCTGAGCAGCCTGAGATCTGA |
| | | AGACACGGCTGTGTATTACTGTGCGATCCACAGC |
| | | TCGGGCTTTACTTACTGGGGCCAGGGCACCCTGG |
| | | TCACCGTCTCCTCAGCTAGCACCAAGGGCCCATC |
| | | GGTCTTCCCCCTGGCACCCTCCTCCAAGAGCACC |
| | | TCTGGGGGCACAGCGGCCCTGGGCTGCCTGGTCA |
| | | AGGACTACTTCCCCGAACCGGTGACGGTGTCGTG |
| | | GAACTCAGGCGCCCTGACCAGCGGCGTGCACAC |
| | | CTTCCCGGCTGTCCTACAGTCCTCAGGACTCTAC |
| | | TCCCTCAGCAGCGTGGTGACCGTGCCCTCCAGCA |
| | | GCTTGGGCACCCAGACCTACATCTGCAACGTGAA |
| | | TCACAAGCCCAGCAACACCAAGGTGGACAAGAG |
| | | AGTTGAGCCCAAATCTTGTGACAAAACTCACACA |
| | | TGCCCACCGTGCCCAGCACCTGAACTCCTGGGGG |
| | | GACCGTCAGTCTTCCTCTTCCCCCCAAAACCCAA |
| | | GGACACCCTCATGATCTCCCGGACCCCTGAGGTC |
| | | ACATGCGTGGTGGTGGACGTGAGCCACGAAGAC |
| | | CCTGAGGTCAAGTTCAACTGGTACGTGGACGGC |
| | | GTGGAGGTGCATAATGCCAAGACAAAGCCGCGG |
| | | GAGGAGCAGTACAACAGCACGTACCGTGTGGTC |
| | | AGCGTCCTCACCGTCCTGCACCAGGACTGGCTGA |
| | | ATGGCAAGGAGTACAAGTGCAAGGTCTCCAACA |
| | | AAGCCCTCCCAGCCCCCATCGAGAAAACCATCTC |
| | | CAAAGCCAAAGGGCAGCCCCGAGAACCACAGGT |
| | | GTACACCCTGCCCCCATCCCGGGAGGAGATGAC |
| | | CAAGAACCAGGTCAGCCTGACCTGCCTGGTCAA |
| | | AGGCTTCTATCCCAGCGACATCGCCGTGGAGTGG |
| | | GAGAGCAATGGGCAGCCGGAGAACAACTACAAG |
| | | ACCACGCCTCCCGTGCTGGACTCCGACGGCTCCT |
| | | TCTTCCTCTACAGCAAGCTCACCGTGGACAAGAG |
| | | CAGGTGGCAGCAGGGGAACGTCTTCTCATGCTCC |
| | | GTGATGCATGAGGCTCTGCACAACCACTACACGC |
| | | AGAAGAGCCTCTCCCTGTCTCCGGGTAAA |
| SEQ ID NO: 332 | LCDR1 | RASQDISDYLN |
| (Kabat) | | |
| SEQ ID NO: 333 | LCDR2 | YTSRLHS |
| (Kabat) | | |
| SEQ ID NO: 334 | LCDR3 | QQTHTLPFT |
| (Kabat) | | |
| SEQ ID NO: 335 | LCDR1 | SQDISDY |
| (Chothia) | | |
| SEQ ID NO: 336 | LCDR2 | YTS |
| (Chothia) | | |
| SEQ ID NO: 337 | LCDR3 | THTLPF |
| (Chothia) | | |
| SEQ ID NO: 338 | VL | DIQMTQSPSSLSASVGDRVTITCRASQDISDYLNWY |
| | | QQKPGKAPKLLIYYTSRLHSGVPSRFSGSGSGTDYT |
| | | LTISSLQPEDFATYFCQQTHTLPFTFGGGTKVEIK |
| SEQ ID NO: 339 | DNA VL | GACATCCAGATGACCCAGTCTCCATCCTCCCTGT |
| | | CTGCATCTGTAGGAGACAGAGTCACCATCACTTG |
| | | CAGGGCAAGTCAGGACATTAGCGATTATTTAAA |
| | | CTGGTATCAGCAGAAACCAGGGAAAGCCCCTAA |
| | | GCTCCTGATCTATTATACATCAAGATTACACTCA |
| | | GGGGTCCCATCAAGGTTCAGTGGCAGTGGATCTG |
| | | GGACAGATTACACTCTCACCATCAGCAGTCTGCA |
| | | ACCTGAAGATTTTGCAACTTACTTCTGTCAACAG |
| | | ACTCATACGCTTCCTTTCACGTTCGGCGGAGGGA |
| | | CCAAGGTGGAGATCAAACG |
| SEQ ID NO: 340 | Light | DIQMTQSPSSLSASVGDRVTITCRASQDISDYLNWY |
| | Chain | QQKPGKAPKLLIYYTSRLHSGVPSRFSGSGSGTDYT |
| | | LTISSLQPEDFATYFCQQTHTLPFTFGGGTKVEIKRT |
| | | VAAPSVFIFPPSDEQLKSGTASVVCLLNNFYPREAK |
| | | VQWKVDNALQSGNSQESVTEQDSKDSTYSLSSTLT |
| | | LSKADYEKHKVYACEVTHQGLSSPVTKSFNRGEC |
| SEQ ID NO: 341 | DNA | GACATCCAGATGACCCAGTCTCCATCCTCCCTGT |
| | Light | CTGCATCTGTAGGAGACAGAGTCACCATCACTTG |
| | Chain | CAGGGCAAGTCAGGACATTAGCGATTATTTAAA |
| | | CTGGTATCAGCAGAAACCAGGGAAAGCCCCTAA |
| | | GCTCCTGATCTATTATACATCAAGATTACACTCA |
| | | GGGGTCCCATCAAGGTTCAGTGGCAGTGGATCTG |
| | | GGACAGATTACACTCTCACCATCAGCAGTCTGCA |
| | | ACCTGAAGATTTTGCAACTTACTTCTGTCAACAG |
| | | ACTCATACGCTTCCTTTCACGTTCGGCGGAGGGA |
| | | CCAAGGTGGAGATCAAACGTACGGTGGCTGCAC |
| | | CATCTGTCTTCATCTTCCCGCCATCTGATGAGCA |
| | | GTTGAAATCTGGAACTGCCTCTGTTGTGTGCCTG |
| | | CTGAATAACTTCTATCCCCGCGAGGCCAAAGTAC |
| | | AGTGGAAGGTGGATAACGCCCTCCAATCGGGTA |
| | | ACTCCCAGGAGAGTGTCACAGAGCAGGACAGCA |
| | | AGGACAGCACCTACAGCCTCAGCAGCACCCTGA |
| | | CGCTGAGCAAAGCAGACTACGAGAAACACAAAG |
| | | TCTACGCCTGCGAAGTCACCCATCAGGGCCTGAG |
| | | CTCGCCCGTCACAAAGAGCTTCAACCGCGGAGA |
| | | GTGT |
| 2075-16-1 | | |
| SEQ ID NO: 342 | HCDR1 | NYWMH |
| (Kabat) | | |
| SEQ ID NO: 343 | HCDR2 | NIYPGSGNTNYGENFKS |
| (Kabat) | | |
| SEQ ID NO: 344 | HCDR3 | SAIYYGYDGHYFAMDY |
| (Kabat) | | |
| SEQ ID NO: 345 | HCDR1 | GYTFTNY |
| (Chothia) | | |
| SEQ ID NO: 346 | HCDR2 | YPGSGN |
| (Chothia) | | |
| SEQ ID NO: 347 | HCDR3 | SAIYYGYDGHYFAMDY |
| (Chothia) | | |
| SEQ ID NO: 348 | VH | QVQLQQPGSELVRPGASVKLSCKASGYTFTNYWM |
| | | HWVKQGHGQGLEWIGNIYPGSGNTNYGENFKSKG |
| | | TLTVDTSSSTAYMHLSRLTSEDSAVYYCSRSAIYYG |
| | | YDGHYFAMDYWGQGTSVTVSS |
| SEQ ID NO: 349 | LCDR1 | KASQDIRKYIA |
| (Kabat) | | |
| SEQ ID NO: 350 | LCDR2 | YTSTLQS |
| (Kabat) | | |
| SEQ ID NO: 351 | LCDR3 | LQYDNILFT |
| (Kabat) | | |
| SEQ ID NO: 352 | LCDR1 | SQDIRKY |
| (Chothia) | | |
| SEQ ID NO: 353 | LCDR2 | YTS |
| (Chothia) | | |
| SEQ ID NO: 354 | LCDR3 | YDNILF |
| (Chothia) | | |
| SEQ ID NO: 355 | VL | DIQMTQSPSSLSASLGGKVTITCKASQDIRKYIAWY |
| | | QHKPGKGPRLLINYTSTLQSGIPSRFRGSGSGRDYS |
| | | FSISNLEPEDIATYYCLQYDNILFTFGTGTKLEIK |
| 2075-456-4 | | |
| SEQ ID NO: 356 | HCDR1 | SCWMN |
| (Kabat) | | |
| SEQ ID NO: 357 | HCDR2 | RIYPGDGDTKYTEKFKD |
| (Kabat) | | |
| SEQ ID NO: 358 | HCDR3 | SGSGLPY |
| (Kabat) | | |
| SEQ ID NO: 359 | HCDR1 | GYSFSSC |
| (Chothia) | | |
| SEQ ID NO: 360 | HCDR2 | YPGDGD |
| (Chothia) | | |
| SEQ ID NO: 361 | HCDR3 | SGSGLPY |
| (Chothia) | | |
| SEQ ID NO: 362 | VH | QVHLQQSGPELVKPGASVTISCKTSGYSFSSCWMN |
| | | WVKQRPGQGLEWIGRIYPGDGDTKYTEKFKDKAT |
| | | LTADKSSSTAYMQLSSLTSVDSALYFCAISGSGLPY |
| | | WGQGTLVTVSE |
| SEQ ID NO: 363 | LCDR1 | RASQDIHNYLN |
| (Kabat) | | |
| SEQ ID NO: 364 | LCDR2 | STSRLHS |
| (Kabat) | | |
| SEQ ID NO: 365 | LCDR3 | QQTHTLPLT |
| (Kabat) | | |
| SEQ ID NO: 366 | LCDR1 | SQDIHNY |
| (Chothia) | | |
| SEQ ID NO: 367 | LCDR2 | STS |
| (Chothia) | | |
| SEQ ID NO: 368 | LCDR3 | THTLPL |
| (Chothia) | | |
| SEQ ID NO: 369 | VL | DIQMTQTTSSLSASLGDRVTISCRASQDIHNYLNWY |
| | | QQKPDGTIKLLIYSTSRLHSGVPSRFSGSGSGTHYSL |
| | | TINNLEQEDIATYFCQQTHTLPLTFGAGTKLELK |
| 2081-36-8 | | |
| SEQ ID NO: 370 | HCDR1 | SYWMN |
| (Kabat) | | |
| SEQ ID NO: 371 | HCDR2 | QIYPGNGDTNYNGKFKG |
| (Kabat) | | |
| SEQ ID NO: 372 | HCDR3 | EARQGYHYAMDY |
| (Kabat) | | |
| SEQ ID NO: 373 | HCDR1 | GYAFSSY |
| (Chothia) | | |
| SEQ ID NO: 374 | HCDR2 | YPGNGD |
| (Chothia) | | |
| SEQ ID NO: 375 | HCDR3 | EARQGYHYAMDY |
| (Chothia) | | |
| SEQ ID NO: 376 | VH | QVQLQQSGAGLVRPGSSVKISCKTSGYAFSSYWM |
| | | NWVKQRPGQGLEWIGQIYPGNGDTNYNGKFKGK |
| | | ATLTADKSSNTAYIQLNSLTSEDSAVYFCAREARQ |
| | | GYHY AMDYWGQGTSVTVSL |
| SEQ ID NO: 377 | LCDR1 | SASSMINSNYLH |
| (Kabat) | | |
| SEQ ID NO: 378 | LCDR2 | RTSNLAS |
| (Kabat) | | |
| SEQ ID NO: 379 | LCDR3 | QQGSNIFT |
| (Kabat) | | |
| SEQ ID NO: 380 | LCDR1 | SSMINSNY |
| (Chothia) | | |
| SEQ ID NO: 381 | LCDR2 | RTS |
| (Chothia) | | |
| SEQ ID NO: 382 | LCDR3 | GSNIF |
| (Chothia) | | |
| SEQ ID NO: 383 | VL | EIVFTQSPTTMAAFPGEKITITCSASSMINSNYLHWY |
| | | QQKPGFSPKVLIYRTSNLASGVPARFSGTGSGTSFS |
| | | LTIGTMEAEDVATYYCQQGSNIFTFGSGTKLEIK |
| 2081-66-5 | | |
| SEQ ID NO: 384 | HCDR1 | NSWMN |
| (Kabat) | | |
| SEQ ID NO: 385 | HCDR2 | RIYPGDGDTQYNEKFKG |
| (Kabat) | | |
| SEQ ID NO: 386 | HCDR3 | SRSGLDY |
| (Kabat) | | |
| SEQ ID NO: 387 | HCDR1 | GFTFSNS |
| (Chothia) | | |
| SEQ ID NO: 388 | HCDR2 | YPGDGD |
| (Chothia) | | |
| SEQ ID NO: 389 | HCDR3 | SRSGLDY |
| (Chothia) | | |
| SEQ ID NO: 390 | VH | QVQLQQSGPELVKPGASVRISCKVSGFTFSNSWMN |
| | | WVKQRPGQGLEWIGRIYPGDGDTQYNEKFKGKAT |
| | | LTADTSSNTAYIQLNSLTSVDSAVFFCARSRSGLDY |
| | | WGQGTTLTVSS |
| SEQ ID NO: 391 | LCDR1 | RASQDIYNYLN |
| (Kabat) | | |
| SEQ ID NO: 392 | LCDR2 | STSRLHS |
| (Kabat) | | |
| SEQ ID NO: 393 | LCDR3 | HQSHTVPFT |
| (Kabat) | | |
| SEQ ID NO: 394 | LCDR1 | SQDIYNY |
| (Chothia) | | |
| SEQ ID NO: 395 | LCDR2 | STS |
| (Chothia) | | |
| SEQ ID NO: 396 | LCDR3 | SHTVPF |
| (Chothia) | | |
| SEQ ID NO: 397 | VL | DIQMTQSTSSLSASLGDRVTISCRASQDIYNYLNWF |
| | | QQKPDGTVKPLIYSTSRLHSGVSSRFSGSGSGTDYS |
| | | LTISNLEREDIATYFCHQSHTVPFTFGSGTKLEIK |
| 2081-38-5 | | |
| SEQ ID NO: 398 | HCDR1 | SSWIN |
| (Kabat) | | |
| SEQ ID NO: 399 | HCDR2 | RIYPGDGDTNYNGKFKG |
| (Kabat) | | |
| SEQ ID NO: 400 | HCDR3 | HSSGFPH |
| (Kabat) | | |
| SEQ ID NO: 401 | HCDR1 | GYTFSSS |
| (Chothia) | | |
| SEQ ID NO: 402 | HCDR2 | YPGDGD |
| (Chothia) | | |
| SEQ ID NO: 403 | HCDR3 | HSSGFPH |
| (Chothia) | | |
| SEQ ID NO: 404 | VH | QVQLQQSGPELVKPGASVKISCKASGYTFSSSWIN |
| | | WVKQRPGQGLEWIGRIYPGDGDTNYNGKFKGKAT |
| | | LTADKSSSTVDMHLSSLTYVDSAVYFCAIHSSGFPH |
| | | WGQGTLVTVSA |
| SEQ ID NO: 405 | LCDR1 | RTSQDISDYLN |
| (Kabat) | | |
| SEQ ID NO: 406 | LCDR2 | YTSRLHS |
| (Kabat) | | |
| SEQ ID NO: 407 | LCDR3 | QQTNTLPFT |
| (Kabat) | | |
| SEQ ID NO: 408 | LCDR1 | SQDISDY |
| (Chothia) | | |
| SEQ ID NO: 409 | LCDR2 | YTS |
| (Chothia) | | |
| SEQ ID NO: 410 | LCDR3 | TNTLPF |
| (Chothia) | | |
| SEQ ID NO: 411 | VL | DIQMTQTTSSLSASLGGRVTISCRTSQDISDYLNWY |
| | | QQKPDGAVKLLIYYTSRLHSGVPSRFSGSGSGTDYS |
| | | LTISNLEQEDIATYFCQQTNTLPFTFGGGTKLEIK |
| 2081-25-6 | | |
| SEQ ID NO: 412 | HCDR1 | RYWMN |
| (Kabat) | | |
| SEQ ID NO: 413 | HCDR2 | QIYPGDGDTKYNGKFKD |
| (Kabat) | | |
| SEQ ID NO: 414 | HCDR3 | YGNYGMDY |
| (Kabat) | | |
| SEQ ID NO: 415 | HCDR1 | GYAFSRY |
| (Chothia) | | |
| SEQ ID NO: 416 | HCDR2 | YPGDGD |
| (Chothia) | | |
| SEQ ID NO: 417 | HCDR3 | YGNYGMDY |
| (Chothia) | | |
| SEQ ID NO: 418 | | QVQLQQSGAELVRPGSSVKISCKASGYAFSRYWM |
| | | NWVKQRPGQGLEWIGQIYPGDGDTKYNGKFKDTA |
| | | TLTADKSSSTAYLQLSSLTSEDSAVYFCAKYGNYG |
| | | MDYWGQGTSVTVSS |
| SEQ ID NO: 419 | LCDR1 | RSSQSLEYGNGNTYLN |
| (Kabat) | | |
| SEQ ID NO: 420 | LCDR2 | RVSNRFS |
| (Kabat) | | |
| SEQ ID NO: 421 | LCDR3 | LQFTHVPYT |
| (Kabat) | | |
| SEQ ID NO: 422 | LCDR1 | SQSLEYGNGNTY |
| (Chothia) | | |
| SEQ ID NO: 423 | LCDR2 | RVS |
| (Chothia) | | |
| SEQ ID NO: 424 | LCDR3 | FTHVPY |
| (Chothia) | | |
| SEQ ID NO: 425 | VL | DAVMTQTPLSLPVSLGDQASISCRSSQSLEYGNGNT |
| | | YLNWYLQKPGQSPQLLIYRVSNRFSGVLDRFSGSG |
| | | SGTDFTLKISRVEAEDLGVYFCLQFTHVPYTFGGGT |
| | | KLEIK |
| 2077-4-1 | | |
| SEQ ID NO: 426 | HCDR1 | GYTMN |
| (Kabat) | | |
| SEQ ID NO: 427 | HCDR2 | LFNPYNGGTRYNQKFKG |
| (Kabat) | | |
| SEQ ID NO: 428 | HCDR3 | LRNYGIGDDFFDY |
| (Kabat) | | |
| SEQ ID NO: 429 | HCDR1 | GYSFTGY |
| (Chothia) | | |
| SEQ ID NO: 430 | HCDR2 | NPYNGG |
| (Chothia) | | |
| SEQ ID NO: 431 | HCDR3 | LRNYGIGDDFFDY |
| (Chothia) | | |
| SEQ ID NO: 432 | VH | EVQLQQSGPELVKPGASMKISCKASGYSFTGYTMN |
| | | WVKQSHGENLEWIGLFNPYNGGTRYNQKFKGKAT |
| | | LTVDKSSSTAYMELLSLTSEDSAVYYCARLRNYGI |
| | | GDDFFDYWGQGTTLTVSS |
| SEQ ID NO: 433 | LCDR1 | KASQDVGTAVA |
| (Kabat) | | |
| SEQ ID NO: 434 | LCDR2 | WASTRHT |
| (Kabat) | | |
| SEQ ID NO: 435 | LCDR3 | QQYSNYPYT |
| (Kabat) | | |
| SEQ ID NO: 436 | LCDR1 | SQDVGTA |
| (Chothia) | | |
| SEQ ID NO: 437 | LCDR2 | WAS |
| (Chothia) | | |
| SEQ ID NO: 438 | LCDR3 | YSNYPY |
| (Chothia) | | |
| SEQ ID NO: 439 | VL | DIVMTQSHKFMSTSVGDRVSITCKASQDVGTAVA |
| | | WYQQKPGQSPKLLIYWASTRHTGVPDRFTGSGSGT |
| | | DFTLTISNVQSEDLTDYFCQQYSNYPYTFGGGTKLE |
| | | IK |
| 2077-7-5 | | |
| SEQ ID NO: 440 | HCDR1 | GYTMN |
| (Kabat) | | |
| SEQ ID NO: 441 | HCDR2 | LFNPYNGGINYNQKFKG |
| (Kabat) | | |
| SEQ ID NO: 442 | HCDR3 | LRYYGIGDDFFDY |
| (Kabat) | | |
| SEQ ID NO: 443 | HCDR1 | GYSFTGY |
| (Chothia) | | |
| SEQ ID NO: 444 | HCDR2 | NPYNGG |
| (Chothia) | | |
| SEQ ID NO: 445 | HCDR3 | LRYYGIGDDFFDY |
| (Chothia) | | |
| SEQ ID NO: 446 | VH | EVQLQQSGPELVKPGASMKISCKASGYSFTGYTMN |
| | | WVKQSHGKNLEWIGLFNPYNGGINYNQKFKGKAT |
| | | LTVDKSSSTAYMELLSLTSEDSAVYYCARLRYYGI |
| | | GDDFFDYWGQGTSLTVSS |
| SEQ ID NO: 447 | LCDR1 | KASRDVGTAVA |
| (Kabat) | | |
| SEQ ID NO: 448 | LCDR2 | WASTRHT |
| (Kabat) | | |
| SEQ ID NO: 449 | LCDR3 | QQYSNYPYT |
| (Kabat) | | |
| SEQ ID NO: 450 | LCDR1 | SRDVGTA |
| (Chothia) | | |
| SEQ ID NO: 451 | LCDR2 | WAS |
| (Chothia) | | |
| SEQ ID NO: 452 | LCDR3 | YSNYPY |
| (Chothia) | | |
| SEQ ID NO: 453 | VL | DIVMTQSHKFMSTSVGDRVSITCKASRDVGTAVA |
| | | WYQQKPGQSPKLLIYWASTRHTGVPDRFTGSGSGT |
| | | DFTLTISNVQSEDLADYFCQQYSNYPYTFGGGTKL |
| | | EMK |
| 2077-10-1 | | |
| SEQ ID NO: 454 | HCDR1 | GYTMN |
| (Kabat) | | |
| SEQ ID NO: 455 | HCDR2 | LFNPYNGGPNYNQKFKG |
| (Kabat) | | |
| SEQ ID NO: 456 | HCDR3 | LRYYGIGDDFFDY |
| (Kabat) | | |
| SEQ ID NO: 457 | HCDR1 | GYSFTGY |
| (Chothia) | | |
| SEQ ID NO: 458 | HCDR2 | NPYNGG |
| (Chothia) | | |
| SEQ ID NO: 459 | HCDR3 | LRYYGIGDDFFDY |
| (Chothia) | | |
| SEQ ID NO: 460 | VH | EVQLQQSGPELVKPGASMKISCKASGYSFTGYTMN |
| | | WMKQGHGKNLEWIGLFNPYNGGPNYNQKFKGKA |
| | | TLTVDKSSSTAYMELLSLTSEDSAVYYCARLRYYG |
| | | IGDDFFDYWGQGTTLTVSS |
| SEQ ID NO: 461 | LCDR1 | KASQDVGTAVA |
| (Kabat) | | |
| SEQ ID NO: 462 | LCDR2 | WASTRHT |
| (Kabat) | | |
| SEQ ID NO: 463 | LCDR3 | QQYSSYPYT |
| (Kabat) | | |
| SEQ ID NO: 464 | LCDR1 | SQDVGTA |
| (Chothia) | | |
| SEQ ID NO: 465 | LCDR2 | WAS |
| (Chothia) | | |
| SEQ ID NO: 466 | LCDR3 | YSSYPY |
| (Chothia) | | |
| SEQ ID NO: 467 | VL | DIVMTQSHKFMSTSVGDRVSITCKASQDVGTAVA |
| | | WYQQKPGQSPKLLIYWASTRHTGVPDRFTGSGSGT |
| | | DFTLTITNVQSEDLTDYFCQQYSSYPYTFGGGTKLE |
| | | IK |
| 2077-26-1 | | |
| SEQ ID NO: 468 | HCDR1 | GYTMN |
| (Kabat) | | |
| SEQ ID NO: 469 | HCDR2 | LFNPYNGGPSYNQKFKG |
| (Kabat) | | |
| SEQ ID NO: 470 | HCDR3 | LRYYGIGDDFFDY |
| (Kabat) | | |
| SEQ ID NO: 471 | HCDR1 | GYSFTGY |
| (Chothia) | | |
| SEQ ID NO: 472 | HCDR2 | NPYNGG |
| (Chothia) | | |
| SEQ ID NO: 473 | HCDR3 | LRYYGIGDDFFDY |
| (Chothia) | | |
| SEQ ID NO: 474 | VH | EVQLQQSGPDLVKPGASMKLSCKASGYSFTGYTM |
| | | NWVKQSHGKNLEWIGLFNPYNGGPSYNQKFKGKA |
| | | TLTVDKSSSTAYMELLSLTPEDSAVYYCARLRYYG |
| | | IGDDFFDYWGQGTTLTVSS |
| SEQ ID NO: 475 | LCDR1 | KASQDVGTAVA |
| (Kabat) | | |
| SEQ ID NO: 476 | LCDR2 | WASTRHT |
| (Kabat) | | |
| SEQ ID NO: 477 | LCDR3 | QQYSNYPYT |
| (Kabat) | | |
| SEQ ID NO: 478 | LCDR1 | SQDVGTA |
| (Chothia) | | |
| SEQ ID NO: 479 | LCDR2 | WAS |
| (Chothia) | | |
| SEQ ID NO: 480 | LCDR3 | YSNYPY |
| (Chothia) | | |
| SEQ ID NO: 481 | VL | DIVMTQSHKFMSTSVGDRVSITCKASQDVGTAVA |
| | | WYQEKPGQSPKLLIYWASTRHTGVPDRFTGSGSGT |
| | | DFTLTISNVQSEDLAYYFCQQYSNYPYTFGGGTKL |
| | | EIK |
| 2077-28-2 | | |
| SEQ ID NO: 482 | HCDR1 | GYTMN |
| (Kabat) | | |
| SEQ ID NO: 483 | HCDR2 | LFNPYNGGATYNQRFKG |
| (Kabat) | | |
| SEQ ID NO: 484 | HCDR3 | LRKYGIGDDFFDY |
| (Kabat) | | |
| SEQ ID NO: 485 | HCDR1 | GYSFTGY |
| (Chothia) | | |
| SEQ ID NO: 486 | HCDR2 | NPYNGG |
| (Chothia) | | |
| SEQ ID NO: 487 | HCDR3 | LRKYGIGDDFFDY |
| (Chothia) | | |
| SEQ ID NO: 488 | VH | EVQLQQSGPELVKPGASMKISCKASGYSFTGYTMN |
| | | WVKQSHGKNLEWIGLFNPYNGGATYNQRFKGKA |
| | | TLTVDKSSSTAYMDLLSLTSEDSAVYYCTRLRKYG |
| | | IGDDFFDYWGQGTTLTVSS |
| SEQ ID NO: 489 | LCDR1 | KASQDVGTAVA |
| (Kabat) | | |
| SEQ ID NO: 490 | LCDR2 | WASTRHT |
| (Kabat) | | |
| SEQ ID NO: 491 | LCDR3 | QQYSTYTYT |
| (Kabat) | | |
| SEQ ID NO: 492 | LCDR1 | SQDVGTA |
| (Chothia) | | |
| SEQ ID NO: 493 | LCDR2 | WAS |
| (Chothia) | | |
| SEQ ID NO: 494 | LCDR3 | YSTYTY |
| (Chothia) | | |
| SEQ ID NO: 495 | VL | DIVMTQSHKFMSTSVGDRVSITCKASQDVGTAVA |
| | | WYQQKPGQSPKLLIYWASTRHTGVPDRFTGSGSGT |
| | | DFTLTISNVQSEDLADYFCQQYSTYTYTFGGGTKL |
| | | EIK |
| P8D11 | | |
| SEQ ID NO: 508 | HCDR1 | GFTFNNYWMT |
| (Combined) | | |
| SEQ ID NO: 509 | HCDR2 | NIKKDGSEKYYVDSVRG |
| (Combined) | | |
| SEQ ID NO: 510 | HCDR3 | VRSGRYFALDD |
| (Combined) | | |
| SEQ ID NO: 511 | LCDR1 | GGDNIGSRPVH |
| (Combined) | | |
| SEQ ID NO: 512 | LCDR2 | DDSNRPS |
| (Combined) | | |
| SEQ ID NO: 513 | LCDR3 | QVWSSSTDHP |
| (Combined) | | |
| P8D11A | | |
| SEQ ID NO: 514 | HCDR1 | GFTFSNYWMT |
| (Combined) | | |
| SEQ ID NO: 515 | HCDR2 | NIKKDGSEKYYVDSVRG |
| (Combined) | | |
| SEQ ID NO: 516 | HCDR3 | VRSGRYFALDD |
| (Combined) | | |
| SEQ ID NO: 517 | LCDR1 | GGDNIGSRPVH |
| (Combined) | | |
| SEQ ID NO: 518 | LCDR2 | DDSNRPS |
| (Combined) | | |
| SEQ ID NO: 519 | LCDR3 | QVWSSSTDHP |
| (Combined) | | |
| P8D11B | | |
| SEQ ID NO: 520 | HCDR1 | GFTFKNYWMT |
| (Combined) | | |
| SEQ ID NO: 521 | HCDR2 | NIKKDGSEKYYVDSVRG |
| (Combined) | | |
| SEQ ID NO: 522 | HCDR3 | VRSGRYFALDD |
| (Combined) | | |
| SEQ ID NO: 523 | LCDR1 | GGDNIGSRPVH |
| (Combined) | | |
| SEQ ID NO: 524 | LCDR2 | DDSNRPS |
| (Combined) | | |
| SEQ ID NO: 525 | LCDR3 | QVWSSSTDHP |
| (Combined) | | |
| P8D11C | | |
| SEQ ID NO: 526 | HCDR1 | GFTFQNYWMT |
| (Combined) | | |
| SEQ ID NO: 527 | HCDR2 | NIKKDGSEKYYVDSVRG |
| (Combined) | | |
| SEQ ID NO: 528 | HCDR3 | VRSGRYFALDD |
| (Combined) | | |
| SEQ ID NO: 529 | LCDR1 | GGDNIGSRPVH |
| (Combined) | | |
| SEQ ID NO: 530 | LCDR2 | DDSNRPS |
| (Combined) | | |
| SEQ ID NO: 531 | LCDR3 | QVWSSSTDHP |
| (Combined) | | |
| P8D11D | | |
| SEQ ID NO: 532 | HCDR1 | GFTFNNYWMT |
| (Combined) | | |
| SEQ ID NO: 533 | HCDR2 | NIKKDGSEKYYVDSVRG |
| (Combined) | | |
| SEQ ID NO: 534 | HCDR3 | VRSGRYFALDD |
| (Combined) | | |
| SEQ ID NO: 535 | LCDR1 | GGDNIGSRPVH |
| (Combined) | | |
| SEQ ID NO: 536 | LCDR2 | DDSNRPS |
| (Combined) | | |
| SEQ ID NO: 537 | LCDR3 | QVWSSSTDHP |
| (Combined) | | |
| P8D11E | | |
| SEQ ID NO: 538 | HCDR1 | GFTFNNYWMT |
| (Combined) | | |
| SEQ ID NO: 539 | HCDR2 | NIKKDGSEKYYVDSVRG |
| (Combined) | | |
| SEQ ID NO: 540 | HCDR3 | VRSGRYFALDD |
| (Combined) | | |
| SEQ ID NO: 541 | LCDR1 | GGDNIGSRPVH |
| (Combined) | | |
| SEQ ID NO: 542 | LCDR2 | DDSNRPS |
| (Combined) | | |
| SEQ ID NO: 543 | LCDR3 | QVWSSSTDHP |
| (Combined) | | |

Other antibodies of the present disclosure include those where the amino acids or nucleic acids encoding the amino acids have been mutated; yet have at least 60, 70, 80, 90 or 95 percent identity to the sequences described in Table 2. In some aspects, it includes mutant amino acid sequences wherein no more than 1, 2, 3, 4 or 5 amino acids have been mutated in the variable regions when compared with the variable regions depicted in the sequence described in Table 2, while retaining substantially the same therapeutic activity.

Since each of these antibodies can bind to VP1, the VH, VL, full length light chain, and full length heavy chain sequences (amino acid sequences and the nucleotide sequences encoding the amino acid sequences) can be “mixed and matched” to create other VP1-binding antibodies. Such “mixed and matched” VP1-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise, a full length light chain sequence from a particular full length heavy chain/full length light chain pairing should be replaced with a structurally similar full length light chain sequence. Accordingly, in one aspect, the disclosure provides for an isolated monoclonal antibody or antigen binding region thereof having: a heavy chain variable region comprising an amino acid sequence selected from the group consisting of SEQ ID NO: 12, 32, 52, 72, 92, 112, 132, 152, 172, 192, 212, 232, 252, 272, 292, 312, 328, 348, 362, 376, 390, 404, 418, 432, 446, 460, 474, and 488 (Table 2); and a light chain variable region comprising an amino acid sequence selected from the group consisting of SEQ ID NO: 22, 42, 62, 82, 102, 122, 142, 162, 182, 202, 222, 242, 262, 282, 302, 320, 338, 355, 369, 383, 397, 411, 425, 439, 453, 467, 481 and 495 (Table 2); wherein the antibody specifically binds to VP1.

In another aspect, the disclosure provides (i) an isolated monoclonal antibody having: a full length heavy chain comprising an amino acid sequence that has been optimized for expression in the cell of a mammalian selected from the group consisting of SEQ ID NOs: 14, 34, 54, 74, 94, 114, 134, 154, 174, 194, 214, 234, 254, 274, 294, 313 and 330; and a full length light chain comprising an amino acid sequence that has been optimized for expression in the cell of a mammalian selected from the group consisting of SEQ ID NOs: 24, 44, 64, 84, 104, 124, 144, 164, 184, 204, 224, 244, 264, 284, 304, 321, 340, or (ii) a functional protein comprising an antigen binding portion thereof.

In another aspect, the present disclosure provides VP1-binding antibodies that comprise the heavy chain and light chain CDR1s, CDR2s and CDR3s as described in Table 2, or combinations thereof. The amino acid sequences of the VH CDR1s of the antibodies are shown in SEQ ID NOs: 6, 26, 46, 66, 86, 106, 126, 146, 166, 186, 206, 226, 246, 266, 286, 306, 322, 342, 356, 370, 384, 398, 412, 426, 440, 454, 468, and 482. The amino acid sequences of the VH CDR2s of the antibodies and are shown in SEQ ID NOs: 7, 27, 47, 67, 87, 107, 127, 147, 167, 187, 207, 227, 247, 267, 287, 307, 323, 343, 357, 371, 385, 399, 413, 427, 441, 455, 469, and 483. The amino acid sequences of the VH CDR3s of the antibodies are shown in SEQ ID NOs: 8, 28, 48, 68, 88, 108, 128, 148, 168, 188, 208, 228, 248, 268, 288, 308, 324, 344, 358, 372, 386, 400, 414, 428, 442, 456, 470, and 484. The amino acid sequences of the VL CDR1s of the antibodies are shown in SEQ ID NOs: 16, 36, 56, 76, 96, 116, 136, 156, 176, 196, 216, 236, 256, 276, 296, 314, 332, 349, 363, 377, 391, 405, 419, 433, 447, 461, 475 and 489. The amino acid sequences of the VL CDR2s of the antibodies are shown in SEQ ID NOs 17, 37, 57, 77, 97, 117, 137, 157, 177, 197, 217, 237, 257, 277, 297, 315, 333, 350, 364, 378, 392, 406, 420, 434, 448, 462, 476 and 490. The amino acid sequences of the VL CDR3s of the antibodies are shown in SEQ ID NOs: 18, 38, 58, 78, 98, 118, 138, 158, 178, 198, 218, 238, 258, 278, 298, 316, 334, 351, 365, 379, 393, 407, 421, 435, 449, 463, 477 and 491.

Given that each of these antibodies can bind to VP1 and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched, although each antibody must contain a VH CDR1, 2 and 3 and a VL CDR1, 2 and 3 to create other VP1-binding binding molecules. Such “mixed and matched” VP1-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosure.

Accordingly, the present disclosure provides an isolated monoclonal antibody or antigen binding region thereof comprising a heavy chain CDR1 comprising an amino acid sequence selected from the group consisting of SEQ ID NOs: 6, 26, 46, 66, 86, 106, 126, 146, 166, 186, 206, 226, 246, 266, 286, 306, 322, 342, 356, 370, 384, 398, 412, 426, 440, 454, 468, and 482; a heavy chain CDR2 comprising an amino acid sequence selected from the group consisting of SEQ ID NOs: 7, 27, 47, 67, 87, 107, 127, 147, 167, 187, 207, 227, 247, 267, 287, 307, 323, 343, 357, 371, 385, 399, 413, 427, 441, 455, 469, and 483; a heavy chain CDR3 comprising an amino acid sequence selected from the group consisting of SEQ ID NOs: 8, 28, 48, 68, 88, 108, 128, 148, 168, 188, 208, 228, 248, 268, 288, 308, 324, 344, 358, 372, 386, 400, 414, 428, 442, 456, 470, and 484; a light chain CDR1 comprising an amino acid sequence selected from the group consisting of SEQ ID NOs: 16, 36, 56, 76, 96, 116, 136, 156, 176, 196, 216, 236, 256, 276, 296, 314, 332, 349, 363, 377, 391, 405, 419, 433, 447, 461, 475 and 489; a light chain CDR2 comprising an amino acid sequence selected from the group consisting of SEQ ID NOs: 17, 37, 57, 77, 97, 117, 137, 157, 177, 197, 217, 237, 257, 277, 297, 315, 333, 350, 364, 378, 392, 406, 420, 434, 448, 462, 476 and 490; and a light chain CDR3 comprising an amino acid sequence selected from the group consisting of SEQ ID NOs: 18, 38, 58, 78, 98, 118, 138, 158, 178, 198, 218, 238, 258, 278, 298, 316, 334, 351, 365, 379, 393, 407, 421, 435, 449, 463, 477 and 491; wherein the antibody specifically binds to VP1.

In certain aspects, an antibody that specifically binds to VP1 is an antibody or antibody fragment (e.g., antigen binding fragment) that is described in Table 2.

### 1. Identification of Epitopes and Antibodies That Bind to the Same Epitope

The present disclosure provides antibodies and antibody fragments (e.g., antigen binding fragments) that bind to an epitope of VP1. In certain aspects the antibodies and antibody fragments can bind to the same epitopewithin all four BKV serotypes and/or JCV.

The present disclosure also provides antibodies and antibody fragments (e.g., antigen binding fragments) that bind to the same epitope as do the anti-VP1 antibodies described in Table 2. Additional antibodies and antibody fragments (e.g., antigen binding fragments) can therefore be identified based on their ability to cross-compete (e.g., to competitively inhibit the binding of, in a statistically significant manner) with other antibodies in binding assays. The ability of a test antibody to inhibit the binding of antibodies and antibody fragments (e.g., antigen binding fragments) of the present disclosure to VP1 (e.g., human BKV or JCV VP1) demonstrates that the test antibody can compete with that antibody or antibody fragment (e.g., antigen binding fragments) for binding to VP1; such an antibody may, according to non-limiting theory, bind to the same or a related (e.g., a structurally similar or spatially proximal) epitope on VP1 as the antibody or antibody fragment (e.g., antigen binding fragments) with which it competes. In a certain aspect, the antibody that binds to the same epitope on VP1 as the antibodies or antibody fragments (e.g., antigen binding fragments) of the present disclosure is a human or humanized monoclonal antibody. Such human or humanized monoclonal antibodies can be prepared and isolated as described herein.

### 2. Further Alteration of the Framework of Fc Region

The present disclosure disclosed specific anti-VP1 antibodies. These antibodies comprise modified antibodies or antigen binding fragments thereof that further comprise modifications to framework residues within VH and/or VL, e.g. to improve the properties of the antibody. Typically such framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis. Such “back-mutated” antibodies are also intended to be encompassed.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies can be engineered to include modifications within the Fc region, typically to alter one or more functional properties of the antibody, such as serum half-life, complement fixation, Fc receptor binding, and/or antigen-dependent cellular cytotoxicity. Furthermore, an antibody can be chemically modified (e.g., one or more chemical moieties can be attached to the antibody) or be modified to alter its glycosylation, again to alter one or more functional properties of the antibody. Each of these aspects is described in further detail below.

In one aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another aspect, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acids selected from amino acid residues can be replaced with a different amino acid residue such that the antibody has altered C1q binding and/or reduced or abolished complement dependent cytotoxicity (CDC). This approach is described in, e.g., U.S. Pat. No. 6,194,551 by Idusogie et al.

In another aspect, one or more amino acid residues are altered to thereby alter the ability of the antibody to fix complement. This approach is described in, e.g., the PCT Publication WO 94/29351 by Bodmer et al. In a specific aspect, one or more amino acids of an antibody or antigen binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In yet another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the PCT Publication WO 00/42072 by Presta. Moreover, the binding sites on human IgG1 for FcγR1, FcγRII, FcγRIII and FcRn have been mapped and variants with improved binding have been described (see Shields et al., J. Biol. Chem. 276:6591-6604, 2001).

In still another aspect, the glycosylation of an antibody is modified. For example, an aglycosylated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in, e.g., U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In order to minimize the ADCC activity of an antibody, specific mutations in the Fc region result in “Fc silent” antibodies that have minimal interaction with effector cells. In general, the “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody.

Silenced effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181: 6664-69) see also Heusser et al., WO2012065950. Examples of silent Fc IgG1 antibodies are the LALA mutant comprising L234A and L235A mutation in the IgG1 Fc amino acid sequence. Another example of a silent IgG1 antibody is the DAPA (D265A, P329A) mutation (U.S. Pat. No. 6,737,056). Another silent IgG1 antibody comprises the N297A mutation, which results in aglycosylated/non-glycosylated antibodies.

Fc silent antibodies result in no or low ADCC activity, meaning that an Fc silent antibody exhibits an ADCC activity that is below 50% specific cell lysis (low ADCC activity), or that is below 1% specific cell lysis (no ADCC activity).

### 3. Production of the Anti-VP1 Antibodies

Anti-VP1 antibodies and antibody fragments (e.g., antigen binding fragments) thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 13, 33, 53, 73, 93, 113, 133, 153, 173, 193, 213, 233, 253, 273 and 293. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs:23, 43, 63, 83, 103, 123, 143, 163, 183, 203, 223, 243, 263, 283 and 303.

In some aspects, the polynucleotide encoding the heavy chain has at least 85%, 89%, 90%, 91%, 92%, 93%, 94%, 95%, 96%, 97%, 98%, 99%, or 100% nucleic acid sequence identity with a polynucleotide of SEQ ID NO: 15, 35, 55, 75, 95, 115, 135, 155, 175, 195, 215, 235, 255, 275 and 295. In some aspects, the polynucleotide encoding the light chain has at least 85%, 89%, 90%, 91%, 92%, 93%, 94%, 95%, 96%, 97%, 98%, 99%, or 100% nucleic acid sequence identity with a polynucleotide of SEQ ID NO: 25, 45, 65, 85, 105, 125, 145, 165, 185, 205, 225, 245, 265, 285 and 305.

The polynucleotides of the present disclosure can encode only the variable region sequence of an anti-VP1 antibody. They can also encode both a variable region and a constant region of the antibody. Some of the polynucleotide sequences encode a polypeptide that comprises variable regions of both the heavy chain and the light chain of one of an exemplified anti-VP1 antibody. Some other polynucleotides encode two polypeptide segments that respectively are substantially identical to the variable regions of the heavy chain and the light chain of one of the mouse antibodies.

The polynucleotide sequences can be produced by de novo solid-phase DNA synthesis or by PCR mutagenesis of an existing sequence (e.g., sequences as described in the Examples below) encoding an anti-VP1 antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Also provided in the present disclosure are expression vectors and host cells for producing the anti-VP1 antibodies described above. Various expression vectors can be employed to express the polynucleotides encoding the anti-VP1 antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the anti-VP1 polynucleotides and polypeptides in mammalian (e.g., human) cells include pThioHis A, B & C, pcDNA3.1/His, pEBVHis A, B & C (Invitrogen, San Diego, CA), MPSV vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-VP1 antibody chain or fragment. In some aspects, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of an anti-VP1 antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-VP1 antibody sequences. More often, the inserted anti-VP1 antibody sequences are linked to a signal sequences before inclusion in the vector. Vectors to be used to receive sequences encoding anti-VP1 antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the anti-VP1 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-VP1 polypeptides. Insect cells in combination with baculovirus vectors can also be used.

In other aspects, mammalian host cells are used to express and produce the anti-VP1 polypeptides of the present disclosure. For example, they can be either a hybridoma cell line expressing endogenous immunoglobulin genes (e.g., the myeloma hybridoma clones as described in the Examples) or a mammalian cell line harboring an exogenous expression vector. These include any normal mortal or normal or abnormal immortal animal or human cell. For example, a number of suitable host cell lines capable of secreting intact immunoglobulins have been developed, including the CHO cell lines, various COS cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-VP1 antibody chains or binding fragments can be prepared using expression vectors which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Therapeutic and Diagnostic Uses

The antibodies, antibody fragments (e.g., antigen binding fragments) of the present disclosure are useful in a variety of applications including, but not limited to, polyoma viral infection and disease. In certain aspects, the antibodies, antibody fragments (e.g., antigen binding fragments), and are useful for neutralizing BKV or JCV infection and the prevention or treatment of BK virus nephropathy, for example, BKVAN). The methods of use can be in vitro, ex vivo, or in vivo methods.

In one aspect, the antibodies, antibody fragments (e.g., antigen binding fragments), are useful for detecting the presence of BKV in a biological sample. The term “detecting” as used herein encompasses quantitative or qualitative detection. In certain aspects, a biological sample comprises a cell or tissue. In certain aspects, such tissues include normal and/or cancerous tissues that express BKV at higher levels relative to other tissues.

In one aspect, the present disclosure provides a method of detecting the presence of BKV in a biological sample. In certain aspects, the method comprises contacting the biological sample with an anti-VP1 antibody under conditions permissive for binding of the antibody to the antigen, and detecting whether a complex is formed between the antibody and the antigen. The biological sample can include, without limitation, urine or blood samples.

Also included is a method of diagnosing a disorder associated with expression of BKV or JCV virus. In certain aspects, the method comprises contacting a test cell with an anti-VP1 antibody; determining the level of expression (either quantitatively or qualitatively) of BK virus in the test cell by detecting binding of the anti-VP1 antibody to the BK virus; and comparing the level of infection in the test cell with the level of infection of BK virus in a control cell (e.g., a normal cell of the same tissue origin as the test cell or a non-BK virus infected cell), wherein a higher level of presence of BK virus in the test cell as compared to the control cell indicates the presence of a disorder associated with infection with BK virus. In certain aspects, the test cell is obtained from an individual suspected of having a BK virus infection.

In certain aspects, a method of diagnosis or detection, such as those described above, comprises detecting binding of an anti-VP1 antibody to a BKV infected cell. An exemplary assay for detecting binding of an anti-VP1 antibody to a BKV infected cell is a “FACS” assay.

Certain other methods can be used to detect binding of anti-VP1 antibodies. Such methods include, but are not limited to, antigen-binding assays that are well known in the art, such as Western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain aspects, anti-VP1 antibodies are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

In certain aspects, anti-VP1 antibodies are immobilized on an insoluble matrix. Immobilization entails separating the anti-VP1 antibody from any BKV or JCV proteins that remains free in solution. This conventionally is accomplished by either insolubilizing the anti-VP1 antibody before the assay procedure, as by adsorption to a water-insoluble matrix or surface (Bennich et al, U.S. Pat. No. 3,720,760), or by covalent coupling (for example, using glutaraldehyde cross-linking), or by insolubilizing the anti-VP1 antibody after formation of a complex between the anti-VP1 antibody and BKV or JCV protein, e.g., by immunoprecipitation.

Any of the above aspects of diagnosis or detection can be carried out using an anti-VP1 antibody of the present disclosure in place of or in addition to another anti-VP1 antibody.

In one aspect, the disclosure provides for a method of treating, reducing the likelihood of or ameliorating a disease comprising administering the antibodies, antibody fragments (e.g., antigen binding fragments), to a patient, thereby treating the disease. In certain aspects, the disease treated with the antibodies, antibody fragments (e.g., antigen binding fragments), is BK viral or JC viral infection. Examples of BKV and JCV diseases which can be treated and/or prevented include, but are not limited to, nephropathy, hemorrhagic cystitis, Progressive Multifocal Leukoencephalopathy (PML), interstitial kidney disease, ureteral stenosis, granule cell neuronopathy (GCN), vasculitis, colitis, retinitis, meningitis, and immune reconstitution inflammatory syndrome (IRIS). In certain aspects, the infection is characterized by BKV or JCV expressing cells to which the anti-VP1 antibodies, antibody fragments (e.g., antigen binding fragments) can specifically bind.

The present disclosure provides for methods of treating BK viral infection and BKVAN comprising administering a therapeutically effective amount of the antibodies, antibody fragments (e.g., antigen binding fragments). In certain aspects, the subject is a human.

In certain aspects, the method of reducing BK viral infection comprises administering to a subject a therapeutically effective amount of antibodies or antibody fragments (e.g., antigen binding fragments). In certain aspects, the subject is a human. In certain aspects, the subject is immunosuppressed. For immunosuppresed subjects, the amount of immunosuppression can be increased or decreased due to the therapeutic effects of the anti-VP1 antibodies.

In certain aspects, the transplanted tissue is infected with BK virus to which the anti-VP1 antibody binds. As the incidence of BK infection in the general population is high, there is a high probability that in the case of kidney transplantation, the patient accepting the kidney is BK virus positive or the donor providing the kidney is BK virus positive or both are BK virus positive. In order to prevent BKVAN, anti-VP1 antibodies can be administered to the kidney transplant recipient, before and/or after the kidney transplant procedure, depending on the seropositivity of the kidney donor or transplant recipient. In another aspect, the anti-VP1 antibodies can be administered to the patient when virus is detected in the urine (viruria), or when virus is detected in the blood (viremia).

For the treatment of BK or JCV viral infection, the appropriate dosage of the antibodies, or antibody fragments (e.g., antigen binding fragments), depend on various factors, such as the type of infection to be treated, the severity and course of the infection, the responsiveness of the infection, the generation of viral resistance to therapy, previous therapy, patient's clinical history, and so on. The antibody can be administered one time or over a series of treatments lasting from several days to several months, or until a cure is effected or a diminution of the infection is achieved (e.g., reduction in viruria or viral damage to the kidney). Optimal dosing schedules can be calculated from measurements of drug accumulation in the body of the patient and will vary depending on the relative potency of an individual antibody or antibody fragment (e.g., antigen binding fragment). In certain aspects, dosage is from 0.01 mg to 10 mg (e.g., 0.01 mg, 0.05 mg, 0.1 mg, 0.5 mg, 1 mg, 2 mg, 3 mg, 4 mg, 5 mg, 7 mg, 8 mg, 9 mg, or 10 mg) per kg of body weight, and can be given once or more daily, weekly, monthly or yearly. In certain aspects, the antibody or antibody fragment (e.g., antigen binding fragment), of the present disclosure is given once every two weeks or once every three weeks. The treating physician can estimate repetition rates for dosing based on measured half-life and concentrations of the antibody in bodily fluids or tissues.

### Combination Therapy

In certain instances, the antibody or antibody fragment (e.g., antigen binding fragment), of the present disclosure is combined with other therapeutic agents, such as other anti-viral agents, anti-allergic agents, anti-nausea agents (or anti-emetics), pain relievers, cytoprotective agents, immunosuppressants and combinations thereof.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or infection described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In one aspect, the present disclosure provides a method of treating BKV or JCV infection by administering to a subject in need thereof an antibody in together with immunosuppressant therapies. The anti-VP1 antibodies will act prophylactically to neutralize BKV or JCV primary infection or viral reactivation resulting from the immunosuppressant therapy prior to or post-transplantation. Examples of immunosuppressant therapy include, but are not limited to; a monophosphate dehydrogenase inhibitor, a purine synthesis inhibitor, a calcineurin inhibitor or an mTOR inhibitor. Specific examples of immunosuppressive therapeutics include but are not limited to; mycophenolate mofetil (MMF), mycophenolate sodium, azathioprine, tacrolimus, sirolimus and cyclosporine.

### Pharmaceutical Compositions

To prepare pharmaceutical or sterile compositions including anti-VP1 antibodies, the antibodies of the present disclosure are mixed with a pharmaceutically acceptable carrier or excipient. The compositions can additionally contain one or more other therapeutic agents that are suitable for neutralizing BKV or JCV infection.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, NY, 1993; Lieberman, et al. (eds.), Pharmaceutical Dosage Forms: Tablets, Marcel Dekker, NY, 1990; Lieberman, et al. (eds.) Pharmaceutical Dosage Forms: Disperse Systems, Marcel Dekker, NY, 1990; Weiner and Kotkoskie, Excipient Toxicity and Safety, Marcel Dekker, Inc., New York, N.Y., 2000).

In a specific aspect, the anti-VP1 antibody is a lyophilisate in a vial containing the antibody. The lyophilisate can be reconstituted with water or a pharmaceutical carrier suitable for injection. For subsequent intravenous administration, the obtained solution will usually be further diluted into a carrier solution.

The antibodies disclosed herein are useful in the neutralization of BKV or JCV in tissue transplant patients who can be immunosuppressed, so a pharmaceutical carrier of sucrose and human albumin as used previously in bone marrow transplant patients receiving CytoGam® can be used (DeRienzo et al. Pharmacotherapy 2000; 20:1175-8). Alternatively, the anti-VP1 antibodies can be introduced into transplant patients via a pharmaceutical carrier as described for another anti-viral antibody, Synagis®, as described in WO2003/105894. In this publication, the pharmaceutical carrier was comprised of histidine and/or glycine, a saccharide (e.g. sucrose) and a polyol (e.g. polysorbate).

Selecting an administration regimen for a therapeutic depends on several factors, including the severity of the infection, the level of symptoms, and the accessibility of the target cells in the biological matrix. In certain aspects, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, UK, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., infusion reactions.

Actual dosage levels of the active ingredients in the pharmaceutical compositions with the anti-VP1 antibodies can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the neutralizing activity of the antibodies, the route of administration, the time of administration, the half-life of the antibody in the patient, the duration of the treatment, other drugs, compounds and/or materials used in combination with the particular compositions employed, the age, sex, weight, condition, general health and prior medical history of the patient being treated, and like factors known in the medical arts.

Compositions comprising antibodies or fragments thereof can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

For the antibodies described herein, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. The dosage of the antibodies or fragments thereof can be calculated using the patient's weight in kilograms (kg) multiplied by the dose to be administered in mg/kg.

Doses of the antibodies then can be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, UK, 2001).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present disclosure can also be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present disclosure can be administered via a non-parenteral route, such as a topical, epidermal or mucosal route of administration, for example, intranasally, orally, vaginally, rectally, sublingually or topically. In one aspect, the antibodies of the present disclosure are administered by infusion. In another aspect, the antibodies are administered subcutaneously.

If the antibodies of the present disclosure are administered in a controlled release or sustained release system, a pump may be used to achieve controlled or sustained release (see Langer, supra; Sefton, CRC Crit. Ref Biomed. Eng. 14:20, 1987; Buchwald et al., Surgery 88:507, 1980; Saudek et al., N. Engl. J. Med. 321:574, 1989). Polymeric materials can be used to achieve controlled or sustained release of the therapies of the antibodies (see e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macromol. Sci. Rev. Macromol. Chem. 23:61, 1983; see also Levy et al., Science 228:190, 1985; During et al., Ann. Neurol. 25:351, 1989; Howard et al., J. Neurosurg. 7 1:105, 1989; U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one aspect, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138, 1984).

Controlled release systems are discussed in the review by Langer, Science 249:1527-1533, 1990). Any technique known to one of skill in the art can be used to produce sustained release formulations comprising one or more antibodies of the present disclosure. See, e.g., U.S. Pat. No. 4,526,938, PCT publication WO 91/05548, PCT publication WO 96/20698, Ning et al., Radiotherapy & Oncology 39:179-189, 1996; Song et al., PDA Journal of Pharmaceutical Science & Technology 50:372-397, 1995; Cleek et al., Pro. Int'l. Symp. Control. Rel. Bioact. Mater. 24:853-854, 1997; and Lam et al., Proc. Int'l. Symp. Control Rel. Bioact. Mater. 24:759-760, 1997, each of which is incorporated herein by reference in their entirety.

If the antibodies of the disclosure are administered topically, they can be formulated in the form of an ointment, cream, transdermal patch, lotion, gel, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the antibodies are administered intranasally, it can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., an immunosuppressant, a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the anti-VP1 antibodies may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the anti-VP1 antibodies of the present disclosure. The two or more therapies may be administered within one same patient visit.

In certain aspects, anti-VP1 antibodies can be formulated to ensure proper distribution in vivo. For example, the blood-brain barrier (BBB) excludes many highly hydrophilic compounds. To ensure that the anti-VP1 antibodies cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The present disclosure provides protocols for the administration of pharmaceutical composition comprising antibodies alone or in combination with other therapies to a subject in need thereof. The combination therapies (e.g., prophylactic or therapeutic agents) can be administered concomitantly or sequentially to a subject. The therapy (e.g., prophylactic or therapeutic agents) of the combination therapies can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof are administered to a subject in a sequence and within a time interval such that the antibodies can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various aspects, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other aspects, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

### EXAMPLES

### Example 1: Generation of Anti-VP1 Antibodies

B cells expressing anti-VP1 antibodies were lysed and the VH (heavy) and VL (light) chains were sequenced by RT-PCR and analyzed to identify critical post translational modification (PTM) sites. Plasmids of the VH and VL chains were then transfected in a CHO mammalian cell line in an IgG1 back bone vector for expression of the full IgG1 antibodies.

Methods for generation of monoclonal antibodies using hybridoma technology are known in the art (Antibody Methods and Protocols, Methods in Molecular Biology vol. 901, 2012, Chapter 7: 117). Briefly, female Balb/c mice were immunized with VLPs from BKV serotype I, serotype IV, and JCV (either individually or in combination) using various prime-boost strategies, doses of immunogen, and adjuvants (including but not limited to Freund's adjuvant and MF59 adjuvant). Supernatant of successfully fused (growing) hybridomas were screened for the presence of anti-VP1 antibodies by ELISA, then for functional activity in neutralization assays. CDRs from select murine IgGs were humanized by grafting onto human framework acceptor templates, cloned into mammalian IgG1 backbone expression vectors and transfected in a CHO mammalian cell line for expression of the full IgG1 antibodies.

Methods for generation of monoclonal antibodies using phage display technology are known in the art (Antibody Methods and Protocols, Methods in Molecular Biology vol. 901, 2012, Chapter 3: 33). Briefly, a human B-cell antibody library in scFv format with Vκ was screened for anti-VP1 antibodies by solution panning with streptavidin-coupled magnetic beads complexed with biotinylated BKV serotype IV VLPs over 3 rounds of selection with increasing stringency. Isolates were first expressed as scFv and screened for binding to both BKV serotype IV VLPs and pentamers by ELISA. Select isolates were then cloned and expressed as IgG1, reanalyzed for binding to VP1 (serotype I and IV) by ELISA and for functional activity in neutralization assays, and transfected in a CHO mammalian cell line for expression of the full IgG1 antibodies.

A summary of the anti-VP1 antibodies is provided in Table 3.

TABLE 3
anti-VP1 antibodies
Antibody

| P165E2 | |
|---|---|
| NEG447 | P165E2 changes: germlined/affinity matured for serotype |
| | IV; VH (T35S, E43A, A73T, S74N), VL (R17K, G61D, |
| | A86V) resulted in no significant change in affinity or |
| | activity. |
| NEG447A | P165E2 changes: germlined NEG447; VL (L10V, V86D) |
| | resulted in ~3-fold greater affinity and ~8-fold more potent |
| | activity (EC90) on serotype II, ~10-fold greater affinity |
| | for serotype IV |
| P7G11 | |
| P7G11A | P7G11 variant: germlined P7G11; VH (A12V, S23T, |
| | I69V, M71I, T85S) resulted in ~150-fold greater affinity |
| | for serotype II, ~14-fold greater affinity for serotype IV |
| P8D11 | |
| P8D11A | P8D11 change to remove post-translational modification: |
| | N30S in HCDR1 resulted in no significant change in |
| | affinity or activity |
| P8D11B | P8D11 change to remove post-translational modification: |
| | N30K in HCDR1 resulted in no significant change in |
| | affinity or activity |
| P8D11C | P8D11 change to remove post-translational modification: |
| | N30Q in HCDR1 resulted in no significant change in |
| | affinity or activity |
| P8D11D | P8D11 change in Heavy Chain framework 1 region to fix |
| | proteolysis/clipping liability (V5Q, G9P, T10G) resulted |
| | in no significant change in affinity or activity |
| P8D11E | P8D11 change in Heavy Chain framework 1 region to fix |
| | proteolysis/clipping liability (T10G) resulted in no |
| | significant change in affinity or activity |
| P46F4 | |
| EBB-C1975- | phage display |
| B5 | |
| EBB-C1975- | phage display |
| A3 | |
| EBB-C1975- | phage display |
| A7 | |
| EBB-C1975- | phage display |
| E7 | |
| 2081-20-8 | mouse hybridoma |
| 2075-16-1 | mouse hybridoma |
| 2075-456-4 | mouse hybridoma |
| 2081-36-8 | mouse hybridoma |
| 2081-66-5 | mouse hybridoma |
| 2081-38-5 | mouse hybridoma |
| 2081-25-6 | mouse hybridoma |
| 2077-4-1 | mouse hybridoma |
| 2077-7-5 | mouse hybridoma |
| 2077-10 1 | mouse hybridoma |
| 2077-26-1 | mouse hybridoma |
| 2077-28-2 | mouse hybridoma |

### Example 2: Affinity Maturation of Anti-VP Antibodies

The anti-VP1 antibodies were affinity matured in yeast by error-prone PCR or CDR-directed mutagenesis. VP1 proteins from each of the four serotypes of BKV (as shown in Table 4) were used as the antigen in up to three rounds of selection by FACS analysis. VH (heavy) and/or VL (light) chains with enhanced binding affinity to VP1 by FACS analysis were then cloned into mammalian IgG1 backbone expression vectors and transfected in a CHO mammalian cell line for expression of the full IgG1 antibodies.

TABLE 4

| Name | VP1 protein | SEQ ID NO |
|---|---|---|
| Serotype 1, | FSLKLSAENDFSSDSPERKMLPCY | (SEQ ID |
| amino acids | STARIPLPNLNEDLTCGNLLMWEA | NO: 496) |
| 66-145 | VTVQTEVIGITSMLNLHAGSQKVH | |
| | EHGGGKPI | |
| Serotype II, | YSLKLTAENAFDSDSPDKKMLPCY | (SEQ ID |
| amino acids | STARIPLPNLNEDLTCGNLLMWEA | NO: 497) |
| 66-145 | VTVKTEVIGITSMLNLHAGSQKVH | |
| | ENGGGKPV | |
| Serotype III, | YSQHLSAENAFDSDSPDKKMLPCY | (SEQ ID |
| amino acids | STARIPLPNLNEDLTCGNLLMWEA | NO: 498) |
| 66-145 | VTVKTEVIGITSMLNLHAGSQKVH | |
| | ENGGGKPV | |
| Serotype IV, | YSLRLTAETAFDSDSPDRKMLPCY | (SEQ ID |
| amino acids | STARIPLPNLNEDLTCGNLLMWEA | NO: 499) |
| 66-145 | VTVKTEVIGITSMLNLHAGSQKVH | |
| | ENGGGKPI | |

### Example 3: BK Virus and Virus-Like Particle (VLP) Generation

Genomic clones of BKV serotype I were obtained from ATCC (pBR322-BKV MM, cat #45026; pBR322-BKV Dunlop, cat #45025). Infectious genomic clones of chimeric viruses for serotype II, III and IV were generated using the cloning strategy described previously (Broekema et al, Virology 2010 407:368-373). Briefly, unique restriction sites (SacII, PmlI) were introduced into BKV serotype I genomes flanking the VP1-VP2-VP3 coding region using site-directed mutagenesis. The coding region for VP1 from serotype II isolate SB (GenBank Accession CAA79596.1), serotype III isolate AS (GenBank Accession AAA46882.1) and serotype IV strain ITA-4 (GenBank Accession BAF75132) were synthesized in the context of VP2/VP3 coding region from the serotype I isolates (Genewiz, La Jolla, CA), such that the synthesized fragments encompassed the SacII-PmlI region to be used for swap combinations as described in Broekema et al., supra. The resulting chimeric genomic clones were then used to generate high titer infectious viral stocks in primary renal proximal tubule epithelial (RPTE) cells (ATCC, cat #PCS-400-010) as previously described (Abend et al, J. Virology 2007 81:272-279).

VLPs representing each of the four BKV serotypes were generated by expression of VP1 in Sf9 insect cells and extracted from frozen cell pellets from 1 L cultures by microtip sonication (3×45 second pulses, rest 5 min between pulses on ice), isolation by pelleting VLPs through a 20% sucrose cushion (116,000 g for 2.5 hours), and purification by anion exchange with a 5 ml GE HiTrap Q HP column (GE Healthcare, Pittsburgh, PA) followed by purification using a 10 ml Capto™ Core700 (GE Healthcare, Pittsburgh, PA) resin-based size exclusion column, and finally purification on a GE Sephacryl S500 26/60 (GE Healthcare, Pittsburgh, PA) size exclusion column. The prepared VLPs were used in ELISA and SPR based binding assays in Examples 6 and 7.

### Example 4: Purification of BKV VP1 Pentamers

VP1 proteins from each of the four serotypes of BKV (sequences shown in Table 5 below) were cloned with N terminal GST-6×His-TEV sequences and subcloned into pGEX destination vector (GE Healthcare, Pittsburgh, PA). GST fusion proteins were expressed in E. coli, extracted from cell pellets using a microfluidizer (15,000 PSI), and purified by immobilized metal ion affinity chromatography (IMAC) using a 20 ml nickel sepharose 6 Fast Flow column (GE Healthcare, Pittsburgh, PA). The GST-6×His-TEV tag was cleaved by overnight incubation with TEV protease and final purification was performed using a 5 ml His-Trap Fast Flow column (GE Heathcare, Pittsburgh, PA), followed by Superdex 200 26/60 size exclusion column (GE Heathcare, Pittsburgh, PA).

TABLE 5

| BKV | | SEQ ID |
|---|---|---|
| Serotype | VP1 Sequence | NO. |
| Serotype I, | KGGVEVLEVKTGVDAITEVECFLNP | SEQ ID |
| amino acids | EMGDPDENLRGFSLKLSAENDFSSD | NO: 502 |
| 30-297 | SPERKMLPCYSTARIPLPNLNEDLT | |
| | CGNLLMWEAVTVQTEVIGITSMLNL | |
| | HAGSQKVHEHGGGKPIQGSNFHFFA | |
| | VGGDPLEMQGVLMNYRTKYPEGTIT | |
| | PKNPTAQSQVMNTDHKAYLDKNNAY | |
| | PVECWIPDPSRNENTRYFGTFTGGE | |
| | NVPPVLHVTNTATTVLLDEQGVGPL | |
| | CKADSLYVSAADICGLFTNSSGTQQ | |
| | WRGLARYFKIRLRKRSVK | |
| Serotype II, | KGGVEVLEVKTGVDAITEVECFLNP | SEQ ID |
| amino acids | EMGDPDDNLRGYSLKLTAENAFDSD | NO: 503 |
| 30-297 | SPDKKMLPCYSTARIPLPNLNEDLT | |
| | CGNLLMWEAVTVKTEVIGITSMLNL | |
| | HAGSQKVHENGGGKPVQGSNFHFFA | |
| | VGGDPLEMQGVLMNYRTKYPQGTIT | |
| | PKNPTAQSQVMNTDHKAYLDKNNAY | |
| | PVECWIPDPSRNENTRYFGTYTGGE | |
| | NVPPVLHVTNTATTVLLDEQGVGPL | |
| | CKADSLYVSAADICGLFTNSSGTQQ | |
| | WRGLARYFKIRLRKRSVK | |
| Serotype III, | KGGVEVLEVKTGVDAITEVECFLNP | SEQ ID |
| amino acids | EMGDPDDHLRGYSQHLSAENAFDSD | NO: 504 |
| 30-297 | SPDKKMLPCYSTARIPLPNLNEDLT | |
| | CGNLLMWEAVTVKTEVIGITSMLNL | |
| | HAGSQKVHENGGGKPVQGSNFHFFA | |
| | VGGDPLEMQGVLMNYRTKYPQGTIT | |
| | PKNPTAQSQVMNTDHKAYLDKNNAY | |
| | PVECWIPDPSKNENTRYFGTYTGGE | |
| | NVPPVLHVTNTATTVLLDEQGVGPL | |
| | CKADSLYVSAADICGLFTNSSGTQQ | |
| | WRGLARYFKIRLRKRSVK | |
| Serotype IV, | KGGVEVLEVKTGVDAITEVECFLNP | SEQ ID |
| amino acids | EMGDPDNDLRGYSLRLTAETAFDSD | NO: 505 |
| 30-297 | SPDRKMLPCYSTARIPLPNLNEDLT | |
| | CGNLLMWEAVTVKTEVIGITSMLNL | |
| | HAGSQKVHENGGGKPIQGSNFHFFA | |
| | VGGDPLEMQGVLMNYRTKYPEGTVT | |
| | PKNPTAQSQVMNTDHKAYLDKNNAY | |
| | PVECWIPDPSRNENTRYFGTYTGGE | |
| | NVPPVLHVTNTATTVLLDEQGVGPL | |
| | CKADSLYVSAADICGLFTNSSGTQQ | |
| | WRGLPRYFKIRLRKRSVK | |

### Example 5: Affinity Measurements of Anti-VP1 Antibodies (SET Assay)

Solution equilibration titration (SET) assay was used to determine the interaction affinities (K_{D}) of antibodies with BKV VP1 pentamers from all four serotypes. Antibodies were assayed at 1 pM concentration (constant), VP1 pentamers were serially diluted from a starting concentration of 10 nM. Antibody:VP1 pentamer solution was incubated overnight, then assayed for unbound antibody using an MSD array plate (Meso Scale Discovery Cat #L21XA, Rockville MD) coated with VP1 pentamer. The K_{D} was determined by fitting the plot with a 1:1 fit model (according to Piehler et al. J. Immunol. Methods. 1997; 201(2):189-206).

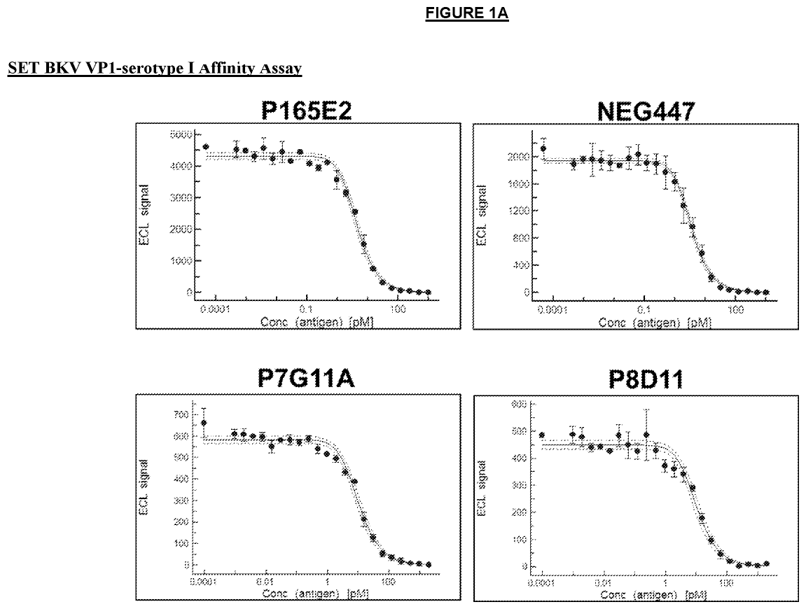

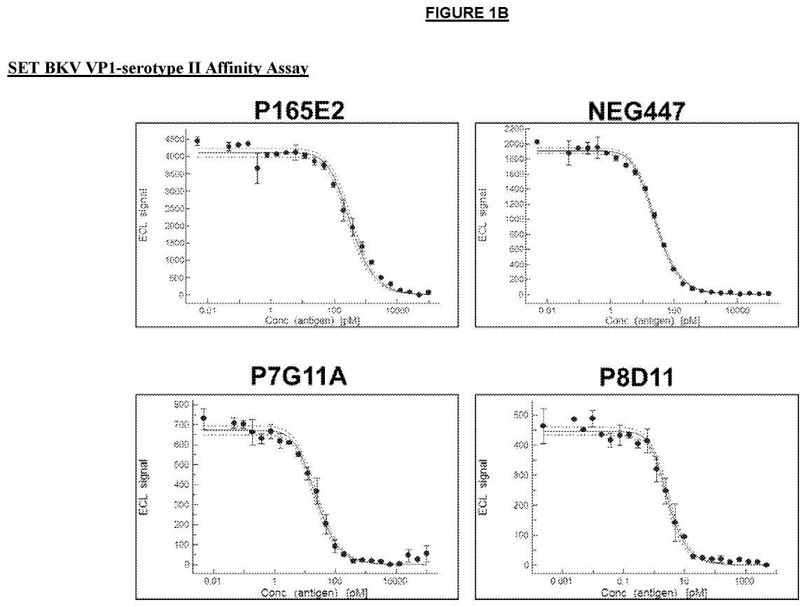

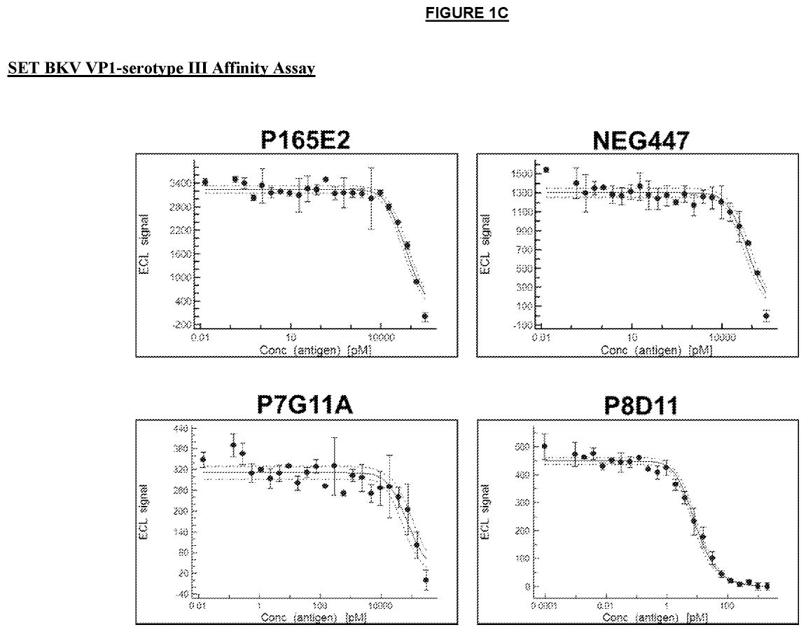

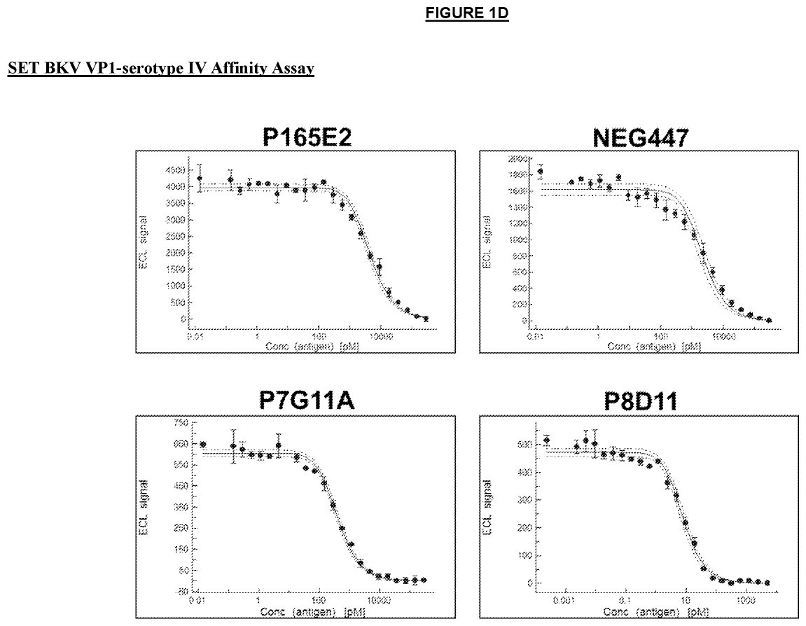

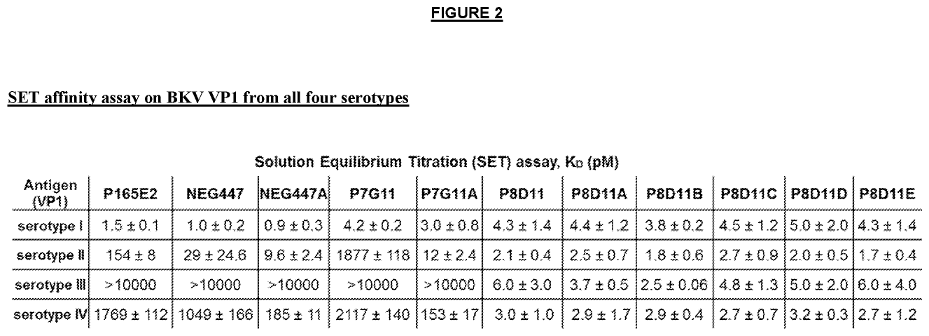

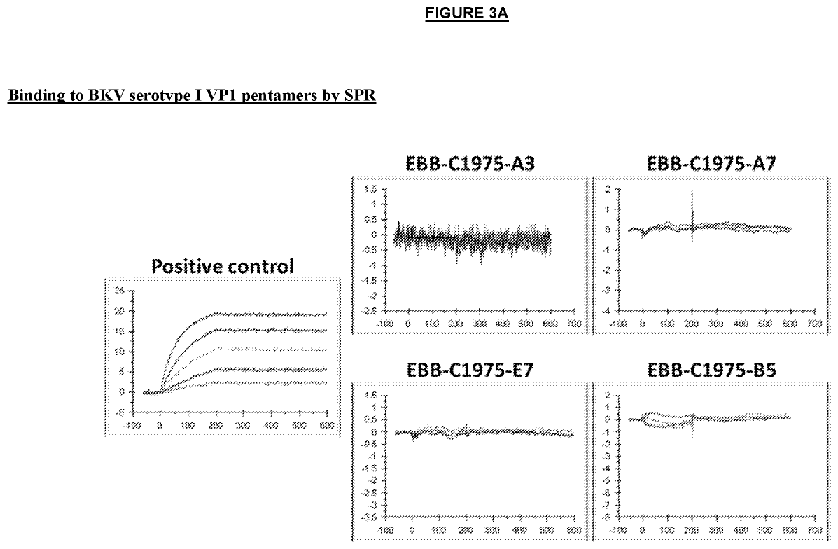

In SET assays, K_{D} values were similar for anti-VP1 antibodies binding to BKV serotype I pentamers, ranging from 0.9 to 5.0 pM. P8D11 and derivatives of P8D11 had comparable K_{D} values for binding to BKV serotype II, III, and IV pentamers, and when compared to the other antibodies, had at least 3.5-fold greater affinity on serotype II pentamers and 47-fold greater affinity on serotype IV pentamers. This is shown in FIG. 1A-1D. In addition, P8D11 and derivatives of P8D11 demonstrated binding affinity ranging from 2.5 to 6.0 pM on serotype III pentamers, whereas the other antibodies had no detectable binding to serotype III pentamers within the tested conditions. A summary of SET affinity data for these anti-VP1 antibodies is found in FIG. 2.

### Example 6: Binding of Anti-VP1 Antibodies to VP1 Pentamers and VLPs (ELISA)

The binding of anti-VP1 antibodies to VP1 pentamers and VLPs were analyzed by ELISA. Briefly, Immulon 2HB plates (VWR, 62402-972) were coated with 100 ng/well BKV VLPs or VP1 pentamers overnight. Antibodies were serially diluted in PBS with 0.5% BSA and allowed to bind antigen-coated plates for 2 h. Plates were washed with PBS and then incubated with secondary antibody (HRP-conjugated rabbit anti-human IgG, Southern Biotech #6140-05) diluted 1:6000 in 0.5% BSA in PBS for 1 h. Plates were washed with PBS and tetramethylbenzidine (TMB) microwell peroxidase substrate (KPL, 52-00-03 IL) was used to develop the reactions.

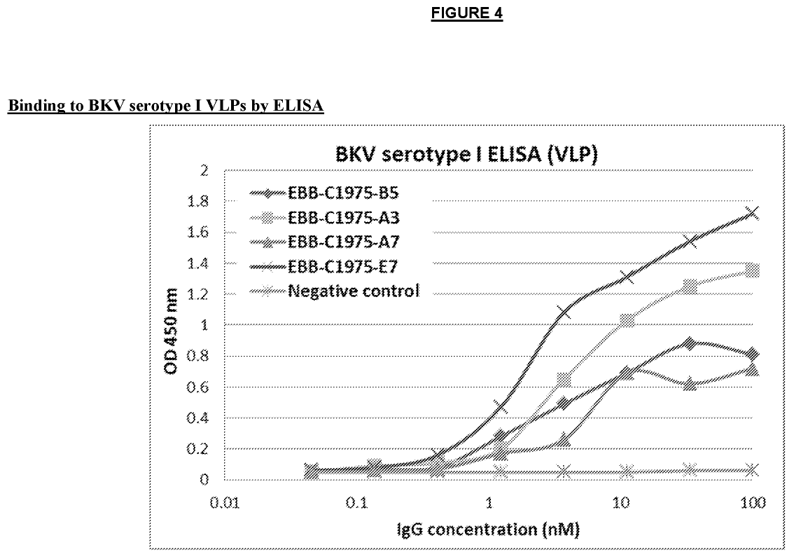

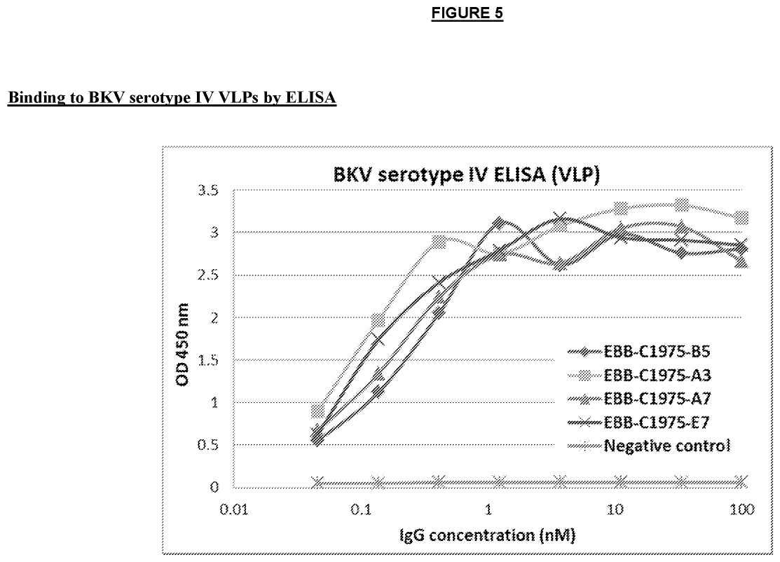

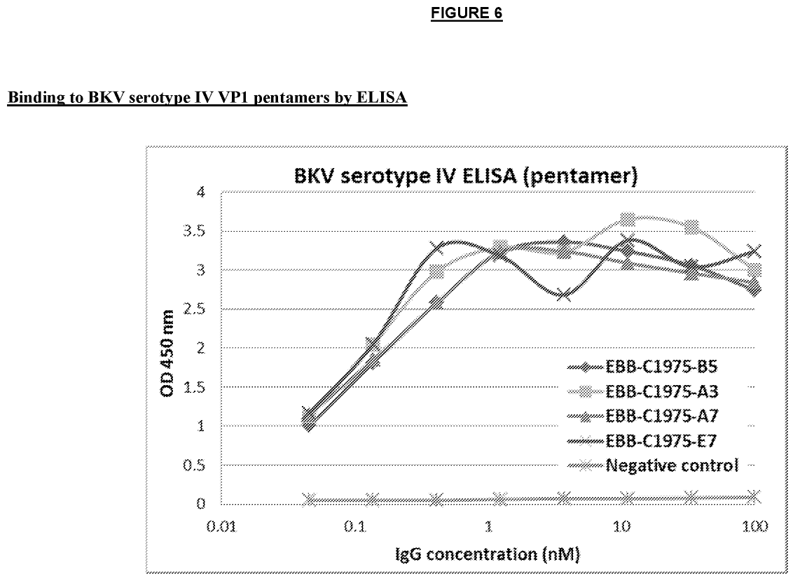

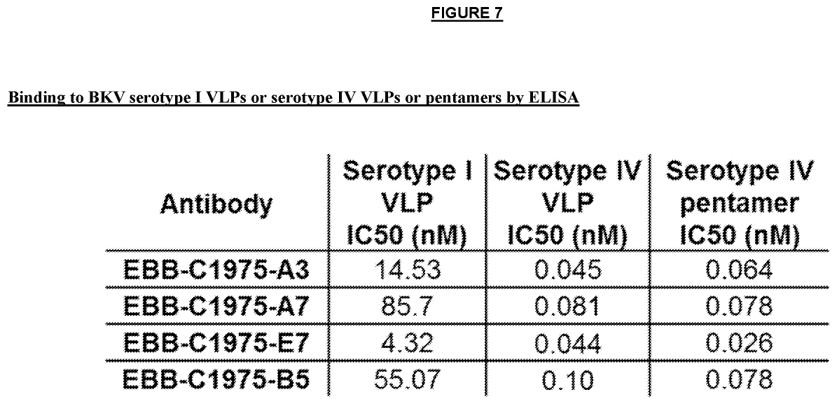

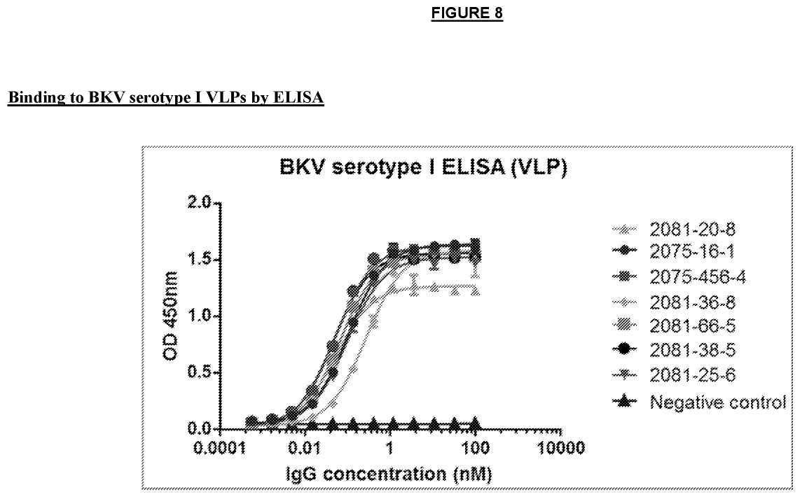

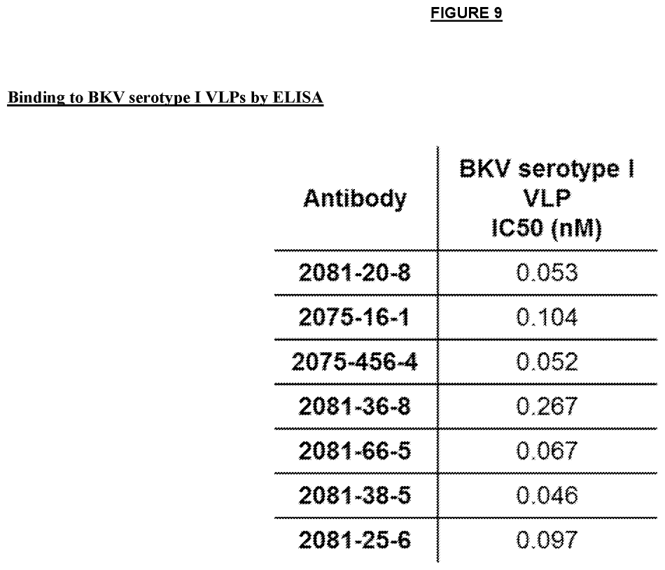

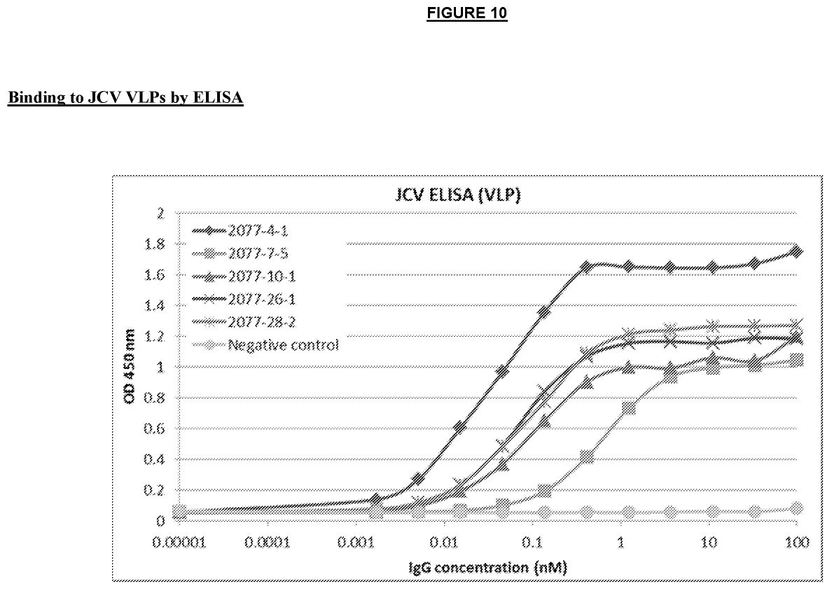

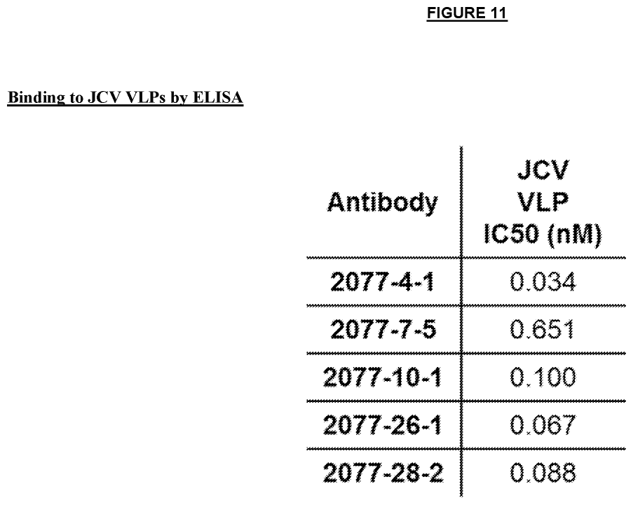

The anti-VP1 antibodies EBB-C1975-A3, A7, E7, and B5 showed similar binding to VLPs (IC50s ranging from 0.044 to 0.1 nM) or VP1 pentamers (IC50s ranging from 0.026 to 0.078 nM) from BKV serotype IV, but reduced and more variable binding activity to serotype I VLPs (IC50s ranging from 4.32 to 85.7 nM). This data is shown graphically in FIGS. 4-6 and summarized in FIG. 7. In contrast, anti-VP1 antibodies from the 2081 and 2075 series showed enhanced binding activity to serotype I VLPs, with IC50s ranging from 0.046 to 0.267 nM and this data is shown in FIGS. 8 and 9. The JCV-specific anti-VP1 antibodies of the 2077 series demonstrated binding activity to JCV VLPs ranging from 0.034 to 0.651 nM and this data is provided in FIGS. 10 and 11.

### Example 7: Binding of Anti-VP1 Antibodies to VP1 Pentamers and VLPs by SPR

The binding of anti-VP1 antibodies to VP1 pentamers and VLPs were analyzed by surface plasmon resonance (SPR). Briefly, biotinylated Protein A is immobilized on a streptavidin-coated SPR chip surface, and anti-VP1 antibodies are captured on the resulting surface by binding to Protein A. BKV VP1 pentamers or VLPs are then flowed over the surface and allowed to bind anti-VP1 antibodies during the association phase, followed by a buffer wash during the dissociation phase.

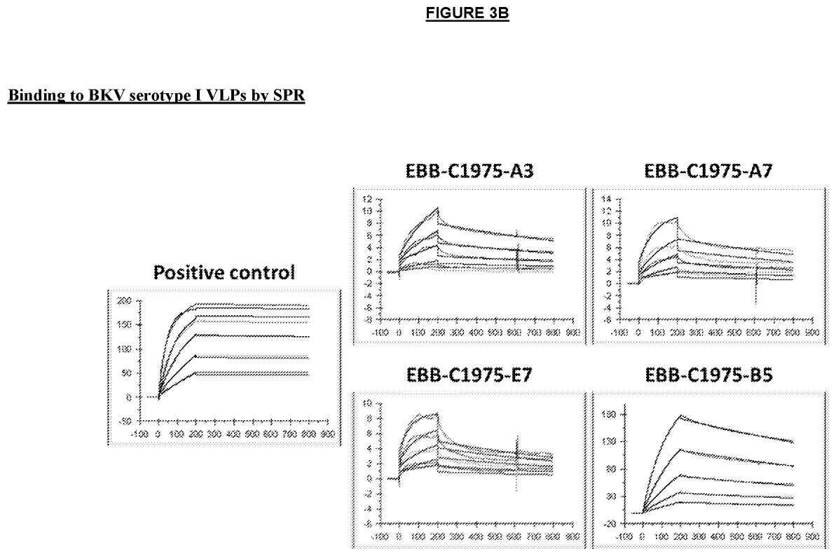

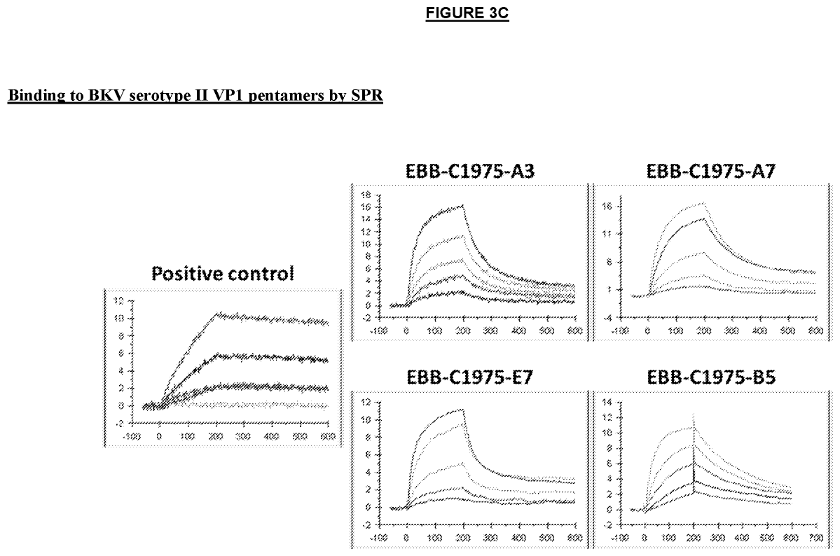

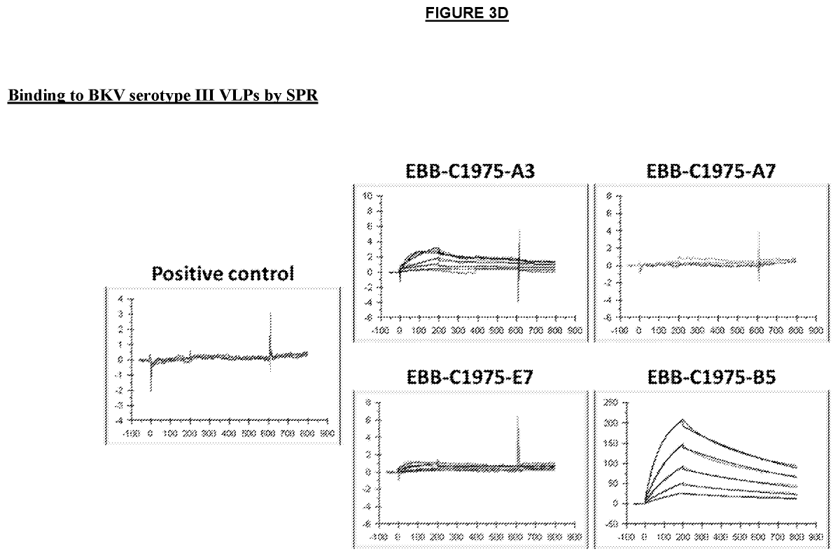

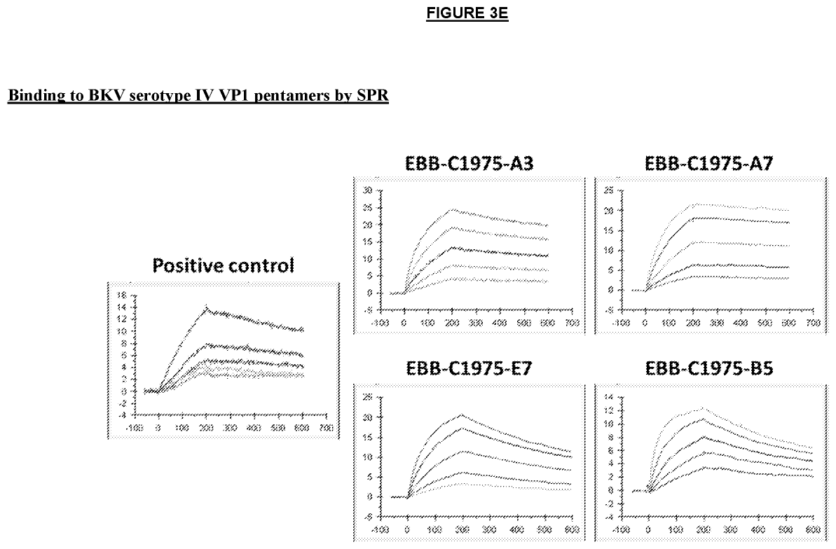

SPR was used to evaluate binding of anti-VP1 antibodies EBB-C1975-A3, A7, E7, and B5 to the four serotypes of BKV, relative to a positive control (P165E2). All four antibodies had very similar binding profiles to VP1 pentamers: no binding to serotype I and III pentamers, atypical binding to serotype II pentamers (large bulk shift and no return to baseline), and binding to serotype IV pentamers similar to P165E2 but with lower affinity (FIGS. 3A, 3C and 3E). For VLPs, EBB-C1975-A3, A7, and E7 shared similar binding profiles: atypical binding to serotype I VLPs and no binding to serotype III VLPs. However, EBB-C1975-B5 binding profile was distinct with significant binding to serotype I and III VLPs, demonstrating binding to a different epitope on VP1 (FIGS. 3B and 3D).

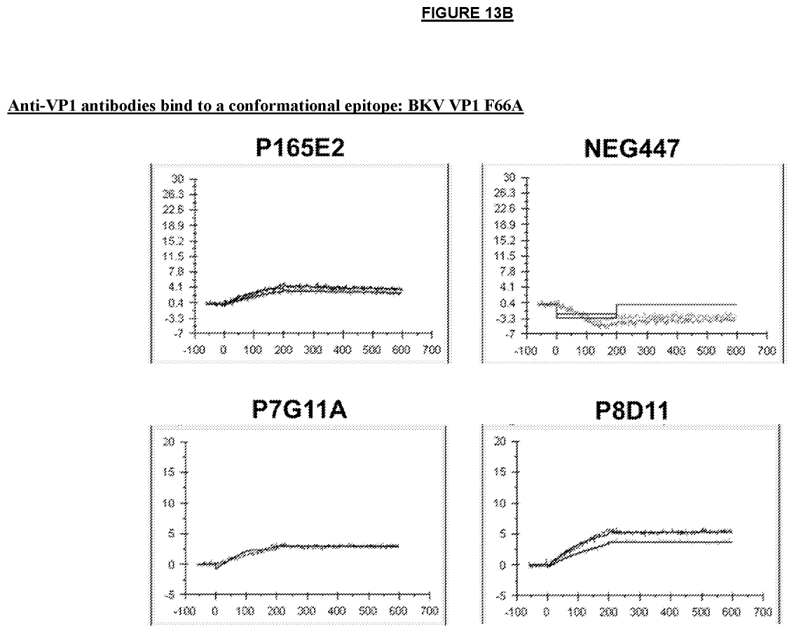

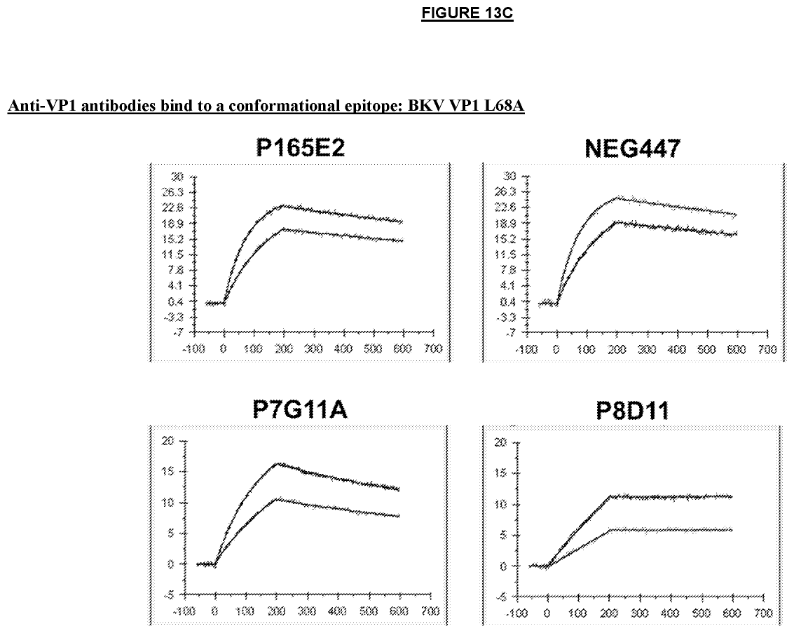

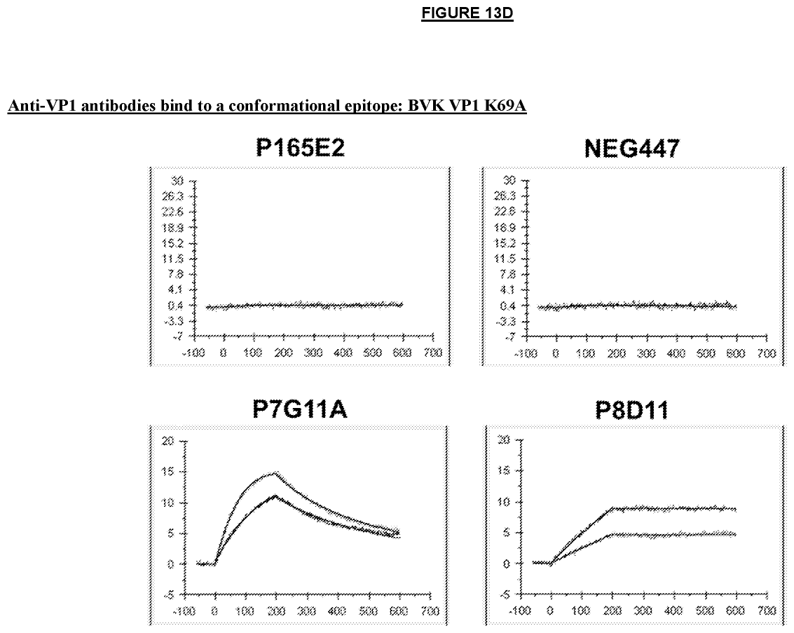

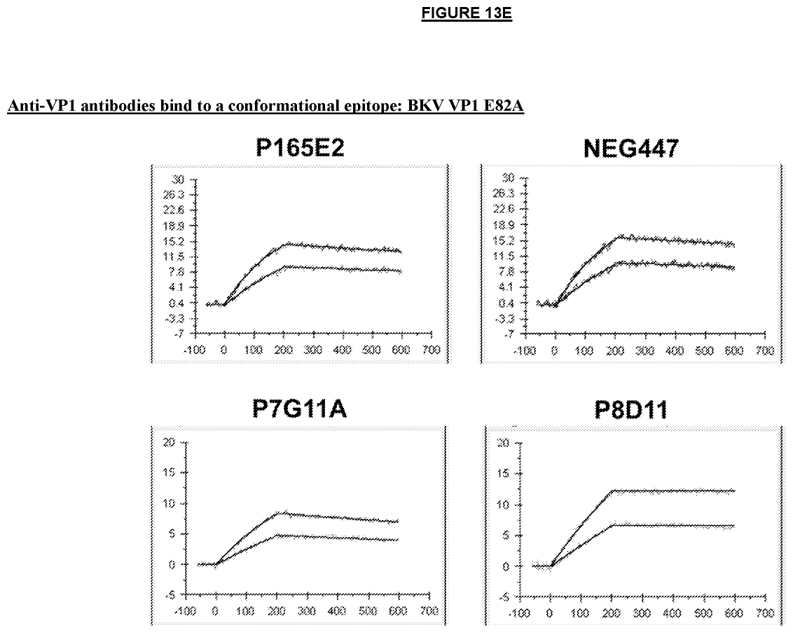

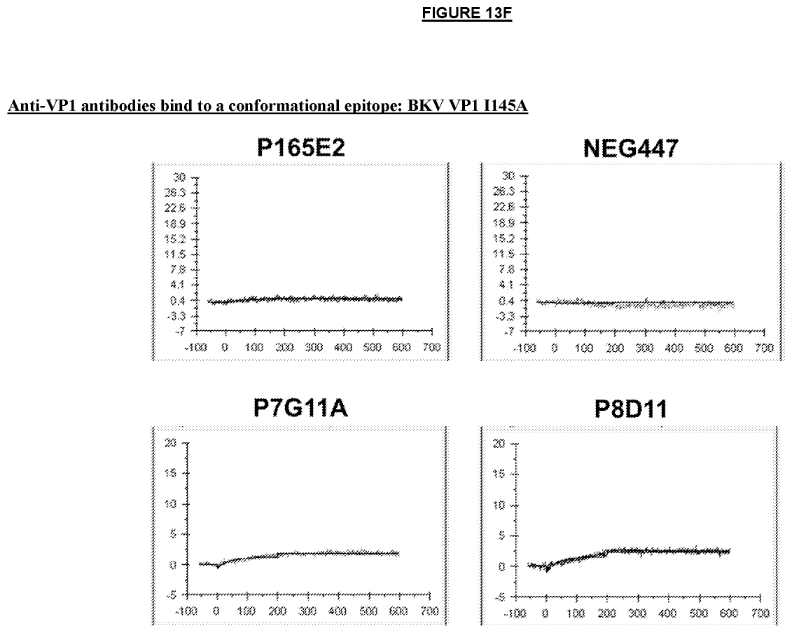

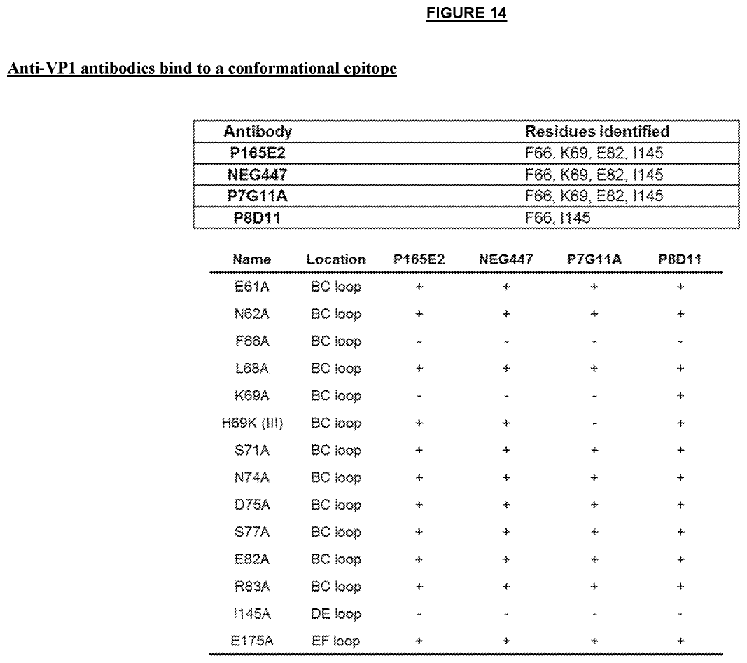

SPR was also used to characterize binding of anti-VP1 antibodies P165E2, NEG447, P7G11A, and P8D11 to VP1 pentamers by scanning alanine mutagenesis (FIGS. 13A-F and FIG. 14). All anti-VP1 antibodies showed reduced binding to F66A and 1145A VP1 mutants, due to an overall impact of the mutation on VP1 pentamer structure (FIGS. 13B and 13F). In addition, K69A and E82A impacted binding of P165E2, NEG447, and P7G11A (FIGS. 13D and 13E).

### Example 8: Anti-VP1 Antibodies Bind to a Conformational Epitope

To determine if the anti-VP1 antibodies bind a conformational epitope, Western blots of denatured protein by SDS-PAGE and dot blots of protein in native conformation were used. Briefly, BKV serotype I or IV VP1-pentamer were run on SDS-PAGE and transferred to nitrocellulose membrane (Western blot) or spotted directly onto nitrocellulose membrane (dot blot). Both membranes were incubated with anti-VP1 antibodies followed by anti-human IgG secondary antibody conjugated to infrared fluorescing dyes for detection using the Licor Odyssey system.

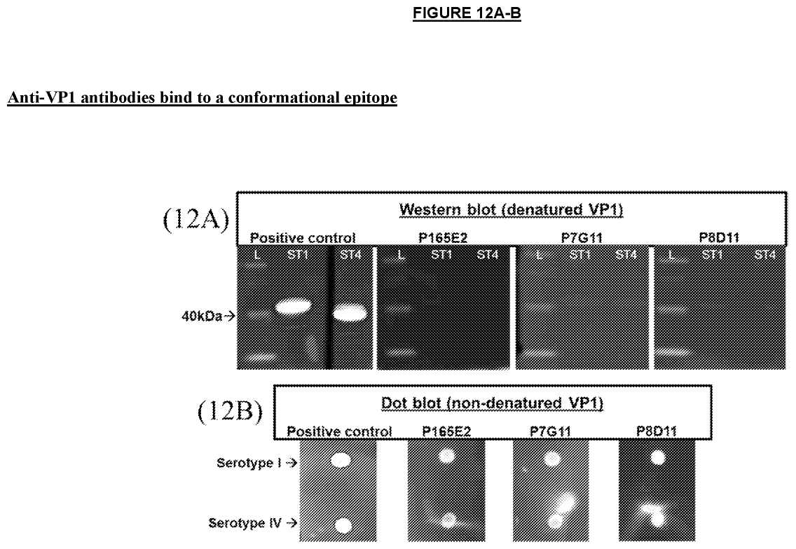

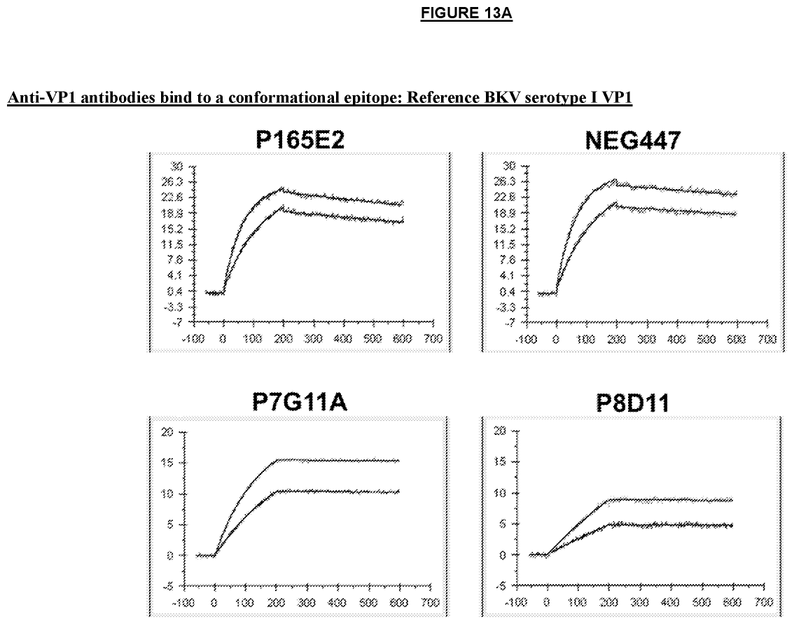

Commercially available positive control antibody (Abcam 53977) known to recognize a linear epitope detected both the denatured and non-denatured VP1. However, P165E2, P7G11 and P8D11 failed to detect denatured VP1 on the Western blot and only recognized native VP1 on the dot blot, indicating that these antibodies bind to a conformational (non-linear) epitope of VP1 (FIGS. 12A and 12B).

To further characterize the epitope of anti-VP1 antibodies, scanning alanine mutagenesis was performed for residues, primarily in the VP1 BC loop, known to be exposed on the virion surface and within a major interaction site for cell surface receptors. These mutant VP1 pentamers were assayed for binding to P8D11 and P7G11A in surface plasmon resonance (SPR) studies as described above in Example 7. Mutations at several positions impacted binding of P7G11A (F66A, K69A, E82A, 1145A) (FIGS. 13A-F and FIG. 14). However, mutations at only two sites resulted in reduction of P8D11 binding (F66A, 1145A) (FIG. 14). As the mutations at F66 and 1145 resulted in a loss of binding of all antibodies tested, without being bound by any one theory, it is likely that these mutations result in a general disruption of the VP1 pentamer structure. All other VP1 pentamers with BC loop mutations tested retained P8D11 binding. In contrast, hydrogen-deuterium exchange studies have identified a protected region within the EF loop of VP1 upon binding of P8D11 Fab fragment. Follow-up scanning alanine mutagenesis studies confirm that key contact residues for P8D11 binding within this region include Y169, R170 and K172, with D/E175, K181, N182, T184 and Q186 to M190 being important residues as determined by deuterium exchange ((YRXKXX(D/E)XXXXXKNXTXQ) (SEQ ID NO: 500)). This is further described in Example 14 through Example 17.

### Example 9: Neutralization of BK Virus by Anti-VP1 Antibodies

Infectious BKV serotype I and chimeric viruses representing serotype II, III, and IV were pre-incubated with purified antibodies for 1 hour to allow for binding and neutralization. Primary renal proximal tubule epithelial (RPTE) cells (ATCC, cat #PCS-400-010) were then exposed to the virus-antibody mixture for 4 hours, replaced with fresh medium, and incubated for 48 hours to allow for viral entry and gene expression. Cells were fixed with 4% paraformaldehyde and analyzed by immunofluorescence to detect TAg expression (Calbiochem DP02, pAb416 mouse anti-SV40 TAg antibody). The immunofluorescence was analyzed by high content image analysis using the Cellomics ArrayScan®VTI HCS Reader to quantify the percent of BKV-infected cells (TAg-positive, DAP1-positive), with data presented as percent inhibition of infection relative to untreated control wells.

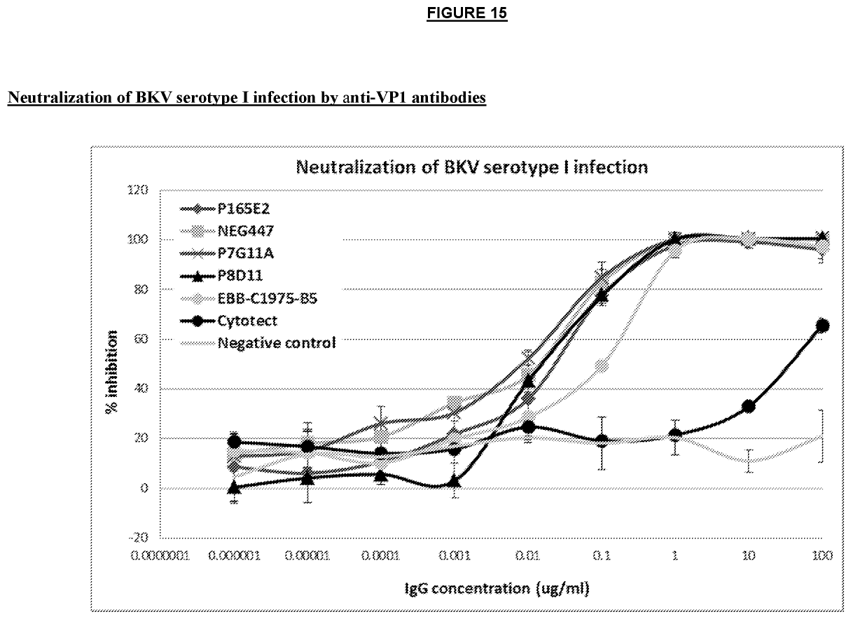

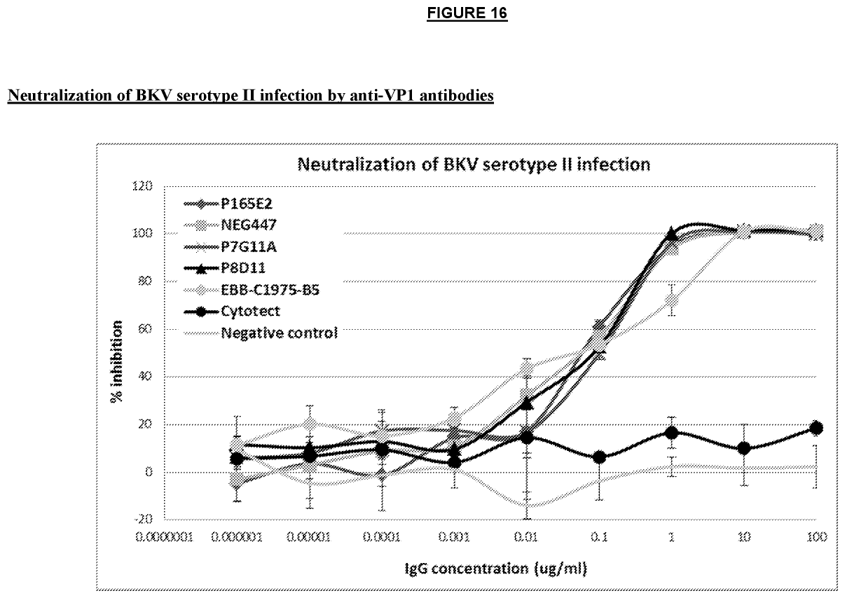

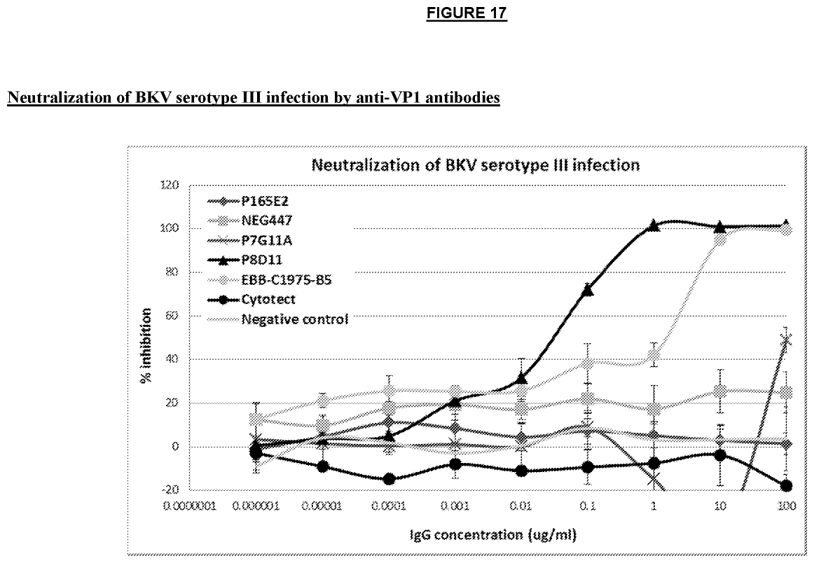

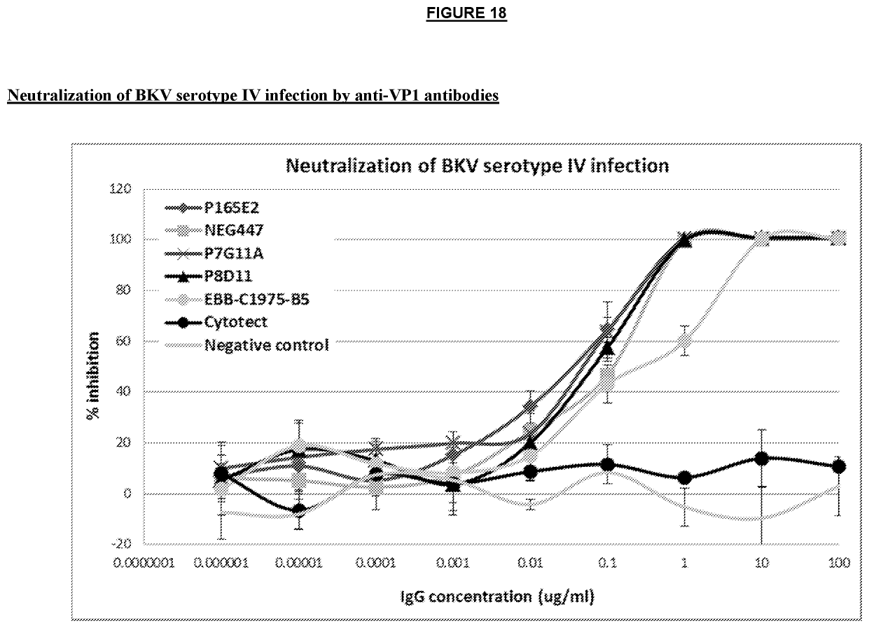

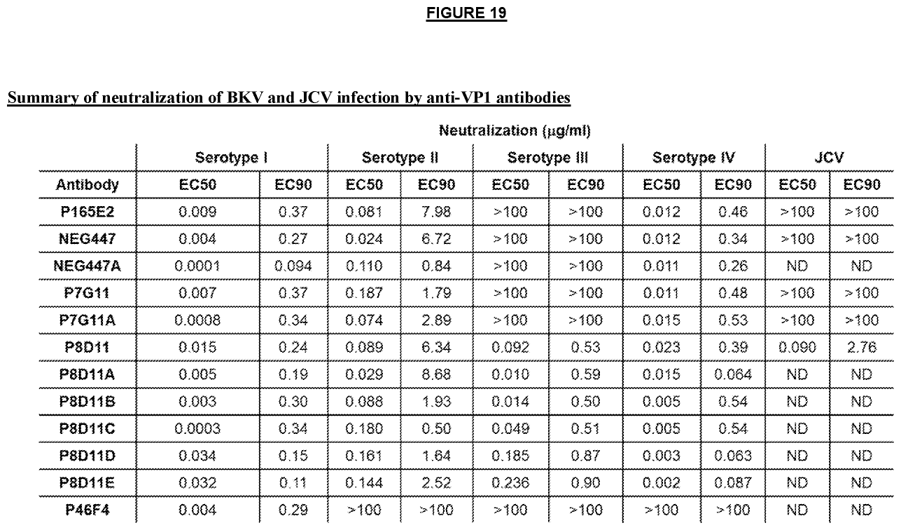

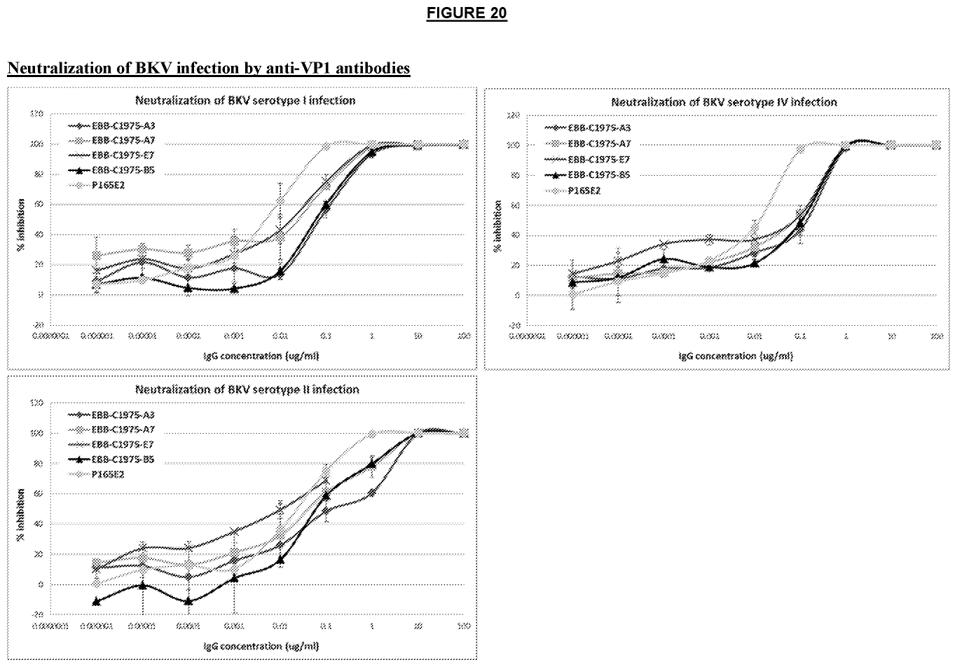

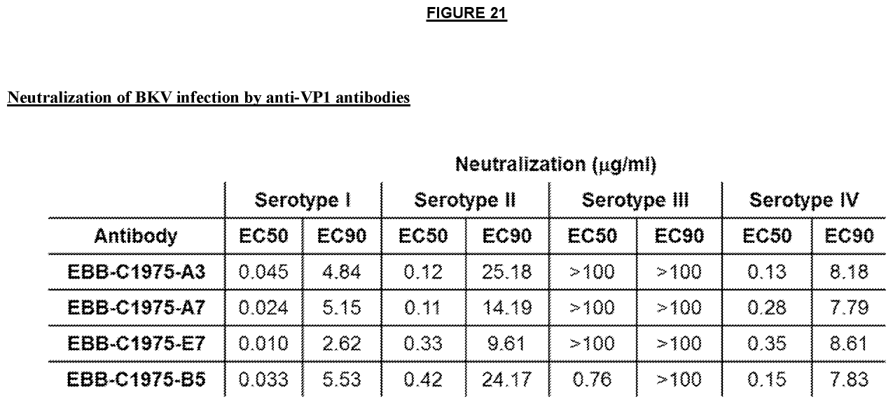

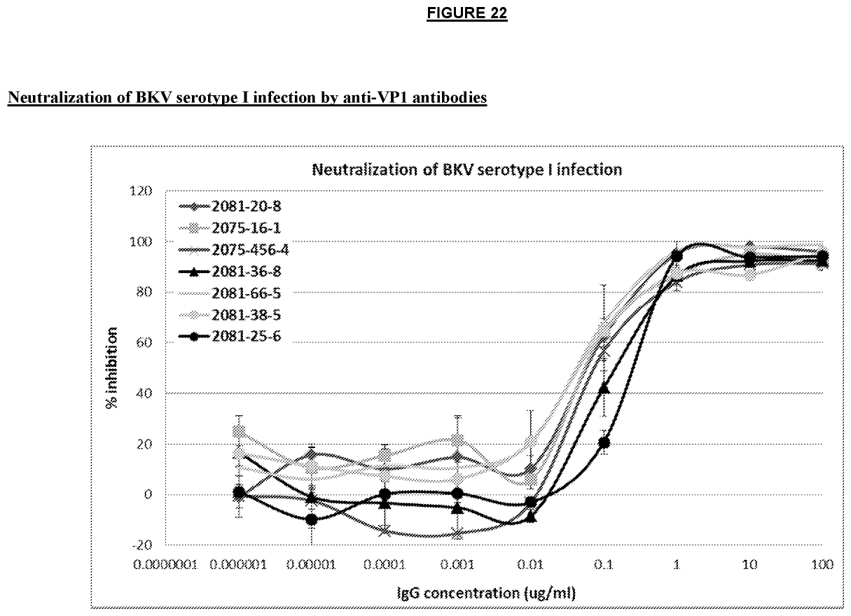

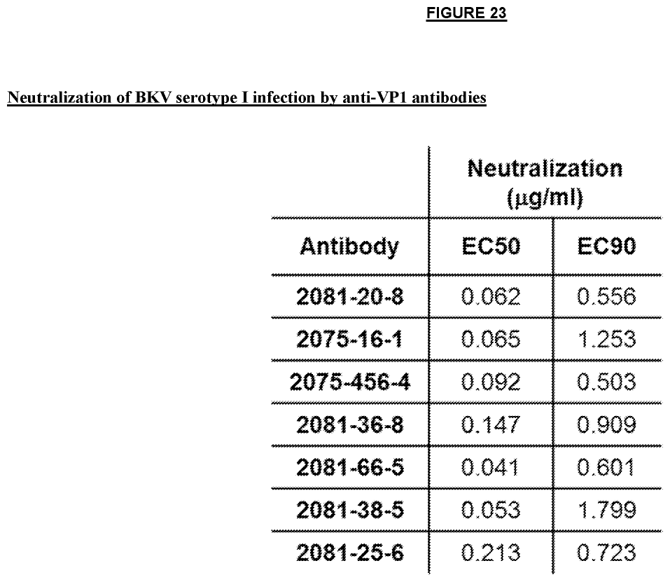

As shown in FIGS. 15-23, anti-VP1 antibodies neutralized infection by BKV, including the subset of antibodies that neutralize infection by all four serotypes of BKV (I-IV). These anti-VP1 antibodies specifically include P8D11, the modifications of P8D11, and EBB-C1975-B5.

### Example 10: Neutralization of JC Virus by Anti-VP1 Virus Antibodies

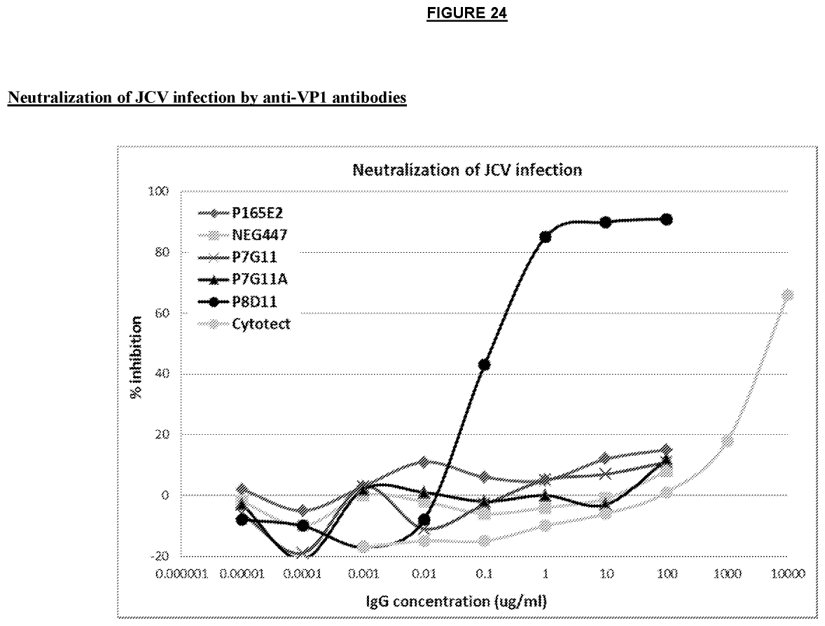

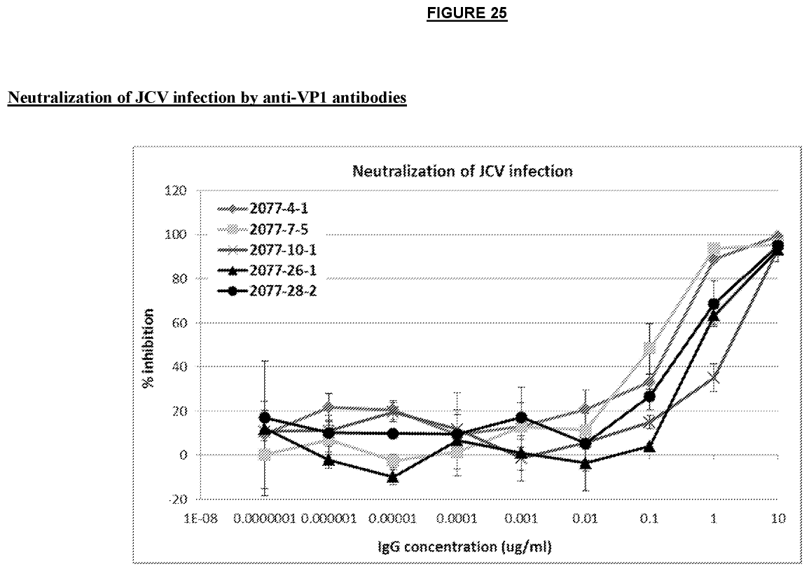

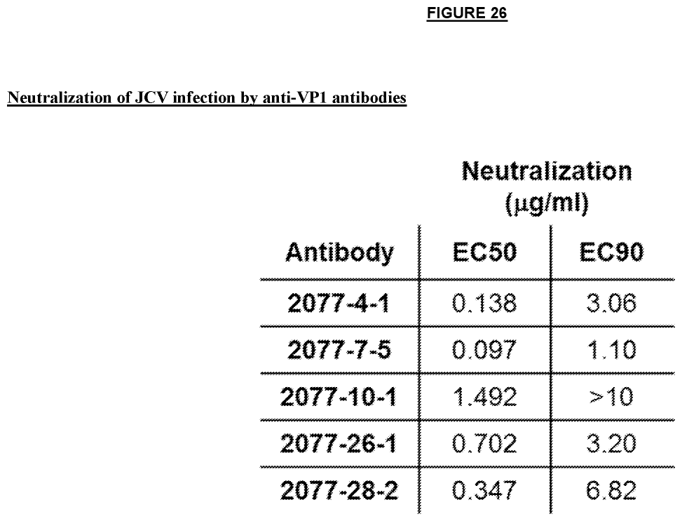

The infectious JCV isolates Mad-1 and Mad-4, have identical VP1 sequences (GenBank Accession NP_043511). These JCV isolates were pre-incubated with purified antibodies for 1 hour to allow for binding and neutralization. COS7 cells (African green monkey kidney fibroblast-like cell line expressing SV40 TAg, ATCC cat #CRL-1651) were then exposed to the virus-antibody mixture for 4 hours, replaced with fresh medium, and incubated for 72 hours to allow for viral entry and gene expression. Cells were fixed with 4% paraformaldehyde and analyzed by immunofluorescence to detect JCV VP1 expression (Abcam 53977, rabbit polyclonal anti-SV40 VP1 antibody). The assay was analyzed by high content image analysis using the Cellomics ArrayScan®VTI HCS Reader (Thermo Fisher, Waltham MA) to quantify the percent of JCV-infected cells (VP1-positive, DAP1-positive), with data presented as percent inhibition of infection relative to untreated control wells. As shown in FIGS. 24-26, a subset of anti-VP1 antibodies neutralize infection by JCV, including P8D11 and the 2077-series of antibodies.

### Example 11: Viral Resistance

Resistance selection experiments with P8D11 antibodies were carried out in renal proximal tubular epithelial (RPTE) cell cultures infected with BKV serotype I or serotype IV. In serotype I studies, no viral breakthrough was observed in cultures with P8D11 out to 6 passages (84 days) and thus no resistance-associated variants (RAVs) were indentified. No further passaging past this point was done, as no virus could be detected. In contrast, viral breakthough was detected at passage 3 (day 42) with another antibody. Sequencing of BKV VP1 from these cultures identified a resistance-associated variant (RAV) with 20 amino acid changes throughout VP1, with no changes clustering around specific amino acids in the VP1 sequence. Subsequent phenotypic characterization of this pooled RAV virus showed a complete loss of neutralization activity (>7,692-fold shift in EC50) when compared to wild-type virus, but little change (3.9 fold) in the EC50 of P8D11. In addition, the VP1 mutant E82K was identified as a RAV during selection with another anti-VP1 antibody (see Example 8), and characterization of a cloned E82K mutant virus showed this variant conferred a 15,880-fold shift in EC50 when compared to wild-type virus, but showed no cross-resistance to P8D11.

Similarly, in BKV serotype IV cultures, resistance was not detected with P8D11 after 6 passages (84 days). Again, No further passaging past this point was done, as no virus could be detected. However, resistance to a different anti-BK antibody was selected as early as passage 1 (day 14). Changes in amino acids L68R and E73K were identified as change-from-reference mutations and conferred 600- and 227-fold shifts in EC50 values respectively, but displayed no cross-resistance to P8D11. In summary, P8D11 has a high barrier-to-resistance and maintains neutralizing activity against resistant variants for both serotypes I and IV.

### Example 12: Toxicity

Because VP1 is an exogenous, non-human target that is not expressed on the cell surface, the anti-VP1 antibodies disclosed herein constitute a low risk for toxicity in human. A TCR study demonstrated there was no staining of 42 human tissues and blood smears by P8D11, supporting the absence of anti-VP1 antibody cross-reactivity with human proteins. The anti-VP1 antibodies have shown no antibody-dependent cell-mediated cytotoxicity (ADCC) in vitro, consistent with the fact that VP1 protein is not expressed on the host cell surface.

### Example 13: SET Affinity Assay of P8D11 on JCV VLPs

Progressive Multifocal Leukoencephalopathy (PML) is a rare, but frequently fatal infection of the brain of immunocomprimised patients by JC virus. The major capsid protein (VP1) of JC virus, is involved in binding sialic acid receptors on the surface of host cells. Certain mutations in the VP1, such as at amino acids L55 and S269, abolish sialic acid recognition and play a role in PML pathogenesis (Chen et al., mAbs 2015; 7(4), 681-692). These two mutations occur frequently in PML patients (Gorelik et al., J.Infect. Dis. 2011 204:103-114 and Reid et al., J. Infect. Dis. 2011; 204:237-244). The antibodies of the disclosure were tested to see if they bound to the mutated JCV VLPs with mutations at those positions. Binding of the anti-VP1 antibodies to these VLPs would indicate that JC virus carrying these common VP1 mutations would not be resistant to therapy.

Two series of twenty-two serial dilution of VLP were prepared in sample buffer. Two constant concentrations of the P8D11 antibody were added. The concentration of P8D11 antibody used was either 9 nM or 1 pM. The concentration range of JCV consensus ranged from 105 μg/ml-72 pg/ml. The concentration range of JCV L55F mutant was 300 μg/ml-143 pg/ml. The concentration range of JCV S269F mutant was 300 μg/ml-143 pg/ml. A volume of 60 μl of each VLP:antibody mix was distributed in duplicates to a 384-well polypropylene microtiter plate (PP MTP). Sample buffer served as negative control and a sample containing no antigen as positive control (Bmax). The plate was sealed and incubated over night (o/n) at room temperature (RT). A 384-well standard MSD array plate was coated o/n with 2 and 0.002 μg/ml of BKV-VP1 serotype I pentameric protein. After three times washing with 50 μl/well washing buffer, the plate was blocked with 50 μl/well blocking buffer for 1 hour at RT. After washing, a volume of 30 μl/well of each VLP:antibody mix was transferred from the PP MTP to the coated MSD plate and incubated for 20 min at RT. After an additional wash step, 30 μl of detection antibody (diluted 1:2000) in sample buffer was added to each well and incubated for 30 min at RT. The MSD plate was washed and 35 μl/well of read buffer was added and incubated for 5 min. ECL signals were measured with the MSD SECTOR Imager 6000.

The reagents used were: Bovine serum albumin (BSA), (VWR Cat #422351S), Phosphate-buffered saline (PBS) 10×, (Teknova Cat #P0195), MSD Read Buffer T 4×, (Meso Scale Discovery Cat #R92TC-1), Tris-buffered saline (TBS) 20×, (Teknova Cat #T1680), Tween-20, (VWR Cat #437082Q). The buffers used were; Blocking buffer: 1×PBS+5% (w/v) BSA, Coating buffer: 1×PBS, Sample buffer: 1×PBS+0.5% (w/v) BSA+0.02% (v/v) Tween-20, Wash buffer: 1×TBS+0.05% (v/v) Tween-20 and Read buffer: 1×MSD Read Buffer.

A solution equilibrium titration (SET) assay was used to determine the interaction affinities (K_{D}) of P8D11 with JCV VLPs as described in Example 5. P8D11 antibody was assayed at either 9 nM or 1 pM concentrations (constant) and JCV VLPs were serially diluted as follows: consensus VLPs ranged from 105 μg/ml-72 pg/ml, and L55F and S269F mutant VLPs both ranged from 300 μg/ml-143 pg/ml. Antibody:VP1 pentamer solution was incubated overnight, then assayed for unbound antibody using an MSD array plate (Meso Scale Discovery, Cat #L21XA, Rockville MD) coated with VP1 pentamer. The K_{D} was determined by fitting the plot with a 1:1 fit model (according to Piehler et al. J. Immunol. Methods. 1997; 201(2):189-206). The analysis was performed by using KinExA® Pro and n-Curve Analysis softwares from Sapidyne (Boise ID).

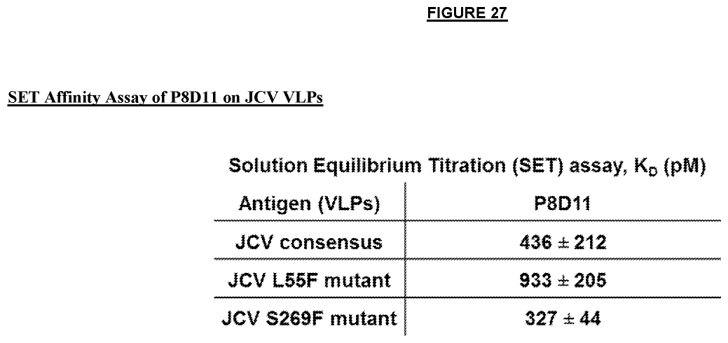

FIG. 27 depicts the results of the SET assay in tabular form. This data provides the affinity determination (K_{D}) of P8D11 antibody to consensus JCV VLPs and VLPs containing VP1 mutations commonly associated with PML. P8D11 showed binding affinities to all JCV VLPs in the low nanomolar range. However, the binding affinity to the L55F mutant was approximately 2-fold lower than the affinity for wild type (consensus) and S269F mutant VLPs. Therefore, this indicates that the P8D11 antibody would still be an effective therapy against either wild type JC virus or JC virus with mutations commonly associated with PML.

### Example 14: Deuterium Exchange Study (P8D11 Fab in Complex with BKV VP1 Pentamers) for Epitope Mapping

Deuterium exchange mass spectrometry (HDx-MS) measures the deuterium uptake on the amide backbone of a protein. These measurements are sensitive to the amide's solvent accessibility and to changes in the hydrogen bonding network of the backbone amides. HDx-MS is often used to compare proteins in two different states, such as apo and ligand-bound, and coupled with rapid digestion with pepsin. In such experiments one can locate regions, typically of 10 to 15 amino acids, that show differential deuterium uptake between two different states. Regions that are protected are either directly involved in ligand binding or allosterically affected by binding of the antibody to the ligand.

In these experiments, the deuterium uptake of BKV VP1 protein (SEQ ID NO:502), was measured in the absence and presence of P8D11 Fab fragment. Regions in VP1 that show a decrease in deuterium uptake upon binding of the Fab fragment are likely to be involved in the epitope; however, due to the nature of the measurement it is also possible to detect changes remote from the direct binding site (allosteric effects). In general, the regions that have the greatest amount of protection are involved in direct binding.

The epitope mapping experiments are performed on a Waters Synapt® G2 HDx-MS platform, which includes LEAP® robot system, nanoACQUITY® UPLC System, and Synapt® G2 mass spectrometer. In this method, triplicate control experiments are carried out as follows. BKV serotype I VP1 pentamer is diluted into 110 μl of 95% deuterated PBS buffer (pH 7.4) and incubated at room temperature on a bench rotator for 25 minutes (% D=85.5%). Deuterium exchange is quenched by 1:1 dilution with cold quench buffer (6M Urea and 1M TCEP pH=2.5) on ice for 5 min. After quenching the tube is transferred onto a LEAP system (Thermo box is set at 2° C.) and the quenched sample is injected by the LEAP system onto the UPLC system for analysis. The UPLC system incorporates an immobilized pepsin column 2.1 mm×30 mm (Life Technologies 2-3131-00) that is maintained at 12° C. An 8-minute 2 to 35% acetonitrile gradient and Waters UPLC CSH C18 1.0×100 mm column is used for separation. Next, triplicate experiments are carried out using the antibody. The P8D11 Fab fragment is immobilized on Protein G agarose beads (Thermo Scientific Cat #22851) using standard techniques. Briefly, the antibody is centrifuged to remove a storage buffer. Then 200 μl of PBS buffer (pH 7.4) and a concentration of VP1 pentamers are added to the immobilized P8D11 Fab fragment and incubated for 30 min at room temperature. After incubation, the complex is centrifuged and washed with 200 μl PBS buffer and centrifuged again. For deuterium exchange, 200 μl of deuterated PBS is added to the antigen-antibody complex for incubation at room temperature for 25 minutes (% D=85.5%). Deuterium buffer is then removed, and immediately, 125 μl ice cold quench buffer is added. After quenching for 5 minutes, the column is centrifuged and the flow-through is transferred into a prechilled HPLC vial. The sample is analyzed using the same on-line pepsin digestion/LC-MS setup as the control experiment.

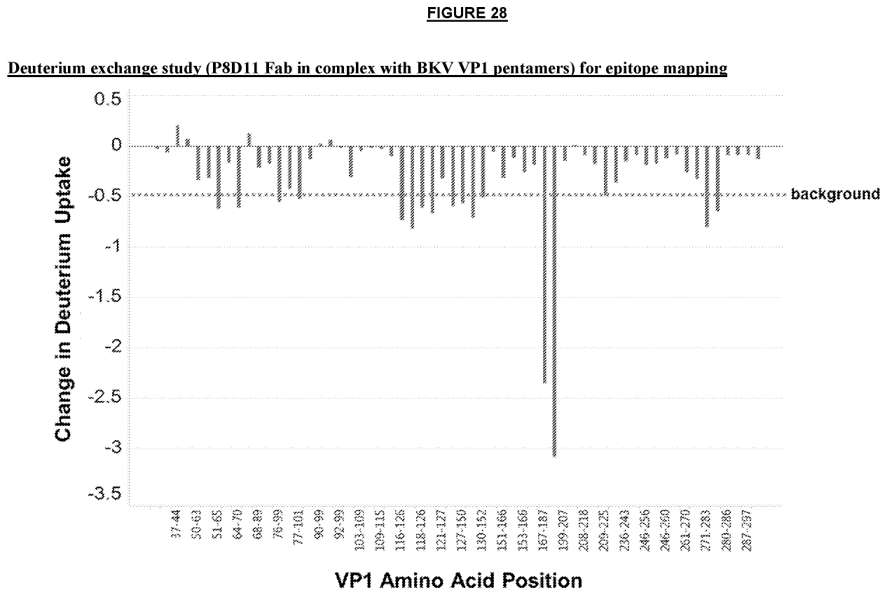

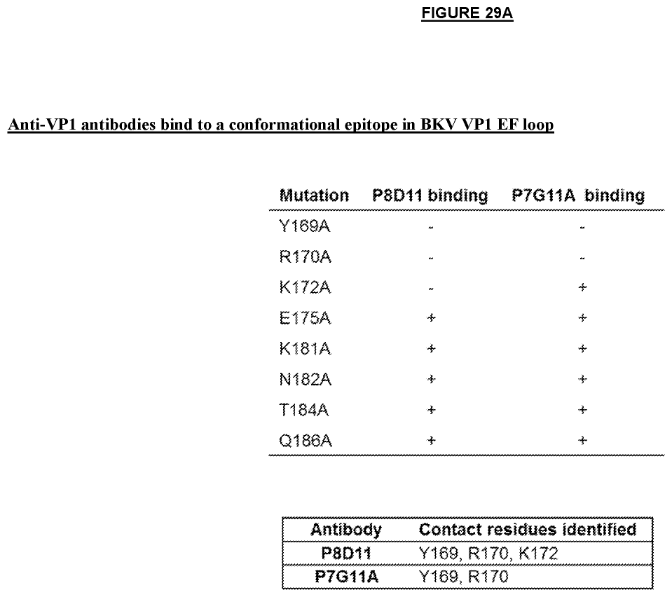

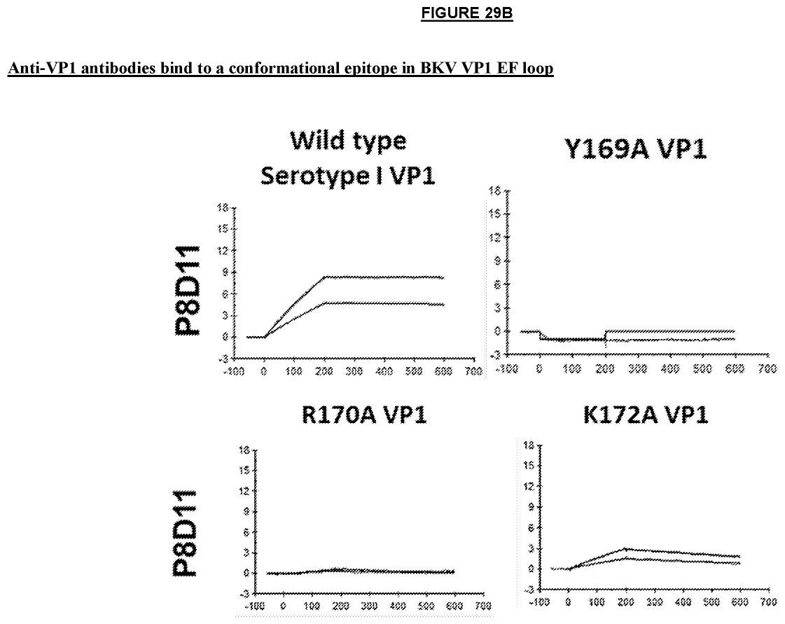

The results of these measurements are summarized in FIG. 28. FIG. 28 shows the baseline corrected differences between the control and P8D11 antibody bound sample divided by the standard error in the measurement. In this plot the more negative value indicates a greater amount of protection in a given region upon binding of P8D11 Fab fragment to BKV serotype I VP1 pentamer. We observe the most significant amounts of protection in amino acids 168-190 of the VP1 protein upon binding of P8D11 Fab fragment. This region of the EF loop is highly conserved across all four serotypes of BK virus and JC virus, as can be seen with the sequences bolded and underlined in Table 1 ((NYRTKYPXGTXXPKNXTXQSQVM) (SEQ ID NO:501)).

In conclusion, the deuterium mapping data indicate that P8D11 antibody binds to an epitope within the EF loop of BKV VP1. This region is highly conserved across all four BKV serotypes and JC virus, and thus supports the result that P8D11 has neutralizing activity across all four BKV serotypes and JC virus.

### Example 15: Targeted Alanine Scanning and SPR for Epitope Mapping of P8D11

Biacore surface plasmon resonance (SPR) was used to characterize the binding of anti-VP1 antibodies to VP1 pentamers generated for epitope mapping by scanning alanine mutagenesis. Experiments were performed at 25° C. in phosphate buffered saline (PBS) supplemented with 0.005% Tween 20 detergent (Calbiochem #655206) and run on a Biacore T-200 instrument (GE Healthcare Life Sciences). Biotinylated protein A (Sigma #P2165) was immobilized onto a Series S streptavidin sensor chip to approximately 1200 response units (RUs) and remaining free streptavidin sites were blocked with biotin-PEG (Pierce EZ-Link #PI21346). Antibodies were captured onto the prepared protein A sensor chip with a 4 second injection at a flow rate of 30 μl/minute. Antibodies were immobilized at 20-40 RUs on flowcells 2, 3 and 4, leaving flowcell 1 as a reference cell without any antibody. VP1 pentamers were then injected over the chip for 200 seconds at 100 μl/min followed by injections of buffer to monitor dissociation. Between each pentamer and pentamer concentration, the sensor chip surface was regenerated with an injection of 25 mM NaOH for 60 seconds at 30 μl/minute to remove antibodies before re-capture of antibodies onto the protein A surfaces for the next cycle. Data analysis was done in the GE BiaEvaluation software where double reference subtraction was applied. Assessment of the effect of alanine mutagenesis on VP1 pentamer binding was achieved by comparison of the binding RU levels and shapes of the binding curves compared to those of the wildtype pentamer.

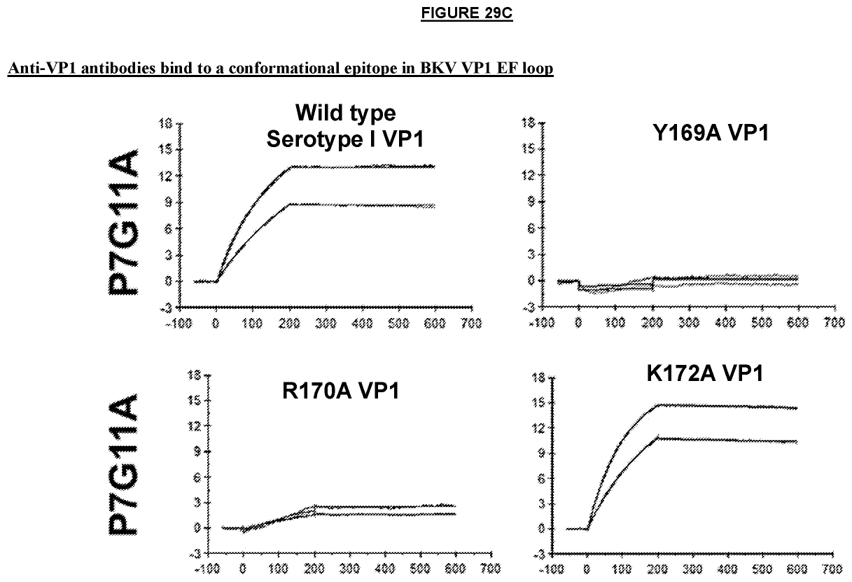

As discussed previously, the epitopes for antibodies P8D11 and P7G11A are conformational and non-contiguous (FIGS. 12A-B). Here, single mutations to alanine at Y169, R170 and K172 in the EF loop of BKV VP1 abolishes binding of P8D11 (FIGS. 29A and 29B). Mutations at Y169 and R170 also abolish binding of the P7G11A antibody, however binding of this antibody is not affected by changes at position K172 of the EF loop of BKV VP1 (FIGS. 29A and 29C).

### Example 16: Epitope Mapping by x-Ray Crystallography

The crystal structure of the scFv chain of the antibody P8D11 bound to the BKV major capsid protein VP1 in its pentameric form was determined. As detailed below, a 5.5:1 solution of scFv:BKV-VP1 pentamer was used to produce a crystallographically suitable complex composed of five scFv chains bound to each pentamer. Protein crystallography was then employed to generate an atomic resolution structure and define the epitope.

### Crystallization and Structure Determination

The P8D11 scFv/BKV-VP1 complex was concentrated to 5.2 mg/ml and screened for crystallization. Crystals for data collection were grown by hanging drop vapor diffusion at 18° C. Crystals were grown by mixing 1.0 μl of the complex with 1.0 μl of reservoir solution containing 25% (w/v) PEG3350, 0.2 M magnesium chloride and 0.1 M Bis-Tris pH 7.0, and equilibrating the drop against 350 μl of the same reservoir solution. Crystals grew overnight and continued to grow for a few days. Before data collection, the crystals were transferred to 75% of reservoir solutions plus 25% glycerol and flash cooled in liquid nitrogen.

Diffraction data were collected in-house on a Rigaku FRE+ copper source and an R-axis X-ray detector. Data was processed and scaled using Autoproc (Global Phasing, LTD). The data of BKV-VP1 was processed to 2.66 Å in space group P42212 with cell dimensions a=224.4 Å, b=224.4 Å, c=144.04 Å, alpha=90°, beta=90°, gamma=90°. The structure of the complex was solved by molecular replacement using Phaser (McCoy et al., (2007) J. Appl. Cryst. 40:658-674) with a BKV-VP1 pentamer as the search model. The final model was built in COOT (Emsley & Cowtan (2004) Acta Cryst. D60:2126-2132) and refined with Buster (Global Phasing, LTD, Cambridge, UK). The Rwork and Rfree values are 17.1% and 21.4%, respectively; and root-mean-square (r.m.s) deviation values of bond lengths and bond angles are 0.010 Å and 1.18°, respectively.

Residues of BKV-VP1-Pentamer that are in contact with the P8D11 scFv, the types of interactions, and the buried surface areas are all identified by PISA (Krissinel et al., (2007) J Mol Biol. 372:774-97) and listed in Table 6 below. It was found the each monomer of the VP1-pentamer contains a single isolated epitope for the P8D11 antibody. Thus five scFv domains bind to each pentamer at five chemically and sterically equivalent positions. Details for the interactions at each epitope are essentially identical so that only one scFv/VP1-epitope interface is analyzed here.

### Epitopes of P8D11-scFv on BKV-VP1

### Overall Structure

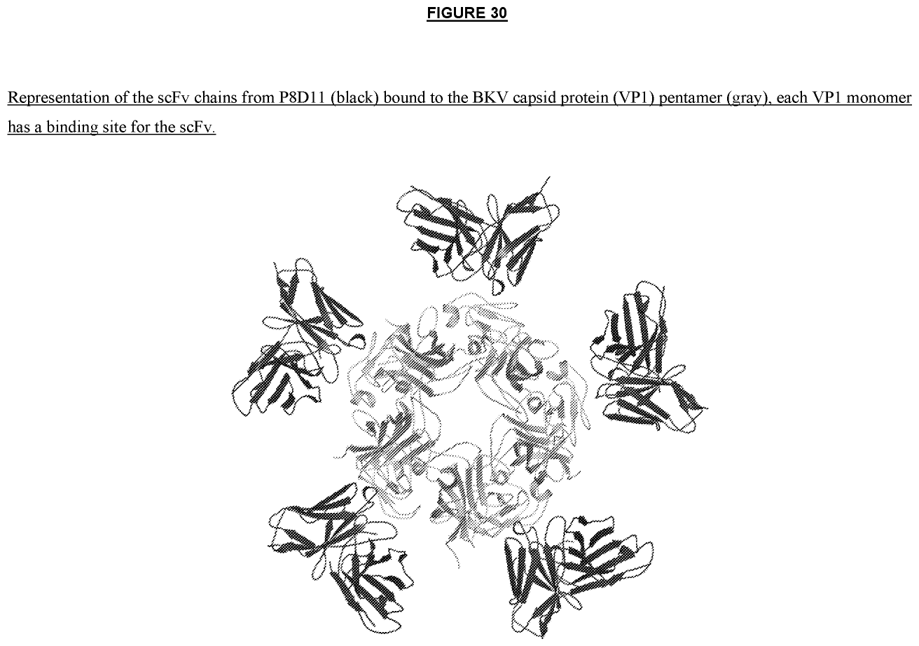

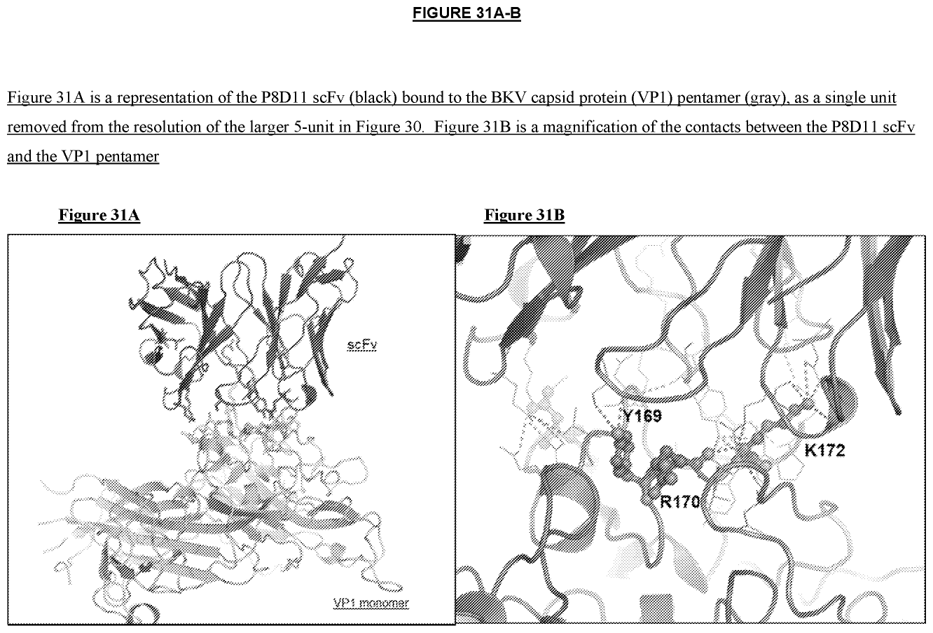

The overall folding of each polyomavirus VP1-pentmer structure is highly homologous at the level of tertiary structure. Primary sequences are well conserved with identity as at 69-85%. Each pentamer is composed of five monomers, each of which is composed by a three-strand β sheet stacking against another five-strand β sheet and then a four-strand β sheet. The P8D11 scFv is a VH-VL fusion protein with a 20 amino acid linker between VH and VL domains. As shown in FIG. 30, the VH-VL fusion protein binds to an epitope located on the lateral, exterior surface of the BKV-VP1-pentamer.

### Epitope of P8D11

The crystal structure of the BKV-VP1/P8D11 complex is used to identify the P8D11 epitope on BKV-VP1. The interaction surface on VP1 by P8D11-scFv is formed by several continuous and discontinuous (i.e. noncontiguous) sequences: namely residues 77-80, 169-186, and 191-192, as detailed in Table 6. These residues form the three-dimensional conformational epitope that is recognized by the P8D11-scFv (FIG. 31A-B). This epitope defined by crystallography is in good agreement with that defined by hydrogen deuterium exchange mass spectrometry (HDx-MS), in which residues 168-190 are substantially protected by P8D11-Fab (FIG. 28). There is also good agreement with the Alanine scan that was done on the key amino acids of the epitope (FIG. 29A-C) which showed that TYR169, ARG170 and LYS172 are contact residues which are part of the epitope of the P8D11 antibody.

P8D11-scFv epitope on BKV-VP1. All residues of BKV-VP1 that are in contact with P8D11-scFv in the crystal structure are identified by PISA, listed and sorted by their buried surface area by P8D11-scFv. Types of interaction are also listed where applicable.

TABLE 6

| Anti-VP1 | | | | | VP1 | | | | |
|---|---|---|---|---|---|---|---|---|---|
| scFv | Hydrogen | Salt | | | Pentamer | Hydrogen | Salt | | |
| residue | bond | Bridge | ASA* | BSA* | residue | bond | Bridge | ASA* | BSA* |
| ASN31 | 0 | 0 | 72.36 | 1.11 | SER77 | 1 | 0 | 74.90 | 25.99 |
| TYR32 | 0 | 0 | 60.89 | 6.59 | SER78 | 0 | 0 | 73.45 | 46.46 |
| TRP33 | 0 | 0 | 42.56 | 39.19 | ASP79 | 1 | 0 | 36.90 | 2.33 |
| LYS52 | 2 | 0 | 66.84 | 59.49 | SER80 | 0 | 0 | 63.82 | 20.03 |
| LYS53 | 0 | 1 | 89.76 | 40.27 | TYR169 | 2 | 0 | 66.99 | 44.33 |
| ASP54 | 0 | 0 | 100.58 | 3.42 | ARG170 | 1 | 0 | 117.97 | 54.91 |
| SER56 | 0 | 0 | 78.38 | 5.60 | THR171 | 1 | 0 | 12.44 | 9.58 |
| GLU57 | 2 | 0 | 63.29 | 46.19 | LYS172 | 2 | 2 | 142.87 | 121.15 |
| TRY59 | 0 | 0 | 55.86 | 13.29 | TYR173 | 3 | 0 | 33.81 | 24.25 |
| VAL99 | 0 | 0 | 19.54 | 17.42 | PRO174 | 1 | 0 | 27.18 | 4.61 |
| ARG100 | 1 | 0 | 97.42 | 14.49 | GLU175 | 1 | 0 | 165.93 | 61.70 |
| GLY102 | 1 | 0 | 45.24 | 23.46 | GLY176 | 0 | 0 | 30.62 | 0.58 |
| ARG103 | 5 | 0 | 190.68 | 157.62 | THR177 | 1 | 0 | 21.14 | 13.74 |
| TYR104 | 4 | 0 | 68.25 | 63.79 | ILE178 | 0 | 0 | 63.25 | 0.67 |
| PHE105 | 0 | 0 | 37.94 | 32.69 | THR179 | 2 | 0 | 29.52 | 22.67 |
| ASN526 | 0 | 0 | 84.174 | 16.28 | PRO180 | 0 | 0 | 11.34 | 7.66 |
| GLY528 | 1 | 0 | 27.52 | 11.48 | LYS181 | 2 | 0 | 164.73 | 85.21 |
| SER529 | 0 | 0 | 62.31 | 55.68 | ASN182 | 1 | 0 | 111.76 | 104.95 |
| ARG530 | 4 | 0 | 83.56 | 70.86 | PRO183 | 0 | 0 | 50.84 | 49.34 |
| PRO531 | 0 | 0 | 14.77 | 13.77 | THR184 | 1 | 0 | 67.62 | 67.45 |
| ASP549 | 0 | 0 | 21.15 | 4.11 | ALA185 | 0 | 0 | 61.12 | 6.85 |
| ASP550 | 0 | 2 | 37.36 | 16.62 | GLN186 | 1 | 0 | 103.62 | 42.67 |
| SER551 | 0 | 0 | 68.46 | 11.52 | ASN191 | 2 | 0 | 13.15 | 12.56 |
| ASN552 | 1 | 0 | 65.13 | 28.37 | THR192 | 0 | 0 | 89.19 | 1.87 |
| TRP590 | 0 | 0 | 51.46 | 50.45 | ASP193 | 0 | 1 | 123.11 | 71.49 |
| SER591 | 0 | 0 | 40.49 | 16.66 | | | | | |
| SER592 | 0 | 0 | 51.90 | 25.61 | | | | | |
| SER593 | 2 | 0 | 45.01 | 33.37 | | | | | |

*ASA: Accessible Surface Area
*BSA: Buried Surface Area

### Example 18: Formulation

The anti-VP1 antibodies described herein are monoclonal antibodies, IgG1 isotype with lambda light chain, and can be lyophilized. These antibodies are soluble and stable in a histidine-sucrose formulation buffer for 4 weeks. In addition, anti-VP1 antibodies were soluble at >200 mg/ml as minimally formulated drug substance (e.g., in histidine buffer in the absence of stabilizers).

For subsequent intravenous administration, the obtained solution will usually be further diluted into a carrier solution to the ready-to-use antibody solution for infusion.

Important stability-indicating analytical methods to select the most stable formulation encompassed, amongst others, size-exclusion chromatography to determine aggregation levels, subvisible particulate matter testing, and potency testing.

It is understood that the examples and aspects described herein are for illustrative purposes only and that various modifications or changes in light thereof will be suggested to persons skilled in the art and are to be included within the spirit and purview of this application and scope of the appended claims.

1-47. (canceled)

48. An isolated antibody or antigen binding fragment thereof comprising:
(i) a heavy chain variable region (vH) that comprises (a) a HCDR1 (CDR-Complementarity Determining Region) of SEQ ID NO: 246, (b) a HCDR2 of SEQ ID NO: 247, and (c) a HCDR3 of SEQ ID NO: 248, and a light chain variable region (vL) that comprises: (d) a LCDR1 of SEQ ID NO: 256, (e) a LCDR2 of SEQ ID NO: 257, and (f) a LCDR3 of SEQ ID NO: 258;
(ii) a heavy chain variable region (vH) that comprises (a) a HCDR1 of SEQ ID NO: 266, (b) a HCDR2 of SEQ ID NO: 267, and (c) a HCDR3 of SEQ ID NO: 268, and a light chain variable region (vL) that comprises: (d) a LCDR1 of SEQ ID NO: 276, (e) a LCDR2 of SEQ ID NO: 277, and (f) a LCDR3 of SEQ ID NO: 278; or
(iii) a heavy chain variable region (vH) that comprises (a) a HCDR1 of SEQ ID NO: 286, (b) a HCDR2 of SEQ ID NO: 287, and (c) a HCDR3 of SEQ ID NO: 288, and a light chain variable region (vL) that comprises: (d) a LCDR1 of SEQ ID NO: 296, (e) a LCDR2 of SEQ ID NO: 297, and (f) a LCDR3 of SEQ ID NO: 298.

49. The antibody or antigen binding fragment thereof of claim 48, wherein the antibody or antigen binding fragment thereof comprises:
(i) a heavy chain variable region (vH) that comprises SEQ ID NO:252, and a light chain variable region (vL) that comprises SEQ ID NO: 262;
(ii) a heavy chain variable region (vH) that comprises SEQ ID NO:272, and a light chain variable region (vL) that comprises SEQ ID NO: 282; or
(iii) a heavy chain variable region (vH) that comprises SEQ ID NO:292, and a light chain variable region (vL) that comprises SEQ ID NO: 302.

50. The antibody or antigen binding fragment thereof of claim 48, wherein the antibody is a monoclonal antibody, a chimeric antibody, a humanized antibody, a human engineered antibody, a human antibody, or a single chain antibody (scFv).

51. The antibody or antigen binding fragment thereof of claim 48, wherein said antibody or antigen binding fragment thereof specifically binds to a VP1 epitope (SEQ ID NO:501).

52. The antibody or antigen binding fragment thereof of claim 48, wherein the antibody or fragment thereof has reduced glycosylation, no glycosylation or is hypofucosylated.

53. A pharmaceutical composition comprising the antibody or antigen binding fragment thereof of claim 48 and further comprising a pharmaceutically acceptable carrier.

54. A pharmaceutical composition comprising a plurality of antibodies or antigen binding fragments thereof of claim 48, wherein at least 0.05%, 0.1%, 0.5%, 1%, 2%, 3%, 5% or more of the antibodies or antigen binding fragments thereof in the composition have an α2,3-linked sialic acid residue.

55. A pharmaceutical composition comprising a plurality of antibodies or antigen binding fragments thereof of claim 48, wherein none of the antibodies or antigen binding fragments thereof comprise a bisecting GlcNAc.

56. A pharmaceutical composition comprising the antibody or fragment thereof of claim 48, wherein the composition is a lyophilisate.

57. A method of neutralizing a BK virus (BKV) or John Cunningham (JC) virus (JCV) infection comprising administering, via injection or infusion to a patient in need thereof, an effective amount of the antibody or antigen binding fragment thereof of claim 48.

58. The method of claim 57, wherein the patient in need thereof is diagnosed with BK viruria or BK viremia.

59. A method of treating or reducing the likelihood of a BK virus or JC virus associated disorder, comprising administering, via injection or infusion to a patient in need thereof, an effective amount of the antibody or antigen binding fragment thereof of claim 48, and wherein the disorder is selected from the group consisting of: a nephropathy, a BKV-associated nephropathy (BKVAN), a hemorrhagic cystitis (HC), Progressive Multifocal Leukoencephalopathy (PML), a granule cell neuronopathy (GCN), an interstitial kidney disease, a ureteral stenosis, a vasculitis, a colitis, a retinitis, a meningitis, and an immune reconstitution inflammatory syndrome (IRIS).

60. The method of claim 59, wherein the antibody or antigen binding fragment thereof is administered in combination with another therapeutic agent.

61. The method of claim 60, wherein the another therapeutic agent is an immunosuppressive agent.

62. An isolated nucleic acid that encodes the antibody or antigen binding fragment thereof of claim 48.

63. A vector comprising the nucleic acid of claim 62.

64. A host cell comprising the vector of claim 63.

65. A process for producing an antibody or antigen binding fragment thereof comprising cultivating the host cell of claim 64 and recovering the antibody or antigen binding fragment thereof from the culture.

66. A diagnostic reagent comprising the antibody or antigen binding fragment thereof of claim 48, wherein the antibody or antigen binding fragment thereof is labeled.

67. The diagnostic reagent of claim 66, wherein the label is selected from the group consisting of: a radiolabel, a fluorophore, a chromophore, an imaging agent, and a metal ion.


SEQUENCE LISTING (543 sequences)

SEQ ID NO: 1 - protein, 362 aa, unidentified
  source 1..362: note = Polyomavirus sp.
MAPTKRKGECPGAAPKKPKEPVQVPKLLIKGGVEVLEVKTGVDAITEVECFLNPEMGDPDENLRGFSLKLSAENDFSSDSPERKMLPCYSTARIPLPNLNEDLTCGNLLMWEAVTVQTEVIGITSMLNLHAGSQKVHEHGGGKPIQGSNFHFFAVGGDPLEMQGVLMNYRTKYPEGTITPKNPTAQSQVMNTDHKAYLDKNNAYPVECWIPDPSRNENTRYFGTFTGGENVPPVLHVTNTATTVLLDEQGVGPLCKADSLYVSAADICGLFTNSSGTQQWRGLARYFKIRLRKRSVKNPYPISFLLSDLINRRTQRVDGQPMYGMESQVEEVRVFDGTERLPGDPDMIRYIDKQGQLQTKML

SEQ ID NO: 2 - protein, 362 aa, unidentified
  source 1..362: note = Polyomavirus sp.
MAPTKRKGECPGAAPKKPKEPVQVPKLLIKGGVEVLEVKTGVDAITEVECFLNPEMGDPDDNLRGYSLKLTAENAFDSDSPDKKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLARYFKIRLRKRSVKNPYPISFLLSDLINRRTQKVDGQPMYGMESQVEEVRVFDGTEQLPGDPDMIRYIDRQGQLQTKMV

SEQ ID NO: 3 - protein, 362 aa, unidentified
  source 1..362: note = Polyomavirus sp.
MAPTKRKGECPGAAPKKPKEPVQVPKLLIKGGVEVLEVKTGVDAITEVECFLNPEMGDPDDHLRGYSQHLSAENAFDSDSPDKKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSKNENTRYFGTYTGGENVPPVLHVTNTATTVLLDEQGVGPLCKADSLYVSAADICGLFTNSSGTQQWRGLARYFKIRLRKRSVKNPYPISFLLSDLINRRTQKVDGQPMYGMESQVEEVRVFDGTEQLPGDPDMIRYIDRQGQLQTKMV

SEQ ID NO: 4 - protein, 362 aa, unidentified
  source 1..362: note = Polyomavirus sp.
MAPTKRKGECPGAAPKKPKEPVQVPKLLIKGGVEVLEVKTGVDAITEVECFLNPEMGDPDNDLRGYSLRLTAETAFDSDSPDRKMLPCYSTARIPLPNLNEDLTCGNLLMWEAVTVKTEVIGITSMLNLHAGSQKVHENGGGKPIQGSNFHFFAVGGDPLEMQGVLMNYRTKYPEGTVTPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLPRYFKIRLRKRSVKNPYPISFLLSDLINRRTQRVDGQPMYGMESQVEEVRVFDGTEQLPGDPDMIRYIDRQGQLQTKMV

SEQ ID NO: 5 - protein, 354 aa, unidentified
  source 1..354: note = Orthopolyomavirus sp.
MAPTKRKGERKDPVQVPKLLIRGGVEVLEVKTGVDSITEVECFLTPEMGDPDEHLRGFSKSISISDTFESDSPNKDMLPCYSVARIPLPNLNEDLTCGNILMWEAVTLKTEVIGVTTLMNVHSNGQATHDNGAGKPVQGTSFHFFSVGGEALELQGVVFNYRTKYPDGTIFPKNATVQSQVMNTEHKAYLDKNKAYPVECWVPDPTRNENTRYFGTLTGGENVPPVLHITNTATTVLLDEFGVGPLCKGDNLYLSAVDVCGMFTNRSGSQQWRGLSRYFKVQLRKRRVKNPYPISFLLTDLINRRTPRVDGQPMYGMDAQVEEVRVFEGTEELPGDPDMMRYVDRYGQLQTKML

SEQ ID NO: 6 - protein, 5 aa, synthetic construct
NYWMT

SEQ ID NO: 7 - protein, 17 aa, synthetic construct
NIKKDGSEKYYVDSVRG

SEQ ID NO: 8 - protein, 11 aa, synthetic construct
VRSGRYFALDD

SEQ ID NO: 9 - protein, 7 aa, synthetic construct
GFTFNNY

SEQ ID NO: 10 - protein, 6 aa, synthetic construct
KKDGSE

SEQ ID NO: 11 - protein, 11 aa, synthetic construct
VRSGRYFALDD

SEQ ID NO: 12 - protein, 120 aa, synthetic construct
QVQLVESGGTLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSS

SEQ ID NO: 13 - DNA, 360 bp, synthetic construct
  misc_feature 1..360: note = source = /note=Description of synthetic construct: Synthetic polynucleotide
  source 1..360: mol_type = other DNA
caggtgcagctggtggaatcaggcggcacactggtgcagcctggcggtagcctgagactgagctgcgctgctagtggcttcacctttaacaactactggatgacctgggttaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagc

SEQ ID NO: 14 - protein, 450 aa, synthetic construct
QVQLVESGGTLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1350 bp, synthetic construct
  misc_feature 1..1350: note = source = /note=Description of synthetic construct: Synthetic polynucleotide
  source 1..1350: mol_type = other DNA
caggtgcagctggtggaatcaggcggcacactggtgcagcctggcggtagcctgagactgagctgcgctgctagtggcttcacctttaacaactactggatgacctgggttaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 16 - protein, 11 aa, synthetic construct
GGDNIGSRPVH

SEQ ID NO: 17 - protein, 7 aa, synthetic construct
DDSNRPS

SEQ ID NO: 18 - protein, 10 aa, synthetic construct
QVWSSSTDHP

SEQ ID NO: 19 - protein, 7 aa, synthetic construct
DNIGSRP

SEQ ID NO: 20 - skipped

SEQ ID NO: 21 - protein, 7 aa, synthetic construct
WSSSTDH

SEQ ID NO: 22 - protein, 107 aa, synthetic construct
QSVLTQPPSVSVAPGKTARITCGGDNIGSRPVHWYQQKPGQAPILVVYDDSNRPSGIPERFSGSNSGNTATLTISRVEAGDEADYYCQVWSSSTDHPFGGGTKVTVL

SEQ ID NO: 23 - DNA, 321 bp, synthetic construct
  misc_feature 1..321: note = source = /note=Description of synthetic construct: Synthetic polynucleotide
  source 1..321: mol_type = other DNA
cagtcagtcctgactcagccccctagcgtcagcgtggcccctggtaaaaccgctagaatcacctgtggcggcgataatatcggctctaggcccgtgcactggtatcagcagaagcccggtcaagcccctatcctggtggtctacgacgactctaatagacctagcggaatccccgagcggtttagcggctctaattctggtaataccgctaccctgactatctctagggtggaagccggcgacgaggccgactactactgtcaagtctggtctagctctaccgatcaccccttcggcggaggcactaaggttacagtgctg

SEQ ID NO: 24 - protein, 213 aa, synthetic construct
QSVLTQPPSVSVAPGKTARITCGGDNIGSRPVHWYQQKPGQAPILVVYDDSNRPSGIPERFSGSNSGNTATLTISRVEAGDEADYYCQVWSSSTDHPFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - DNA, 639 bp, synthetic construct
  misc_feature 1..639: note = source = /note=Description of synthetic construct: Synthetic polynucleotide
  source 1..639: mol_type = other DNA
cagtcagtcctgactcagccccctagcgtcagcgtggcccctggtaaaaccgctagaatcacctgtggcggcgataatatcggctctaggcccgtgcactggtatcagcagaagcccggtcaagcccctatcctggtggtctacgacgactctaatagacctagcggaatccccgagcggtttagcggctctaattctggtaataccgctaccctgactatctctagggtggaagccggcgacgaggccgactactactgtcaagtctggtctagctctaccgatcaccccttcggcggaggcactaaggttacagtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 26 - protein, 5 aa, synthetic construct
  REGION 1..5: note = source = /note=Description of synthetic construct: Synthetic peptide
NYWMT

SEQ ID NO: 27 - protein, 17 aa, synthetic construct
NIKKDGSEKYYVDSVRG

SEQ ID NO: 28 - protein, 11 aa, synthetic construct
VRSGRYFALDD

SEQ ID NO: 29 - protein, 7 aa, synthetic construct
GFTFSNY

SEQ ID NO: 30 - protein, 6 aa, synthetic construct
KKDGSE

SEQ ID NO: 31 - protein, 11 aa, synthetic construct
VRSGRYFALDD

SEQ ID NO: 32 - protein, 120 aa, synthetic construct
QVQLVESGGTLVQPGGSLRLSCAASGFTFSNYWMTWVRQAPGKGLEWVANIKKDGSEKYYVDSVRGRFTISRDNAKNSLFLQMNSLRPEDTAVYFCATVRSGRYFALDDWGQGTLVTVSS

SEQ ID NO: 33 - DNA, 360 bp, synthetic construct
  misc_feature 1..360: note = source = /note=Description of synthetic construct: Synthetic polynucleotide
  source 1..360: mol_type = other DNA
caggtgcagctggtggaatcaggcggcacactggtgcagcctggcggtagcctgagactgagctgcgctgctagtggcttcaccttctctaactactggatgacctgggtcaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagc

SEQ ID NO: 34 - protein, 450 aa, synthetic construct
QVQLVESGGTLVQPGGSLRLSCAASGFTFSNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1350 bp, synthetic construct
  misc_feature 1..1350: note = source = /note=Description of synthetic construct: Synthetic polynucleotide
  source 1..1350: mol_type = other DNA
caggtgcagctggtggaatcaggcggcacactggtgcagcctggcggtagcctgagactgagctgcgctgctagtggcttcaccttctctaactactggatgacctgggtcaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 36 - protein, 11 aa, synthetic construct
GGDNIGSRPVH

SEQ ID NO: 37 - protein, 7 aa, synthetic construct
DDSNRPS

SEQ ID NO: 38 - protein, 10 aa, synthetic construct
QVWSSSTDHP

SEQ ID NO: 39 - protein, 7 aa, synthetic construct
DNIGSRP

SEQ ID NO: 40 - skipped

SEQ ID NO: 41 - protein, 7 aa, synthetic construct
WSSSTDH

SEQ ID NO: 42 - protein, 107 aa, synthetic construct; identical to SEQ ID NO: 22

SEQ ID NO: 43 - DNA, 321 bp, synthetic construct; identical to SEQ ID NO: 23
  misc_feature 1..321: note = source = /note=Description of synthetic construct: Synthetic polynucleotide
  source 1..321: mol_type = other DNA

SEQ ID NO: 44 - protein, 213 aa, synthetic construct; identical to SEQ ID NO: 24

SEQ ID NO: 45 - DNA, 639 bp, synthetic construct; identical to SEQ ID NO: 25
  misc_feature 1..639: note = source = /note=Description of synthetic construct: Synthetic polynucleotide
  source 1..639: mol_type = other DNA

SEQ ID NO: 46 - protein, 5 aa, synthetic construct
NYWMT

SEQ ID NO: 47 - protein, 17 aa, synthetic construct
NIKKDGSEKYYVDSVRG

SEQ ID NO: 48 - protein, 11 aa, synthetic construct
VRSGRYFALDD

SEQ ID NO: 49 - protein, 7 aa, synthetic construct
GFTFKNY

SEQ ID NO: 50 - protein, 6 aa, synthetic construct
KKDGSE

SEQ ID NO: 51 - protein, 11 aa, synthetic construct
VRSGRYFALDD

SEQ ID NO: 52 - protein, 120 aa, synthetic construct
QVQLVESGGTLVQPGGSLRLSCAASGFTFKNYWMTWVRQAPGKGLEWVANIKKDGSEKYYVDSVRGRFTISRDNAKNSLFLQMNSLRPEDTAVYFCATVRSGRYFALDDWGQGTLVTVSS

SEQ ID NO: 53 - DNA, 360 bp, synthetic construct
  misc_feature 1..360: note = source = /note=Description of synthetic construct: Synthetic polynucleotide
  source 1..360: mol_type = other DNA
caggtgcagctggtggaatcaggcggcacactggtgcagcctggcggtagcctgagactgagctgcgctgctagtggcttcacctttaagaactactggatgacctgggtcaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagc

SEQ ID NO: 54 - protein, 450 aa, synthetic construct
QVQLVESGGTLVQPGGSLRLSCAASGFTFKNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 1350 bp, synthetic construct
  misc_feature 1..1350: note = source = /note=Description of synthetic construct: Synthetic polynucleotide
  source 1..1350: mol_type = other DNA
caggtgcagctggtggaatcaggcggcacactggtgcagcctggcggtagcctgagactgagctgcgctgctagtggcttcacctttaagaactactggatgacctgggtcaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 56 - protein, 11 aa, synthetic construct
GGDNIGSRPVH

SEQ ID NO: 57 - protein, 7 aa, synthetic construct
DDSNRPS

SEQ ID NO: 58 - protein, 10 aa, synthetic construct
QVWSSSTDHP

SEQ ID NO: 59 - protein, 7 aa, synthetic construct
DNIGSRP

SEQ ID NO: 60 - skipped

SEQ ID NO: 61 - protein, 7 aa, synthetic construct
WSSSTDH

SEQ ID NO: 62 - protein, 107 aa, synthetic construct; identical to SEQ ID NO: 22

SEQ ID NO: 63 - DNA, 321 bp, synthetic construct; identical to SEQ ID NO: 23
  misc_feature 1..321: note = source = /note=Description of synthetic construct: Synthetic polynucleotide
  source 1..321: mol_type = other DNA

SEQ ID NO: 64 - protein, 213 aa, synthetic construct; identical to SEQ ID NO: 24

SEQ ID NO: 65 - DNA, 639 bp, synthetic construct; identical to SEQ ID NO: 25
  misc_feature 1..639: note = source = /note=Description of synthetic construct: Synthetic polynucleotide
  source 1..639: mol_type = other DNA

SEQ ID NO: 66 - protein, 5 aa, synthetic construct
NYWMT

SEQ ID NO: 67 - protein, 17 aa, synthetic construct
NIKKDGSEKYYVDSVRG

SEQ ID NO: 68 - protein, 11 aa, synthetic construct
VRSGRYFALDD

SEQ ID NO: 69 - protein, 7 aa, synthetic construct
GFTFQNY

SEQ ID NO: 70 - protein, 6 aa, synthetic construct
KKDGSE

SEQ ID NO: 71 - protein, 11 aa, synthetic construct
VRSGRYFALDD

SEQ ID NO: 72 - protein, 120 aa, synthetic construct
QVQLVESGGTLVQPGGSLRLSCAASGFTFQNYWMTWVRQAPGKGLEWVANIKKDGSEKYYVDSVRGRFTISRDNAKNSLFLQMNSLRPEDTAVYFCATVRSGRYFALDDWGQGTLVTVSS

SEQ ID NO: 73 - DNA, 360 bp, synthetic construct
  misc_feature 1..360: note = source = /note=Description of synthetic construct: Synthetic polynucleotide
  source 1..360: mol_type = other DNA
caggtgcagctggtggaatcaggcggcacactggtgcagcctggcggtagcctgagactgagctgcgccgctagtggattcacctttcagaactactggatgacctgggtcagacaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagc

SEQ ID NO: 74 - protein, 450 aa, synthetic construct
QVQLVESGGTLVQPGGSLRLSCAASGFTFQNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1350 bp, synthetic construct
  misc_feature 1..1350: note = source = /note=Description of synthetic construct: Synthetic polynucleotide
  source 1..1350: mol_type = other DNA
caggtgcagctggtggaatcaggcggcacactggtgcagcctggcggtagcctgagactgagctgcgccgctagtggattcacctttcagaactactggatgacctgggtcagacaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 76 - protein, 11 aa, synthetic construct
GGDNIGSRPVH

SEQ ID NO: 77 - protein, 7 aa, synthetic construct
DDSNRPS

SEQ ID NO: 78 - protein, 10 aa, synthetic construct
QVWSSSTDHP

SEQ ID NO: 79 - protein, 7 aa, synthetic construct
DNIGSRP

SEQ ID NO: 80 - skipped

SEQ ID NO: 81 - protein, 7 aa, synthetic construct
WSSSTDH

SEQ ID NO: 82 - protein, 107 aa, synthetic construct; identical to SEQ ID NO: 22

SEQ ID NO: 83 - DNA, 321 bp, synthetic construct; identical to SEQ ID NO: 23
  misc_feature 1..321: note = source = /note=Description of synthetic construct: Synthetic polynucleotide
  source 1..321: mol_type = other DNA

SEQ ID NO: 84 - protein, 213 aa, synthetic construct; identical to SEQ ID NO: 24

SEQ ID NO: 85 - DNA, 639 bp, synthetic construct; identical to SEQ ID NO: 25
  misc_feature 1..639: note = source = /note=Description of synthetic construct: Synthetic polynucleotide
  source 1..639: mol_type = other DNA

SEQ ID NO: 86 - protein, 5 aa, synthetic construct
NYWMT

SEQ ID NO: 87 - protein, 17 aa, synthetic construct
NIKKDGSEKYYVDSVRG

SEQ ID NO: 88 - protein, 11 aa, synthetic construct
VRSGRYFALDD

SEQ ID NO: 89 - protein, 7 aa, synthetic construct
GFTFNNY

SEQ ID NO: 90 - protein, 6 aa, synthetic construct
KKDGSE

SEQ ID NO: 91 - protein, 11 aa, synthetic construct
VRSGRYFALDD

SEQ ID NO: 92 - protein, 120 aa, synthetic construct
QVQLQESGPGLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSS

SEQ ID NO: 93 - DNA, 360 bp, synthetic construct
  misc_feature 1..360: note = source = /note=Description of synthetic construct: Synthetic polynucleotide
  source 1..360: mol_type = other DNA
caggtgcagctgcaggaatcaggcccaggactggtgcagcctggcggtagcctgagactgagctgcgctgctagtggcttcacctttaacaactactggatgacctgggtccgccaggcccctggcaaaggcctggagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggccagggcaccctggtcaccgtgtcttcc

SEQ ID NO: 94 - protein, 450 aa, synthetic construct
QVQLQESGPGLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1350 bp, synthetic construct
  misc_feature 1..1350: note = source = /note=Description of synthetic construct: Synthetic polynucleotide
  source 1..1350: mol_type = other DNA
caggtgcagctgcaggaatcaggcccaggactggtgcagcctggcggtagcctgagactgagctgcgctgctagtggcttcacctttaacaactactggatgacctgggtccgccaggcccctggcaaaggcctggagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggccagggcaccctggtcaccgtgtcttccgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 96 - protein, 11 aa, synthetic construct
GGDNIGSRPVH

SEQ ID NO: 97 - protein, 7 aa, synthetic construct
DDSNRPS

SEQ ID NO: 98 - protein, 10 aa, synthetic construct
QVWSSSTDHP

SEQ ID NO: 99 - protein, 7 aa, synthetic construct
DNIGSRP

SEQ ID NO: 100 - skipped

SEQ ID NO: 101 - protein, 7 aa, synthetic construct
WSSSTDH

SEQ ID NO: 102 - protein, 107 aa, synthetic construct; identical to SEQ ID NO: 22

SEQ ID NO: 103 - DNA, 321 bp, synthetic construct; identical to SEQ ID NO: 23
  misc_feature 1..321: note = source = /note=Description of synthetic construct: Synthetic polynucleotide
  source 1..321: mol_type = other DNA

SEQ ID NO: 104 - protein, 213 aa, synthetic construct; identical to SEQ ID NO: 24

SEQ ID NO: 105 - DNA, 639 bp, synthetic construct; identical to SEQ ID NO: 25
  misc_feature 1..639: note = source = /note=Description of synthetic construct: Synthetic polynucleotide
  source 1..639: mol_type = other DNA

SEQ ID NO: 106 - protein, 5 aa, synthetic construct
NYWMT

SEQ ID NO: 107 - protein, 17 aa, synthetic construct
NIKKDGSEKYYVDSVRG

SEQ ID NO: 108 - protein, 11 aa, synthetic construct
VRSGRYFALDD

SEQ ID NO: 109 - protein, 7 aa, synthetic construct
GFTFNNY

SEQ ID NO: 110 - protein, 6 aa, synthetic construct
KKDGSE

SEQ ID NO: 111 - protein, 11 aa, synthetic construct
VRSGRYFALDD

SEQ ID NO: 112 - protein, 120 aa, synthetic construct
  REGION 1..120: note = source = /note=Description of synthetic construct: Synthetic polypeptide
QVQLVESGGGLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSS

SEQ ID NO: 113 - DNA, 360 bp, synthetic construct
  misc_feature 1..360: note = source = /note=Description of synthetic construct: Synthetic polynucleotide
  source 1..360: mol_type = other DNA
caggtgcagctggtggaatcaggcggcggactggtgcagcctggcggtagcctgagactgagctgcgctgctagtggcttcacctttaacaactactggatgacctgggttaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagc

SEQ ID NO: 114 - protein, 450 aa, synthetic construct
QVQLVESGGGLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - DNA, 1350 bp, synthetic construct
  misc_feature 1..1350: note = source = /note=Description of synthetic construct: Synthetic polynucleotide
  source 1..1350: mol_type = other DNA
caggtgcagctggtggaatcaggcggcggactggtgcagcctggcggtagcctgagactgagctgcgctgctagtggcttcacctttaacaactactggatgacctgggttaggcaggcccctggtaaaggcctcgagtgggtggcaaatatcaagaaggacggtagcgagaagtactacgtggactcagtcagaggccggttcactatctctagggataacgctaagaatagcctgttcctgcagatgaactcactgaggcccgaggataccgccgtctacttctgtgctaccgtcagatcaggccgctacttcgccctggacgactggggtcaaggcacactggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 116 - protein, 11 aa, synthetic construct
GGDNIGSRPVH

SEQ ID NO: 117 - protein, 7 aa, synthetic construct
DDSNRPS

SEQ ID NO: 118 - protein, 10 aa, synthetic construct
QVWSSSTDHP

SEQ ID NO: 119 - protein, 7 aa, synthetic construct
DNIGSRP

SEQ ID NO: 120 - skipped

SEQ ID NO: 121 - protein, 7 aa, synthetic construct
WSSSTDH

SEQ ID NO: 122 - protein, 107 aa, synthetic construct; identical to SEQ ID NO: 22

SEQ ID NO: 123 - DNA, 321 bp, synthetic construct; identical to SEQ ID NO: 23
  misc_feature 1..321: note = source = /note=Description of synthetic construct: Synthetic polynucleotide
  source 1..321: mol_type = other DNA

SEQ ID NO: 124 - protein, 213 aa, synthetic construct; identical to SEQ ID NO: 24

SEQ ID NO: 125 - DNA, 639 bp, synthetic construct; identical to SEQ ID NO: 25
  misc_feature 1..639: note = source = /note=Description of synthetic construct: Synthetic polynucleotide
  source 1..639: mol_type = other DNA

SEQ ID NO: 126 - protein, 5 aa, synthetic construct
RDYWT

SEQ ID NO: 127 - protein, 16 aa, synthetic construct
NIYYSGSTNYNPSLKS

SEQ ID NO: 128 - protein, 16 aa, synthetic construct
VPGCSSTSCIDGWFDP

SEQ ID NO: 129 - protein, 7 aa, synthetic construct
GGSISRD

SEQ ID NO: 130 - protein, 5 aa, synthetic construct
YYSGS

SEQ ID NO: 131 - protein, 16 aa, synthetic construct
VPGCSSTSCIDGWFDP

SEQ ID NO: 132 - protein, 124 aa, synthetic construct
QVQLQESGPGLVKPSETLSLTCTVSGGSISRDYWTWVRQPPGEGLEWIGNIYYSGSTNYNPSLKSRVTISVAASKKQFSLKLTSVTAADTAVYYCARVPGCSSTSCIDGWFDPWGQGILVTVSS

SEQ ID NO: 133 - DNA, 372 bp, synthetic construct
  misc_feature 1..372: note = source = /note=Description of synthetic construct: Synthetic polynucleotide
  source 1..372: mol_type = other DNA
caggtgcagctgcaagaatcaggccctggcctggtcaagcctagcgagacactgagcctgacctgcaccgtcagcggcggctctatctctagggactactggacctgggtccgacagcctcctggcgagggcctcgagtggatcggtaatatctactatagcggctctactaactataaccctagcctgaagtctagggtcacaattagcgtggccgcctctaagaagcagtttagcctgaagctgactagcgtgaccgccgctgacaccgccgtctactactgcgctagagtgcccggctgctctagcactagctgtatcgacggctggtttgacccttggggtcaagggatcctggtcaccgtgtctagc

SEQ ID NO: 134 - protein, 454 aa, synthetic construct
QVQLQESGPGLVKPSETLSLTCTVSGGSISRDYWTWVRQPPGEGLEWIGNIYYSGSTNYNPSLKSRVTISVAASKKQFSLKLTSVTAADTAVYYCARVPGCSSTSCIDGWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 1362 bp, synthetic construct
  misc_feature 1..1362: note = source = /note=Description of synthetic construct: Synthetic polynucleotide
  source 1..1362: mol_type = other DNA
caggtgcagctgcaagaatcaggccctggcctggtcaagcctagcgagacactgagcctgacctgcaccgtcagcggcggctctatctctagggactactggacctgggtccgacagcctcctggcgagggcctcgagtggatcggtaatatctactatagcggctctactaactataaccctagcctgaagtctagggtcacaattagcgtggccgcctctaagaagcagtttagcctgaagctgactagcgtgaccgccgctgacaccgccgtctactactgcgctagagtgcccggctgctctagcactagctgtatcgacggctggtttgacccttggggtcaagggatcctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 136 - protein, 13 aa, synthetic construct
SGSSSNIGNTYVS

SEQ ID NO: 137 - protein, 7 aa, synthetic construct
DNNKRPS

SEQ ID NO: 138 - protein, 11 aa, synthetic construct
GTWDSSLSAWV

SEQ ID NO: 139 - protein, 9 aa, synthetic construct
SSSNIGNTY

SEQ ID NO: 140 - skipped

SEQ ID NO: 141 - protein, 8 aa, synthetic construct
WDSSLSAW

SEQ ID NO: 142 - protein, 110 aa, synthetic construct
QSVLTQPPSLSAAPGQRVTISCSGSSSNIGNTYVSWYQQLPGTAPKLLIYDNNKRPSGIPGRFSGSKSGTSATLGITGLQTGDEAAYYCGTWDSSLSAWVFGGGTRLTVL

SEQ ID NO: 143 - DNA, 330 bp, synthetic construct
  misc_feature 1..330: note = source = /note=Description of synthetic construct: Synthetic polynucleotide
  source 1..330: mol_type = other DNA
cagtcagtcctgactcagccccctagcctgagcgccgctcccggtcaaagagtgactattagctgtagcggctctagctctaatatcggtaatacctacgtcagctggtatcagcagctgcccggcaccgcccctaagctgctgatctacgataacaacaagcggcctagcggaatccctggtcgctttagcggatctaaatcaggcactagcgctaccctgggaatcaccggcctgcagaccggcgacgaagccgcctactactgcggcacctgggactctagtctgagcgcctgggtgttcggcggaggcactagactgaccgtgctg

SEQ ID NO: 144 - protein, 216 aa, synthetic construct
QSVLTQPPSLSAAPGQRVTISCSGSSSNIGNTYVSWYQQLPGTAPKLLIYDNNKRPSGIPGRFSGSKSGTSATLGITGLQTGDEAAYYCGTWDSSLSAWVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 145 - DNA, 648 bp, synthetic construct
  misc_feature 1..648: note = source = /note=Description of synthetic construct: Synthetic polynucleotide
  source 1..648: mol_type = other DNA
cagtcagtcctgactcagccccctagcctgagcgccgctcccggtcaaagagtgactattagctgtagcggctctagctctaatatcggtaatacctacgtcagctggtatcagcagctgcccggcaccgcccctaagctgctgatctacgataacaacaagcggcctagcggaatccctggtcgctttagcggatctaaatcaggcactagcgctaccctgggaatcaccggcctgcagaccggcgacgaagccgcctactactgcggcacctgggactctagtctgagcgcctgggtgttcggcggaggcactagactgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 146 - protein, 5 aa, synthetic construct
RDYWS

SEQ ID NO: 147 - protein, 16 aa, synthetic construct
NIYYSGSTNYNPSLKS

SEQ ID NO: 148 - protein, 16 aa, synthetic construct
VPGCSSTSCIDGWFDP

SEQ ID NO: 149 - protein, 7 aa, synthetic construct
GGSISRD

SEQ ID NO: 150 - protein, 5 aa, synthetic construct
YYSGS

SEQ ID NO: 151 - protein, 16 aa, synthetic construct
VPGCSSTSCIDGWFDP

SEQ ID NO: 152 - protein, 124 aa, synthetic construct
QVQLQESGPGLVKPSETLSLTCTVSGGSISRDYWSWVRQPPGAGLEWIGNIYYSGSTNYNPSLKSRVTISVATNKKQFSLKLTSVTAADTAVYYCARVPGCSSTSCIDGWFDPWGQGILVTVSS

SEQ ID NO: 153 - DNA, 372 bp, synthetic construct
  misc_feature 1..372: note = source = /note=Description of synthetic construct: Synthetic polynucleotide
  source 1..372: mol_type = other DNA
caggtgcagctgcaagaatcaggccctggcctggtcaagcctagcgagacactgagcctgacctgcaccgtcagcggcggctctatctctagggactactggtcctgggtccgacaacctcctggcgctggcctcgagtggatcggtaatatctactatagcggctctactaactataaccctagcctgaagtctagggtcacaattagtgtggctactaacaagaagcagtttagcctgaagctgactagcgtgaccgccgctgacaccgccgtctactactgcgctagagtgcccggctgctctagcactagctgtatcgacggttggtttgacccttggggtcaagggatcctggtcaccgtgtctagc

SEQ ID NO: 154 - protein, 454 aa, synthetic construct
QVQLQESGPGLVKPSETLSLTCTVSGGSISRDYWSWVRQPPGAGLEWIGNIYYSGSTNYNPSLKSRVTISVATNKKQFSLKLTSVTAADTAVYYCARVPGCSSTSCIDGWFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - DNA, 1362 bp, synthetic construct
  misc_feature 1..1362: note = source = /note=Description of synthetic construct: Synthetic polynucleotide
  source 1..1362: mol_type = other DNA
caggtgcagctgcaagaatcaggccctggcctggtcaagcctagcgagacactgagcctgacctgcaccgtcagcggcggctctatctctagggactactggtcctgggtccgacaacctcctggcgctggcctcgagtggatcggtaatatctactatagcggctctactaactataaccctagcctgaagtctagggtcacaattagtgtggctactaacaagaagcagtttagcctgaagctgactagcgtgaccgccgctgacaccgccgtctactactgcgctagagtgcccggctgctctagcactagctgtatcgacggttggtttgacccttggggtcaagggatcctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 156 - protein, 13 aa, synthetic construct
SGSSSNIGNTYVS

SEQ ID NO: 157 - protein, 7 aa, synthetic construct
DNNKRPS

SEQ ID NO: 158 - protein, 11 aa, synthetic construct
GTWDSSLSAWV

SEQ ID NO: 159 - protein, 9 aa, synthetic construct
SSSNIGNTY

SEQ ID NO: 160 - skipped

SEQ ID NO: 161 - protein, 8 aa, synthetic construct
WDSSLSAW

SEQ ID NO: 162 - protein, 110 aa, synthetic construct
QSVLTQPPSLSAAPGQKVTISCSGSSSNIGNTYVSWYQQLPGTAPKLLIYDNNKRPSGIPDRFSGSKSGTSATLGITGLQTGDEAVYYCGTWDSSLSAWVFGGGTRLTVL

SEQ ID NO: 163 - DNA, 330 bp, synthetic construct
  misc_feature 1..330: note = source = /note=Description of synthetic construct: Synthetic polynucleotide
  source 1..330: mol_type = other DNA
cagtcagtcctgactcagccccctagcctgagcgccgctcccggtcaaaaagtgactattagctgtagcggctctagctctaatatcggtaatacctacgtcagctggtatcagcagctgcccggcaccgcccctaagctgctgatctacgataacaacaagcggcctagcggaatccccgataggtttagcggatctaagtcaggcactagcgctaccctgggaatcaccggcctgcagaccggcgacgaggccgtctactactgcggcacctgggactctagtctgagcgcctgggtgttcggcggaggcactagactgaccgtgctg

SEQ ID NO: 164 - protein, 216 aa, synthetic construct
QSVLTQPPSLSAAPGQKVTISCSGSSSNIGNTYVSWYQQLPGTAPKLLIYDNNKRPSGIPDRFSGSKSGTSATLGITGLQTGDEAVYYCGTWDSSLSAWVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 165 - DNA, 648 bp, synthetic construct
  misc_feature 1..648: note = source = /note=Description of synthetic construct: Synthetic polynucleotide
  source 1..648: mol_type = other DNA
cagtcagtcctgactcagccccctagcctgagcgccgctcccggtcaaaaagtgactattagctgtagcggctctagctctaatatcggtaatacctacgtcagctggtatcagcagctgcccggcaccgcccctaagctgctgatctacgataacaacaagcggcctagcggaatccccgataggtttagcggatctaagtcaggcactagcgctaccctgggaatcaccggcctgcagaccggcgacgaggccgtctactactgcggcacctgggactctagtctgagcgcctgggtgttcggcggaggcactagactgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 166 - protein, 5 aa, synthetic construct
RDYWS

SEQ ID NO: 167 - protein, 16 aa, synthetic construct
NIYYSGSTNYNPSLKS

SEQ ID NO: 168 - protein, 16 aa, synthetic construct
VPGCSSTSCIDGWFDP

SEQ ID NO: 169 - protein, 7 aa, synthetic construct
GGSISRD

SEQ ID NO: 170 - protein, 5 aa, synthetic construct
YYSGS

SEQ ID NO: 171 - protein, 16 aa, synthetic construct
VPGCSSTSCIDGWFDP

SEQ ID NO: 172 - protein, 124 aa, synthetic construct; identical to SEQ ID NO: 152

SEQ ID NO: 173 - DNA, 372 bp, synthetic construct
  misc_feature 1..372: note = source = /note=Description of synthetic construct: Synthetic polynucleotide
  source 1..372: mol_type = other DNA
caggtgcaattgcaggaaagcggccctggcctcgtgaagcccagcgagacactgagcctgacctgtaccgtgtccggcggcagcatcagcagagactactggagctgggttcgccagcctccaggcgcaggactggaatggatcggcaacatctactacagcggcagcaccaactacaaccccagcctgaagtccagagtgaccatcagcgtggccacaaacaagaaacagttctccctgaagctgaccagcgtgacagccgccgataccgccgtgtactactgcgccagagtgcctggctgtagcagcaccagctgcatcgacggatggttcgacccttggggccagggcattctcgtgaccgtcagctca

SEQ ID NO: 174 - protein, 454 aa, synthetic construct; identical to SEQ ID NO: 154

SEQ ID NO: 175 - DNA, 1362 bp, synthetic construct
  misc_feature 1..1362: note = source = /note=Description of synthetic construct: Synthetic polynucleotide
  source 1..1362: mol_type = other DNA
caggtgcaattgcaggaaagcggccctggcctcgtgaagcccagcgagacactgagcctgacctgtaccgtgtccggcggcagcatcagcagagactactggagctgggttcgccagcctccaggcgcaggactggaatggatcggcaacatctactacagcggcagcaccaactacaaccccagcctgaagtccagagtgaccatcagcgtggccacaaacaagaaacagttctccctgaagctgaccagcgtgacagccgccgataccgccgtgtactactgcgccagagtgcctggctgtagcagcaccagctgcatcgacggatggttcgacccttggggccagggcattctcgtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 176 - protein, 13 aa, synthetic construct
SGSSSNIGNTYVS

SEQ ID NO: 177 - protein, 7 aa, synthetic construct
DNNKRPS

SEQ ID NO: 178 - protein, 11 aa, synthetic construct
GTWDSSLSAWV

SEQ ID NO: 179 - protein, 9 aa, synthetic construct
SSSNIGNTY

SEQ ID NO: 180 - skipped

SEQ ID NO: 181 - protein, 8 aa, synthetic construct
WDSSLSAW

SEQ ID NO: 182 - protein, 110 aa, synthetic construct
QSVLTQPPSVSAAPGQKVTISCSGSSSNIGNTYVSWYQQLPGTAPKLLIYDNNKRPSGIPDRFSGSKSGTSATLGITGLQTGDEADYYCGTWDSSLSAWVFGGGTRLTVL

SEQ ID NO: 183 - DNA, 330 bp, synthetic construct
  misc_feature 1..330: note = source = /note=Description of synthetic construct: Synthetic polynucleotide
  source 1..330: mol_type = other DNA
caaagcgtgctgacccagcctcctagcgtgtctgctgcccctggccagaaggtgaccatcagctgtagcggcagcagctccaacatcggcaacacctacgtgtcctggtatcagcagctgcccggcaccgcccccaaactgctgatctacgacaacaacaagcggcccagcggcatccccgatagattttctggcagcaagagcggcaccagcgccaccctgggaatcacaggactgcagacaggggacgaggccgattactactgtggcacctgggattctagcctgagcgcctgggtgttcggcggaggcacaagactgacagtgctg

SEQ ID NO: 184 - protein, 216 aa, synthetic construct
QSVLTQPPSVSAAPGQKVTISCSGSSSNIGNTYVSWYQQLPGTAPKLLIYDNNKRPSGIPDRFSGSKSGTSATLGITGLQTGDEADYYCGTWDSSLSAWVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 185 - DNA, 648 bp, synthetic construct
  misc_feature 1..648: note = source = /note=Description of synthetic construct: Synthetic polynucleotide
  source 1..648: mol_type = other DNA
caaagcgtgctgacccagcctcctagcgtgtctgctgcccctggccagaaggtgaccatcagctgtagcggcagcagctccaacatcggcaacacctacgtgtcctggtatcagcagctgcccggcaccgcccccaaactgctgatctacgacaacaacaagcggcccagcggcatccccgatagattttctggcagcaagagcggcaccagcgccaccctgggaatcacaggactgcagacaggggacgaggccgattactactgtggcacctgggattctagcctgagcgcctgggtgttcggcggaggcacaagactgacagtgctgggtcagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 186 - protein, 7 aa, synthetic construct
SGGYSWS

SEQ ID NO: 187 - protein, 16 aa, synthetic construct
YIYYRGTTYYNPSLKS

SEQ ID NO: 188 - protein, 13 aa, synthetic construct
ALTHLVGVGWFDP

SEQ ID NO: 189 - protein, 9 aa, synthetic construct
GGSISSGGY

SEQ ID NO: 190 - protein, 5 aa, synthetic construct
YYRGT

SEQ ID NO: 191 - protein, 13 aa, synthetic construct
ALTHLVGVGWFDP

SEQ ID NO: 192 - protein, 123 aa, synthetic construct
QVQLQESGPGLAKPSQTLSLTCSVSGGSISSGGYSWSWIRQPPGKGLEYIGYIYYRGTTYYNPSLKSRITMSVDTSNNQISLKLTSVTAADTAVYYCARALTHLVGVGWFDPWGQGTMVTVSS

SEQ ID NO: 193 - DNA, 369 bp, synthetic construct
  misc_feature 1..369: note = source = /note=Description of synthetic construct: Synthetic polynucleotide
  source 1..369: mol_type = other DNA
caggtgcagctgcaagaatcaggccctggcctggctaagcctagtcagaccctgagcctgacctgtagcgtcagcggaggctctatctctagcggcggctatagctggtcctggattagacagcccccaggtaaaggcctcgagtatatcggctatatctactataggggcactacctactataaccctagcctgaagtctaggatcactatgagcgtggacacctctaacaatcagattagcctgaagctgactagcgtgaccgccgctgacaccgccgtctactactgcgctagagccctgactcacctcgttggagtgggctggtttgacccttggggtcaaggcactatggtcaccgtgtctagc

SEQ ID NO: 194 - protein, 453 aa, synthetic construct
QVQLQESGPGLAKPSQTLSLTCSVSGGSISSGGYSWSWIRQPPGKGLEYIGYIYYRGTTYYNPSLKSRITMSVDTSNNQISLKLTSVTAADTAVYYCARALTHLVGVGWFDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 1359 bp, synthetic construct
  misc_feature 1..1359: note = source = /note=Description of synthetic construct: Synthetic polynucleotide
  source 1..1359: mol_type = other DNA
caggtgcagctgcaagaatcaggccctggcctggctaagcctagtcagaccctgagcctgacctgtagcgtcagcggaggctctatctctagcggcggctatagctggtcctggattagacagcccccaggtaaaggcctcgagtatatcggctatatctactataggggcactacctactataaccctagcctgaagtctaggatcactatgagcgtggacacctctaacaatcagattagcctgaagctgactagcgtgaccgccgctgacaccgccgtctactactgcgctagagccctgactcacctcgttggagtgggctggtttgacccttggggtcaaggcactatggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 196 - protein, 13 aa, synthetic construct
SGGSSNLGSNYVS

SEQ ID NO: 197 - protein, 7 aa, synthetic construct
DNNKRPS

SEQ ID NO: 198 - protein, 11 aa, synthetic construct
GTWDGSLSAWV

SEQ ID NO: 199 - protein, 9 aa, synthetic construct
GSSNLGSNY

SEQ ID NO: 200 - skipped

SEQ ID NO: 201 - protein, 8 aa, synthetic construct
WDGSLSAW

SEQ ID NO: 202 - protein, 110 aa, synthetic construct
QSVLTQPPSVSAAPGQKVTISCSGGSSNLGSNYVSWYQQLPGTAPKLLIYDNNKRPSGIPDRFSGSKSGTSATLGITGLQTGDEADYYCGTWDGSLSAWVFGGGTKVTVL

SEQ ID NO: 203 - DNA, 330 bp, synthetic construct
  misc_feature 1..330: note = source = /note=Description of synthetic construct: Synthetic polynucleotide
  source 1..330: mol_type = other DNA
cagtcagtcctgactcagccccctagcgtcagcgccgctcccggtcaaaaagtgactattagctgtagcggcggctcctctaacctgggctctaactacgtcagctggtatcagcagctgcccggcaccgcccctaagctgctgatctacgataacaacaagcggcctagcggaatccccgataggtttagcggctctaagtcaggcactagcgctaccctgggaatcaccggcctgcagaccggcgacgaggccgactactactgtggcacctgggacggtagcctgagcgcctgggtgttcggcggaggcactaaagtcacagtgctg

SEQ ID NO: 204 - protein, 216 aa, synthetic construct
QSVLTQPPSVSAAPGQKVTISCSGGSSNLGSNYVSWYQQLPGTAPKLLIYDNNKRPSGIPDRFSGSKSGTSATLGITGLQTGDEADYYCGTWDGSLSAWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 205 - DNA, 648 bp, synthetic construct
  misc_feature 1..648: note = source = /note=Description of synthetic construct: Synthetic polynucleotide
  source 1..648: mol_type = other DNA
cagtcagtcctgactcagccccctagcgtcagcgccgctcccggtcaaaaagtgactattagctgtagcggcggctcctctaacctgggctctaactacgtcagctggtatcagcagctgcccggcaccgcccctaagctgctgatctacgataacaacaagcggcctagcggaatccccgataggtttagcggctctaagtcaggcactagcgctaccctgggaatcaccggcctgcagaccggcgacgaggccgactactactgtggcacctgggacggtagcctgagcgcctgggtgttcggcggaggcactaaagtcacagtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 206 - protein, 7 aa, synthetic construct
SGGYSWS

SEQ ID NO: 207 - protein, 16 aa, synthetic construct
YIYYRGTTYYNPSLKS

SEQ ID NO: 208 - protein, 13 aa, synthetic construct
ALTHLVGVGWFDP

SEQ ID NO: 209 - protein, 9 aa, synthetic construct
GGSISSGGY

SEQ ID NO: 210 - protein, 5 aa, synthetic construct
YYRGT

SEQ ID NO: 211 - protein, 13 aa, synthetic construct
ALTHLVGVGWFDP

SEQ ID NO: 212 - protein, 123 aa, synthetic construct
QVQLQESGPGLVKPSQTLSLTCTVSGGSISSGGYSWSWIRQPPGKGLEYIGYIYYRGTTYYNPSLKSRVTISVDTSNNQISLKLSSVTAADTAVYYCARALTHLVGVGWFDPWGQGTMVTVSS

SEQ ID NO: 213 - DNA, 369 bp, synthetic construct
  misc_feature 1..369: note = source = /note=Description of synthetic construct: Synthetic polynucleotide
  source 1..369: mol_type = other DNA
caggtgcagctgcaagaatcaggccctggcctggtcaagcctagtcagaccctgagcctgacctgcaccgtcagcggaggctctatctctagcggcggctatagctggtcctggattagacagcccccaggtaaaggcctcgagtatatcggctatatctactataggggcactacctactataaccctagcctgaagtctagggtcacaattagcgtggacacctctaacaatcagattagcctgaagctgtctagcgtgaccgccgctgacaccgccgtctactactgcgctagagccctgactcacctcgtcggagtgggctggtttgacccttggggtcaaggcactatggtcaccgtgtctagc

SEQ ID NO: 214 - protein, 453 aa, synthetic construct
QVQLQESGPGLVKPSQTLSLTCTVSGGSISSGGYSWSWIRQPPGKGLEYIGYIYYRGTTYYNPSLKSRVTISVDTSNNQISLKLSSVTAADTAVYYCARALTHLVGVGWFDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - DNA, 1359 bp, synthetic construct
  misc_feature 1..1359: note = source = /note=Description of synthetic construct: Synthetic polynucleotide
  source 1..1359: mol_type = other DNA
caggtgcagctgcaagaatcaggccctggcctggtcaagcctagtcagaccctgagcctgacctgcaccgtcagcggaggctctatctctagcggcggctatagctggtcctggattagacagcccccaggtaaaggcctcgagtatatcggctatatctactataggggcactacctactataaccctagcctgaagtctagggtcacaattagcgtggacacctctaacaatcagattagcctgaagctgtctagcgtgaccgccgctgacaccgccgtctactactgcgctagagccctgactcacctcgtcggagtgggctggtttgacccttggggtcaaggcactatggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 216 - protein, 13 aa, synthetic construct
SGGSSNLGSNYVS

SEQ ID NO: 217 - protein, 7 aa, synthetic construct
DNNKRPS

SEQ ID NO: 218 - protein, 11 aa, synthetic construct
GTWDGSLSAWV

SEQ ID NO: 219 - protein, 9 aa, synthetic construct
GSSNLGSNY

SEQ ID NO: 220 - skipped

SEQ ID NO: 221 - protein, 8 aa, synthetic construct
WDGSLSAW

SEQ ID NO: 222 - protein, 110 aa, synthetic construct; identical to SEQ ID NO: 202

SEQ ID NO: 223 - DNA, 330 bp, synthetic construct; identical to SEQ ID NO: 203
  misc_feature 1..330: note = source = /note=Description of synthetic construct: Synthetic polynucleotide
  source 1..330: mol_type = other DNA

SEQ ID NO: 224 - protein, 216 aa, synthetic construct; identical to SEQ ID NO: 204

SEQ ID NO: 225 - DNA, 648 bp, synthetic construct; identical to SEQ ID NO: 205
  misc_feature 1..648: note = source = /note=Description of synthetic construct: Synthetic polynucleotide
  source 1..648: mol_type = other DNA

SEQ ID NO: 226 - protein, 5 aa, synthetic construct
AYYWT

SEQ ID NO: 227 - protein, 16 aa, synthetic construct
YISHSGSTNYNPSLKS

SEQ ID NO: 228 - protein, 16 aa, synthetic construct
LGDTASLSRFYYYIDV

SEQ ID NO: 229 - protein, 7 aa, synthetic construct
GGSTSAY

SEQ ID NO: 230 - protein, 5 aa, synthetic construct
SHSGS

SEQ ID NO: 231 - protein, 16 aa, synthetic construct
LGDTASLSRFYYYIDV

SEQ ID NO: 232 - protein, 124 aa, synthetic construct
QVQLVQSGPGLVKPSETLSLTCTVSGGSTSAYYWTWIRQPPGKGLEWIGYISHSGSTNYNPSLKSRVTISADTSKNQLSLKVNSVTAADTAVYYCARLGDTASLSRFYYYIDVWGKGTTVTVSS

SEQ ID NO: 233 - DNA, 372 bp, synthetic construct
  misc_feature 1..372: note = source = /note=Description of synthetic construct: Synthetic polynucleotide
  source 1..372: mol_type = other DNA
caggtgcagctggtgcagtctggcccaggactggtgaagccttcggagaccctgtccctcacctgcactgtctctggtggctccaccagtgcttactactggacctggattcggcagcccccagggaagggactggagtggattgggtatatctctcacagtgggagcaccaactacaacccctccctcaagagtcgagtcaccatatcagcagacacgtccaagaaccagctctccctgaaggtgaactctgtgaccgccgcagacacggccgtgtattactgtgcgagacttggggatacagcttcacttagccgcttctactactacattgacgtctggggcaaagggaccacggtcaccgtctcctca

SEQ ID NO: 234 - protein, 454 aa, synthetic construct
QVQLVQSGPGLVKPSETLSLTCTVSGGSTSAYYWTWIRQPPGKGLEWIGYISHSGSTNYNPSLKSRVTISADTSKNQLSLKVNSVTAADTAVYYCARLGDTASLSRFYYYIDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - DNA, 1362 bp, synthetic construct
  misc_feature 1..1362: note = source = /note=Description of synthetic construct: Synthetic polynucleotide
  source 1..1362: mol_type = other DNA
caggtgcagctggtgcagtctggcccaggactggtgaagccttcggagaccctgtccctcacctgcactgtctctggtggctccaccagtgcttactactggacctggattcggcagcccccagggaagggactggagtggattgggtatatctctcacagtgggagcaccaactacaacccctccctcaagagtcgagtcaccatatcagcagacacgtccaagaaccagctctccctgaaggtgaactctgtgaccgccgcagacacggccgtgtattactgtgcgagacttggggatacagcttcacttagccgcttctactactacattgacgtctggggcaaagggaccacggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 236 - protein, 12 aa, synthetic construct
RASQSVSSNYLA

SEQ ID NO: 237 - protein, 7 aa, synthetic construct
GASSRAT

SEQ ID NO: 238 - protein, 10 aa, synthetic construct
QQYGSSPPYT

SEQ ID NO: 239 - protein, 8 aa, synthetic construct
SQSVSSNY

SEQ ID NO: 240 - skipped

SEQ ID NO: 241 - protein, 7 aa, synthetic construct
YGSSPPY

SEQ ID NO: 242 - protein, 109 aa, synthetic construct
EIVMTQSPDTLSLSPGERATLSCRASQSVSSNYLAWYQQKPGEAPRLLIYGASSRATGIPDRFSGSGSGTDFTLTISRLEPEDFAVYYCQQYGSSPPYTFGQGTRLEIK

SEQ ID NO: 243 - DNA, 328 bp, synthetic construct
  misc_feature 1..328: note = source = /note=Description of synthetic construct: Synthetic polynucleotide
  source 1..328: mol_type = other DNA
gaaattgtaatgacgcagtctccagacaccctgtctttgtctccaggggaaagagccaccctctcctgcagggccagtcagagtgttagcagcaactacttagcctggtaccagcagaaacctggcgaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctccgtacacttttggccaggggacacgactggagattaaac

SEQ ID NO: 244 - protein, 216 aa, synthetic construct
EIVMTQSPDTLSLSPGERATLSCRASQSVSSNYLAWYQQKPGEAPRLLIYGASSRATGIPDRFSGSGSGTDFTLTISRLEPEDFAVYYCQQYGSSPPYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - DNA, 648 bp, synthetic construct
  misc_feature 1..648: note = source = /note=Description of synthetic construct: Synthetic polynucleotide
  source 1..648: mol_type = other DNA
gaaattgtaatgacgcagtctccagacaccctgtctttgtctccaggggaaagagccaccctctcctgcagggccagtcagagtgttagcagcaactacttagcctggtaccagcagaaacctggcgaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctccgtacacttttggccaggggacacgactggagattaaacgtacggtggctgcaccatctgtcttcatcttcccgccatctgatgagcagttgaaatctggaactgcctctgttgtgtgcctgctgaataacttctatccccgcgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaaccgcggagagtgt

SEQ ID NO: 246 - protein, 5 aa, synthetic construct
RNYMS

SEQ ID NO: 247 - protein, 16 aa, synthetic construct
GIYSGGSTYYADSVKG

SEQ ID NO: 248 - protein, 13 aa, synthetic construct
EDEFWSGYSAGVD

SEQ ID NO: 249 - protein, 7 aa, synthetic construct
GFTVRRN

SEQ ID NO: 250 - protein, 5 aa, synthetic construct
YSGGS

SEQ ID NO: 251 - protein, 13 aa, synthetic construct
EDEFWSGYSAGVD

SEQ ID NO: 252 - protein, 121 aa, synthetic construct
EVQLVETGGGLVQPGGSLRLSCAASGFTVRRNYMSWVRQAPGKGLEWVSGIYSGGSTYYADSVKGRFTISRDYSKNTLSLQMNTLRVEDTAVYFCAREDEFWSGYSAGVDWGQGTLVTVSS

SEQ ID NO: 253 - DNA, 364 bp, synthetic construct
  misc_feature 1..364: note = source = /note=Description of synthetic construct: Synthetic polynucleotide
  source 1..364: mol_type = other DNA
gaggtgcagctggtggagactggaggaggcttggtccagccgggggggtccctgagactctcatgtgcagcctctggattcaccgtcagacgcaattacatgagttgggtccgccaggctccggggaagggactggagtgggtctcagggatctacagtggtggtagcacatactacgcagactccgtgaagggccgattcaccatctccagagactattccaagaacacactgtctcttcaaatgaacaccctgagagtcgaggacacggccgtgtatttctgtgcgagagaagacgaattttggagcgggtattccgctggggtcgactggggccagggaaccctggtcaccgtctcctcag

SEQ ID NO: 254 - protein, 451 aa, synthetic construct
EVQLVETGGGLVQPGGSLRLSCAASGFTVRRNYMSWVRQAPGKGLEWVSGIYSGGSTYYADSVKGRFTISRDYSKNTLSLQMNTLRVEDTAVYFCAREDEFWSGYSAGVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - DNA, 1353 bp, synthetic construct
  misc_feature 1..1353: note = source = /note=Description of synthetic construct: Synthetic polynucleotide
  source 1..1353: mol_type = other DNA
gaggtgcagctggtggagactggaggaggcttggtccagccgggggggtccctgagactctcatgtgcagcctctggattcaccgtcagacgcaattacatgagttgggtccgccaggctccggggaagggactggagtgggtctcagggatctacagtggtggtagcacatactacgcagactccgtgaagggccgattcaccatctccagagactattccaagaacacactgtctcttcaaatgaacaccctgagagtcgaggacacggccgtgtatttctgtgcgagagaagacgaattttggagcgggtattccgctggggtcgactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 256 - protein, 11 aa, synthetic construct
RASQSISSYLN

SEQ ID NO: 257 - protein, 7 aa, synthetic construct
AASSLQS

SEQ ID NO: 258 - protein, 9 aa, synthetic construct
QQSYNTPRT

SEQ ID NO: 259 - protein, 7 aa, synthetic construct
SQSISSY

SEQ ID NO: 260 - skipped

SEQ ID NO: 261 - protein, 6 aa, synthetic construct
SYNTPR

SEQ ID NO: 262 - protein, 107 aa, synthetic construct
DIRLTQSPSSLSASVGDRVTITCRASQSISSYLNWYQQKPGKAPKLLIYAASSLQSGVPSRFSGSGSGTDFTLTISSLQPEDFATYYCQQSYNTPRTFGQGTKVEIK

SEQ ID NO: 263 - DNA, 323 bp, synthetic construct
  misc_feature 1..323: note = source = /note=Description of synthetic construct: Synthetic polynucleotide
  source 1..323: mol_type = other DNA
gacatccggttgacccagtctccatcctccctgtctgcatctgtaggagacagagtcaccatcacttgccgggcaagtcagagcattagcagctatttgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacaatacccctcgaacgttcggccaagggaccaaggtggagatcaaacg

SEQ ID NO: 264 - protein, 214 aa, synthetic construct
DIRLTQSPSSLSASVGDRVTITCRASQSISSYLNWYQQKPGKAPKLLIYAASSLQSGVPSRFSGSGSGTDFTLTISSLQPEDFATYYCQQSYNTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - DNA, 642 bp, synthetic construct
  misc_feature 1..642: note = source = /note=Description of synthetic construct: Synthetic polynucleotide
  source 1..642: mol_type = other DNA
gacatccggttgacccagtctccatcctccctgtctgcatctgtaggagacagagtcaccatcacttgccgggcaagtcagagcattagcagctatttgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacaatacccctcgaacgttcggccaagggaccaaggtggagatcaaacgtacggtggctgcaccatctgtcttcatcttcccgccatctgatgagcagttgaaatctggaactgcctctgttgtgtgcctgctgaataacttctatccccgcgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaaccgcggagagtgt

SEQ ID NO: 266 - protein, 5 aa, synthetic construct
RNYMS

SEQ ID NO: 267 - protein, 16 aa, synthetic construct
GIYSGGSTYYADSVKG

SEQ ID NO: 268 - protein, 13 aa, synthetic construct
EDEFWSGYSAGVD

SEQ ID NO: 269 - protein, 7 aa, synthetic construct
GFTVSRN

SEQ ID NO: 270 - protein, 5 aa, synthetic construct
YSGGS

SEQ ID NO: 271 - protein, 13 aa, synthetic construct
EDEFWSGYSAGVD

SEQ ID NO: 272 - protein, 121 aa, synthetic construct
QVQLVESGGGLVQPGGSLRLSCAASGFTVSRNYMSWVRQAPGKGLEWVSGIYSGGSTYYADSVKGRFTISRDYSKNTLSLQMNTLRVEDTAVYFCAREDEFWSGYSAGVDWGQGTLVTVSS

SEQ ID NO: 273 - DNA, 365 bp, synthetic construct
  misc_feature 1..365: note = source = /note=Description of synthetic construct: Synthetic polynucleotide
  source 1..365: mol_type = other DNA
caggtgcagctggtggaatctggaggaggcttggtccagcctggggggtccctgagactctcatgtgcagcctctggattcaccgtcagtcgcaattacatgagttgggtccgccaggctccggggaagggactggagtgggtctcagggatttacagtggtggtagcacatactacgcagactccgtgaagggccgattcaccatctccagagactattccaagaacacactgtctcttcaaatgaacaccctgagagtcgaggacacggccgtgtatttctgtgcgagagaagacgaattttggagtgggtattccgctggggtcgactggggccagggaaccctggtcaccgtctcctcagc

SEQ ID NO: 274 - protein, 451 aa, synthetic construct
QVQLVESGGGLVQPGGSLRLSCAASGFTVSRNYMSWVRQAPGKGLEWVSGIYSGGSTYYADSVKGRFTISRDYSKNTLSLQMNTLRVEDTAVYFCAREDEFWSGYSAGVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - DNA, 1353 bp, synthetic construct
  misc_feature 1..1353: note = source = /note=Description of synthetic construct: Synthetic polynucleotide
  source 1..1353: mol_type = other DNA
caggtgcagctggtggaatctggaggaggcttggtccagcctggggggtccctgagactctcatgtgcagcctctggattcaccgtcagtcgcaattacatgagttgggtccgccaggctccggggaagggactggagtgggtctcagggatttacagtggtggtagcacatactacgcagactccgtgaagggccgattcaccatctccagagactattccaagaacacactgtctcttcaaatgaacaccctgagagtcgaggacacggccgtgtatttctgtgcgagagaagacgaattttggagtgggtattccgctggggtcgactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 276 - protein, 11 aa, synthetic construct
RASQSISSYLN

SEQ ID NO: 277 - protein, 7 aa, synthetic construct
AASSLQS

SEQ ID NO: 278 - protein, 9 aa, synthetic construct
QQSYSTPRT

SEQ ID NO: 279 - protein, 7 aa, synthetic construct
SQSISSY

SEQ ID NO: 280 - skipped

SEQ ID NO: 281 - protein, 6 aa, synthetic construct
SYSTPR

SEQ ID NO: 282 - protein, 107 aa, synthetic construct
DIRMTQSPSSLSASVGDRVTITCRASQSISSYLNWYQQKPGKAPTLLIYAASSLQSGVPSRFSGSGSGTDFTLTISSLQPEDFATYYCQQSYSTPRTFGQGTKVEIK

SEQ ID NO: 283 - DNA, 322 bp, synthetic construct
  misc_feature 1..322: note = source = /note=Description of synthetic construct: Synthetic polynucleotide
  source 1..322: mol_type = other DNA
gacatccggatgacccagtctccatcctccctgtctgcatctgtaggagacagagtcaccatcacttgccgggcaagtcagagcattagcagctatttaaattggtatcagcagaaaccagggaaagcccctacgctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcggacgttcggccaagggaccaaggtggagatcaaac

SEQ ID NO: 284 - protein, 214 aa, synthetic construct
DIRMTQSPSSLSASVGDRVTITCRASQSISSYLNWYQQKPGKAPTLLIYAASSLQSGVPSRFSGSGSGTDFTLTISSLQPEDFATYYCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - DNA, 642 bp, synthetic construct
  misc_feature 1..642: note = source = /note=Description of synthetic construct: Synthetic polynucleotide
  source 1..642: mol_type = other DNA
gacatccggatgacccagtctccatcctccctgtctgcatctgtaggagacagagtcaccatcacttgccgggcaagtcagagcattagcagctatttaaattggtatcagcagaaaccagggaaagcccctacgctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcggacgttcggccaagggaccaaggtggagatcaaacgtacggtggctgcaccatctgtcttcatcttcccgccatctgatgagcagttgaaatctggaactgcctctgttgtgtgcctgctgaataacttctatccccgcgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaaccgcggagagtgt

SEQ ID NO: 286 - protein, 5 aa, synthetic construct
RNYMS

SEQ ID NO: 287 - protein, 16 aa, synthetic construct
GIYGGGRTYYAESVKG

SEQ ID NO: 288 - protein, 13 aa, synthetic construct
EDEFWSGYSAGVD

SEQ ID NO: 289 - protein, 7 aa, synthetic construct
GFTVSRN

SEQ ID NO: 290 - protein, 5 aa, synthetic construct
YGGGR

SEQ ID NO: 291 - protein, 13 aa, synthetic construct
EDEFWSGYSAGVD

SEQ ID NO: 292 - protein, 121 aa, synthetic construct
EVQLLESGGGLVRPGGSLRVSCAASGFTVSRNYMSWVRQAPGKGLEWVSGIYGGGRTYYAESVKGRFTISRDYSKNTLFLQMNTLRVEDTALYFCAREDEFWSGYSAGVDWGQGTLVTVSS

SEQ ID NO: 293 - DNA, 363 bp, synthetic construct
  misc_feature 1..363: note = source = /note=Description of synthetic construct: Synthetic polynucleotide
  source 1..363: mol_type = other DNA
gaggtgcagctgttggagtccgggggaggcttggtccggcctggggggtccctgagagtctcatgtgcagcctctggattcaccgtcagtcgcaattacatgagttgggtccgccaggctccggggaagggactggagtgggtctcagggatttacggtggtggtaggacttactacgcagagtccgtgaagggccgattcaccatctccagagactattccaagaacacactgtttcttcaaatgaacaccctgagagtcgaggacacggccctgtatttctgtgcgagagaagacgaattttggagtgggtattctgctggggtcgactggggccagggaaccctggtcactgtctcctca

SEQ ID NO: 294 - protein, 451 aa, synthetic construct
EVQLLESGGGLVRPGGSLRVSCAASGFTVSRNYMSWVRQAPGKGLEWVSGIYGGGRTYYAESVKGRFTISRDYSKNTLFLQMNTLRVEDTALYFCAREDEFWSGYSAGVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - DNA, 1353 bp, synthetic construct
  misc_feature 1..1353: note = source = /note=Description of synthetic construct: Synthetic polynucleotide
  source 1..1353: mol_type = other DNA
gaggtgcagctgttggagtccgggggaggcttggtccggcctggggggtccctgagagtctcatgtgcagcctctggattcaccgtcagtcgcaattacatgagttgggtccgccaggctccggggaagggactggagtgggtctcagggatttacggtggtggtaggacttactacgcagagtccgtgaagggccgattcaccatctccagagactattccaagaacacactgtttcttcaaatgaacaccctgagagtcgaggacacggccctgtatttctgtgcgagagaagacgaattttggagtgggtattctgctggggtcgactggggccagggaaccctggtcactgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 296 - protein, 11 aa, synthetic construct
RASQSISSYLN

SEQ ID NO: 297 - protein, 7 aa, synthetic construct
AASTLQT

SEQ ID NO: 298 - protein, 9 aa, synthetic construct
QQSYNTPRT

SEQ ID NO: 299 - protein, 7 aa, synthetic construct
SQSISSY

SEQ ID NO: 300 - skipped

SEQ ID NO: 301 - protein, 6 aa, synthetic construct
SYNTPR

SEQ ID NO: 302 - protein, 107 aa, synthetic construct
DIQVTQSPSSLSASVGDRVTITCRASQSISSYLNWYQQEPGKAPKLLIYAASTLQTGVPSRFSGSGSGTDFTLTISSLQPEDFATYYCQQSYNTPRTFGQGTKVEIK

SEQ ID NO: 303 - DNA, 323 bp, synthetic construct
  misc_feature 1..323: note = source = /note=Description of synthetic construct: Synthetic polynucleotide
  source 1..323: mol_type = other DNA
gacatccaggtgacccagtctccatcctccctgtctgcatctgtaggagacagagtcaccatcacttgccgggcaagtcagagcattagcagctatttaaattggtatcagcaggaaccagggaaagcccctaaactcctgatctacgctgcatccactttgcaaactggggtcccatcacggttcagtggtagtggatctgggacagatttcactctcaccatcagcagtctgcaacctgaagattttgcaacttattactgtcaacagagttacaatacccctcgaaccttcggccaagggaccaaggtggaaatcaaacg

SEQ ID NO: 304 - protein, 214 aa, synthetic construct
DIQVTQSPSSLSASVGDRVTITCRASQSISSYLNWYQQEPGKAPKLLIYAASTLQTGVPSRFSGSGSGTDFTLTISSLQPEDFATYYCQQSYNTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 305 - DNA, 642 bp, synthetic construct
  misc_feature 1..642: note = source = /note=Description of synthetic construct: Synthetic polynucleotide
  source 1..642: mol_type = other DNA
gacatccaggtgacccagtctccatcctccctgtctgcatctgtaggagacagagtcaccatcacttgccgggcaagtcagagcattagcagctatttaaattggtatcagcaggaaccagggaaagcccctaaactcctgatctacgctgcatccactttgcaaactggggtcccatcacggttcagtggtagtggatctgggacagatttcactctcaccatcagcagtctgcaacctgaagattttgcaacttattactgtcaacagagttacaatacccctcgaaccttcggccaagggaccaaggtggaaatcaaacgtacggtggctgcaccatctgtcttcatcttcccgccatctgatgagcagttgaaatctggaactgcctctgttgtgtgcctgctgaataacttctatccccgcgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaaccgcggagagtgt

SEQ ID NO: 306 - protein, 7 aa, synthetic construct
NGGYYWS

SEQ ID NO: 307 - protein, 16 aa, synthetic construct
CIHYSGGTYYNPSLKS

SEQ ID NO: 308 - protein, 14 aa, synthetic construct
ALIAAPGISDWFDP

SEQ ID NO: 309 - protein, 9 aa, synthetic construct
GGSISNGGY

SEQ ID NO: 310 - protein, 5 aa, synthetic construct
HYSGG

SEQ ID NO: 311 - protein, 14 aa, synthetic construct
ALIAAPGISDWFDP

SEQ ID NO: 312 - protein, 124 aa, synthetic construct
QVQLQESGPGLVKPSQTLSLTCTVSGGSISNGGYYWSWIRLHPGKGLEWIGCIHYSGGTYYNPSLKSRVTVSLDTSKNQFSLNLISVTAADTAIYFCARALIAAPGISDWFDPWGQGTLVTVSS

SEQ ID NO: 313 - protein, 454 aa, synthetic construct
QVQLQESGPGLVKPSQTLSLTCTVSGGSISNGGYYWSWIRLHPGKGLEWIGCIHYSGGTYYNPSLKSRVTVSLDTSKNQFSLNLISVTAADTAIYFCARALIAAPGISDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 13 aa, synthetic construct
SGSNSNVGHNYVS

SEQ ID NO: 315 - protein, 7 aa, synthetic construct
DNNKRPS

SEQ ID NO: 316 - protein, 11 aa, synthetic construct
GTWDSSLSAGV

SEQ ID NO: 317 - protein, 9 aa, synthetic construct
SNSNVGHNY

SEQ ID NO: 318 - skipped

SEQ ID NO: 319 - protein, 8 aa, synthetic construct
WDSSLSAG

SEQ ID NO: 320 - protein, 110 aa, synthetic construct
QSVLTQPPSVSAAPGQKVTISCSGSNSNVGHNYVSWYQQLPGTAPKLLIYDNNKRPSGIPDRFSGSKSGTSATLGITGLQTGDEADYYCGTWDSSLSAGVFGGGTKVTVL

SEQ ID NO: 321 - protein, 216 aa, synthetic construct
QSVLTQPPSVSAAPGQKVTISCSGSNSNVGHNYVSWYQQLPGTAPKLLIYDNNKRPSGIPDRFSGSKSGTSATLGITGLQTGDEADYYCGTWDSSLSAGVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 322 - protein, 5 aa, synthetic construct
SSWMN

SEQ ID NO: 323 - protein, 17 aa, synthetic construct
RIYPGDADTYYSGKFKG

SEQ ID NO: 324 - protein, 7 aa, synthetic construct
HSSGFTY

SEQ ID NO: 325 - protein, 7 aa, synthetic construct
GYTFSSS

SEQ ID NO: 326 - protein, 6 aa, synthetic construct
YPGDAD

SEQ ID NO: 327 - protein, 7 aa, synthetic construct
HSSGFTY

SEQ ID NO: 328 - protein, 116 aa, synthetic construct
QVQLVQSGAEVKKPGASVKVSCKASGYTFSSSWMNWVRQAPGQRLEWMGRIYPGDADTYYSGKFKGRVTITADSSARTAYMELSSLRSEDTAVYYCAIHSSGFTYWGQGTLVTVSS

SEQ ID NO: 329 - DNA, 350 bp, synthetic construct
  misc_feature 1..350: note = source = /note=Description of synthetic construct: Synthetic polynucleotide
  source 1..350: mol_type = other DNA
caggtccagcttgtgcagtctggggctgaggtgaagaagcctggggcctcagtgaaggtttcctgcaaggcttctggctatacattcagcagctcttggatgaactgggtgcgccaggcccccggacaaaggcttgagtggatgggacggatctatccaggagacgccgatacttactacagtgggaaattcaagggcagagtcaccattaccgccgacagctccgcgagaacagcctacatggagctgagcagcctgagatctgaagacacggctgtgtattactgtgcgatccacagctcgggctttacttactggggccagggcaccctggtcaccgtctcctcagc

SEQ ID NO: 330 - protein, 446 aa, synthetic construct
QVQLVQSGAEVKKPGASVKVSCKASGYTFSSSWMNWVRQAPGQRLEWMGRIYPGDADTYYSGKFKGRVTITADSSARTAYMELSSLRSEDTAVYYCAIHSSGFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - DNA, 1338 bp, synthetic construct
  misc_feature 1..1338: note = source = /note=Description of synthetic construct: Synthetic polynucleotide
  source 1..1338: mol_type = other DNA
caggtccagcttgtgcagtctggggctgaggtgaagaagcctggggcctcagtgaaggtttcctgcaaggcttctggctatacattcagcagctcttggatgaactgggtgcgccaggcccccggacaaaggcttgagtggatgggacggatctatccaggagacgccgatacttactacagtgggaaattcaagggcagagtcaccattaccgccgacagctccgcgagaacagcctacatggagctgagcagcctgagatctgaagacacggctgtgtattactgtgcgatccacagctcgggctttacttactggggccagggcaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 332 - protein, 11 aa, synthetic construct
RASQDISDYLN

SEQ ID NO: 333 - protein, 7 aa, synthetic construct
YTSRLHS

SEQ ID NO: 334 - protein, 9 aa, synthetic construct
QQTHTLPFT

SEQ ID NO: 335 - protein, 7 aa, synthetic construct
SQDISDY

SEQ ID NO: 336 - skipped

SEQ ID NO: 337 - protein, 6 aa, synthetic construct
THTLPF

SEQ ID NO: 338 - protein, 107 aa, synthetic construct
  REGION 1..107: note = source = /note=Description of synthetic construct: Synthetic polypeptide
DIQMTQSPSSLSASVGDRVTITCRASQDISDYLNWYQQKPGKAPKLLIYYTSRLHSGVPSRFSGSGSGTDYTLTISSLQPEDFATYFCQQTHTLPFTFGGGTKVEIK

SEQ ID NO: 339 - DNA, 323 bp, synthetic construct
  misc_feature 1..323: note = source = /note=Description of synthetic construct: Synthetic polynucleotide
  source 1..323: mol_type = other DNA
gacatccagatgacccagtctccatcctccctgtctgcatctgtaggagacagagtcaccatcacttgcagggcaagtcaggacattagcgattatttaaactggtatcagcagaaaccagggaaagcccctaagctcctgatctattatacatcaagattacactcaggggtcccatcaaggttcagtggcagtggatctgggacagattacactctcaccatcagcagtctgcaacctgaagattttgcaacttacttctgtcaacagactcatacgcttcctttcacgttcggcggagggaccaaggtggagatcaaacg

SEQ ID NO: 340 - protein, 214 aa, synthetic construct
DIQMTQSPSSLSASVGDRVTITCRASQDISDYLNWYQQKPGKAPKLLIYYTSRLHSGVPSRFSGSGSGTDYTLTISSLQPEDFATYFCQQTHTLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - DNA, 642 bp, synthetic construct
  misc_feature 1..642: note = source = /note=Description of synthetic construct: Synthetic polynucleotide
  source 1..642: mol_type = other DNA
gacatccagatgacccagtctccatcctccctgtctgcatctgtaggagacagagtcaccatcacttgcagggcaagtcaggacattagcgattatttaaactggtatcagcagaaaccagggaaagcccctaagctcctgatctattatacatcaagattacactcaggggtcccatcaaggttcagtggcagtggatctgggacagattacactctcaccatcagcagtctgcaacctgaagattttgcaacttacttctgtcaacagactcatacgcttcctttcacgttcggcggagggaccaaggtggagatcaaacgtacggtggctgcaccatctgtcttcatcttcccgccatctgatgagcagttgaaatctggaactgcctctgttgtgtgcctgctgaataacttctatccccgcgaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaaccgcggagagtgt

SEQ ID NO: 342 - protein, 5 aa, synthetic construct
NYWMH

SEQ ID NO: 343 - protein, 17 aa, synthetic construct
NIYPGSGNTNYGENFKS

SEQ ID NO: 344 - protein, 16 aa, synthetic construct
  REGION 1..16: note = source = /note=Description of synthetic construct: Synthetic peptide
SAIYYGYDGHYFAMDY

SEQ ID NO: 345 - protein, 7 aa, synthetic construct
GYTFTNY

SEQ ID NO: 346 - protein, 6 aa, synthetic construct
YPGSGN

SEQ ID NO: 347 - protein, 16 aa, synthetic construct
SAIYYGYDGHYFAMDY

SEQ ID NO: 348 - protein, 125 aa, synthetic construct
QVQLQQPGSELVRPGASVKLSCKASGYTFTNYWMHWVKQGHGQGLEWIGNIYPGSGNTNYGENFKSKGTLTVDTSSSTAYMHLSRLTSEDSAVYYCSRSAIYYGYDGHYFAMDYWGQGTSVTVSS

SEQ ID NO: 349 - protein, 11 aa, synthetic construct
KASQDIRKYIA

SEQ ID NO: 350 - protein, 7 aa, synthetic construct
YTSTLQS

SEQ ID NO: 351 - protein, 9 aa, synthetic construct
LQYDNILFT

SEQ ID NO: 352 - protein, 7 aa, synthetic construct
SQDIRKY

SEQ ID NO: 353 - skipped

SEQ ID NO: 354 - protein, 6 aa, synthetic construct
YDNILF

SEQ ID NO: 355 - protein, 107 aa, synthetic construct
DIQMTQSPSSLSASLGGKVTITCKASQDIRKYIAWYQHKPGKGPRLLINYTSTLQSGIPSRFRGSGSGRDYSFSISNLEPEDIATYYCLQYDNILFTFGTGTKLEIK

SEQ ID NO: 356 - protein, 5 aa, synthetic construct
SCWMN

SEQ ID NO: 357 - protein, 17 aa, synthetic construct
RIYPGDGDTKYTEKFKD

SEQ ID NO: 358 - protein, 7 aa, synthetic construct
SGSGLPY

SEQ ID NO: 359 - protein, 7 aa, synthetic construct
GYSFSSC

SEQ ID NO: 360 - protein, 6 aa, synthetic construct
YPGDGD

SEQ ID NO: 361 - protein, 7 aa, synthetic construct
SGSGLPY

SEQ ID NO: 362 - protein, 116 aa, synthetic construct
QVHLQQSGPELVKPGASVTISCKTSGYSFSSCWMNWVKQRPGQGLEWIGRIYPGDGDTKYTEKFKDKATLTADKSSSTAYMQLSSLTSVDSALYFCAISGSGLPYWGQGTLVTVSE

SEQ ID NO: 363 - protein, 11 aa, synthetic construct
RASQDIHNYLN

SEQ ID NO: 364 - protein, 7 aa, synthetic construct
STSRLHS

SEQ ID NO: 365 - protein, 9 aa, synthetic construct
QQTHTLPLT

SEQ ID NO: 366 - protein, 7 aa, synthetic construct
SQDIHNY

SEQ ID NO: 367 - skipped

SEQ ID NO: 368 - protein, 6 aa, synthetic construct
THTLPL

SEQ ID NO: 369 - protein, 107 aa, synthetic construct
DIQMTQTTSSLSASLGDRVTISCRASQDIHNYLNWYQQKPDGTIKLLIYSTSRLHSGVPSRFSGSGSGTHYSLTINNLEQEDIATYFCQQTHTLPLTFGAGTKLELK

SEQ ID NO: 370 - protein, 5 aa, synthetic construct
SYWMN

SEQ ID NO: 371 - protein, 17 aa, synthetic construct
QIYPGNGDTNYNGKFKG

SEQ ID NO: 372 - protein, 12 aa, synthetic construct
EARQGYHYAMDY

SEQ ID NO: 373 - protein, 7 aa, synthetic construct
GYAFSSY

SEQ ID NO: 374 - protein, 6 aa, synthetic construct
YPGNGD

SEQ ID NO: 375 - protein, 12 aa, synthetic construct
EARQGYHYAMDY

SEQ ID NO: 376 - protein, 121 aa, synthetic construct
QVQLQQSGAGLVRPGSSVKISCKTSGYAFSSYWMNWVKQRPGQGLEWIGQIYPGNGDTNYNGKFKGKATLTADKSSNTAYIQLNSLTSEDSAVYFCAREARQGYHYAMDYWGQGTSVTVSL

SEQ ID NO: 377 - protein, 12 aa, synthetic construct
SASSMINSNYLH

SEQ ID NO: 378 - protein, 7 aa, synthetic construct
RTSNLAS

SEQ ID NO: 379 - protein, 8 aa, synthetic construct
QQGSNIFT

SEQ ID NO: 380 - protein, 8 aa, synthetic construct
SSMINSNY

SEQ ID NO: 381 - skipped

SEQ ID NO: 382 - protein, 5 aa, synthetic construct
GSNIF

SEQ ID NO: 383 - protein, 107 aa, synthetic construct
EIVFTQSPTTMAAFPGEKITITCSASSMINSNYLHWYQQKPGFSPKVLIYRTSNLASGVPARFSGTGSGTSFSLTIGTMEAEDVATYYCQQGSNIFTFGSGTKLEIK

SEQ ID NO: 384 - protein, 5 aa, synthetic construct
NSWMN

SEQ ID NO: 385 - protein, 17 aa, synthetic construct
RIYPGDGDTQYNEKFKG

SEQ ID NO: 386 - protein, 7 aa, synthetic construct
SRSGLDY

SEQ ID NO: 387 - protein, 7 aa, synthetic construct
GFTFSNS

SEQ ID NO: 388 - protein, 6 aa, synthetic construct
YPGDGD

SEQ ID NO: 389 - protein, 7 aa, synthetic construct
SRSGLDY

SEQ ID NO: 390 - protein, 116 aa, synthetic construct
QVQLQQSGPELVKPGASVRISCKVSGFTFSNSWMNWVKQRPGQGLEWIGRIYPGDGDTQYNEKFKGKATLTADTSSNTAYIQLNSLTSVDSAVFFCARSRSGLDYWGQGTTLTVSS

SEQ ID NO: 391 - protein, 11 aa, synthetic construct
RASQDIYNYLN

SEQ ID NO: 392 - protein, 7 aa, synthetic construct
STSRLHS

SEQ ID NO: 393 - protein, 9 aa, synthetic construct
HQSHTVPFT

SEQ ID NO: 394 - protein, 7 aa, synthetic construct
SQDIYNY

SEQ ID NO: 395 - skipped

SEQ ID NO: 396 - protein, 6 aa, synthetic construct
SHTVPF

SEQ ID NO: 397 - protein, 107 aa, synthetic construct
DIQMTQSTSSLSASLGDRVTISCRASQDIYNYLNWFQQKPDGTVKPLIYSTSRLHSGVSSRFSGSGSGTDYSLTISNLEREDIATYFCHQSHTVPFTFGSGTKLEIK

SEQ ID NO: 398 - protein, 5 aa, synthetic construct
SSWIN

SEQ ID NO: 399 - protein, 17 aa, synthetic construct
RIYPGDGDTNYNGKFKG

SEQ ID NO: 400 - protein, 7 aa, synthetic construct
HSSGFPH

SEQ ID NO: 401 - protein, 7 aa, synthetic construct
GYTFSSS

SEQ ID NO: 402 - protein, 6 aa, synthetic construct
YPGDGD

SEQ ID NO: 403 - protein, 7 aa, synthetic construct
HSSGFPH

SEQ ID NO: 404 - protein, 116 aa, synthetic construct
QVQLQQSGPELVKPGASVKISCKASGYTFSSSWINWVKQRPGQGLEWIGRIYPGDGDTNYNGKFKGKATLTADKSSSTVDMHLSSLTYVDSAVYFCAIHSSGFPHWGQGTLVTVSA

SEQ ID NO: 405 - protein, 11 aa, synthetic construct
RTSQDISDYLN

SEQ ID NO: 406 - protein, 7 aa, synthetic construct
YTSRLHS

SEQ ID NO: 407 - protein, 9 aa, synthetic construct
QQTNTLPFT

SEQ ID NO: 408 - protein, 7 aa, synthetic construct
SQDISDY

SEQ ID NO: 409 - skipped

SEQ ID NO: 410 - protein, 6 aa, synthetic construct
TNTLPF

SEQ ID NO: 411 - protein, 107 aa, synthetic construct
DIQMTQTTSSLSASLGGRVTISCRTSQDISDYLNWYQQKPDGAVKLLIYYTSRLHSGVPSRFSGSGSGTDYSLTISNLEQEDIATYFCQQTNTLPFTFGGGTKLEIK

SEQ ID NO: 412 - protein, 5 aa, synthetic construct
RYWMN

SEQ ID NO: 413 - protein, 17 aa, synthetic construct
QIYPGDGDTKYNGKFKD

SEQ ID NO: 414 - protein, 8 aa, synthetic construct
YGNYGMDY

SEQ ID NO: 415 - protein, 7 aa, synthetic construct
GYAFSRY

SEQ ID NO: 416 - protein, 6 aa, synthetic construct
YPGDGD

SEQ ID NO: 417 - protein, 8 aa, synthetic construct
YGNYGMDY

SEQ ID NO: 418 - protein, 117 aa, synthetic construct
QVQLQQSGAELVRPGSSVKISCKASGYAFSRYWMNWVKQRPGQGLEWIGQIYPGDGDTKYNGKFKDTATLTADKSSSTAYLQLSSLTSEDSAVYFCAKYGNYGMDYWGQGTSVTVSS

SEQ ID NO: 419 - protein, 16 aa, synthetic construct
RSSQSLEYGNGNTYLN

SEQ ID NO: 420 - protein, 7 aa, synthetic construct
RVSNRFS

SEQ ID NO: 421 - protein, 9 aa, synthetic construct
LQFTHVPYT

SEQ ID NO: 422 - protein, 12 aa, synthetic construct
SQSLEYGNGNTY

SEQ ID NO: 423 - skipped

SEQ ID NO: 424 - protein, 6 aa, synthetic construct
FTHVPY

SEQ ID NO: 425 - protein, 112 aa, synthetic construct
DAVMTQTPLSLPVSLGDQASISCRSSQSLEYGNGNTYLNWYLQKPGQSPQLLIYRVSNRFSGVLDRFSGSGSGTDFTLKISRVEAEDLGVYFCLQFTHVPYTFGGGTKLEIK

SEQ ID NO: 426 - protein, 5 aa, synthetic construct
GYTMN

SEQ ID NO: 427 - protein, 17 aa, synthetic construct
LFNPYNGGTRYNQKFKG

SEQ ID NO: 428 - protein, 13 aa, synthetic construct
LRNYGIGDDFFDY

SEQ ID NO: 429 - protein, 7 aa, synthetic construct
GYSFTGY

SEQ ID NO: 430 - protein, 6 aa, synthetic construct
NPYNGG

SEQ ID NO: 431 - protein, 13 aa, synthetic construct
LRNYGIGDDFFDY

SEQ ID NO: 432 - protein, 122 aa, synthetic construct
EVQLQQSGPELVKPGASMKISCKASGYSFTGYTMNWVKQSHGENLEWIGLFNPYNGGTRYNQKFKGKATLTVDKSSSTAYMELLSLTSEDSAVYYCARLRNYGIGDDFFDYWGQGTTLTVSS

SEQ ID NO: 433 - protein, 11 aa, synthetic construct
KASQDVGTAVA

SEQ ID NO: 434 - protein, 7 aa, synthetic construct
WASTRHT

SEQ ID NO: 435 - protein, 9 aa, synthetic construct
QQYSNYPYT

SEQ ID NO: 436 - protein, 7 aa, synthetic construct
SQDVGTA

SEQ ID NO: 437 - skipped

SEQ ID NO: 438 - protein, 6 aa, synthetic construct
YSNYPY

SEQ ID NO: 439 - protein, 107 aa, synthetic construct
DIVMTQSHKFMSTSVGDRVSITCKASQDVGTAVAWYQQKPGQSPKLLIYWASTRHTGVPDRFTGSGSGTDFTLTISNVQSEDLTDYFCQQYSNYPYTFGGGTKLEIK

SEQ ID NO: 440 - protein, 5 aa, synthetic construct
GYTMN

SEQ ID NO: 441 - protein, 17 aa, synthetic construct
LFNPYNGGINYNQKFKG

SEQ ID NO: 442 - protein, 13 aa, synthetic construct
LRYYGIGDDFFDY

SEQ ID NO: 443 - protein, 7 aa, synthetic construct
GYSFTGY

SEQ ID NO: 444 - protein, 6 aa, synthetic construct
NPYNGG

SEQ ID NO: 445 - protein, 13 aa, synthetic construct
LRYYGIGDDFFDY

SEQ ID NO: 446 - protein, 122 aa, synthetic construct
EVQLQQSGPELVKPGASMKISCKASGYSFTGYTMNWVKQSHGKNLEWIGLFNPYNGGINYNQKFKGKATLTVDKSSSTAYMELLSLTSEDSAVYYCARLRYYGIGDDFFDYWGQGTSLTVSS

SEQ ID NO: 447 - protein, 11 aa, synthetic construct
KASRDVGTAVA

SEQ ID NO: 448 - protein, 7 aa, synthetic construct
WASTRHT

SEQ ID NO: 449 - protein, 9 aa, synthetic construct
QQYSNYPYT

SEQ ID NO: 450 - protein, 7 aa, synthetic construct
SRDVGTA

SEQ ID NO: 451 - skipped

SEQ ID NO: 452 - protein, 6 aa, synthetic construct
YSNYPY

SEQ ID NO: 453 - protein, 107 aa, synthetic construct
DIVMTQSHKFMSTSVGDRVSITCKASRDVGTAVAWYQQKPGQSPKLLIYWASTRHTGVPDRFTGSGSGTDFTLTISNVQSEDLADYFCQQYSNYPYTFGGGTKLEMK

SEQ ID NO: 454 - protein, 5 aa, synthetic construct
GYTMN

SEQ ID NO: 455 - protein, 17 aa, synthetic construct
LFNPYNGGPNYNQKFKG

SEQ ID NO: 456 - protein, 13 aa, synthetic construct
LRYYGIGDDFFDY

SEQ ID NO: 457 - protein, 7 aa, synthetic construct
GYSFTGY

SEQ ID NO: 458 - protein, 6 aa, synthetic construct
NPYNGG

SEQ ID NO: 459 - protein, 13 aa, synthetic construct
LRYYGIGDDFFDY

SEQ ID NO: 460 - protein, 122 aa, synthetic construct
EVQLQQSGPELVKPGASMKISCKASGYSFTGYTMNWMKQGHGKNLEWIGLFNPYNGGPNYNQKFKGKATLTVDKSSSTAYMELLSLTSEDSAVYYCARLRYYGIGDDFFDYWGQGTTLTVSS

SEQ ID NO: 461 - protein, 11 aa, synthetic construct
KASQDVGTAVA

SEQ ID NO: 462 - protein, 7 aa, synthetic construct
WASTRHT

SEQ ID NO: 463 - protein, 9 aa, synthetic construct
QQYSSYPYT

SEQ ID NO: 464 - protein, 7 aa, synthetic construct
SQDVGTA

SEQ ID NO: 465 - skipped

SEQ ID NO: 466 - protein, 6 aa, synthetic construct
YSSYPY

SEQ ID NO: 467 - protein, 107 aa, synthetic construct
DIVMTQSHKFMSTSVGDRVSITCKASQDVGTAVAWYQQKPGQSPKLLIYWASTRHTGVPDRFTGSGSGTDFTLTITNVQSEDLTDYFCQQYSSYPYTFGGGTKLEIK

SEQ ID NO: 468 - protein, 5 aa, synthetic construct
GYTMN

SEQ ID NO: 469 - protein, 17 aa, synthetic construct
LFNPYNGGPSYNQKFKG

SEQ ID NO: 470 - protein, 13 aa, synthetic construct
LRYYGIGDDFFDY

SEQ ID NO: 471 - protein, 7 aa, synthetic construct
GYSFTGY

SEQ ID NO: 472 - protein, 6 aa, synthetic construct
NPYNGG

SEQ ID NO: 473 - protein, 13 aa, synthetic construct
LRYYGIGDDFFDY

SEQ ID NO: 474 - protein, 122 aa, synthetic construct
EVQLQQSGPDLVKPGASMKLSCKASGYSFTGYTMNWVKQSHGKNLEWIGLFNPYNGGPSYNQKFKGKATLTVDKSSSTAYMELLSLTPEDSAVYYCARLRYYGIGDDFFDYWGQGTTLTVSS

SEQ ID NO: 475 - protein, 11 aa, synthetic construct
KASQDVGTAVA

SEQ ID NO: 476 - protein, 7 aa, synthetic construct
WASTRHT

SEQ ID NO: 477 - protein, 9 aa, synthetic construct
QQYSNYPYT

SEQ ID NO: 478 - protein, 7 aa, synthetic construct
SQDVGTA

SEQ ID NO: 479 - skipped

SEQ ID NO: 480 - protein, 6 aa, synthetic construct
YSNYPY

SEQ ID NO: 481 - protein, 107 aa, synthetic construct
DIVMTQSHKFMSTSVGDRVSITCKASQDVGTAVAWYQEKPGQSPKLLIYWASTRHTGVPDRFTGSGSGTDFTLTISNVQSEDLAYYFCQQYSNYPYTFGGGTKLEIK

SEQ ID NO: 482 - protein, 5 aa, synthetic construct
GYTMN

SEQ ID NO: 483 - protein, 17 aa, synthetic construct
LFNPYNGGATYNQRFKG

SEQ ID NO: 484 - protein, 13 aa, synthetic construct
LRKYGIGDDFFDY

SEQ ID NO: 485 - protein, 7 aa, synthetic construct
GYSFTGY

SEQ ID NO: 486 - protein, 6 aa, synthetic construct
NPYNGG

SEQ ID NO: 487 - protein, 13 aa, synthetic construct
LRKYGIGDDFFDY

SEQ ID NO: 488 - protein, 122 aa, synthetic construct
EVQLQQSGPELVKPGASMKISCKASGYSFTGYTMNWVKQSHGKNLEWIGLFNPYNGGATYNQRFKGKATLTVDKSSSTAYMDLLSLTSEDSAVYYCTRLRKYGIGDDFFDYWGQGTTLTVSS

SEQ ID NO: 489 - protein, 11 aa, synthetic construct
KASQDVGTAVA

SEQ ID NO: 490 - protein, 7 aa, synthetic construct
WASTRHT

SEQ ID NO: 491 - protein, 9 aa, synthetic construct
QQYSTYTYT

SEQ ID NO: 492 - protein, 7 aa, synthetic construct
SQDVGTA

SEQ ID NO: 493 - skipped

SEQ ID NO: 494 - protein, 6 aa, synthetic construct
YSTYTY

SEQ ID NO: 495 - protein, 107 aa, synthetic construct
DIVMTQSHKFMSTSVGDRVSITCKASQDVGTAVAWYQQKPGQSPKLLIYWASTRHTGVPDRFTGSGSGTDFTLTISNVQSEDLADYFCQQYSTYTYTFGGGTKLEIK

SEQ ID NO: 496 - protein, 80 aa, synthetic construct
FSLKLSAENDFSSDSPERKMLPCYSTARIPLPNLNEDLTCGNLLMWEAVTVQTEVIGITSMLNLHAGSQKVHEHGGGKPI

SEQ ID NO: 497 - protein, 80 aa, synthetic construct
YSLKLTAENAFDSDSPDKKMLPCYSTARIPLPNLNEDLTCGNLLMWEAVTVKTEVIGITSMLNLHAGSQKVHENGGGKPV

SEQ ID NO: 498 - protein, 80 aa, synthetic construct
YSQHLSAENAFDSDSPDKKMLPCYSTARIPLPNLNEDLTCGNLLMWEAVTVKTEVIGITSMLNLHAGSQKVHENGGGKPV

SEQ ID NO: 499 - protein, 80 aa, synthetic construct
YSLRLTAETAFDSDSPDRKMLPCYSTARIPLPNLNEDLTCGNLLMWEAVTVKTEVIGITSMLNLHAGSQKVHENGGGKPI

SEQ ID NO: 500 - protein, 18 aa, synthetic construct
  VARIANT 3: note = Any amino acid
  VARIANT 5..6: note = Any amino acid
  VARIANT 7: note = D or E
  VARIANT 7: note = Variant residue given in the sequence has no preference with respect to that in the annotation for variant position
  VARIANT 8..12: note = Any amino acid
  VARIANT 15: note = Any amino acid
  VARIANT 17: note = Any amino acid
YRXKXXDXXXXXKNXTXQ

SEQ ID NO: 501 - protein, 23 aa, synthetic construct
  VARIANT 8: note = Any amino acid
  VARIANT 11..12: note = Any amino acid
  VARIANT 16: note = Any amino acid
  VARIANT 18: note = Any amino acid
  REGION 1..23: note = source = /note=Description of synthetic construct: Synthetic peptide
NYRTKYPXGTXXPKNXTXQSQVM

SEQ ID NO: 502 - protein, 268 aa, synthetic construct
KGGVEVLEVKTGVDAITEVECFLNPEMGDPDENLRGFSLKLSAENDFSSDSPERKMLPCYSTARIPLPNLNEDLTCGNLLMWEAVTVQTEVIGITSMLNLHAGSQKVHEHGGGKPIQGSNFHFFAVGGDPLEMQGVLMNYRTKYPEGTITPKNPTAQSQVMNTDHKAYLDKNNAYPVECWIPDPSRNENTRYFGTFTGGENVPPVLHVTNTATTVLLDEQGVGPLCKADSLYVSAADICGLFTNSSGTQQWRGLARYFKIRLRKRSVK

SEQ ID NO: 503 - protein, 268 aa, synthetic construct
KGGVEVLEVKTGVDAITEVECFLNPEMGDPDDNLRGYSLKLTAENAFDSDSPDKKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLARYFKIRLRKRSVK

SEQ ID NO: 504 - protein, 268 aa, synthetic construct
KGGVEVLEVKTGVDAITEVECFLNPEMGDPDDHLRGYSQHLSAENAFDSDSPDKKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSKNENTRYFGTYTGGENVPPVLHVTNTATTVLLDEQGVGPLCKADSLYVSAADICGLFTNSSGTQQWRGLARYFKIRLRKRSVK

SEQ ID NO: 505 - protein, 268 aa, synthetic construct
KGGVEVLEVKTGVDAITEVECFLNPEMGDPDNDLRGYSLRLTAETAFDSDSPDRKMLPCYSTARIPLPNLNEDLTCGNLLMWEAVTVKTEVIGITSMLNLHAGSQKVHENGGGKPIQGSNFHFFAVGGDPLEMQGVLMNYRTKYPEGTVTPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLPRYFKIRLRKRSVK

SEQ ID NO: 506 - protein, 6 aa, synthetic construct
  REGION 1..6: note = Synthetic 6xHis tag
HHHHHH

SEQ ID NO: 507 - protein, 10 aa, synthetic construct
  VARIANT 5: note = Any amino acid
GFTFXNYWMT

SEQ ID NO: 508 - protein, 10 aa, synthetic construct
GFTFNNYWMT

SEQ ID NO: 509 - protein, 17 aa, synthetic construct
NIKKDGSEKYYVDSVRG

SEQ ID NO: 510 - protein, 11 aa, synthetic construct
VRSGRYFALDD

SEQ ID NO: 511 - protein, 11 aa, synthetic construct
GGDNIGSRPVH

SEQ ID NO: 512 - protein, 7 aa, synthetic construct
DDSNRPS

SEQ ID NO: 513 - protein, 10 aa, synthetic construct
QVWSSSTDHP

SEQ ID NO: 514 - protein, 10 aa, synthetic construct
GFTFSNYWMT

SEQ ID NO: 515 - protein, 17 aa, synthetic construct
NIKKDGSEKYYVDSVRG

SEQ ID NO: 516 - protein, 11 aa, synthetic construct
VRSGRYFALDD

SEQ ID NO: 517 - protein, 11 aa, synthetic construct
GGDNIGSRPVH

SEQ ID NO: 518 - protein, 7 aa, synthetic construct
DDSNRPS

SEQ ID NO: 519 - protein, 10 aa, synthetic construct
QVWSSSTDHP

SEQ ID NO: 520 - protein, 10 aa, synthetic construct
GFTFKNYWMT

SEQ ID NO: 521 - protein, 17 aa, synthetic construct
NIKKDGSEKYYVDSVRG

SEQ ID NO: 522 - protein, 11 aa, synthetic construct
VRSGRYFALDD

SEQ ID NO: 523 - protein, 11 aa, synthetic construct
GGDNIGSRPVH

SEQ ID NO: 524 - protein, 7 aa, synthetic construct
DDSNRPS

SEQ ID NO: 525 - protein, 10 aa, synthetic construct
QVWSSSTDHP

SEQ ID NO: 526 - protein, 10 aa, synthetic construct
GFTFQNYWMT

SEQ ID NO: 527 - protein, 17 aa, synthetic construct
NIKKDGSEKYYVDSVRG

SEQ ID NO: 528 - protein, 11 aa, synthetic construct
VRSGRYFALDD

SEQ ID NO: 529 - protein, 11 aa, synthetic construct
GGDNIGSRPVH

SEQ ID NO: 530 - protein, 7 aa, synthetic construct
DDSNRPS

SEQ ID NO: 531 - protein, 10 aa, synthetic construct
QVWSSSTDHP

SEQ ID NO: 532 - protein, 10 aa, synthetic construct
GFTFNNYWMT

SEQ ID NO: 533 - protein, 17 aa, synthetic construct
NIKKDGSEKYYVDSVRG

SEQ ID NO: 534 - protein, 11 aa, synthetic construct
VRSGRYFALDD

SEQ ID NO: 535 - protein, 11 aa, synthetic construct
GGDNIGSRPVH

SEQ ID NO: 536 - protein, 7 aa, synthetic construct
DDSNRPS

SEQ ID NO: 537 - protein, 10 aa, synthetic construct
QVWSSSTDHP

SEQ ID NO: 538 - protein, 10 aa, synthetic construct
GFTFNNYWMT

SEQ ID NO: 539 - protein, 17 aa, synthetic construct
NIKKDGSEKYYVDSVRG

SEQ ID NO: 540 - protein, 11 aa, synthetic construct
VRSGRYFALDD

SEQ ID NO: 541 - protein, 11 aa, synthetic construct
GGDNIGSRPVH

SEQ ID NO: 542 - protein, 7 aa, synthetic construct
DDSNRPS

SEQ ID NO: 543 - protein, 10 aa, synthetic construct
QVWSSSTDHP